SEQUENCE LISTING (21 sequences)

SEQ ID NO: 1 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 9 aa, Homo sapiens
IEVMYPPPY

SEQ ID NO: 3 - DNA, 22 bp, Artificial Sequence
  Synthetic primer
caccatggcgctcttcgtgcgg

SEQ ID NO: 4 - DNA, 18 bp, Artificial Sequence
  Synthetic primer
ctaatgcttcatcctctc

SEQ ID NO: 5 - DNA, 20 bp, Artificial Sequene
  Synthtic
agctggtagggcgacttggc

SEQ ID NO: 6 - DNA, 20 bp, Artificial Sequence
  Synthetic
cgacttggcgggacccgcca

SEQ ID NO: 7 - DNA, 20 bp, Artificial Sequence
  Synthetic
catgctcactatcaacggga

SEQ ID NO: 8 - DNA, 22 bp, Artificial Sequence
  Synthetic Oligonucleotide
caccatggcgctcttcgtgcgg

SEQ ID NO: 9 - DNA, 18 bp, Artificial Sequence
  Synthetic Oligonucleotide
ctaatgcttcatcctctc

SEQ ID NO: 10 - DNA, 25 bp, Artificial Sequence
  Synthetic Oligonucleotide
caccgagctggtagggcgacttggc

SEQ ID NO: 11 - DNA, 25 bp, Artificial Sequence
  Synthetic Oligonucleotide
aaacgccaagtcgccctaccagctc

SEQ ID NO: 12 - DNA, 25 bp, Artificial Sequence
  Synthetic Oligonucleotide
caccgcgacttggcgggacccgcca

SEQ ID NO: 13 - DNA, 25 bp, Artificial Sequence
  Synthetic Oligonucleotide
aaactggcgggtcccgccaagtcgc

SEQ ID NO: 14 - DNA, 25 bp, Artificial Sequence
  Synthetic Oligonucleotide
caccgcatgctcactatcaacggga

SEQ ID NO: 15 - DNA, 25 bp, Artificial Sequence
  Synthetic Oligonucleotide
aaactcccgttgatagtgagcatgc

SEQ ID NO: 16 - DNA, 18 bp, Artificial Sequence
  Synthetic Oligonucleotide
tccatggcgctcttcgtg

SEQ ID NO: 17 - DNA, 20 bp, Artificial Sequence
  Synthetic Oligonucleotide
gactacctgaccttccgcag

SEQ ID NO: 18 - DNA, 20 bp, Artificial Sequence
  Synthetic Oligonucleotide
gtggaccctgacttgacctg

SEQ ID NO: 19 - DNA, 20 bp, Artificial Sequence
  Synthetic Oligonucleotide
gtagtggatcaccccgttgg

SEQ ID NO: 20 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 21 - protein, 183 aa, Artificial Sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

(19) United States
(12) Patent Application Publication
(10) Pub. No.: US 2020/0071773 A1
(43) Pub. Date: Mar. 5, 2020

(54) TUMOR SIGNATURE FOR METASTASIS, COMPOSITIONS OF MATTER METHODS OF USE THEREOF

(71) Applicants: Massachusetts Eye and Ear Infirmary, Boston, MA (US); The General Hospital Corporation, Boston, MA (US); The Broad Institute, Inc., Cambridge, MA (US); Massachusetts Institute of Technology, Cambridge, MA (US)

(72) Inventors: Sidharth Puram, Boston, MA (US); Itay Tirosh, Cambridge, MA (US); Anuraag Parikh, Boston, MA (US); Derrick Lin, Boston, MA (US); Aviv Regev, Cambridge, MA (US); Bradley Bernstein, Boston, MA (US)

(21) Appl. No.: 16/604,651

(22) PCT Filed: Apr. 12, 2018
(86) PCT No.: PCT/US2018/027383

§ 371 Date: Oct. 11, 2019

Related U.S. Application Data
(60) Provisional application No. 62/484,709, filed on Apr. 12, 2017.
(60) Provisional application No. 62/586,126, filed on Nov. 14, 2017.

(51) Int. Cl. C12Q 1/6886; A61P 35/04; C12Q 1/6837; G16B 25/10; G16B 40/00

(52) U.S. Cl. CPC C12Q 1/6886; A61P 35/04; C12Q 1/6837; C12Q 2600/158; G16B 40/00; C12Q 2600/106; G16B 25/10

(57) ABSTRACT

The present invention advantageously provides for novel gene signatures, tools and methods for the treatment and prognosis of epithelial tumors. Applicants have used single cell RNA-seq to reveal novel expression programs of malignant, stromal and immune cells in the HNSCC tumor ecosystem. Malignant cells varied in expression of programs related to stress, hypoxia and epithelial differentiation. A partial EMT-like program (p-EMT) was discovered that was expressed in cells residing at the leading edge of tumors. Applicants unexpectedly linked the p-EMT state to metastasis and adverse clinical features that may be used to direct treatment of epithelial cancers (e.g., HNSCC). Applicants also show that metastases are dynamically regulated by the tumor microenvironment (TME). Finally, a computational modeling approach was developed that allows analysis of malignant cells in bulk sequencing samples.

### CROSS-REFERENCE TO RELATED APPLICATIONS

This application claims the benefit of U.S. Provisional Application Nos. 62/484,709, filed Apr. 12, 2017 and 62/586,126, filed Nov. 14, 2017. The entire contents of the above-identified applications are hereby fully incorporated herein by reference.

### STATEMENT REGARDING FEDERALLY SPONSORED RESEARCH

This invention was made with government support under grant numbers CA216873, CA180922, CA202820 and CA14051 awarded by the National Institutes of Health. The government has certain rights in the invention.

### TECHNICAL FIELD

The subject matter disclosed herein is generally directed to methods of using gene expression profiles representative of cell sub-types present in head and neck squamous cell carcinoma (HNSCC). Specifically, the gene signatures may be used for diagnosing, pro gnosing and/or staging of tumors and designing and selecting appropriate treatment regimens. Furthermore, novel signatures determined by single cell analysis of HNSCC are leveraged to provide for methods and systems for deconvolution of bulk sequencing data from tumors.

### BACKGROUND

Genomic and transcriptomic studies have revealed driver mutations, identified aberrant regulatory programs, and redefined disease subtypes for major human tumors (Stratton et al., 2009; Weinberg, 2014). However, these studies relied on profiling technologies that measure the entire tumor in bulk, limiting their ability to capture intra-tumoral heterogeneity, including malignant cells in distinct genetic, epigenetic, and functional states, as well as diverse non-malignant cells such as immune cells, fibroblasts, and endothelial cells. Substantial evidence indicates that intra-tumoral heterogeneity among malignant and non-malignant cells, and their interactions within the tumor microenvironment (TME) are critical to many aspects of tumor biology, including self-renewal, immune surveillance, drug resistance and metastasis (Meacham and Morrison, 2013; Weinberg, 2014).

Recent advances in single-cell genomics provide an avenue to explore genetic and functional heterogeneity at a cellular resolution (Navin, 2015; Tanay and Regev, 2017; Wagner et al., 2016). In particular, single-cell RNA-seq (scRNA-seq) studies of human tumors, circulating tumor cells and patient-derived xenografts have revealed new insights into tumor composition, cancer stem cells, and drug resistance.

Despite these promising results, scRNA-seq studies have not extensively characterized epithelial tumors, in spite of their predominance. In these tumors, metastasis to nearby draining lymph nodes (locoregional metastasis) and to other organs (distant metastasis) represents a major cause of morbidity and mortality. However, lymph node (LN) and distant metastases are often treated based on molecular and pathologic features of the primary tumor, raising the question of whether metastases share the same genetics, epigenetics, and vulnerabilities (Lambert et al., 2017). The potentially different composition of primary tumors and metastases hinders the straightforward comparison of bulk tumor profiles. Single-cell expression profiling studies would, in principle, offer a compelling alternative.

Epithelial-to-mesenchymal transition (EMT) has been suggested as a driver of local and distant spread of epithelial tumors (Gupta and Massague, 2006; Lambert et al., 2017). The process of EMT is fundamental to embryonic development and other physiologic processes and may be co-opted by malignant epithelial cells to facilitate invasion and dissemination (Thiery et al., 2009; Ye and Weinberg, 2015). EMT markers have been detected on circulating tumor cells (CTCs) associated with metastatic disease (Ting et al., 2014; Yu et al., 2013). However, since most EMT studies have focused on laboratory models, the nature, extent, and significance of EMT in primary human tumors and metastases remains controversial (Lambert et al., 2017; Nieto et al., 2016). For example, although mesenchymal subtypes have been identified in multiple tumor types (Cancer Genome Atlas, 2015; Cancer Genome Atlas Research, 2011; Verhaak et al., 2010), it remains unclear whether they reflect mesenchymal cancer cells or, alternatively, contributions of non-malignant, mesenchymal cell types in the TME.

Head and neck squamous cell carcinoma (HNSCC) is an epithelial tumor with strong associations to chronic alcohol and tobacco exposure (Puram and Rocco, 2015). Like many epithelial cancers, HNSCC tumors are highly heterogeneous within and between patients. Metastatic disease remains a central challenge, with patients often presenting at an advanced stage with LN metastases. Thus, there is a need for biomarkers and therapeutic targets capable of guiding treatment and predicting disease progression (e.g., metastasis) in epithelial tumors.

### SUMMARY

The diverse malignant, stromal, and immune cells in tumors affect growth, metastasis and response to therapy. It is an objective of the present invention to understand intra-tumoral heterogeneity, invasion and metastasis in an epithelial human cancer. It is another objective of the present to provide for novel tools and methods for diagnosing, prognosing and treating tumors. Applicants investigated primary HNSCC tumors and matched lymph nodes. Specifically, Applicants profiled transcriptomes of ˜6,000 single cells from 18 head and neck squamous cell carcinoma (HNSCC) patients, including five matched pairs of primary tumors and lymph node metastases. Stromal and immune cells had consistent expression programs across patients. Conversely, malignant cells varied within and between tumors in their expression of signatures related to cell cycle, stress, hypoxia, epithelial differentiation, and partial epithelial-to-mesenchymal transition (p-EMT). Cells expressing the p-EMT program spatially localized to the leading edge of primary tumors. By integrating single-cell transcriptomes with bulk expression profiles for hundreds of tumors, Applicants refined HNSCC subtypes by their malignant and stromal composition, and established p-EMT as an independent predictor of nodal metastasis, tumor grade, and adverse pathologic features (e.g., extracapsular extension). The results provide insight into the HNSCC ecosystem, define stromal interactions and define a p-EMT program associated with metastasis.

In one aspect, the present invention provides for a method of detecting an EMT-like (p-EMT) gene signature in epithelial tumors comprising, detecting in tumor cells obtained from a subject suffering from an epithelial tumor, the expression or activity of a EMT-like (p-EMT) gene signature, said signature comprising one or more genes or polypeptides selected from the group consisting of SERPINE1, TGFBI, MMP10, LAMC2, P4HA2, PDPN, ITGA5, LAMA3, CDH13, TNC, MMP2, EMP3, INHBA, LAMB3, SNAIL2 and VIM; or one or more genes or polypeptides selected from the group consisting of SERPINE1, TGFBI, MMP10, LAMC2, P4HA2, PDPN, ITGA5, LAMA3, CDH13, TNC, MMP2, EMP3, INHBA, LAMB3, VIM, SEMA3C, PRKCDBP, ANXA5, DHRS7, ITGB1, ACTN1, CXCR7, ITGB6, IGFBP7, THBS1, PTHLH, TNFRSF6B, PDLIM7, CAV1, DKK3, COL17A1, LTBP1, COL5A2, COL1A1, FHL2, TIMP3, PLAU, LGALS1, PSMD2, CD63, HERPUD1, TPM1, SLC39A14, C1S, MMP1, EXT2, COL4A2, PRSS23, SLC7A8, SLC31A2, ARPC1B, APP, MFAP2, MPZL1, DFNA5, MT2A, MAGED2, ITGA6, FSTL1, TNFRSF12A, IL32, COPB2, PTK7, OCIAD2, TAX1BP3, SEC13, SERPINH1, TPM4, MYH9, ANXA8L1, PLOD2, GALNT2, LEPREL1, MAGED1, SLC38A5, FSTL3, CD99, F3, PSAP, NMRK1, FKBP9, DSG2, ECM1, HTRA1, SERINC1, CALU, TPST1, PLOD3, IGFBP3, FRMD6, CXCL14, SERPINE2, RABAC1, TMED9, NAGK, BMP1, ESYT1, STON2, TAGLN and GJA1. The signature may not comprise ZEB1/2, TWIST1/2, or SNAIL1. Thus, the signature unexpectedly does not include most classical EMT transcription factors.

In one embodiment, detecting a p-EMT gene signature may indicate that the subject is less likely to respond to therapy. In certain embodiments, the therapy is a therapy consistent with the standard of care for the epithelial tumor. In certain embodiments, the therapy is an immunotherapy, such as checkpoint blockade therapy. Detecting a p-EMT gene signature may indicate that the subject requires more aggressive treatment. The method may further comprise treating the subject with one or more of lymph node dissection, adjuvant chemotherapy, adjuvant radiation, neoadjuvant therapy, chemoradiation, and an agent that inhibits TGF beta signaling upon detecting the p-EMT gene signature. The epithelial tumor may be head and neck squamous cell carcinoma (HNSCC). In certain example embodiments, “less likely to respond” indicates the likelihood of response is less than the likelihood of an individual without a p-EMT gene signature of p-EMT^{lo} signature as measured using standard statistical analysis, such as those used and described in the examples section below.

In another embodiment, not detecting a p-EMT gene signature may indicate that the subject is more likely to respond to therapy. Not detecting a p-EMT gene signature may indicate that the subject should avoid aggressive treatment. Not being bound by a theory, an unnecessary aggressive treatment may lead to increased mortality and morbidity. In certain embodiments, if a p-EMT signature is not detected a subject may be treated according to a less aggressive standard of care as described herein.

In another aspect, the present invention provides for a method of treatment for a subject in need thereof suffering from an epithelial tumor comprising: a) detecting expression or activity of a p-EMT gene signature for a tumor sample obtained from the subject, wherein the p-EMT signature comprises one or more genes or polypeptides selected from the group consisting of SERPINE1, TGFBI, MMP10, LAMC2, P4HA2, PDPN, ITGA5, LAMA3, CDH13, TNC, MMP2, EMP3, INHBA, LAMB3, SNAIL2 and VIM; and b) treating the subject, wherein if a p-EMT signature is detected the treatment comprises: i) lymph node dissection of the subject; ii) adjuvant chemotherapy; iii) adjuvant radiation or postoperative radiation treatment (PORT); iv) neoadjuvant therapy; v) chemoradiation; or vi) administering an agent that inhibits TGF beta signaling, wherein if a p-EMT signature is not detected the treatment comprises delaying lymph node dissection.

In certain embodiments, the method may further comprise: detecting expression or activity of an epithelial gene signature for a tumor sample obtained from the subject, wherein the epithelial signature comprises: one or more genes or polypeptides selected from the group consisting of IL1RN, SLPI, CLDN4, CLDN7, S100A9, SPRR1B, PVRL4, RHCG, SDCBP2, S100A8, APOBEC3A, LY6D, KRT16, KRT6B, KRT6A, LYPD3, KRT6C, KLK10, KLK11, TYMP, FABP5, SCO2, FGFBP1 and JUP; or one or more genes or polypeptides selected from the group consisting of SPRR1B, KRT16, KRT6B, KRT6C, KRT6A, KLK10, KLK11 and CLDN7; or one or more genes or polypeptides selected from the group consisting of IL1RN, SLPI, CLDN4, S100A9, SPRR1B, PVRL4, RHCG, SDCBP2, S100A8, APOBEC3A, GRHL1, SULT2B1, ELF3, KRT16, PRSS8, MXD1, S100A7, KRT6B, LYPD3, TACSTD2, CDKN1A, KLK11, GPRC5A, KLK10, TMBIM1, PLAUR, CLDN7, DUOXA1, PDZK1IP1, NCCRP1, IDS, PPL, ZNF750, EMP1, CLDN1, CRB3, CYB5R1, DSC2, S100P, GRHL3, SPINT1, SDR16C5, SPRR1A, WBP2, GRB7, KLK7, TMEM79, SBSN, PIM1, CLIC3, MALAT1, TRIP10, CAST, TMPRSS4, TOM1, A2ML1, MBOAT2, LGALS3, ERO1L, EHF, LCN2, YPEL5, ALDH3B2, DMKN, PIK3IP1, CEACAM6, OVOL1, TMPRSS11E, CD55, KLK6, SPRR2D, NDRG2, CD24, HIST1H1C, LY6D, CLIP1, HIST1H2AC, BNIPL, QSOX1, ECM1, DHRS3, PPP1R15A, TRIM16, AQP3, IRF6, CSTA, RAB25, HOPX, GIPC1, RAB11FIP1, CSTB, KRT6C, PKP1, JUP, MAFF, DSG3, AKTIP, KLF3, HSPB8 and H1F0; or one or more genes or polypeptides selected from the group consisting of LY6D, KRT16, KRT6B, LYPD3, KRT6C, TYMP, FABP5, SCO2, FGFBP1, JUP, IMP4, DSC2, TMBIM1, KRT14, C1QBP, SFN, S100A14, RAB38, GJB5, MRPL14, TRIM29, ANXA8L2, KRT6A, PDHB, AKR1B10, LAD1, DSG3, MRPL21, NDUFS7, PSMD6, AHCY, GBP2, TXN2, PSMD13, NOP16, EIF4EBP1, MRPL12, HSD17B10, LGALS7B, THBD, EXOSC4, APRT, ANXA8L1, ATP5G1, S100A2, TBRG4, MAL2, NHP2L1, DDX39A, ZNF750, UBE2L6, WDR74, PPIF, PRMT5, VSNL1, VPS25, SNRNP40, ADRM1, NDUFS8, TUBA1C, TMEM79, UQCRFS1, EIF3K, NME2, PKP3, SERPINB1, RPL26L1, EIF6, DSP, PHLDA2, S100A16, LGALS7, MT1X, UQCRC2, EIF3I, MRPL24, CCT7, RHOV, ECE2, SSBP1, POLDIP2, FIS1, CKMT1A, GJB3, NME1, MRPS12, GPS1, ALG3, MRPL20, EMC6, SRD5A1, PA2G4, ECSIT, MRPL23, NAA20, HMOX2, COA4, DCXR, PSMD8 and WBSCR22; and treating the subject as above if a p-EMT signature is detected above a p-EMT high reference level and the epithelial signature is detected below an epithelial low reference. Chemoradiation may comprise cisplatin. The treatment may comprise administering an agent that inhibits TGF beta signaling. Applicants describe herein data showing that the p-EMT signature is regulated by TGF beta signaling. The epithelial tumor may be head and neck squamous cell carcinoma (HNSCC).

In another aspect, the present invention provides for a method of treating an epithelial tumor, comprising administering to a subject in need thereof suffering from an epithelial tumor a therapeutically effective amount of an agent: a) capable of reducing the expression or inhibiting the activity of one or more p-EMT signature genes or polypeptides; or b) capable of targeting or binding to one or more cell surface exposed p-EMT signature genes or polypeptides, wherein the p-EMT signature comprises one or more genes or polypeptides selected from the group consisting of SERPINE1, TGFBI, MMP10, LAMC2, P4HA2, PDPN, ITGA5, LAMA3, CDH13, TNC, MMP2, EMP3, INHBA, LAMB3, SNAIL2 and VIM. The epithelial tumor may comprise HNSCC. The agent capable of reducing the expression or inhibiting the activity of one or more p-EMT signature genes or polypeptides may comprise a therapeutic antibody, antibody fragment, antibody-like protein scaffold, aptamer, genetic modifying agent or small molecule. The agent capable of targeting or binding to one or more cell surface exposed EMT-like signature polypeptides may comprise a CAR T cell capable of targeting or binding to one or more cell surface exposed p-EMT signature genes or polypeptides.

In another aspect, the present invention provides for a method of deconvoluting bulk gene expression data obtained from an epithelial tumor, wherein the tumor comprises both malignant and non-malignant cells, said method comprising: a) defining, by a processor, the relative frequency of a set of cell types in the tumor from the bulk gene expression data, wherein the frequency of the cell types is determined by cell type specific gene expression, and wherein the set of cell types comprises one or more cell types selected from the group consisting of T cells, fibroblasts, macrophages, mast cells, B/plasma cells, endothelial cells, myocytes and dendritic cells; and b) defining, by a processor, a linear relationship between the frequency of the non-malignant cell types and the expression of a set of genes, wherein the set of genes comprises genes highly expressed by malignant cells and at most two non-malignant cell types, wherein the set of genes are derived from gene expression analysis of single cells in at least one epithelial tumor, and wherein the residual of the linear relationship defines the malignant cell-specific (MCS) expression profile. The epithelial tumor may be HNSCC. The method may further comprise assigning genes to a specific malignant cell sub-type. In other words, a tumor sample is analyzed for types of nonmalignant cells within the tumor based on known cell type markers. This is followed by assigning the detected gene expression to the nonmalignant cells. The residual gene expression data is then assigned to the malignant cell specific sub-population (MCS) in the tumor sample. The malignant cell sub-type may be an EMT-like subtype. Not being bound by a theory, the MCS expression comprising a p-EMT signature can only have been derived from the EMT-like sub-type. In certain embodiments, a p-EMT high tumor has a larger fraction of p-EMT cells than cells of an epithelial differentiation sub-type.

The method may further comprise determining a p-EMT score, wherein said score is based on expression of a p-EMT signature for the malignant cell-specific (MCS) expression profile, wherein said p-EMT signature comprises one or more genes or polypeptides selected from the group consisting of SERPINE1, TGFBI, MMP10, LAMC2, P4HA2, PDPN, ITGA5, LAMA3, CDH13, TNC, MMP2, EMP3, INHBA, LAMB3, SNAIL2 and VIM, and wherein a high p-EMT score has higher expression of the p-EMT signature as compared to expression in a reference data set obtained from a subject with a non-invasive epithelial tumor (see, e.g., FIG. 15). A reference sample may be any known sample where the subject the sample was obtained from did not have lymph node metastasis. A reference sample may be obtained from a database comprising gene expression data and patient histories, such as, but not limited to The Cancer Genome Atlas (TCGA). The reference sample subject may have had a neck dissection and upon analysis of the dissected tissue no tumor cells were observed. Not being bound by a theory, this subject had an unnecessary neck dissection and the present invention would have prevented the unnecessary procedure. The reference data set preferably includes more than one sample from more than one subject. In certain embodiments, a p-EMT low sample will not express a detectable p-EMT signature.

In another aspect, the present invention provides for a method of treatment for a subject in need thereof suffering from an epithelial tumor comprising: a) determining a p-EMT score according to any method described herein for a tumor sample obtained from the subject; and b) treating the subject, wherein if a high p-EMT score is determined the treatment comprises: i) lymph node dissection of the subject; ii) adjuvant chemotherapy; iii) adjuvant radiation or postoperative radiation treatment (PORT); iv) neoadjuvant therapy; v) chemoradiation; or vi) administering an agent that inhibits TGF beta signaling, wherein if the subject does not have a high p-EMT score the treatment comprises delaying lymph node dissection. The chemoradiation may comprise cisplatin. The treatment may comprise administering an agent that inhibits TGF beta signaling.

In another aspect, the present invention provides for a kit comprising reagents to detect at least one gene or gene expression program defined in Table S7. The gene expression program may be a p-EMT program, wherein the p-EMT program comprises one or more genes or polypeptides selected from the group consisting of SERPINE1, TGFBI, MMP10, LAMC2, P4HA2, PDPN, ITGA5, LAMA3, CDH13, TNC, MMP2, EMP3, INHBA, LAMB3, SNAIL2 and VIM. The kit may comprise antibodies and reagents for immunohistochemistry. The kit may further comprise an HNSCC specific antibody. The HNSCC specific antibody may be a p63 antibody. The kit may comprise primers and/or probes for quantitative RT-PCR, PCR, and/or sequencing. The kit may comprise fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25). In certain example embodiments, the kits may further comprise reagents needed to carry out the assays described herein.

In another aspect, the present invention provides for a method of detecting an epithelial gene signature in epithelial tumors comprising detecting in tumor cells obtained from a subject suffering from an epithelial tumor, the expression or activity of an epithelial gene signature, said signature comprising: one or more genes or polypeptides selected from the group consisting of IL1RN, SLPI, CLDN4, CLDN7, S100A9, SPRR1B, PVRL4, RHCG, SDCBP2, S100A8, APOBEC3A, LY6D, KRT16, KRT6B, KRT6A, LYPD3, KRT6C, KLK10, KLK11, TYMP, FABP5, SCO2, FGFBP1 and JUP; or one or more genes or polypeptides selected from the group consisting of SPRR1B, KRT16, KRT6B, KRT6C, KRT6A, KLK10, KLK11 and CLDN7; or one or more genes or polypeptides selected from the group consisting of IL1RN, SLPI, CLDN4, S100A9, SPRR1B, PVRL4, RHCG, SDCBP2, S100A8, APOBEC3A, GRHL1, SULT2B1, ELF3, KRT16, PRSS8, MXD1, S100A7, KRT6B, LYPD3, TACSTD2, CDKN1A, KLK11, GPRC5A, KLK10, TMBIM1, PLAUR, CLDN7, DUOXA1, PDZK1IP1, NCCRP1, IDS, PPL, ZNF750, EMP1, CLDN1, CRB3, CYB5R1, DSC2, S100P, GRHL3, SPINT1, SDR16C5, SPRR1A, WBP2, GRB7, KLK7, TMEM79, SBSN, PIM1, CLIC3, MALAT1, TRIP10, CAST, TMPRSS4, TOM1, A2ML1, MBOAT2, LGALS3, ERO1L, EHF, LCN2, YPEL5, ALDH3B2, DMKN, PIK3IP1, CEACAM6, OVOL1, TMPRSS11E, CD55, KLK6, SPRR2D, NDRG2, CD24, HIST1H1C, LY6D, CLIP1, HIST1H2AC, BNIPL, QSOX1, ECM1, DHRS3, PPP1R15A, TRIM16, AQP3, IRF6, CSTA, RAB25, HOPX, GIPC1, RAB11FIP1, CSTB, KRT6C, PKP1, JUP, MAFF, DSG3, AKTIP, KLF3, HSPB8 and H1F0; or one or more genes or polypeptides selected from the group consisting of LY6D, KRT16, KRT6B, LYPD3, KRT6C, TYMP, FABP5, SCO2, FGFBP1, JUP, IMP4, DSC2, TMBIM1, KRT14, C1QBP, SFN, S100A14, RAB38, GJB5, MRPL14, TRIM29, ANXA8L2, KRT6A, PDHB, AKR1B10, LAD1, DSG3, MRPL21, NDUFS7, PSMD6, AHCY, GBP2, TXN2, PSMD13, NOP16, EIF4EBP1, MRPL12, HSD17B10, LGALS7B, THBD, EXOSC4, APRT, ANXA8L1, ATP5G1, S100A2, TBRG4, MAL2, NHP2L1, DDX39A, ZNF750, UBE2L6, WDR74, PPIF, PRMT5, VSNL1, VPS25, SNRNP40, ADRM1, NDUFS8, TUBA1C, TMEM79, UQCRFS1, EIF3K, NME2, PKP3, SERPINB1, RPL26L1, EIF6, DSP, PHLDA2, S100A16, LGALS7, MT1X, UQCRC2, EIF3I, MRPL24, CCT7, RHOV, ECE2, SSBP1, POLDIP2, FIS1, CKMT1A, GJB3, NME1, MRPS12, GPS1, ALG3, MRPL20, EMC6, SRD5A1, PA2G4, ECSIT, MRPL23, NAA20, HMOX2, COA4, DCXR, PSMD8 and WBSCR22. Detecting an epithelial gene signature may indicate that the subject is more likely to respond to therapy. In certain embodiments, the therapy is a therapy consistent with the standard of care for the epithelial tumor. In certain embodiments, the therapy is an immunotherapy, such as checkpoint blockade therapy. Detecting an epithelial gene signature may indicate that the subject does not require more aggressive treatment. The epithelial tumor may be head and neck squamous cell carcinoma (HNSCC).

In another aspect, the present invention provides for a method for characterizing epithelial tumor composition comprising: detecting the presence of one or more expression programs in a sample, wherein each expression program comprises a set of biomarkers as defined in Table S7. The programs may comprise cell cycle, stress, epithelial differentiation, hypoxia or p-EMT programs.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Characterizing intra-tumoral expression heterogeneity in HNSCC by single-cell RNA-seq. (A) Workflow shows collection and processing of fresh biopsy samples of primary oral cavity HNSCC tumors and matched metastatic LNs for scRNA-seq. (B) Heat map shows large-scale CNVs for individual cells (rows) from a representative tumor (MEEI5), inferred based on the average expression of 100 genes surrounding each chromosomal position (columns). Red: amplifications; Blue: deletions. (C) Heatmap shows expression of epithelial marker genes across 5,902 single cells (columns), sorted by the average expression of these genes. (D) Violin plot shows distributions of epithelial scores (average expression of epithelial marker genes) for cells categorized as malignant or non-malignant based on CNVs. See also FIG. 8 and Tables S1-S4.

FIG. 2—Expression heterogeneity of malignant and non-malignant cells in the HNSCC ecosystem. (A) t-distributed stochastic neighbor embedding (t-SNE) plot of non-malignant cells from 10 patients reveals consistent clusters of stromal and immune cells across tumors. Clusters are assigned to indicated cell types by differentially expressed genes (see also FIG. 9B). (B) (Left) Zoomed in t-SNE plot of T-cells with distinct naïve-like, regulatory, cytotoxic, and exhausted populations as identified by DBscan clustering. (Right) Zoomed in t-SNE plot of fibroblasts with myofibroblasts, non-activated resting fibroblasts, and activated CAFs (cancer associated fibroblasts), which can be seen to further divide into two sub-clusters. Differentially expressed genes are listed for key subsets (see also FIG. 9). (C) t-SNE plot of malignant cells from 10 patients (indicated by colors) reveals tumor-specific clusters. Clustering patterns for malignant and non-malignant cells are not driven by transcriptome complexity (see also FIG. 9J). (D) Heatmap shows genes (rows) that are differentially expressed across 10 individual primary tumors (columns). For five tumors, expression is also shown for matched LNs. Red: high expression; Blue: low expression. Selected genes are highlighted. Two classical subtype tumors (MEEI6 and MEEI20; see also FIG. 6A) preferentially expressed genes associated with detoxification and drug metabolism (e.g. GPX2, GSTMs, CYPs, ABCC1). See also FIG. 9 and Table S5.

FIG. 3—Unbiased clustering reveals a common program of partial EMT (p-EMT) in HNSCC tumors. (A) Heatmap shows differentially-expressed genes (rows) identified by non-negative matrix factorization (NNMF) clustered by their expression across single cells (columns) from a representative tumor (MEEI25). The gene clusters reveal intra-tumoral programs that are differentially expressed in MEEI25. The corresponding gene signatures are numbered and selected genes indicated (right). (B) Heatmap depicts pairwise correlations of 60 intra-tumoral programs derived from 10 tumors, as in (A). Clustering identifies seven coherent expression programs across tumors. Rows in the heatmap that correspond to programs derived from MEEI25 are indicated by arrows and numbered as in (A). (C) Heatmap shows NNMF gene scores (rows) for common (top) and tumor-specific (bottom) genes within the p-EMT program by tumor (columns). (D) Representative images of SCC9 HNSCC cells sorted by p-EMT marker TGFBI into p-EMT^{high} and p-EMT^{low} populations and analyzed by matrigel invasion assay. (E) Bar plot depicts relative invasiveness of p-EMT^{high} and p-EMT^{low} SCC9 cells sorted and analyzed as in (D) (representative experiment; error bars reflect SEM; ANOVA, p<0.005, n=3). (F) Bar plot depicts relative proliferation of p-EMT^{high} and p-EMT^{low} SCC9 cells sorted as in (D) (representative experiment; error bars reflect SEM; ANOVA, p<0.0001, n=4). (G) (Left) Fluorescence-activated cell sorting plot identifies p-EMT^{high} and p-EMT^{low} SCC9 cells isolated based on TGFBI expression. (Right) Histogram (offset) reveals the distribution (x-axis) of TGFBI expression across cells from the respective isolates (p-EMT^{high}, p and unsorted; separated by dashed lines). After 7 days in culture, p-EMT^{high}, p-EMT^{low}, and unsorted cells have similar distributions of p-EMT marker expression. Additional experiments with the p-EMT marker CXADR demonstrate similar findings (data not shown). (H) Violin plot depicts p-EMT scores for unsorted, p-EMT^{low}, and p-EMT^{high} SCC9 cell sorted and cultured as in (G). Respective isolates largely recapitulate the initial distribution of p-EMT scores. See also FIGS. 10 and 11 and Tables S6 and S7.

FIG. 4—p-EMT cells at the leading edge engage in cross-talk with CAFs. (A-C) IHC images of representative HNSCC tumors (MEEI5, MEEI16, MEEI17, MEEI25, MEEI28) stained for p-EMT markers (PDPN, LAMB3, LAMC2) and the malignant cell-specific marker p63 (A and B) or the epithelial program marker SPRR1B (C). Scale bar=100 μM. (D) Scatter plot shows the Pearson correlation between the p-EMT program and other expression programs underlying HNSCC intra-tumoral heterogeneity (FIG. 3). Blue circles depict the correlations within individual tumors; black circles and error-bars represent the average and standard error, respectively, across the different tumors. (E) Bar plot depicts numbers of putative receptor-ligand interactions between malignant HNSCC cells and indicated cell types. Interaction numbers were calculated based on expression of receptors and corresponding ligands in scRNA-seq data. Outgoing interactions refer to the sum of ligands from malignant cells that interact with receptors on the indicated cell type. Incoming interactions refer to the opposite. CAFs express a significantly greater number of ligands whose receptors are expressed by malignant cells (hypergeometric test, p<0.05). (F) Heatmap depicts expression of ligands expressed by in vivo and in vitro CAFs. Relative expression is shown for all in vivo CAFs, MEEI18 in vivo CAFs, and in vitro CAFs derived from MEEI18. (G) Heatmap depicts relative expression of genes that were differentially regulated when SCC9 cells were treated with TGF4β3 or TGFβ pathway inhibitors. Panel includes all genes with significantly higher expression upon TGF4β3 treatment and lower expression upon TGFβ inhibition, relative to vehicle (t-test, p<0.05). Heat intensity reflects relative expression of indicated genes in bulk RNA-seq profiles for nine samples in each group, corresponding to distinct dosage or time points (see Materials and Methods). Selected genes are labeled and overlap with the in vivo p-EMT program (bold). (H) Violin plot depicts distributions of the p-EMT gene expression score across SCC9 cells treated as in (G) and profiled by scRNA-seq. p-EMT scores were increased with TGF4β3 treatment and decreased upon TGFβ inhibition, relative to vehicle (t-test, p<10⁻¹⁶) (I) Bar plot shows relative invasiveness of SCC9 cells treated as in (G) (representative experiment; error bars reflect SEM; ANOVA, p<0.0001, n=3). In vitro treatment of HNSCC cells with the CAF-related ligand TGFβ causes coherent induction of the p-EMT program and increases invasiveness, while TGFβ inhibition has the opposite effect. See also FIG. 12.

FIG. 5—Intra-tumoral HNSCC heterogeneity recapitulated in nodal metastases. (A) t-SNE plot of malignant cells (as in FIG. 2) from five primary tumors (black) and their matched LNs (red). Malignant cells cluster by tumor rather than by site. (B) t-SNE plot of non-malignant cells (as in FIG. 2) from five primary tumors (black) and their matched LNs (red). Non-malignant cells are consistent across tumors but their representation and expression states vary between sites (see also FIG. 9). See also FIG. 13.

FIG. 6—HNSCC subtypes revised by deconvolution of expression profiles from hundreds of tumors. (A) t-SNE plot of malignant cells from ten tumors (as in FIG. 2). Each cluster of cells corresponds to a different tumor. Cells are colored according to the TCGA expression subtype that they match. Black indicates no match. Each tumor can be clearly assigned to one of three subtypes: basal, atypical, or classical. (B) t-SNE plot of non-malignant cells from ten tumors (as in FIG. 2). Each cluster of cells corresponds to a different cell type. Cells are colored according to the TCGA expression subtype that they match. Black indicates no match. Fibroblasts and myocytes highly express signature genes of the mesenchymal subtype, which likely reflects tumor profiles with high stromal representation. (C) For each TCGA subtype (columns), heatmap shows relative expression of gene signatures for non-malignant cell types (rows), which were used as estimates of cell type abundances. Tumors classified as mesenchymal highly expressed genes specific to CAFs and myocytes, while atypical tumors were enriched for T- and B-cells. (D) Heatmap depicts pairwise correlations between TCGA expression profiles ordered by their subtype annotations. This analysis included all genes and recovered all four subtypes. (E) Schematic of linear regression used to subtract the influence of non-malignant cell frequency from bulk TCGA expression profiles, and thereby infer malignant cell-specific expression profiles. (F) Heatmap depicts pairwise correlations between TCGA expression profiles ordered by their subtype annotations. This analysis was based on the inferred malignant cell-specific expression profiles in (E). Classical and atypical subtypes are maintained. However, basal and mesenchymal subtypes collapse to a single subtype, which Applicants term ‘malignant-basal.’ See also FIG. 14.

FIG. 7—p-EMT predicts nodal metastasis and adverse pathologic features. (A) PC1 and PC2 gene scores based on PCA of inferred malignant cell-specific profiles from all malignant-basal TCGA tumors (n=225). p-EMT genes (red) and epithelial differentiation genes (green) underlie variance among malignant-basal tumors. (B) PC1 and PC2 gene scores based on PCA of inferred malignant cell-specific profiles from all classical and atypical TCGA tumors (n=156). p-EMT (red) and epithelial differentiation (green) genes are weakly associated with variance in these tumors. (C) Plot depicts percentage of p-EMT high and p-EMT low malignant-basal tumors associated with each clinical feature. Higher p-EMT scores were associated with positive LNs, advanced nodal stage, high grade, extracapsular extension (ECE), and lymphovascular invasion (LVI) (hypergeometric test, p<0.05). Advanced local disease (T3/T4) as determined by T-stage did not correlate with p-EMT score. (D) Volcano plot depicts gene expression differences between malignant-basal TCGA tumors with multiple LNs versus those without positive LNs. p-EMT genes (red) have increased expression, while epithelial differentiation genes (green) have decreased expression in metastatic tumors. (E) Model of the in vivo p-EMT program associated with invasion and metastasis in malignant-basal HNSCC tumors. See also FIG. 14.

FIG. 8—Cells are classified as malignant and non-malignant based on CNVs and epithelial marker expression, Related to FIG. 1. (A) Histograms show distribution of cells ordered by numbers of reads (Left; median 1.34 million reads), percent of reads mapped to the transcriptome (Middle; median 52.2%), and number of unique genes detected (Right; median 3,880 detected genes). (B) Heatmap shows large-scale CNVs for individual cells (rows) from 18 tumors, inferred based on the average expression of 100 genes surrounding each chromosomal position (columns). Red: Amplifications; Blue: Deletions. (C) Large-scale CNVs of seven samples (rows) from three patients as defined by whole exome sequencing analysis. (D) Stacked bar plots of 27 clusters show percent of malignant (blue) and non-malignant (red) cells, as classified by one (light color) or two (dark color) independent methods: epithelial marker scoring and CNVs. 22 of 27 clusters contain >95% malignant or non-malignant cells; cells in the remaining five clusters were excluded from further analysis.

FIG. 9—Expression heterogeneity of stromal and immune cells in the HNSCC ecosystem, Related to FIG. 2. (A) t-SNE plot of non-malignant cells (as shown in FIG. 2A) colored by their assignment to 14 clusters by SC3 (Bacher et al., 2017) with default parameters, demonstrating high consistency between SC3 clusters and tSNE coordinates. (B) t-SNE plot of non-malignant cells from 10 tumors (same as FIG. 2A) with cells colored based on the average expression of sets of marker genes for particular cell types (marker genes and associated cell types are indicated next to each plot). Zero expression level (for all markers of a given cell type) is indicated with small circles, and positive expression is indicated by larger circles, with higher levels indicated by shades of red. (C) (Top) Zoomed in t-SNE plot of T-cells with four distinct clusters identified. (Bottom) Heat map of differentially expressed genes (rows) facilitates annotation of the four clusters (columns) as naïve-like, regulatory, cytotoxic, and exhausted. (D) Bar plot shows percent of exhausted CD8+ T-cells in six tumors. Asterisks indicate a significant deviation from the mean (hypergeometric test, p<0.01). (E) (Top) Zoomed in t-SNE plot of fibroblasts with two distinct clusters and a set of intermediates identified. (Bottom) Heat map of differentially expressed genes (rows) facilitates annotation of the clusters (columns) as myofibroblasts, activated CAFs, and intermediate (resting) fibroblasts lacking coherent expression of genes consistent with either myofibroblasts or CAFs. (F) PC1 and PC2 from a principal component analysis of all fibroblasts, colored based on their assignments to the three clusters as in (D), demonstrates that PC2 further separates the CAF cluster into two subpopulations (CAF1 and CAF2, defined as CAFs with PC2>0, and PC2<0, respectively). (G) Heatmap of differentially expressed genes (rows) between the CAF1 and CAF2 subpopulations. Selected genes are indicated by name. (H) Heatmap shows distribution of relative CNVs (columns) for upregulated genes from 10 tumors (rows). Relative CNVs are calculated as the CNV value in the respective tumor minus the average CNVs of all other tumors. (I) Bar plot shows percentage of upregulated genes (blue) and other genes (red) with relative CNV>0.15 in each tumor, demonstrating a significant enrichment of upregulated genes with high CNVs in all cases (hypergeometric test with Bonferroni correction, p<0.05). (J) t-SNE plots of malignant (Left; same as FIG. 2C) and non-malignant (Right; same as FIG. 2A) cells colored by number of unique genes detected. These plots show that clustering is not driven by the detected number of genes. Additional analyses with clusters annotated by batch demonstrate clusters are not determined by batch effects (data not shown). (K) (Top) Heatmap shows absolute expression of housekeeping (positive) genes (top rows) and immune marker (negative) genes (bottom rows) in single cells (columns) from MEEI25 (same as FIG. 3A). (Middle) Heatmap shows absolute expression of genes defining distinct meta-programs (rows) identified by NNMF in single cells (columns) from MEEI25. (Bottom) Bar plot shows number of detected genes in single cells (columns) from MEEI25, with cells ordered as in top and middle panels. Variability in the number of genes detected is not linked to the expression programs identified.

FIG. 10—Defining the p-EMT program in HNSCC tumors and cell lines, Related to FIG. 3. (A) Each panel (from top to bottom) shows the meta-signature scores (top section of panel) and a heat map with expression of the top 10 genes for that meta-signature (bottom section of panel) for each of the six coherent expression programs in malignant cells. Cells from ten HNSCC tumors are included and sorted (left to right) first by tumor, within a tumor by sample (primary followed by LN, when applicable), and within a sample by the corresponding meta-signature score (black line). (B) Each panel (from top to bottom) shows violin plots that depict scores for one of the six meta-signatures in (A) for malignant cells from ten tumors. Violin plots in the second panel depict p-EMT scores, revealing distinct cohorts of p-EMT low (blue) and p-EMT high (red) tumors. Tumors in all panels are ordered identically. (C-F) Line graphs show smoothed expression (moving average with a window of 100 cells) for selected genes (as labeled); cells from ten HNSCC tumors were included and rank ordered by p-EMT program expression. The selected genes include six of the top p-EMT genes (C), eight epithelial genes negatively correlated with p-EMT scores (D), six epithelial genes not correlated with p-EMT scores (E), and canonical EMT transcription factors (TFs) (F). (G) Heatmap depicts pairwise Pearson correlations of global expression profiles of malignant cells from ten tumors and five oral cavity HNSCC cell lines. Correlations were calculated across all genes with average expression (Eₐ) above four in at least one of the tumors or cell lines and after centering the expression levels of genes across all samples included. Clustering indicates that cell lines are more similar to one another than to primary tumor samples and also illustrates the distinction between tumor samples of different subtypes. (H) Heatmaps show pairwise correlations of expression profiles from individual cells in five oral cavity HNSCC cell lines, ordered by hierarchical clustering. SCC9 includes a subpopulation of cells with an expression profile reminiscent of the p-EMT program, while SCC25 has a subpopulation with an expression profile similar to the stress program. Selected genes preferentially expressed within these subpopulations are highlighted, with markers used for sorting experiments (TGFBI, CXADR) in bold.

FIG. 11—Distinguishing the p-EMT program in HNSCC tumors from previously described EMT programs and modeling p-EMT in vitro, Related to FIG. 3. (A) Correlation plot demonstrates pairwise Pearson correlations between EMT and p-EMT programs, including signatures from previous work, as well as this work. Previously described TCGA-Mesenchymal genes (“Mes”), EMT signatures from tumors (“Tumor”), and cell lines (“Culture”) strongly correlate with the expression program of CAFs. These programs weakly correlate with the p-EMT program (“Orig.”) described in this study. Focusing on malignant-specific p-EMT genes (“Malig.”) and p-EMT genes identified after deconvolution (“Decon.”) reveals a more limited correlation of p-EMT with TCGA-Mes and previous EMT signatures, indicating this program is distinct from prior EMT descriptions. (B) Scatter plot demonstrates three cohorts of TCGA tumors, with (1) high TCGA-mes/intermediate p-EMT, (2) high p-EMT, and (3) low p-EMT scores. (C) Heatmap demonstrates relative expression of TCGA-Mes, CAF, and p-EMT genes (rows) in TCGA tumors (columns) from the cohorts described in (B), with the eight malignant-specific p-EMT genes (“Malig.”) shown at the bottom. (D) Bar plots show average expression of each of the gene sets described in (C) in CAFs, malignant cells, and all other immune and stromal cell types detected in this cohort. The p-EMT signature is highly specific to malignant cells, while the TCGA-mes signature is associated with CAFs. (E) Line graphs show percentage of cycling malignant cells within a sliding window of 20 cells, rank ordered by p-EMT scores. Seven p-EMT high tumors are included; in each tumor, a p-value is shown (permutation test), corresponding to the enrichment of cycling cells among the 30% of cells with lowest p-EMT scores in that tumor. Low p-EMT is significantly enriched with cycling cells among the three tumors with the highest p-EMT scores (MEEI16, MEEI17, and MEEI25). (F) Bar plot depicts relative invasiveness of SCC9 cells transfected with TGFBI or vector in matrigel invasion assays (error bars reflect SEM; t-test, p<0.005, n=3). (G) Bar plot shows relative proliferation of SCC9 treated as in (F) (error bars reflect SEM; ANOVA, p<0.0001, n=4). (H) (Top left) Fluorescence-activated cell sorting plot identifies p-EMT^{high} and p-EMT^{low} SCC9 cells isolated based on TGFBI expression. (Top right) Histogram (offset) reveals the distribution of TGFBI expression across cells from the respective isolates (p-EMT^{high} and p-EMT^{low}; separated by dashed line) immediately after sorting. (Bottom) Histograms (offset) reveal the distribution of TGFBI expression across cells from the respective isolates (p-EMT^{high} and p-EMT^{low}; separated by dashed line) after 4 hours, 24 hours, 4 days, and 7 days in culture. The p-EMT^{high} and p-EMT^{low} populations remained distinct 4 hours and 24 hours after sorting (representative experiment; t-test, p<0.0001, n=3).

FIG. 12—p-EMT program is localized at the leading edge, distinct from the epithelial differentiation program at the core, Related to FIG. 4. (A-C) Immunohistochemical staining of representative tumors (MEEI5, MEEI16, MEEI17, MEEI25, MEEI28) for p-EMT (LAMC2, MMP10, TGFBI) with the malignant cell-specific marker p63. Scale bar=100 μM. The leading edges of tumors co-stain with p63 and p-EMT markers. Additional staining with the marker p-EMT marker ITGA5 further validated localization of p-EMT at the leading edge (data not shown). (D) Immunohistochemical staining of representative tumors (MEEI17, MEEI28) for multiple p-EMT markers (LAMC2, TGFBI). p-EMT markers co-localize at the leading edge. (E-G) Immunohistochemical staining of representative p-EMT low tumors (MEEI20, MEEI26) for p-EMT (PDPN, LAMB3, LAMC2) with the malignant cell-specific marker p63. p-EMT low tumors show minimal staining for p-EMT markers at the leading edge. Additional staining with the marker ITGA5 confirmed minimal staining for the p-EMT program in these tumors (data not shown). (H and I) Immunohistochemical staining of representative tumors (MEEI16, MEEI17) for epithelial differentiation (SPRR1B, CLDN4) and the malignant cell-specific marker p63. (J and K) Immunohistochemical staining of representative tumor (MEEI17) for p-EMT (LAMC2, PDPN) and epithelial differentiation (CLDN4). Markers demonstrate distinct spatial localization of p-EMT and epithelial differentiation programs, at the leading edge and core, respectively. (L) Bar plot shows statistical significance (minus log 10 of p-value defined by hypergeometric test) of number of observed outgoing interactions between ten listed cell types and malignant cells. Bars above the x-axis indicate a greater number of interactions than expected, while bars below the x-axis indicate fewer interactions than expected. (M) Immunohistochemical staining of representative tumors (MEEI16, MEEI18) for p-EMT and CAFs (FAP) with the malignant cell-specific marker p63. FAP staining is present both at the leading edge of tumors nests and in the stroma, highlighting activated CAFs. (N) Bar plot depicts relative proliferation of SCC9 cells treated with vehicle, TGFβ, or TGFβ pathway inhibitors (error bars reflect SEM; ANOVA, p<0.0001, n=4). (0) Histograms show percent of sequencing reads with insertions or deletions (indels) of specified size in mock infected SCC9 cells (Top left) and SCC9 TGFBI CRISPR knockout cells (other panels). Each of the TGFBI-targeting sgRNAs resulted in >98.8% of reads containing indels, indicating efficient knockout of TGFBI. (P) Bar plot depicts relative invasiveness of mock infected SCC9 cells or SCC9 TGFBI CRISPR knockout cells after treatment with vehicle or TGFβ in matrigel invasion assays (error bars reflect SEM; ANOVA, p<0.0001, n=3). (Q) Violin plot depicts hypoxia program scoring of SCC9 cells grown in normoxic or hypoxic conditions. Hypoxic conditions are associated with significantly increased hypoxia score (t-test, p<0.05). (R) Violin plot depicts scoring of SCC9 cells for p-EMT scores after growth in standard conditions (control), hypoxic conditions, or in co-culture with CAFs derived from MEEI18. p-EMT expression is not significantly changed across these conditions.

FIG. 13—Variability in the p-EMT program and cancer-associated fibroblasts across tumor subsites (primary and lymph node), Related to FIG. 5. (A) Comparison of point mutations between primary and LN samples in three individual tumors (MEEI26, MEEI20, and MEEI25 from top to bottom) as detected by whole exome sequencing. In each tumor, Applicants examined all mutations identified in at least one of the samples (primary or LN) and assigned it one of three values in each sample: “detected” (black), “not detected” (white), or unresolved due to “low coverage.” A single mutant read was sufficient to define a mutation as “detected,” but zero mutant reads were defined as “not detected” only if the probability of detecting zero mutant reads in that sample was below 0.05 (as defined by binomial test, given the number of reads covering that base and assuming the same frequency of the mutant reads as in the sample(s) where it is detected). Mutations were then ordered by their identification across the samples and assigned to four classes: shared among primary and LN, specific to primary, specific to LN, and unresolved. Note that for MEEI26 two LN samples are included corresponding to the left (ipsilateral) and right (contralateral) LNs, denoted as LN_{L} and LN_{R}, respectively. (B) Heatmap of differentially expressed genes between primary and LN samples across multiple patients. For each of the five patients with matched primary and LN samples, Applicants identified significant differentially expressed genes (defined by p<0.001 and fold-change>2). All genes defined as upregulated in at least two patients (left panel) or downregulated in at least two patients (right panel) are shown. Red: upregulated; Blue: downregulated. Darker shades indicate significant differential expression, while lighter shades denote borderline differential expression (p<0.05 and fold-change>1.5). (C) Violin plot depicts p-EMT score of malignant cells from five primary tumors and matched LN. (D) Scatter plot shows the average (x-axis) and the variability (y-axis) of p-EMT scores across individual malignant cells within each sample; five primary tumors (black) and matched LNs (red) are included and matched samples are connected with lines. p-EMT high tumors display both higher average and higher variability of p-EMT scores. (E) Fibroblasts from primary (black) and LN (red) samples, scored by the relative expression of gene-sets distinguishing CAFs from myofibroblasts (x-axis) and those distinguishing the CAF1 and CAF2 subsets (y-axis), demonstrating that LN CAFs are biased towards the CAF1 subset (hypergeometric test, p<0.05). (F and G) Immunohistochemical staining of representative LN metastases (MEEI25, MEEI28) for p-EMT (PDPN, LAMB3) with the malignant-cell specific marker p63.

FIG. 14—p-EMT program is negatively correlated with epithelial differentiation and may predict nodal metastasis, Related to FIGS. 6 and 7. (A) Hematoxylin-eosin (H&E) stained sections from representative mesenchymal (Left) and basal (Right) TCGA tumors demonstrate substantially more stromal infiltrate in mesenchymal than basal tumors. Scale bar=400 μM. (B) (Left) Bar plot shows significantly higher percent of stromal infiltrate in mesenchymal tumors compared to basal tumors (t-test, p<0.0001; n=203 tumors). (Right) Bar plot shows number of tumors with H&E stromal scores ranging from 0 (lowest) to 4 (highest) for mesenchymal and basal subtype TCGA tumors. (C and D) Scatter plots demonstrate a correlation between H&E stromal score (indicated by dot color) with CAF and TCGA mesenchymal scores (C), but not p-EMT scores (D). (E) Line graph shows distribution of p-EMT scores across TCGA tumors of each subtype. (F) Scatter plot shows scoring of TCGA basal and mesenchymal tumors for epithelial differentiation and p-EMT which are significantly negatively correlated in this subset of tumors (Pearson correlation, p<0.05); black lines indicate linear regression. (G) Scatter plot shows scoring of TCGA classical and atypical tumors for epithelial differentiation and p-EMT, which are not significantly correlated in this subset of tumors; black lines indicate linear regression. (H) Bar plot shows direction and statistical significance (p-value based on a t-test) of the association between each of six coherent meta-signatures and the presence of multiple versus no metastatic LNs in TCGA malignant-basal tumors. The p-EMT and epithelial differentiation programs, which were inversely correlated in expression studies, had opposite associations with metastasis. The other programs show no significant association with LN metastases. (I) (Top) Bar plot shows the percent of patients with adverse clinical features (positive LNs, multiple LNs, advanced N stage, grade III, extranodal extension, lymphovascular invasion, and advanced local disease) in cohorts with high and low p-EMT scores stratified by high and low CAF scores. (Bottom) Heatmap shows the statistical significance of p-EMT and CAF effects on adverse clinical features based on a binomial logistic regression with two predictive variables (p-EMT and variable scores) and an interaction effect. Only the p-EMT effect is predictive of clinical features associated with metastasis and invasion (positive LNs, multiple LNs, advanced nodal stage, extracapsular extension, and lymphovascular invasion) (Bottom, first row). In contrast, the CAF effect has no significant predictive value for features associated with metastasis, but instead, predicts high grade disease and advanced local disease (T3/T4) (Bottom, second row). The p-EMT and CAF effects did act cooperatively to influence the risk of nodal metastasis (Bottom, third row), consistent with a putative ligand-receptor interaction between CAFs and p-EMT cells. (J) Percent of patients from TCGA for which neck dissection was justified using varying thresholds of p-EMT scores and stratified by tumor (T) stage. Justified neck dissection refers to patients with initial clinical diagnosis of lymph node-negative (cN0) for which neck dissection revealed a positive metastatic lymph node (pN1-N3); the percentage of justified neck dissections was calculated out of all patients with clinical node-negative disease that underwent neck dissection. A higher p-EMT threshold is associated with a higher rate of justified neck dissection, regardless of T-stage (permutation test, p<0.05). (K) Correlations of genes with the p-EMT program within (x-axis) and across (y-axis) tumors in the cohort of ten patients. Within-tumor correlations were calculated separately in each tumor and averaged; across-tumor correlations were calculated between the average levels of genes and those of the p-EMT program across all malignant cells in each tumor. Selected genes are indicated. (L) Scatter plot shows the correlations of genes with p-EMT (x-axis) and epithelial differentiation (y-axis) programs based on inferred malignant cell-specific profiles from TCGA malignant-basal tumors. Genes of the p-EMT (red) and epithelial differentiation (green) programs as well as EMT TFs (black) are indicated, demonstrating a high p-EMT correlation with SNAIL2 but not of other EMT TFs.

FIG. 15—block diagram depicting a method for generating a p-EMT score in a tumor using bulk RNA-seq data obtained from a sample of the tumor.

FIG. 16—p-EMT predicts adverse pathologic features in an independent MEEI cohort of patients by IHC. Higher p-EMT scores were associated with positive LNs, advanced nodal stage, perineural invasion, lymphovascular invasion (LVI) and high grade. Advanced local disease (T2/T4) as determined by T-stage did not correlate with high p-EMT score.

FIG. 17—quantification of marker staining.

FIG. 18—classification of tumors as basal subtype. Tumors were classified as non-basal subtype and eliminated from analysis (20%) if staining was 1+ for multiple markers. p-EMT quantification in malignant-basal subtype tumors correlated with pathologic features.

The figures herein are for illustrative purposes only and are not necessarily drawn to scale. The following manuscript contains complete color versions of the figures described above and is hereby fully incorporated herein by reference: Puram et al., Single-Cell Transcriptomic Analysis of Primary and Metastatic Tumor Ecosystems in Head and Neck Cancer, Cell. 2017 Dec. 14; 171(7):1611-1624.e24. doi: 10.1016/j.cell.2017.10.044. Epub 2017 Nov. 30.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +1-0.1% or less of and from the specified value, insofar such variations are appropriate to perform in the disclosed invention. It is to be understood that the value to which the modifier “about” or “approximately” refers is itself also specifically, and preferably, disclosed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Human tumors are composed of diverse malignant, stromal and immune cell states, which are masked when bulk samples are profiled. Applicants investigated primary HNSCC tumors and matched LNs in order to better understand intra-tumoral heterogeneity, invasion, and metastasis in an epithelial human cancer. By analyzing 18 tumors, including five matched pairs of primary tumors and LN metastases, Applicants profiled ˜6,000 individual tumor cells, revealing expression programs that distinguish diverse malignant, stromal, and immune cells. Malignant cells vary in their expression of programs related to cell cycle, stress, hypoxia and epithelial differentiation. A subset also express a partial EMT (p-EMT) program with extracellular matrix proteins, but lacking classical EMT transcription factors (TFs). p-EMT cells localized to the leading edge of primary tumors in close proximity to cancer-associated fibroblasts. A similar tumor-stromal interaction was evident in matched lymph nodes in structured tumor nests. Knowledge of HNSCC expression cell states allowed Applicants to deconvolve bulk RNA-seq data from The Cancer Genome Atlas (TCGA), and thereby redefine HNSCC subtypes by their malignant and stromal components. Notably, the p-EMT program is largely specific to the most prevalent HNSCC subtype, where it is associated with adverse clinical and pathologic features such as metastasis, tumor grade, and extracapsular extension. These data define inter-tumoral and intra-tumoral heterogeneity in HNSCC, and provide insight into in vivo EMT-like changes and stromal interactions relevant to tumor invasion and metastasis.

Embodiments disclosed herein provide for a p-EMT signature in epithelial tumors capable of guiding treatment of the tumors. Embodiments disclosed herein provide tools and methods for prognosing and stratifying epithelial tumors. The methods leverage a novel gene signature program detectable in HNSCC tumors. Applicants have discovered several malignant cell gene expression programs and have defined the tumor microenvironment in HNSCC using single cell RNA-seq. The discovery enables the deconvolution of bulk sequencing gene expression data of a HNSCC sample to identify the malignant gene expression programs and determine the gene expression attributed to the tumor microenvironment (TME). Deconvolution utilizes a novel algorithm constructed based on the insight obtained from the single cell sequencing, such as malignant cell sub-types and non-malignant cell types. Specifically, applicants identified an EMT-like meta-signature (p-EMT) that correlates with lymph node metastasis. Thus, applicants have developed methods and systems for analyzing bulk sequencing data from a subject and classifying it based on a p-EMT high signature score. The EMT-signature score can then be used to predict lymph node (LN) metastasis and direct treatment decisions. The p-EMT signature genes or polypeptides may also be therapeutically targeted in order to prevent unfavorable clinical outcomes (e.g., metastasis). In one embodiment, a tumor biopsy is obtained from a subject in need thereof and the sample is analyzed by RNA-seq. The expression data can then be denconvoluted to determine a p-EMT score. The subject may then be treated according to the pEMT score.

### Cancer

In certain embodiments, the systems and methods may be used for any epithelial cancer. Studies have suggested that EMT is a process that occurs in all epithelial tumors. Not being bound by a theory, epithelial tumors all express similar p-EMT programs as described herein. HNSCC is one of many common epithelial tumors. Not being bound by a theory, detection of the p-EMT signature described herein in any epithelial tumor predicts 1) risk of having lymph node or distant metastasis, 2) tumor stage, 3) adverse pathologic features, 4) need for adjuvant (radiation/chemotherapy) treatment, 5) treatment response, and 6) overall survival. The examples described herein show that the p-EMT signature is a strong genetic predictor of having lymph node (LN) involvement and that the signature predicts the need for a neck dissection (removal of LN).

Cancers may include, but are not limited to, breast cancer, colon cancer, lung cancer, prostate cancer, testicular cancer, brain cancer, skin cancer, rectal cancer, gastric cancer, esophageal cancer, tracheal cancer, head and neck cancer, pancreatic cancer, liver cancer, ovarian cancer, lymphoid cancer, cervical cancer, vulvar cancer, melanoma, mesothelioma, renal cancer, bladder cancer, thyroid cancer, bone cancers, cutaneous squamous cell carcinoma, carcinomas, sarcomas, and soft tissue cancers. Thus, the disclosure is generally applicable to any type of cancer in which expression of an EMT program occurs. In certain embodiments, the signature is useful for all epithelial tumors, including but not limited to lung, breast, prostate, colon, cutaneous squamous cell carcinoma and esophageal carcinoma.

### Use of Signature Genes

As used herein a “signature” or “gene signature” may encompass any gene or genes, protein or proteins, or epigenetic element(s) whose expression profile or whose occurrence is associated with a specific cell type, subtype, or cell state of a specific cell type or subtype within a population of cells. For ease of discussion, when discussing gene expression, any of gene or genes, protein or proteins, or epigenetic element(s) may be substituted. As used herein, the terms “signature”, “expression profile”, or “expression program” may be used interchangeably. It is to be understood that also when referring to proteins (e.g. differentially expressed proteins), such may fall within the definition of “gene” signature. Levels of expression or activity or prevalence may be compared between different cells in order to characterize or identify for instance signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and down-regulated genes that are representative of a cell type or subtype. A gene signature as used herein, may also refer to any set of up- and down-regulated genes between different cells or cell (sub)populations derived from a gene-expression profile. For example, a gene signature may comprise a list of genes differentially expressed in a distinction of interest.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single-cells within a population of cells from isolated samples (e.g. tumor samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of cancer cells that are linked to particular pathological condition (e.g. cancer grade), or linked to a particular outcome or progression of the disease (e.g. metastasis), or linked to a particular response to treatment of the disease.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

In certain embodiments, a signature is characterized as being specific for a particular tumor cell or tumor cell (sub)population if it is upregulated or only present, detected or detectable in that particular tumor cell or tumor cell (sub)population, or alternatively is downregulated or only absent, or undetectable in that particular tumor cell or tumor cell (sub)population. In this context, a signature consists of one or more differentially expressed genes/proteins or differential epigenetic elements when comparing different cells or cell (sub)populations, including comparing different tumor cells or tumor cell (sub)populations, as well as comparing tumor cells or tumor cell (sub)populations with non-tumor cells or non-tumor cell (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of tumor cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized, and is preferably characterized by the signature as discussed herein. A cell (sub)population as referred to herein may constitute a (sub)population of cells of a particular cell type characterized by a specific cell state.

When referring to induction, or alternatively suppression of a particular signature, preferable is meant induction or alternatively suppression (or upregulation or downregulation) of at least one gene/protein and/or epigenetic element of the signature, such as for instance at least to, at least three, at least four, at least five, at least six, or all genes/proteins and/or epigenetic elements of the signature.

Various aspects and embodiments of the invention may involve analyzing gene signatures, protein signature, and/or other genetic or epigenetic signature based on single cell analyses (e.g. single cell RNA sequencing) or alternatively based on cell population or bulk analyses, as is defined herein elsewhere.

In further aspects, the invention relates to gene signatures, protein signature, and/or other genetic or epigenetic signature of particular tumor cell subpopulations, as defined herein elsewhere. The invention hereto also further relates to particular tumor cell subpopulations, which may be identified based on the methods according to the invention as discussed herein; as well as methods to obtain such cell (sub)populations and screening methods to identify agents capable of inducing or suppressing particular tumor cell (sub)populations.

The invention further relates to various uses of the gene signatures, protein signature, and/or other genetic or epigenetic signature as defined herein, as well as various uses of the tumor cells or tumor cell (sub)populations as defined herein. Particular advantageous uses include methods for identifying agents capable of inducing or suppressing particular tumor cell (sub)populations based on the gene signatures, protein signature, and/or other genetic or epigenetic signature as defined herein. The invention further relates to agents capable of inducing or suppressing particular tumor cell (sub)populations based on the gene signatures, protein signature, and/or other genetic or epigenetic signature as defined herein, as well as their use for modulating, such as inducing or repressing, a particular gene signature, protein signature, and/or other genetic or epigenetic signature. In one embodiment, genes in one population of cells may be activated or suppressed in order to affect the cells of another population. In related aspects, modulating, such as inducing or repressing, a particular gene signature, protein signature, and/or other genetic or epigenetic signature may modify overall tumor composition, such as tumor cell composition, such as tumor cell subpopulation composition or distribution, or functionality.

The signature genes of the present invention were discovered by analysis of expression profiles of single-cells within a population of cells from freshly isolated tumors, thus allowing the discovery of novel cell subtypes that were previously invisible in a population of cells within a tumor. The presence of subtypes may be determined by subtype specific signature genes. The presence of these specific cell types may be determined by applying the signature genes to bulk sequencing data in a patient tumor. Not being bound by a theory, a tumor is a conglomeration of many cells that make up a tumor microenvironment, whereby the cells communicate and affect each other in specific ways. As such, specific cell types within this microenvironment may express signature genes specific for this microenvironment. Not being bound by a theory the signature genes of the present invention may be microenvironment specific, such as their expression in a tumor. Not being bound by a theory, signature genes determined in single cells that originated in a tumor are specific to other tumors. Not being bound by a theory, a combination of cell subtypes in a tumor may indicate an outcome. Not being bound by a theory, the signature genes can be used to deconvolute the network of cells present in a tumor based on comparing them to data from bulk analysis of a tumor sample. Not being bound by a theory the presence of specific cells and cell subtypes may be indicative of tumor growth, invasiveness and resistance to treatment. The signature gene may indicate the presence of one particular cell type. The presence of cell types within a tumor may indicate that the tumor will be resistant to a treatment. In one embodiment, the signature genes of the present invention are applied to bulk sequencing data from a tumor sample obtained from a subject, such that information relating to disease outcome and personalized treatments is determined. In one embodiment, the novel signature genes are used to detect multiple cell states that occur in a subpopulation of tumor cells that are linked to resistance to targeted therapies, progressive tumor growth and metastasis.

The gene signatures described herein are useful in methods of monitoring a cancer in a subject by detecting a level of expression, activity and/or function of one or more signature genes or one or more products of one or more signature genes at a first time point, detecting a level of expression, activity and/or function of one or more signature genes or one or more products of one or more signature genes at a second time point, and comparing the first detected level of expression, activity and/or function with the second detected level of expression, activity and/or function, wherein a change in the first and second detected levels indicates a change in the cancer in the subject.

One unique aspect of the invention is the ability to relate expression of one gene or a gene signature in one cell type to that of another gene or signature in another cell type in the same tumor. In one embodiment, the methods and signatures of the invention are useful in patients with complex cancers, heterogeneous cancers or more than one cancer.

In an embodiment of the invention, these signatures are useful in monitoring subjects undergoing treatments and therapies for cancer to determine efficaciousness of the treatment or therapy. In an embodiment of the invention, these signatures are useful in monitoring subjects undergoing treatments and therapies for cancer to determine whether the patient is responsive to the treatment or therapy. In an embodiment of the invention, these signatures are also useful for selecting or modifying therapies and treatments that would be efficacious in treating, delaying the progression of or otherwise ameliorating a symptom of cancer. In an embodiment of the invention, the signatures provided herein are used for selecting a group of patients at a specific state of a disease with accuracy that facilitates selection of treatments.

In one embodiment, the signature genes are detected by immunofluorescence, immunohistochemistry, fluorescence activated cell sorting (FACS), mass cytometry (CyTOF), RNA-seq, scRNA-seq, Drop-seq, InDrop, single cell qPCR, MERFISH (multiplex (in situ) RNA FISH) and/or by in situ hybridization. Other methods including absorbance assays and colorimetric assays are known in the art and may be used herein.

In one embodiment, tumor cells are stained for one or more cell subtype specific signature genes. In one embodiment, the cells are fixed. In another embodiment, the cells are formalin fixed and paraffin embedded. Not being bound by a theory, the presence of the cell subtypes in a tumor indicate outcome and personalized treatments. Not being bound by a theory, the cell subtypes may be quantitated in a section of a tumor and the number of cells indicates an outcome and personalized treatment. In preferred embodiments, EMT high cells according to the present invention are detected.

In certain embodiments, the invention involves targeted nucleic acid profiling (e.g., sequencing, quantitative reverse transcription polymerase chain reaction, and the like). In certain embodiments, a target nucleic acid molecule (e.g., RNA molecule), may be sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others.

In certain embodiments, the invention involves high-throughput single-cell RNA-sequencing where the RNAs from different cells are tagged individually, allowing a single library to be created while retaining the cell identity of each read. In this regard reference is made to Picelli, S. et al., 2014, “Full-length RNA-seq from single cells using Smart-seq2” Nature protocols 9, 171-181, doi:10.1038/nprot.2014.006; Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO 2014210353 A2; Zilionis, et al., 2017, “Single-cell barcoding and sequencing using droplet microfluidics” Nat Protoc. Jan; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; and Rosenberg et al., 2017, “Scaling single cell transcriptomics through split pool barcoding” bioRxiv preprint first posted online Feb. 2, 2017, doi: dx.doi.org/10.1101/105163, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; and Habib et al., 2016, “Div-Seq: Single-nucleus RNA-Seq reveals dynamics of rare adult newborn neurons” Science, Vol. 353, Issue 6302, pp. 925-928, both of which are herein incorporated by reference in their entirety.

In certain embodiments, single cells of a subject are sequenced to determine cell types and gene signatures present in a tumor. In one embodiment, sequencing is targeted for gene signatures of a specific cell type. Cells may be quantitated based on the sequencing of a cell specific gene signature. In certain embodiments, the depth of sequencing may be adjusted, such that cells having a particular gene signature can be detected. The term “depth (coverage)” as used herein refers to the number of times a nucleotide is read during the sequencing process.

### Treatment

It will be understood by the skilled person that treating as referred to herein encompasses enhancing treatment, or improving treatment efficacy. Treatment may include tumor regression as well as inhibition of tumor growth, metastasis or tumor cell proliferation, or inhibition or reduction of otherwise deleterious effects associated with the tumor.

Efficaciousness of treatment is determined in association with any known method for diagnosing or treating the particular cancer. The invention comprehends a treatment method comprising any one of the methods or uses herein discussed.

The phrase “therapeutically effective amount” as used herein refers to a nontoxic but sufficient amount of a drug, agent, or compound to provide a desired therapeutic effect.

As used herein “patient” refers to any human being receiving or who may receive medical treatment.

Therapy or treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the cancer, and how the patient responds to the treatment. Additionally, a person having a greater risk of developing a cancer (e.g., a person who is genetically predisposed) may receive prophylactic treatment to inhibit or delay symptoms of the disease.

As described herein, the p-EMT signature may be regulated by TGFβ signaling. Not being bound by a theory, detection of a p-EMT signature indicates that a therapy targeting the TGFβ pathway should be used in treating cancer. Therapies targeting TGFβ signaling have been described (see e.g., Neuzilleta, et al., Targeting the TGFβ pathway for cancer therapy, Pharmacology & Therapeutics, Volume 147, March 2015, Pages 22-31). In certain embodiments, an epithelial tumor with a high p-EMT score is treated with a known therapy targeting TGFβ signaling. Exemplary inhibitors are provided in Table 1. Not being bound by a theory, a high p-EMT score may indicate a patient population is more responsive to a therapy targeting TGFβ signaling.

TABLE 1
TGFβ pathway inhibitors in development in cancer.

| | | | |
|---|---|---|---|
| Name | Targets | Trial identifier | Current status |

TGFβ ligand inhibitors

| | | | |
|---|---|---|---|
| Lerdelimumab | TGFβ2 | | Development stopped |
| (CAT-152) | | | |
| Genzyme ® | | | |
| Metelimumab | TGFβ1 | | Development stopped |
| Genzyme ® | | | |
| Fresolimumab | TGFβ1, | NCT00356460 | Results in RCC, melanoma, mesothelioma and |
| (GC1008) | -β2, -β3 | NCT00923169 | glioma; combination phase I/II in progress in |
| Genzyme ®/Aventis ® | | NCT01472731 | breast cancer |
| | | NCT01112293 | |
| | | NCT01401062 | |
| LY2382770 | TGFβ1 | | In progress outside oncology |
| Eli Lilly ® | | | |
| Trabedersen | TGFβ2 | NCT00844064 | Results in glioma, PDAC, CRC, melanoma and |
| (AP12009) | | NCT00431561 | glioblastoma |
| Antisens Pharma ® | | NCT00761280 | |
| Lucanix | TGFβ2 | NCT01058785 | Results in glioma and NSCLC; combination |
| (Belagenpumatucel-L) | | NCT00676507 | phase I in progress |
| NovaRx Corporation ® | | | |
| FANG ™ Vaccine | TGFβ1, | NCT01061840 | In progress in melanoma, CRC and ovarian |
| (rhGMCSF/shRNAfurin) | -β2 | NCT01309230 | cancer |
| Gradalis ® | | NCT01505166 | |
| | | NCT01453361 | |
| Disitertide | TGFβ1 | | In progress outside oncology |
| (P144) | | | |
| Digna Biotech ® | | | |

TGFβ receptor inhibitors

| | | | |
|---|---|---|---|
| Galunisertib | TGFβRI | NCT01246986 | Phase II in progress in PDAC, HCC, glioma |
| (LY2157299) | | NCT01373164 | and glioblastoma |
| Eli Lilly ® | | NCT01220271 | |
| | | NCT02178358 | |
| | | NCT01582269 | |
| TEW-7197 | TGFβRI | NCT02160106 | Phase I in progress |
| MedPacto ® | | | |
| PF-03446962 | ALK-1 | NCT00557856 | Results of phase I; phase II results pending in |
| Pfizer ® | (TGFβRI) | NCT01337050 | HCC and in progress in malignant pleural |
| | | NCT01911273 | mesothelioma and refractory urothelial |
| | | NCT01486368 | carcinoma; combination phase I in progress |
| | | NCT01620970 | with regorafenib in CRC |
| | | NCT02116894 | |
| IMC-TR1 | TGFβRII | NCT01646203 | Phase I in progress |
| (LY3022859) | | | |
| Eli Lilly ® | | | |

CRC: colorectal carcinoma; HCC: hepatocellular carcinoma; NSCLC: non-small cell lung carcinoma; PDAC: pancreatic ductal adenocarcinoma; RCC: Renal cell carcinoma.

### Standard of Care

Aspects of the invention involve modifying the therapy within a standard of care based on the detection of a p-EMT signature as described herein. In one embodiment, therapy comprising an agent is administered within a standard of care where addition of the agent is synergistic within the steps of the standard of care. In one embodiment, the agent targets TGFβ signaling. In one embodiment, the agent inhibits expression or activity of a gene or polypeptide selected from the p-EMT signature. In one embodiment, the agent targets tumor cells expressing a gene or polypeptide selected from the p-EMT signature. The term “standard of care” as used herein refers to the current treatment that is accepted by medical experts as a proper treatment for a certain type of disease and that is widely used by healthcare professionals. Standard of care is also called best practice, standard medical care, and standard therapy. Standards of care for cancer generally include surgery, lymph node removal, radiation, chemotherapy, targeted therapies, antibodies targeting the tumor, and immunotherapy. Immunotherapy can include checkpoint blockers (CBP), chimeric antigen receptors (CARs), and adoptive T-cell therapy. The standards of care for the most common cancers can be found on the website of National Cancer Institute (www.cancer.gov/cancertopics). A treatment clinical trial is a research study meant to help improve current treatments or obtain information on new treatments for patients with cancer. When clinical trials show that a new treatment is better than the standard treatment, the new treatment may be considered the new standard treatment.

The term “Adjuvant therapy” as used herein refers to any treatment given after primary therapy to increase the chance of long-term disease-free survival. The term “Neoadjuvant therapy” as used herein refers to any treatment given before primary therapy. The term “Primary therapy” as used herein refers to the main treatment used to reduce or eliminate the cancer.

In exemplary embodiments, two types of standard treatment are used to treat HNSCC. In certain embodiments, the standard treatment is surgery or radiation therapy.

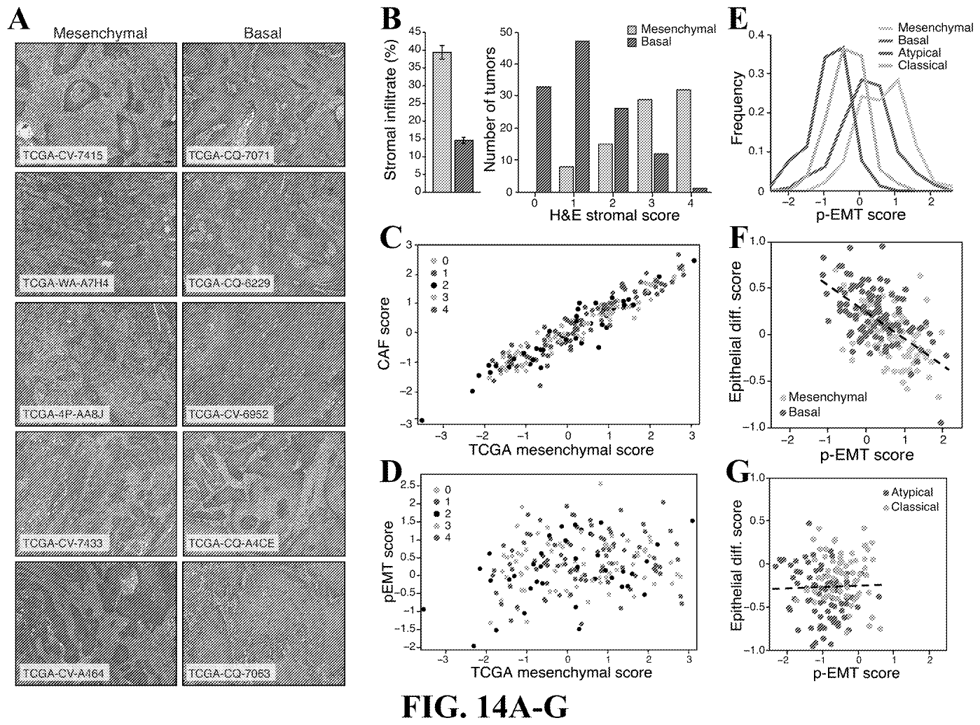

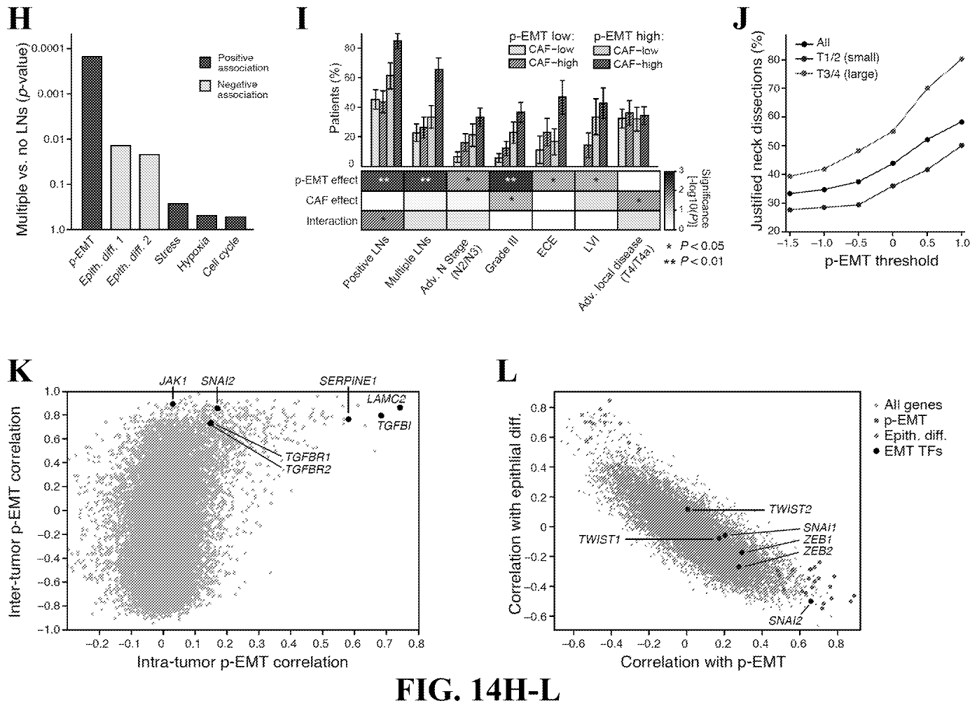

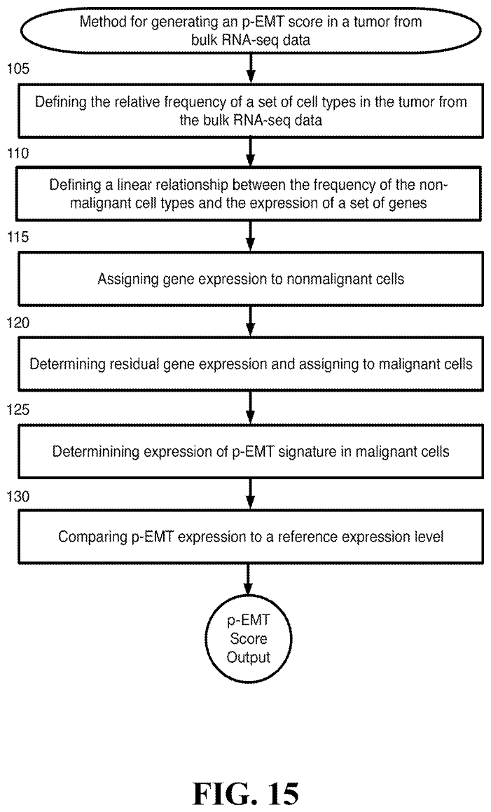

Surgery may include neck dissection. Not being bound by a theory, the current standard of care cannot predict whether a tumor has spread to the lymph nodes and unnecessary neck dissections may be performed (see, e.g., FIG. 14J). Not being bound by a theory, only after performing a neck dissection and examination of the dissected tissue can it be determined that the dissection was necessary. In preferred embodiments, neck dissection is used when a p-EMT signature, preferably a p-EMT high signature, as described herein is detected in a sample obtained from a subject in need thereof. The sample is preferably from a primary tumor. Neck dissection may be delayed when a p-EMT signature is not detected. Not being bound by a theory, unnecessary neck dissections may be avoided by incorporating the methods and gene signatures described herein into the standard of care. It will be appreciated by one of ordinary skill in the art that avoiding unnecessary aggressive interventions such as neck dissection also avoids the related potential co-morbidities and mortality associated with such procedures. The invention thus provides a substantial improvement in care of such patients.

There are different types of neck dissection based on the amount of tissue that is removed. Radical neck dissection may comprise surgery to remove tissues in one or both sides of the neck between the jawbone and the collarbone, including the following: 1) all lymph nodes, 2) the jugular vein, and 3) the muscles and nerves that are used for face, neck, and shoulder movement, speech, and swallowing. In most cases, radical neck dissection is used when cancer has spread widely in the neck. However, detection of cancer in the lymph nodes and detection of a p-EMT high signature may indicate that radical neck dissection is required. Modified radical neck dissection may comprise surgery to remove all the lymph nodes in one or both sides of the neck without removing the neck muscles. The nerves and/or the jugular vein may be removed. Partial neck dissection may comprise surgery to remove some of the lymph nodes in the neck. This is also called selective neck dissection. In certain embodiments, radical neck dissection, modified radical neck dissection, or partial neck dissection is used when a p-EMT signature as described herein is detected in a sample obtained from a subject in need thereof. In preferred embodiments, the sample is obtained from a primary tumor. Not being bound by a theory, detection of a p-EMT signature indicates that a partial neck dissection should be performed due to the high correlation to negative outcomes (e.g., metastasis) and absence of a p-EMT signature indicates that surgery may be delayed. In preferred embodiments, partial neck dissection is used when a p-EMT signature as described herein is detected in a sample obtained from a subject in need thereof. In other preferred embodiments, radical neck dissection or modified radical neck dissection is used instead of partial neck dissection when a p-EMT signature as described herein is detected in a sample obtained from a subject in need thereof. Not being bound by a theory, detection of a p-EMT signature indicates that the more aggressive choice of surgery should be selected. In certain embodiments, the type of neck dissection is performed based on the detection of a p-EMT signature. Not being bound by a theory, if the standard of care indicates a choice between an aggressive surgery and a less aggressive surgery, detection or lack of detection of a p-EMT signature may inform the choice between two options.

In certain embodiments, if a physician removes all of the cancer from a patient that can be seen at the time of surgery, some patients may be given radiation therapy after surgery to destroy any remaining cancer cells. Treatment given after surgery, to lower the risk that the cancer will come back, is called adjuvant therapy. Adjuvant therapy may comprise radiation or chemotherapy. Not being bound by a theory, detection of a p-EMT signature indicates that adjuvant therapy should be given and absence of a p-EMT signature indicates that further treatment may be delayed or reduced.

As used herein the term “radiation therapy” refers to a cancer treatment that uses high-energy x-rays or other types of radiation to kill cancer cells or keep them from growing. There are two types of radiation therapy. External radiation therapy uses a machine outside the body to send radiation toward the cancer. Certain ways of giving external radiation therapy can help keep radiation from damaging nearby healthy tissue. Intensity-modulated radiation therapy (IMRT) is a type of 3-dimensional (3-D) radiation therapy that uses a computer to make pictures of the size and shape of the tumor. Thin beams of radiation of different intensities (strengths) are aimed at the tumor from many angles. This type of radiation therapy is less likely to cause dry mouth, trouble swallowing, and damage to the skin. Intensity-modulated radiation therapy (IMRT) has become a standard technique for head and neck radiation therapy. IMRT allows a dose-painting technique also known as a simultaneous-integrated-boost (SIB) technique with a dose per fraction slightly higher than 2 Gy, which allows slight shortening of overall treatment time and increases the biologically equivalent dose to the tumor. Internal radiation therapy uses a radioactive substance sealed in needles, seeds, wires, or catheters that are placed directly into or near the cancer. In certain embodiments, an aggressive radiation therapy is used to treat HNSCC where a p-EMT signature is detected.

In certain embodiments, detection of a p-EMT signature is used to determine whether hyperfractionated radiation therapy is used. Hyperfractionated radiation therapy is a type of external radiation treatment in which a smaller than usual total daily dose of radiation is divided into two doses and the treatments are given twice a day. Hyperfractionated radiation therapy is given over the same period of time (days or weeks) as standard radiation therapy.

In addition to surgery and radiation, in certain embodiments detection of a p-EMT signature is used to determine whether chemotherapy should be administered. Chemotherapy is a cancer treatment that uses drugs to stop the growth of cancer cells, either by killing the cells or by stopping them from dividing. When chemotherapy is taken by mouth or injected into a vein or muscle, the drugs enter the bloodstream and can reach cancer cells throughout the body (systemic chemotherapy). When chemotherapy is placed directly into, e.g., the cerebrospinal fluid, an organ, or a body cavity such as the abdomen, the drugs mainly affect cancer cells in those areas (regional chemotherapy).

Treatment of HNSCC may include radiation therapy, surgery, radiation therapy followed by surgery, chemotherapy followed by radiation therapy, or chemotherapy given at the same time as hyperfractionated radiation therapy. Not being bound by a theory, radiation alone is the least aggressive treatment option, followed by surgery, radiation therapy followed by surgery, chemotherapy followed by radiation therapy, or chemotherapy given at the same time as hyperfractionated radiation therapy. Not being bound by a theory, detection of a p-EMT signature can guide the aggressiveness of a treatment to be administered to a subject in need thereof. In certain embodiments, combined-modality treatment is considered more aggressive treatment. When used in conjunction with surgery, radiation therapy is typically administered postoperatively, postoperative radiation treatment (PORT). Alternative strategies using neoadjuvant chemotherapy and radiation therapy may increase the chance for local control in selected advanced presentations to a level approaching that of resection and PORT. Neoadjuvant chemotherapy as given in clinical trials has been used to shrink tumors and render them more definitively treatable with either surgery or radiation. Chemotherapy is given before the other modalities, hence the designation, neoadjuvant, to distinguish it from standard adjuvant therapy, which is given after or during definitive therapy with radiation or after surgery. Many drug combinations have been used in neoadjuvant chemotherapy. Neoadjuvant chemotherapy is commonly used to treat patients who present with advanced disease to improve locoregional control or survival.

For locally advanced disease, concurrent chemoradiation approaches are superior to radiation therapy alone (Denis, et al., Final results of the 94-01 French Head and Neck Oncology and Radiotherapy Group randomized trial comparing radiotherapy alone with concomitant radiochemotherapy in advanced-stage oropharynx carcinoma. J Clin Oncol 22 (1): 69-76, 2004). This treatment approach emphasizes organ preservation and functionality.

Depending on pathological findings after primary surgery, PORT or postoperative chemoradiation is used in the adjuvant setting for the following histological findings including: T4 disease, Perineural invasion, Lymphovascular invasion, Positive margins or margins less than 5 mm, Extracapsular extension of a lymph node, Two or more involved lymph nodes. In certain embodiments, pathological findings may be combined with detection of a p-EMT signature to a treat a patient in need thereof with postoperative chemoradiation.

The benefit for overall survival has been demonstrated with postoperative chemoradiation therapy using cisplatin; an overall survival benefit has also been found for positive margins and extracapsular extension (Bernier J, et al.: Defining risk levels in locally advanced head and neck cancers: a comparative analysis of concurrent postoperative radiation plus chemotherapy trials of the EORTC (#22931) and RTOG (#9501). Head Neck 27 (10): 843-50, 2005; Cooper J S, et al.: Long-term follow-up of the RTOG 9501/intergroup phase III trial: postoperative concurrent radiation therapy and chemotherapy in high-risk squamous cell carcinoma of the head and neck. Int J Radiat Oncol Biol Phys 84 (5): 1198-205, 2012; Cooper J S, et al.: Postoperative concurrent radiotherapy and chemotherapy for high-risk squamous-cell carcinoma of the head and neck. N Engl J Med 350 (19): 1937-44, 2004; and Bernier J, et al.: Postoperative irradiation with or without concomitant chemotherapy for locally advanced head and neck cancer. N Engl J Med 350 (19): 1945-52, 2004). Not being bound by a theory, detection of a p-EMT signature may be used to select candidates for postoperative chemoradiation therapy.

The present invention, advantageously provides a p-EMT signature that positively correlates with the histological features of HNSCC and can be used to predict negative pathological features (e.g., extracapsular extension and lymphovascular invasion) (see, e.g., FIG. 14 H-J), which are clear indications for administering chemoradiation to a surgical intervention. Thus, the signature can predict which patients need chemotherapy and radiation and in some cases this may affect the decision to perform surgery in the first place. In one embodiment, surgery may not be performed and a patient may be first treated with a chemoradiation regimen.

In a randomized trial of locally advanced head and neck cancer patients, curative-intent radiation therapy alone (213 patients) was compared with radiation therapy plus weekly cetuximab (211 patients) (Bonner J A, Harari P M, Giralt J, et al.: Radiotherapy plus cetuximab for squamous-cell carcinoma of the head and neck. N Engl J Med 354 (6): 567-78, 2006). Cetuximab is an epidermal growth factor receptor (EGFR) inhibitor used for the treatment of metastatic colorectal cancer, metastatic non-small cell lung cancer and head and neck cancer. Cetuximab is a chimeric (mouse/human) monoclonal antibody given by intravenous infusion. The initial dose was 400 mg per square meter of body-surface area 1 week before starting radiation therapy followed by 250 mg per square meter weekly for the duration of the radiation therapy. At a median follow up of 54 months, patients treated with cetuximab and radiation therapy demonstrated significantly higher progression-free survival (hazard ratio for disease progression or death, 0.70; P=0.006). Patients in the cetuximab arm experienced higher rates of acneiform rash and infusion reactions, although the incidence of other grade 3 or higher toxicities, including mucositis, did not differ significantly between the two groups. In certain embodiments, radiation therapy plus weekly cetuximab may be administered before metastasis or locally advanced cancer is detected in patients positive for a p-EMT signature.

Aspects of the invention involve targeting proliferating cell types. In certain embodiments, targeting reduces the viability or reduces the invasiveness of p-EMT high cells comprised by the epithelial tumor. In one embodiment, the cells are killed or removed by targeting. In another embodiment, the cells no longer express a p-EMT signature. Not being bound by a theory, reducing the activity or inhibiting the expression of a p-EMT signature gene may cause loss of the p-EMT signature and improve prognosis. Targeting may be by use of small molecules, antibodies, antibody fragments, antibody like platforms and antibody drug conjugates. Targeting agents may include, but are not limited to single-chain immunotoxins reactive with human epithelial tumor cells. Antibody drug conjugates are well known in the art.

### Adoptive Cell Therapy

In certain embodiments, cells are targeted by using Adoptive cell therapy or Adoptive cell transfer (ACT). In certain embodiments, pathological features and detection of a p-EMT signature indicate that adoptive cell transfer may be used as a treatment. Adoptive cell therapy can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314 (5796) 126-9) has been used to successfully treat patients with advanced solid tumors, including melanoma and colorectal carcinoma, as well as patients with CD19-expressing hematologic malignancies (Kalos et al., (2011) Science Translational Medicine 3 (95): 95ra73). In one embodiment, ACT is performed before surgery or radiation therapy to shrink a tumor before primary treatment. In another embodiment ACT is performed after surgery or radiation to remove any remaining metastatic cancer cells. In one embodiment, transferred cells may be tumor infiltrating cells reactive to an epithelial tumor. In one embodiment, transferred cells may specifically target p-EMT high cells. Not being bound by a theory, ACT may eliminate or reduce cells having a p-EMT signature.

Aspects of the invention involve the adoptive transfer of immune system cells, such as T cells. In certain embodiments, immune cells are specific for cell surface markers present on cells having a p-EMT signature as described herein. The immune cells may be modified to express a chimeric antigen receptor specific for a marker. In other embodiments, cells specific for tumor cells having a p-EMT signature as described herein are activated and transferred to the patient. Immune cells may also be specific for selected antigens, such as tumor associated antigens or tumor specific neoantigens (see Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: B cell maturation antigen (BCMA); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostate; Prostatic acid phosphatase (PAP); elongation factor 2 mutant (ELF2M); Insulin-like growth factor 1 receptor (IGF-1R); gp1OO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDG1cp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDG1cp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRLS); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (Dl), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia. For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CIVIL), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1⁺ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto⁺ epithelial ovarian, fallopian tube or primary peritoneal cancer.

Various strategies may for example be employed to genetically modify T cells by altering the specificity of the T cell receptor (TCR) for example by introducing new TCR a and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a V_{L} linked to a V_{H} of a specific antibody, linked by a flexible linker, for example by a CD8α hinge domain and a CD8α transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, CD2, CD7, LIGHT, LFA-1, NKG2C, B7-H3, CD30, CD40, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon R1b), CD79a, CD79b, Fc gamma RIIa, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3): IEVMYPPPYLDNEKSNGTIIHVKGKHLCPSPLFPGPSKPFWVLVVVGGVLACYSLLVT VAFIIFWVRSKRSRLLHSDYMNMTPRRPGPTRKHYQPYAPPRDFAAYRS) (SEQ. I.D. No. 1). Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects.

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-ζ molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ. I.D. No. 2) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor a-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY and continuing all the way to the carboxy-terminus of the protein. The sequence is reproduced herein: IEVMYPPPYLDNEKSNGTIIHVKGKHLCPSPLFPGPSKPFWVLVVVGGVLACYSLLVT VAFIIFWVRSKRSRLLHSDYMNMTPRRPGPTRKHYQPYAPPRDFAAYRS. Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in WO2015187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signalling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcεRT gamma chain; or CD28-FcεRT gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signalling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with y-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR⁺ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In certain embodiments, ACT includes co-transferring CD4+ Th1 cells and CD8+ CTLs to induce a synergistic antitumour response (see, e.g., Li et al., Adoptive cell therapy with CD4+ T helper 1 cells and CD8+ cytotoxic T cells enhances complete rejection of an established tumour, leading to generation of endogenous memory responses to non-targeted tumour epitopes. Clin Transl Immunology. 2017 October; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4⁺ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13,2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment. The cells or population of cells, may be made resistant to at least one immunosuppressive agent due to the inactivation of a gene encoding a receptor for such immunosuppressive agent. Not being bound by a theory, the immunosuppressive treatment should help the selection and expansion of the immunoresponsive or T cells according to the invention within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2016, Multiplex genome editing to generate universal CAR T cells resistant to PD1 inhibition, Clin Cancer Res. 2016 Nov. 4; and Qasim et al., 2017, Molecular remission of infant B-ALL after infusion of universal TALEN gene-edited CAR T cells, Sci Transl Med. 2017 Jan. 25; 9(374)). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell; to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8⁺ T-cells (see PCT Patent Publications: WO2013176915, WO2014059173, WO2014172606, WO2014184744, and WO2014191128). Editing may result in inactivation of a gene.

By inactivating a gene it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci.

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as TIM-3, BTLA, LAG3, ICOS, PDL1 or KIR.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT2 inhibitors to increase proliferation and/or activity of exhausted CD8⁺ T-cells and to decrease CD8⁺ T-cell exhaustion (e.g., decrease functionally exhausted or unresponsive CD8⁺ immune cells). In certain embodiments, metallothioneins are targeted by gene editing in adoptively transferred T cells.

In certain embodiments, editing of cells (such as by CRISPR/Cas), particularly cells intended for adoptive cell therapies, more particularly immunoresponsive cells such as T cells, may be performed to enhance or maintain expression of co-stimulatory receptors (co-stimulatory immune checkpoint molecule), such as a member of the TNFR superfamily including, but not limited to CD40, OX40, CD137 (4-1BB), GITR or CD27.

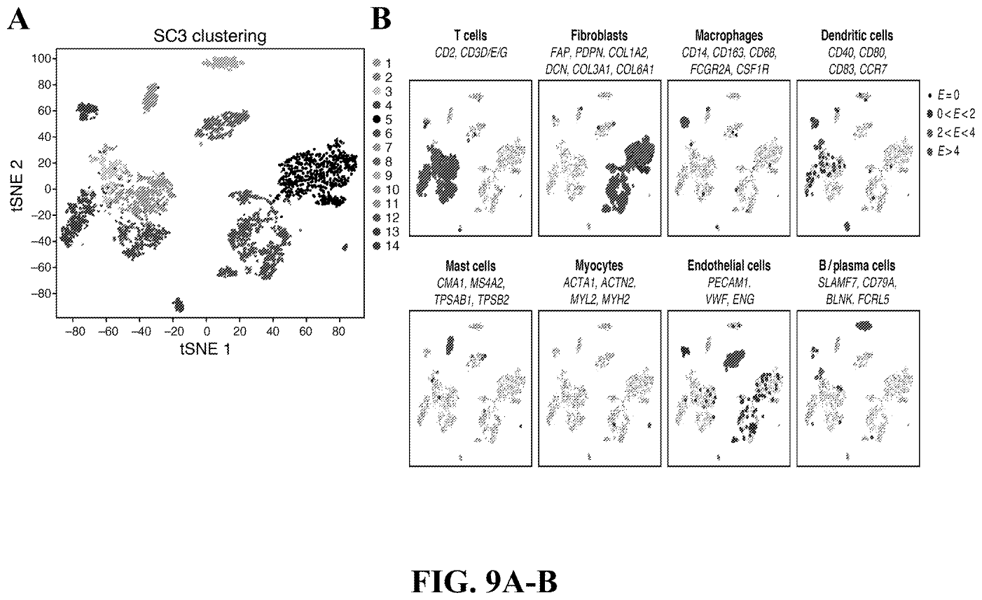

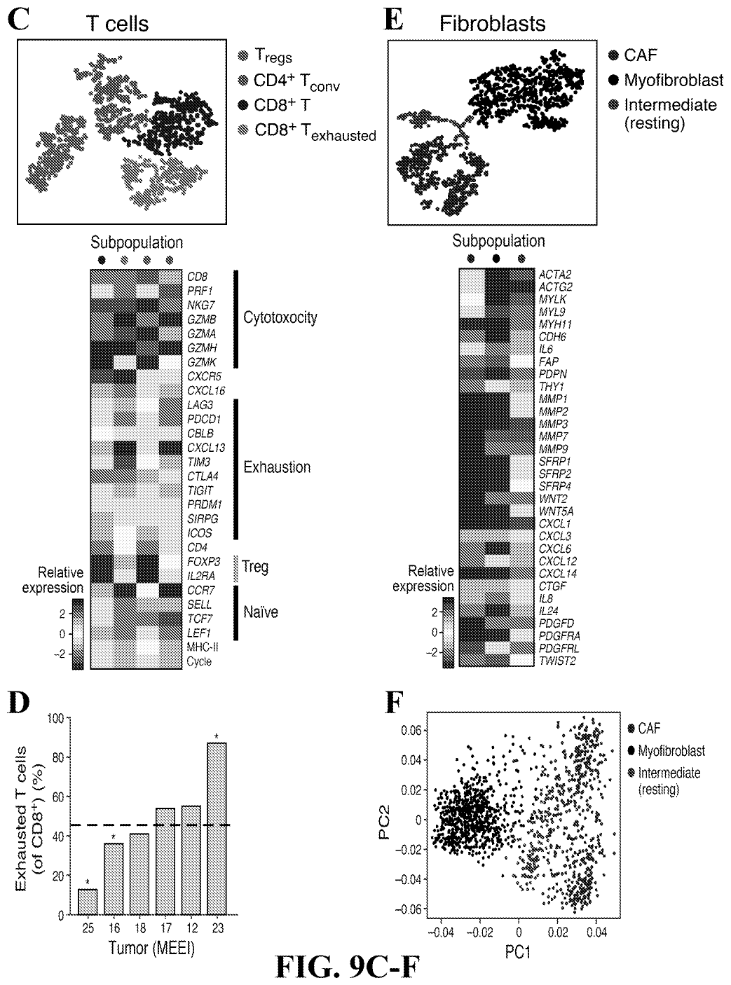

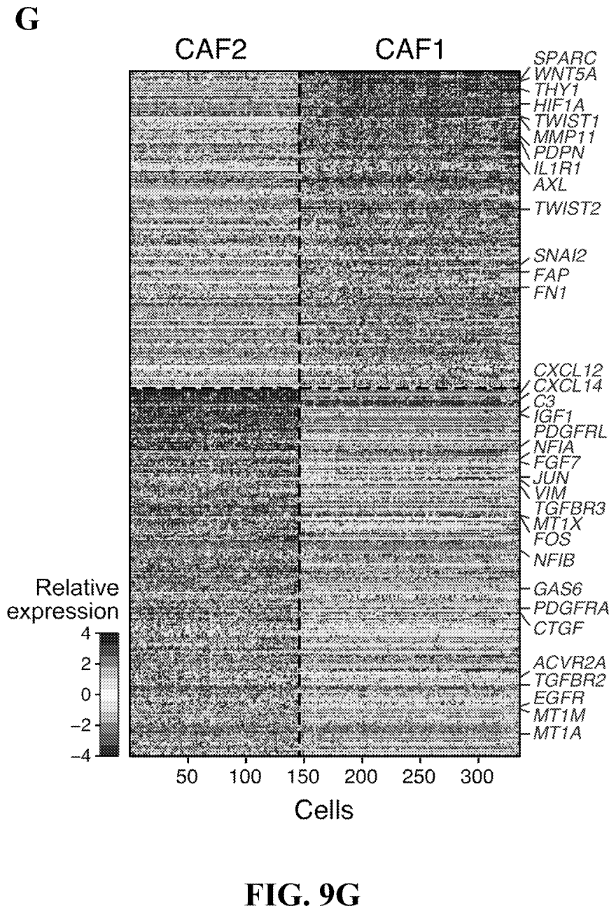

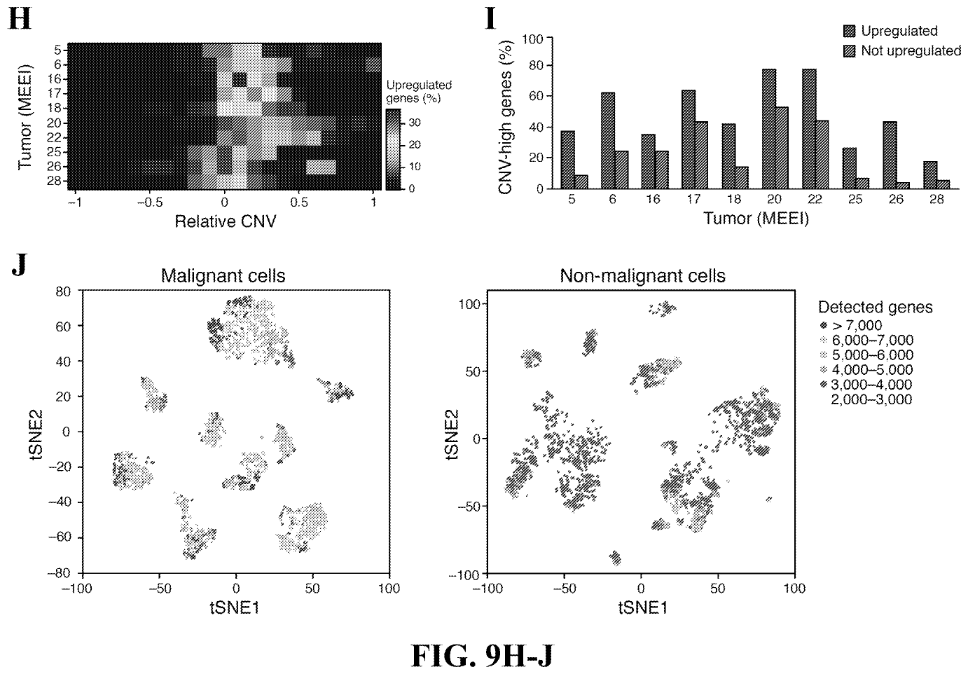

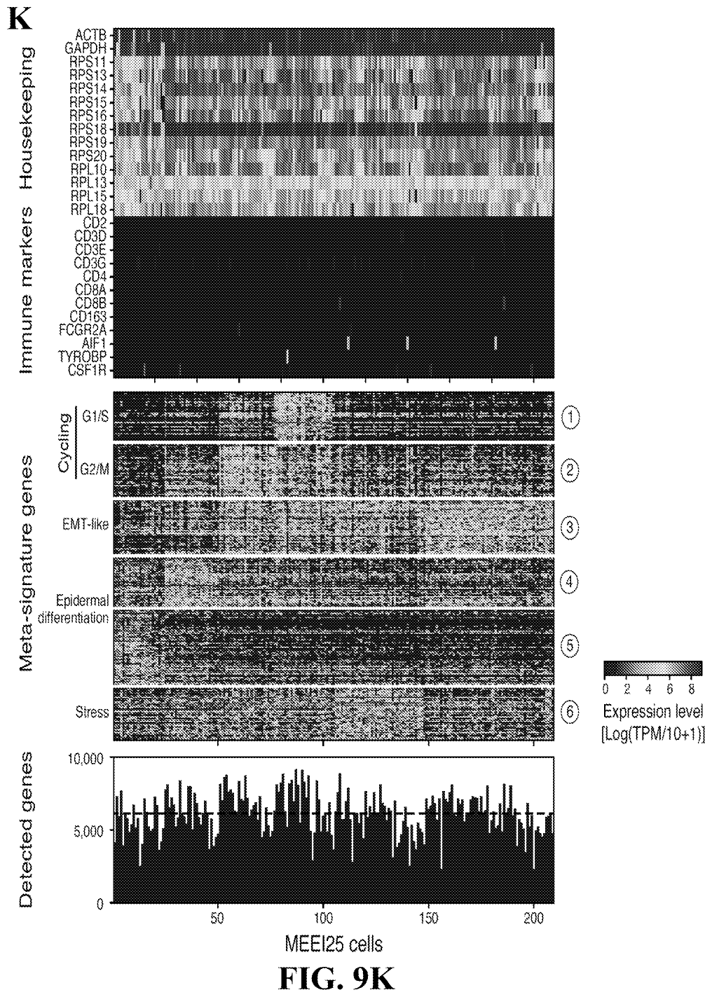

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT. In other preferred embodiments, HNSCC specific T-cell exhaustion markers are targeted (see, e.g., FIG. 9C).

By means of an example and without limitation, WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MEW constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment T cells that have infiltrated a tumor are isolated. T cells may be removed during surgery. T cells may be isolated after removal of tumor tissue by biopsy. T cells may be isolated by any means known in the art. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells, bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient.

A specific subpopulation of T cells can be further isolated by positive or negative selection techniques. For example, in one preferred embodiment, T cells are isolated by incubation with antibody-conjugated beads (e.g., specific for any marker described herein), such as DYNABEADS® for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8⁺ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected.

Further, monocyte populations (i.e., CD14⁺ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest or from samples where there are many tumor cells present (i.e., leukemic blood, tumor tissue, etc). Such populations of cells may have therapeutic value and would be desirable to obtain.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. In one embodiment, the concentration of cells used is 5×10⁶/ml. In other embodiments, the concentration used can be from about 1×10⁵/ml to 1×10⁶/ml, and any integer value in between.

In certain embodiments, T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274(5284):94-6). In another embodiment the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MEW molecule of interest and any antigen of interest as described herein. Specific epitopes to be used in this context can be identified using numerous assays known in the art. For example, the ability of a polypeptide to bind to MEW class I may be evaluated indirectly by monitoring the ability to promote incorporation of ¹²⁵I labeled β2-microglobulin (β2m) into MEW class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one T cells are isolated by contacting the T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in WO2016191756 comprising administering to the patient a dose of cyclophosphamide between 200 mg/m²/day and 2000 mg/m²/day and a dose of fludarabine between 20 mg/m²/day and 900 mg/m²/day.

### Therapeutic Agents and Formulations

Therapeutic formulations of the invention, which includes an agent that is capable of reducing the expression or inhibiting the activity of one or more p-EMT signature genes or polypeptides, a T cell modulating agent, targeted therapies and checkpoint inhibitors, are used to treat or alleviate a symptom associated with a cancer. An agent that is capable of reducing the expression or inhibiting the activity of one or more p-EMT signature genes may include, but is not limited to antisense oligonucleotides, shRNAs, RNAi, microRNAs, a CRISPR system, a therapeutic protein, therapeutic antibody, or small molecule. The present invention also provides methods of treating or alleviating a symptom associated with cancer. A therapeutic regimen is carried out by identifying a subject, e.g., a human patient suffering from an epithelial cancer, using standard methods in combination with the methods of using the p-EMT signature as described herein.

In certain embodiments, agents capable of modulating expression of the p-EMT signature are identified by signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature really was the proxy for a phenotype of interest, it could be used to find small molecules that effect that phenotype without knowledge of a validated drug target. The p-EMT signature of the present invention may be used to screen for drugs that reduce the signature in cancer cells or cell lines.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, cmap can be used to screen for agents capable of modulating the p-EMT signature in silico.

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The medicaments of the invention are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of a disease (e.g., metastatic disease). The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

### Perturb-seq

Previously developed methods and tools for genome-scale screening of perturbations in single cells using CRISPR-Cas9, herein referred to as Perturb-seq, may be used to determine networks regulating or disrupted in cells expressing a p-EMT signature (see e.g., Dixit et al., “Perturb-Seq: Dissecting Molecular Circuits with Scalable Single-Cell RNA Profiling of Pooled Genetic Screens” 2016, Cell 167, 1853-1866; Adamson et al., “A Multiplexed Single-Cell CRISPR Screening Platform Enables Systematic Dissection of the Unfolded Protein Response” 2016, Cell 167, 1867-1882; and International publication serial number WO/2017/075294). The present invention is compatible with Perturb-seq, such that signature genes may be perturbed and the perturbation may be identified and assigned to the gene expression readouts of single cells.

The perturbation methods and tools allow reconstructing of a cellular network or circuit. In one embodiment, the method comprises (1) introducing single-order or combinatorial perturbations to a population of cells, (2) measuring genomic, genetic, proteomic, epigenetic and/or phenotypic differences in single cells and (3) assigning a perturbation(s) to the single cells. Not being bound by a theory, a perturbation may be linked to a phenotypic change, preferably changes in gene or protein expression. In preferred embodiments, measured differences that are relevant to the perturbations are determined by applying a model accounting for co-variates to the measured differences. The model may include the capture rate of measured signals, whether the perturbation actually perturbed the cell (phenotypic impact), the presence of subpopulations of either different cells or cell states, and/or analysis of matched cells without any perturbation. In certain embodiments, the measuring of phenotypic differences and assigning a perturbation to a single cell is determined by performing single cell RNA sequencing (RNA-seq). In preferred embodiments, the single cell RNA-seq is performed as described herein. In certain embodiments, unique barcodes are used to perform Perturb-seq. In certain embodiments, a guide RNA is detected by RNA-seq using a transcript expressed from a vector encoding the guide RNA. The transcript may include a unique barcode specific to the guide RNA. Not being bound by a theory, a guide RNA and guide RNA barcode is expressed from the same vector and the barcode may be detected by RNA-seq. Not being bound by a theory, detection of a guide RNA barcode is more reliable than detecting a guide RNA sequence and reduces the chance of false guide RNA assignment. Thus, a perturbation may be assigned to a single cell by detection of a guide RNA barcode in the cell. In certain embodiments, a cell barcode is added to the RNA in single cells, such that the RNA may be assigned to a single cell. Generating cell barcodes is described herein. In certain embodiments, a Unique Molecular Identifier (UMI) is added to each individual transcript and protein capture oligonucleotide. Not being bound by a theory, the UMI allows for determining the capture rate of measured signals, or preferably the binding events or the number of transcripts captured. Not being bound by a theory, the data is more significant if the signal observed is derived from more than one protein binding event or transcript. In preferred embodiments, Perturb-seq is performed using a guide RNA barcode expressed as a polyadenylated transcript, a cell barcode, and a UMI.

Perturb-seq combines emerging technologies in the field of genome engineering, and single-cell analysis, in particular the CRISPR-Cas9 system and droplet single-cell sequencing analysis. In certain embodiments, a CRISPR system is used to create an INDEL at a target gene. In other embodiments, epigenetic screening is performed by applying CRISPRa/i/x technology (see, e.g., Konermann et al. “Genome-scale transcriptional activation by an engineered CRISPR-Cas9 complex” Nature. 2014 Dec. 10. doi: 10.1038/nature14136; Qi, L. S., et al. (2013). “Repurposing CRISPR as an RNA-guided platform for sequence-specific control of gene expression”. Cell. 152 (5): 1173-83; Gilbert, L. A., et al., (2013). “CRISPR-mediated modular RNA-guided regulation of transcription in eukaryotes”. Cell. 154 (2): 442-51; Komor et al., 2016, Programmable editing of a target base in genomic DNA without double-stranded DNA cleavage, Nature 533, 420-424; Nishida et al., 2016, Targeted nucleotide editing using hybrid prokaryotic and vertebrate adaptive immune systems, Science 353(6305); Yang et al., 2016, Engineering and optimising deaminase fusions for genome editing, Nat Commun. 7:13330; Hess et al., 2016, Directed evolution using dCas9-targeted somatic hypermutation in mammalian cells, Nature Methods 13, 1036-1042; and Ma et al., 2016, Targeted AID-mediated mutagenesis (TAM) enables efficient genomic diversification in mammalian cells, Nature Methods 13, 1029-1035). Numerous genetic variants associated with disease phenotypes are found to be in non-coding region of the genome, and frequently coincide with transcription factor (TF) binding sites and non-coding RNA genes. Not being bound by a theory, CRISPRa/i/x approaches may be used to achieve a more thorough and precise understanding of the implication of epigenetic regulation.

In certain embodiments, whole genome screens can be used for understanding the phenotypic readout of perturbing potential target genes. In preferred embodiments, perturbations target expressed genes as defined by RNA-seq or the signature described herein using a focused sgRNA library. Libraries may be focused on expressed genes in specific networks or pathways (e.g. p-EMT signature). Not being bound by a theory, this approach will accelerate the development of therapeutics for human disorders, in particular cancer.

### Genetic Modifying Agents

In certain embodiments, the one or more modulating agents may be a genetic modifying agent. The genetic modifying agent may comprise a CRISPR system, a zinc finger nuclease system, a TALEN, or a meganuclease.

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein H is A, C or U.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

In certain example embodiments, the CRISPR effector protein may be delivered using a nucleic acid molecule encoding the CRISPR effector protein. The nucleic acid molecule encoding a CRISPR effector protein, may advantageously be a codon optimized CRISPR effector protein. An example of a codon optimized sequence, is in this instance a sequence optimized for expression in eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words samples comprising a masking construct may be delivered to a cell, for example in a suitable delivery vesicle and if the target is present in the delivery vesicle the CRISPR effector is activated and a detectable signal generated.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector, is to express an array of promoter-RNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53. short and nature.com/mt/journal/v16/n9/abs/mt2008144a.html). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters-especially as to the numbers of RNAs or guides discussed herein, without any undue experimentation.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

Additional effectors for use according to the invention can be identified by their proximity to cas1 genes, for example, though not limited to, within the region 20 kb from the start of the cas1 gene and 20 kb from the end of the cas1 gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the C2c2 effector protein is naturally present in a prokaryotic genome within 20kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related.

### Guide Molecules

The methods described herein may be used to screen inhibition of CRISPR systems employing different types of guide molecules. As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23-25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In some embodiments, it is of interest to reduce the susceptibility of the guide molecule to RNA cleavage, such as to cleavage by Cas13. Accordingly, in particular embodiments, the guide molecule is adjusted to avoide cleavage by Cas13 or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′ phosphorothioate (MS), S-constrained ethyl(cEt), or 2′-O-methyl 3′ thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucletides and/or nucleotide analogs in a region that binds to Cas13. In an embodiment of the invention, deoxyribonucleotides and/or nucleotide analogs are incorporated in engineered guide structures, such as, without limitation, stem-loop regions, and the seed region. For Cas13 guide, in certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (me1Ψ), 5-methoxyuridine(5moU), inosine, 7-methylguanosine, 2′-O-methyl 3′phosphorothioate (MS), S-constrained ethyl(cEt), phosphorothioate (PS), or 2′-O-methyl 3′thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 to 10 nucleotides in the 3′-terminus are chemically modified. Such chemical modifications at the 3′-terminus of the Cas13 CrRNA may improve Cas13 activity. In a specific embodiment, 1, 2, 3, 4, 5, 6, 7, 8, 9 or 10 nucleotides in the 3′-terminus are replaced with 2′-fluoro analogues. In a specific embodiment, 1, 2, 3, 4, 5, 6, 7, 8, 9 or 10 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the modified loop comprises 3, 4, or 5 nucleotides. In certain embodiments, the loop comprises the sequence of UCUU, UUUU, UAUU, or UGUU.

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) from the guide sequence. In a particular embodiment the seed sequence (i.e. the sequence essential critical for recognition and/or hybridization to the sequence at the target locus) of th guide sequence is approximately within the first 10 nucleotides of the guide sequence.

In a particular embodiment the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments the guide molecule comprises or consists of the guide sequence linked to all or part of the natural direct repeat sequence. A typical Type V or Type VI CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g. an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments the natural hairpin or stemloop structure of the guide molecule is extended or replaced by an extended stemloop. It has been demonstrated that extension of the stem can enhance the assembly of the guide molecule with the CRISPR-Cas proten (Chen et al. Cell. (2013); 155(7): 1479-1491). In particular embodiments the stem of the stemloop is extended by at least 1, 2, 3, 4, 5 or more complementary basepairs (i.e. corresponding to the addition of 2, 4, 6, 8, 10 or more nucleotides in the guide molecule). In particular embodiments these are located at the end of the stem, adjacent to the loop of the stemloop.

In particular embodiments, the susceptibility of the guide molecule to RNAses or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA may be selected to target any target nucleic acid sequence. The target sequence may be mRNA.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments of the present invention where the CRISPR-Cas protein is a Cas13 protein, the complementary sequence of the target sequence is downstream or 3′ of the PAM or upstream or 5′ of the PAM. The precise sequence and length requirements for the PAM differ depending on the Cas13 protein used, but PAMs are typically 2-5 base pair sequences adjacent the protospacer (that is, the target sequence). Examples of the natural PAM sequences for different Cas13 orthologues are provided herein below and the skilled person will be able to identify further PAM sequences for use with a given Cas13 protein.

Further, engineering of the PAM Interacting (PI) domain may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously.

In particular embodiment, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green flourescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends an guide molecule that responds to normal or pathological physiological conditions, including without limitation pH, hypoxia, O₂ concentration, temperature, protein concentration, enzymatic concentration, lipid structure, light exposure, mechanical disruption (e.g. ultrasound waves), magnetic fields, electric fields, or electromagnetic radiation.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm². In a preferred embodiment, a stimulation paradigm of as low as 0.25 sec every 15 sec should result in maximal activation.

The chemical or energy sensitive guide may undergo a conformational change upon induction by the binding of a chemical source or by the energy allowing it act as a guide and have the Cas13 CRISPR-Cas system or complex function. The invention can involve applying the chemical source or energy so as to have the guide function and the Cas13 CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans; 4/164/r52), 2. FKBP-FRB based system inducible by rapamycin (or related chemicals based on rapamycin) (see, e.g., www.nature.com/nmeth/journal/v2/n6/full/nmeth763.html), 3. GID1-GAI based system inducible by Gibberellin (GA) (see, e.g., www.nature.com/nchembio/journal/v8/n5/full/nchembio.922.html).

A chemical inducible system can be an estrogen receptor (ER) based system inducible by 4-hydroxytamoxifen (4OHT) (see, e.g., www.pnas.org/content/104/3/1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogren receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the Cas13 CRISPR-Cas complex or system, and the binding will induce the change of sub-cellular localization of the polypeptide, leading to the entire polypeptide entering the nucleus of cells. Once inside the nucleus, the guide protein and the other components of the Cas13 CRISPR-Cas complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc, as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100·mu·s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz′ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm−2. Even more preferably, the exposure to an ultrasound energy source is at a power density of from about 1 to about 15 Wcm−2.

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm−2 to about 10 Wcm−2 with a frequency ranging from about 0.015 to about 10 MHz (see WO 98/52609). However, alternatives are also possible, for example, exposure to an ultrasound energy source at an acoustic power density of above 100 Wcm−2, but for reduced periods of time, for example, 1000 Wcm−2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm−2 or 1.25 Wcm−2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extended length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide molecule, the guide molecule is considered protected and results in improved specific binding of the CRISPR-Cas complex, while maintaining specific activity.

In particular embodiments, use is made of a truncated guide (tru-guide), i.e. a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

### CRISPR RNA-Targeting Effector Proteins

In one example embodiment, the CRISPR system effector protein is an RNA-targeting effector protein. In certain embodiments, the CRISPR system effector protein is a Type VI CRISPR system targeting RNA (e.g., Cas13a, Cas13b, Cas13c or Cas13d). Example RNA-targeting effector proteins include Cas13b and C2c2 (now known as Cas13a). It will be understood that the term “C2c2” herein is used interchangeably with “Cas13a”. “C2c2” is now referred to as “Cas13a”, and the terms are used interchangeably herein unless indicated otherwise. As used herein, the term “Cas13” refers to any Type VI CRISPR system targeting RNA (e.g., Cas13a, Cas13b, Cas13c or Cas13d). When the CRISPR protein is a C2c2 protein, a tracrRNA is not required. C2c2 has been described in Abudayyeh et al. (2016) “C2c2 is a single-component programmable RNA-guided RNA-targeting CRISPR effector”; Science; DOI: 10.1126/science.aaf5573; and Shmakov et al. (2015) “Discovery and Functional Characterization of Diverse Class 2 CRISPR-Cas Systems”, Molecular Cell, DOI: dx.doi.org/10. 1016/j.molcel.2015.10.008; which are incorporated herein in their entirety by reference. Cas13b has been described in Smargon et al. (2017) “Cas13b Is a Type VI-B CRISPR-Associated RNA-Guided RNases Differentially Regulated by Accessory Proteins Csx27 and Csx28,” Molecular Cell. 65, 1-13; dx.doi.org/10.1016/j.molcel.2016.12.023., which is incorporated herein in its entirety by reference.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of C2c2 or Cas13b orthologs provided herein. In certain example embodiments, the effector protein comprises a single HEPN domain. In certain other example embodiments, the effector protein comprises two HEPN domains.

In one example embodiment, the effector protein comprise one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application entitled “Novel Type VI CRISPR Orthologs and Systems,” labeled as attorney docket number 47627-05-2133 and filed on Apr. 12, 2017.

In certain other example embodiments, the CRISPR system effector protein is a C2c2 nuclease. The activity of C2c2 may depend on the presence of two HEPN domains. These have been shown to be RNase domains, i.e. nuclease (in particular an endonuclease) cutting RNA. C2c2 HEPN may also target DNA, or potentially DNA and/or RNA. On the basis that the HEPN domains of C2c2 are at least capable of binding to and, in their wild-type form, cutting RNA, then it is preferred that the C2c2 effector protein has RNase function. Regarding C2c2 CRISPR systems, reference is made to U.S. Provisional 62/351,662 filed on Jun. 17, 2016 and U.S. Provisional 62/376,377 filed on Aug. 17, 2016. Reference is also made to U.S. Provisional 62/351,803 filed on Jun. 17, 2016. Reference is also made to U.S. Provisional entitled “Novel Crispr Enzymes and Systems” filed Dec. 8, 2016 bearing Broad Institute No. 10035.PA4 and Attorney Docket No. 47627.03.2133. Reference is further made to East-Seletsky et al. “Two distinct RNase activities of CRISPR-C2c2 enable guide-RNA processing and RNA detection” Nature doi:10/1038/nature19802 and Abudayyeh et al. “C2c2 is a single-component programmable RNA-guided RNA targeting CRISPR effector” bioRxiv doi:10.1101/054742.

In certain embodiments, the C2c2 effector protein is from an organism of a genus selected from the group consisting of: Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira, or the C2c2 effector protein is an organism selected from the group consisting of: Leptotrichia shahii, Leptotrichia. wadei, Listeria seeligeri, Clostridium aminophilum, Carnobacterium gallinarum, Paludibacter propionicigenes, Listeria weihenstephanensis, or the C2c2 effector protein is a L. wadei F0279 or L. wadei F0279 (Lw2) C2C2 effector protein. In another embodiment, the one or more guide RNAs are designed to detect a single nucleotide polymorphism, splice variant of a transcript, or a frameshift mutation in a target RNA or DNA.

In certain example embodiments, the RNA-targeting effector protein is a Type VI-B effector protein, such as Cas13b and Group 29 or Group 30 proteins. In certain example embodiments, the RNA-targeting effector protein comprises one or more HEPN domains. In certain example embodiments, the RNA-targeting effector protein comprises a C-terminal HEPN domain, a N-terminal HEPN domain, or both. Regarding example Type VI-B effector proteins that may be used in the context of this invention, reference is made to U.S. application Ser. No. 15/331,792 entitled “Novel CRISPR Enzymes and Systems” and filed Oct. 21, 2016, International Patent Application No. PCT/US2016/058302 entitled “Novel CRISPR Enzymes and Systems”, and filed Oct. 21, 2016, and Smargon et al. “Cas13b is a Type VI-B CRISPR-associated RNA-Guided RNase differentially regulated by accessory proteins Csx27 and Csx28” Molecular Cell, 65, 1-13 (2017); dx.doi.org/10.1016/j.molcel.2016.12.023, and U.S. Provisional Application No. to be assigned, entitled “Novel Cas13b Orthologues CRISPR Enzymes and System” filed Mar. 15, 2017. In particular embodiments, the Cas13b enzyme is derived from Bergeyella zoohelcum.

In certain example embodiments, the RNA-targeting effector protein is a Cas13c effector protein as disclosed in U.S. Provisional Patent Application No. 62/525,165 filed Jun. 26, 2017, and PCT Application No. US 2017/047193 filed Aug. 16, 2017.

In some embodiments, one or more elements of a nucleic acid-targeting system is derived from a particular organism comprising an endogenous CRISPR RNA-targeting system. In certain embodiments, the CRISPR RNA-targeting system is found in Eubacterium and Ruminococcus. In certain embodiments, the effector protein comprises targeted and collateral ssRNA cleavage activity. In certain embodiments, the effector protein comprises dual HEPN domains. In certain embodiments, the effector protein lacks a counterpart to the Helical-1 domain of Cas13a. In certain embodiments, the effector protein is smaller than previously characterized class 2 CRISPR effectors, with a median size of 928 aa. This median size is 190 aa (17%) less than that of Cas13c, more than 200 aa (18%) less than that of Cas13b, and more than 300 aa (26%) less than that of Cas13a. In certain embodiments, the effector protein has no requirement for a flanking sequence (e.g., PFS, PAM).

In certain embodiments, the effector protein locus structures include a WYL domain containing accessory protein (so denoted after three amino acids that were conserved in the originally identified group of these domains; see, e.g., WYL domain IPR026881). In certain embodiments, the WYL domain accessory protein comprises at least one helix-turn-helix (HTH) or ribbon-helix-helix (RHH) DNA-binding domain. In certain embodiments, the WYL domain containing accessory protein increases both the targeted and the collateral ssRNA cleavage activity of the RNA-targeting effector protein. In certain embodiments, the WYL domain containing accessory protein comprises an N-terminal RHH domain, as well as a pattern of primarily hydrophobic conserved residues, including an invariant tyrosine-leucine doublet corresponding to the original WYL motif. In certain embodiments, the WYL domain containing accessory protein is WYL1. WYL1 is a single WYL-domain protein associated primarily with Ruminococcus.

In other example embodiments, the Type VI RNA-targeting Cas enzyme is Cas13d. In certain embodiments, Cas13d is Eubacterium siraeum DSM 15702 (EsCas13d) or Ruminococcus sp. N15.MGS-57 (RspCas13d) (see, e.g., Yan et al., Cas13d Is a Compact RNA-Targeting Type VI CRISPR Effector Positively Modulated by a WYL-Domain-Containing Accessory Protein, Molecular Cell (2018), doi.org/10.1016/j.molcel.2018.02.028). RspCas13d and EsCas13d have no flanking sequence requirements (e.g., PFS, PAM).

### Cas13 RNA Editing

In one aspect, the invention provides a method of modifying or editing a target transcript in a eukaryotic cell. In some embodiments, the method comprises allowing a CRISPR-Cas effector module complex to bind to the target polynucleotide to effect RNA base editing, wherein the CRISPR-Cas effector module complex comprises a Cas effector module complexed with a guide sequence hybridized to a target sequence within said target polynucleotide, wherein said guide sequence is linked to a direct repeat sequence. In some embodiments, the Cas effector module comprises a catalytically inactive CRISPR-Cas protein. In some embodiments, the guide sequence is designed to introduce one or more mismatches to the RNA/RNA duplex formed between the target sequence and the guide sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytindine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

The present application relates to modifying a target RNA sequence of interest (see, e.g, Cox et al., Science. 2017 Nov. 24; 358(6366):1019-1027). Using RNA-targeting rather than DNA targeting offers several advantages relevant for therapeutic development. First, there are substantial safety benefits to targeting RNA: there will be fewer off-target events because the available sequence space in the transcriptome is significantly smaller than the genome, and if an off-target event does occur, it will be transient and less likely to induce negative side effects. Second, RNA-targeting therapeutics will be more efficient because they are cell-type independent and not have to enter the nucleus, making them easier to deliver.

A further aspect of the invention relates to the method and composition as envisaged herein for use in prophylactic or therapeutic treatment, preferably wherein said target locus of interest is within a human or animal and to methods of modifying an Adenine or Cytidine in a target RNA sequence of interest, comprising delivering to said target RNA, the composition as described herein. In particular embodiments, the CRISPR system and the adenonsine deaminase, or catalytic domain thereof, are delivered as one or more polynucleotide molecules, as a ribonucleoprotein complex, optionally via particles, vesicles, or one or more viral vectors. In particular embodiments, the invention thus comprises compositions for use in therapy. This implies that the methods can be performed in vivo, ex vivo or in vitro. In particular embodiments, when the target is a human or animal target, the method is carried out ex vivo or in vitro.

A further aspect of the invention relates to the method as envisaged herein for use in prophylactic or therapeutic treatment, preferably wherein said target of interest is within a human or animal and to methods of modifying an Adenine or Cytidine in a target RNA sequence of interest, comprising delivering to said target RNA, the composition as described herein. In particular embodiments, the CRISPR system and the adenonsine deaminase, or catalytic domain thereof, are delivered as one or more polynucleotide molecules, as a ribonucleoprotein complex, optionally via particles, vesicles, or one or more viral vectors.

In one aspect, the invention provides a method of generating a eukaryotic cell comprising a modified or edited gene. In some embodiments, the method comprises (a) introducing one or more vectors into a eukaryotic cell, wherein the one or more vectors drive expression of one or more of: Cas effector module, and a guide sequence linked to a direct repeat sequence, wherein the Cas effector module associate one or more effector domains that mediate base editing, and (b) allowing a CRISPR-Cas effector module complex to bind to a target polynucleotide to effect base editing of the target polynucleotide within said disease gene, wherein the CRISPR-Cas effector module complex comprises a Cas effector module complexed with the guide sequence that is hybridized to the target sequence within the target polynucleotide, wherein the guide sequence may be designed to introduce one or more mismatches between the RNA/RNA duplex formed between the guide sequence and the target sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytidine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

A further aspect relates to an isolated cell obtained or obtainable from the methods described herein comprising the composition described herein or progeny of said modified cell, preferably wherein said cell comprises a hypoxanthine or a guanine in replace of said Adenine in said target RNA of interest compared to a corresponding cell not subjected to the method. In particular embodiments, the cell is a eukaryotic cell, preferably a human or non-human animal cell, optionally a therapeutic T cell or an antibody-producing B-cell.

In some embodiments, the modified cell is a therapeutic T cell, such as a T cell suitable for adoptive cell transfer therapies (e.g., CAR-T therapies). The modification may result in one or more desirable traits in the therapeutic T cell, as described further herein.

The invention further relates to a method for cell therapy, comprising administering to a patient in need thereof the modified cell described herein, wherein the presence of the modified cell remedies a disease in the patient. In one embodiment, the modified cell for cell therapy is a CAR-T cell capable of recognizing and/or attacking a tumor cell.

The present invention may be further illustrated and extended based on aspects of CRISPR-Cas development and use as set forth in the following articles and particularly as relates to delivery of a CRISPR protein complex and uses of an RNA guided endonuclease in cells and organisms:
Multiplex genome engineering using CRISPR-Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR-Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13) 01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013);
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR-Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546): 186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR-Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
BCL11A enhancer dissection by Cas9-mediated in situ saturating mutagenesis, Canver et al., Nature 527(7577):192-7 (Nov. 12, 2015) doi: 10.1038/nature15521. Epub 2015 Sep. 16.
Cpf1 Is a Single RNA-Guided Endonuclease of a Class 2 CRISPR-Cas System, Zetsche et al., Cell 163, 759-71 (Sep. 25, 2015).
Discovery and Functional Characterization of Diverse Class 2 CRISPR-Cas Systems, Shmakov et al., Molecular Cell, 60(3), 385-397 doi: 10.1016/j.molcel.2015.10.008 Epub Oct. 22, 2015.
Rationally engineered Cas9 nucleases with improved specificity, Slaymaker et al., Science 2016 Jan. 1 351(6268): 84-88 doi: 10.1126/science.aad5227. Epub 2015 Dec. 1.
Gao et al, “Engineered Cpf1 Enzymes with Altered PAM Specificities,” bioRxiv 091611; doi: http://dx.doi.org/10.1101/091611 (Dec. 4, 2016).
Cox et al., “RNA editing with CRISPR-Cas13,” Science. 2017 Nov. 24; 358(6366):1019-1027. doi: 10.1126/science.aaq0180. Epub 2017 Oct. 25.

each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR-Cas system for the one-step generation of mice carrying mutations in multiple genes which were traditionally generated in multiple steps by sequential recombination in embryonic stem cells and/or time-consuming intercrossing of mice with a single mutation. The CRISPR-Cas system will greatly accelerate the in vivo study of functionally redundant genes and of epistatic gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and guide RNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays.
Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR-Cas9 knockout and nucleotide preference at the cleavage site. The authors also found that the sequence preference for CRISPRi/a is substantially different from that for CRISPR-Cas9 knockout.

Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.
Canver et al. (2015) demonstrated a CRISPR-Cas9-based functional investigation of non-coding genomic elements. The authors we developed pooled CRISPR-Cas9 guide RNA libraries to perform in situ saturating mutagenesis of the human and mouse BCL11A enhancers which revealed critical features of the enhancers.
Zetsche et al. (2015) reported characterization of Cpf1, a class 2 CRISPR nuclease from Francisella novicida U112 having features distinct from Cas9. Cpf1 is a single RNA-guided endonuclease lacking tracrRNA, utilizes a T-rich protospacer-adjacent motif, and cleaves DNA via a staggered DNA double-stranded break.
Shmakov et al. (2015) reported three distinct Class 2 CRISPR-Cas systems. Two system CRISPR enzymes (C2c1 and C2c3) contain RuvC-like endonuclease domains distantly related to Cpf1. Unlike Cpf1, C2c1 depends on both crRNA and tracrRNA for DNA cleavage. The third enzyme (C2c2) contains two predicted HEPN RNase domains and is tracrRNA independent.
Slaymaker et al (2016) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.
Cox et al., (2017) reported the use of catalytically inactive Cas13 (dCas13) to direct adenosine-to-inosine deaminase activity by ADAR2 (adenosine deaminase acting on RNA type 2) to transcripts in mammalian cells. The system, referred to as RNA Editing for Programmable A to I Replacement (REPAIR), has no strict sequence constraints and can be used to edit full-length transcripts. The authors further engineered the system to create a high-specificity variant and minimized the system to facilitate viral delivery.

The methods and tools provided herein are may be designed for use with or Cas13, a type II nuclease that does not make use of tracrRNA. Orthologs of Cas13 have been identified in different bacterial species as described herein. Further type II nucleases with similar properties can be identified using methods described in the art (Shmakov et al. 2015, 60:385-397; Abudayeh et al. 2016, Science, 5; 353(6299)). In particular embodiments, such methods for identifying novel CRISPR effector proteins may comprise the steps of selecting sequences from the database encoding a seed which identifies the presence of a CRISPR Cas locus, identifying loci located within 10 kb of the seed comprising Open Reading Frames (ORFs) in the selected sequences, selecting therefrom loci comprising ORFs of which only a single ORF encodes a novel CRISPR effector having greater than 700 amino acids and no more than 90% homology to a known CRISPR effector. In particular embodiments, the seed is a protein that is common to the CRISPR-Cas system, such as Cas1. In further embodiments, the CRISPR array is used as a seed to identify new effector proteins.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells.

With respect to general information on CRISPR/Cas Systems, components thereof, and delivery of such components, including methods, materials, delivery vehicles, vectors, particles, and making and using thereof, including as to amounts and formulations, as well as CRISPR-Cas-expressing eukaryotic cells, CRISPR-Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, and 8,945,839; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO2014/093661 (PCT/US2013/074743), WO2014/093694 (PCT/US2013/074790), WO2014/093595 (PCT/US2013/074611), WO2014/093718 (PCT/US2013/074825), WO2014/093709 (PCT/US2013/074812), WO2014/093622 (PCT/US2013/074667), WO2014/093635 (PCT/US2013/074691), WO2014/093655 (PCT/US2013/074736), WO2014/093712 (PCT/US2013/074819), WO2014/093701 (PCT/US2013/074800), WO2014/018423 (PCT/US2013/051418), WO2014/204723 (PCT/US2014/041790), WO2014/204724 (PCT/US2014/041800), WO2014/204725 (PCT/US2014/041803), WO2014/204726 (PCT/US2014/041804), WO2014/204727 (PCT/US2014/041806), WO2014/204728 (PCT/US2014/041808), WO2014/204729 (PCT/US2014/041809), WO2015/089351 (PCT/US2014/069897), WO2015/089354 (PCT/US2014/069902), WO2015/089364 (PCT/US2014/069925), WO2015/089427 (PCT/US2014/070068), WO2015/089462 (PCT/US2014/070127), WO2015/089419 (PCT/US2014/070057), WO2015/089465 (PCT/US2014/070135), WO2015/089486 (PCT/US2014/070175), WO2015/058052 (PCT/US2014/061077), WO2015/070083 (PCT/US2014/064663), WO2015/089354 (PCT/US2014/069902), WO2015/089351 (PCT/US2014/069897), WO2015/089364 (PCT/US2014/069925), WO2015/089427 (PCT/US2014/070068), WO2015/089473 (PCT/US2014/070152), WO2015/089486 (PCT/US2014/070175), WO2016/049258 (PCT/US2015/051830), WO2016/094867 (PCT/US2015/065385), WO2016/094872 (PCT/US2015/065393), WO2016/094874 (PCT/US2015/065396), WO2016/106244 (PCT/US2015/067177).

Mention is also made of U.S. application 62/180,709, 17 Jun. 2015, PROTECTED GUIDE RNAS (PGRNAS); U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. applications 62/091,462, 12 Dec. 2014, 62/096,324, 23 Dec. 2014, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 2014 and 62/180,692, 17 Jun. 2015, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, 62/181,641, 18 Jun. 2015, and 62/181,667, 18 Jun. 2015, RNA-TARGETING SYSTEM; U.S. application 62/096,656, 24 Dec. 2014 and 62/181,151, 17 Jun. 2015, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12-F

EB-14, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 2014 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, 62/285,349, 22 Oct. 2015, 62/296,522, 17 Feb. 2016, and 62/320,231, 8 Apr. 2016, NOVEL CRISPR ENZYMES AND SYSTEMS, U.S. application 62/232,067, 24 Sep. 2015, U.S. application Ser. No. 14/975,085, 18 Dec. 2015, European application No. 16150428.7, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES.

Each of these patents, patent publications, and applications, and all documents cited therein or during their prosecution (“appin cited documents”) and all documents cited or referenced in the appin cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appin cited documents) are incorporated herein by reference to the same extent as if each individual document was specifically and individually indicated to be incorporated by reference.

In particular embodiments, pre-complexed guide RNA and CRISPR effector protein, (optionally, adenosine deaminase fused to a CRISPR protein or an adaptor) are delivered as a ribonucleoprotein (RNP). RNPs have the advantage that they lead to rapid editing effects even more so than the RNA method because this process avoids the need for transcription. An important advantage is that both RNP delivery is transient, reducing off-target effects and toxicity issues. Efficient genome editing in different cell types has been observed by Kim et al. (2014, Genome Res. 24(6):1012-9), Paix et al. (2015, Genetics 204(1):47-54), Chu et al. (2016, BMC Biotechnol. 16:4), and Wang et al. (2013, Cell. 9; 153 (4): 910-8).

In particular embodiments, the ribonucleoprotein is delivered by way of a polypeptide-based shuttle agent as described in WO2016161516. WO2016161516 describes efficient transduction of polypeptide cargos using synthetic peptides comprising an endosome leakage domain (ELD) operably linked to a cell penetrating domain (CPD), to a histidine-rich domain and a CPD. Similarly these polypeptides can be used for the delivery of CRISPR-effector based RNPs in eukaryotic cells.

### Tale Systems

As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle EL. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In advantageous embodiments of the invention, the methods provided herein use isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

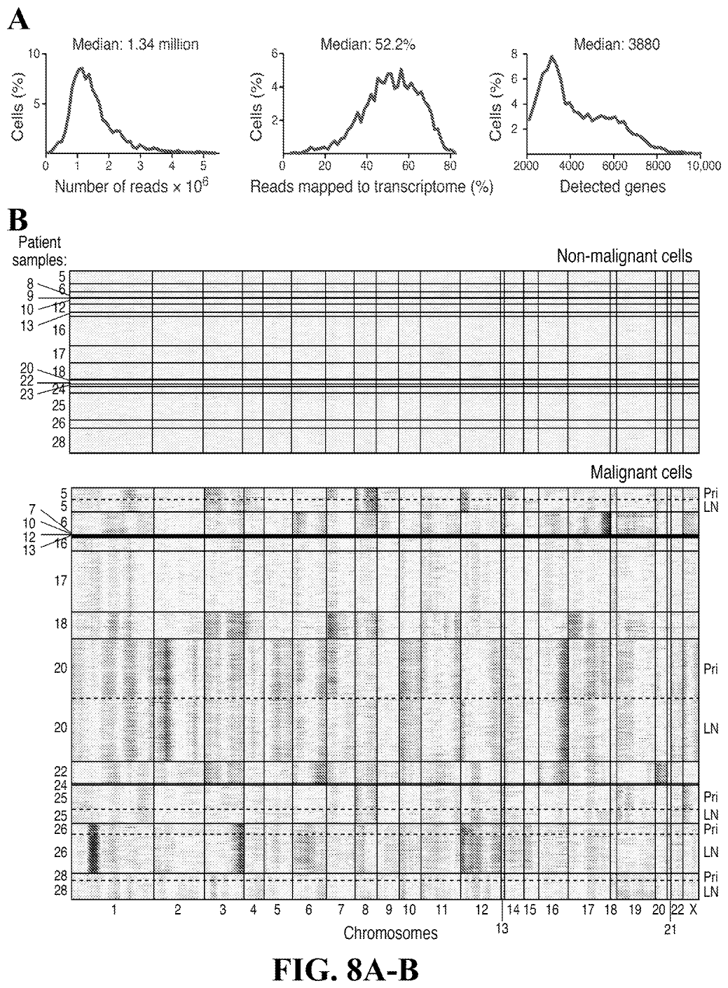

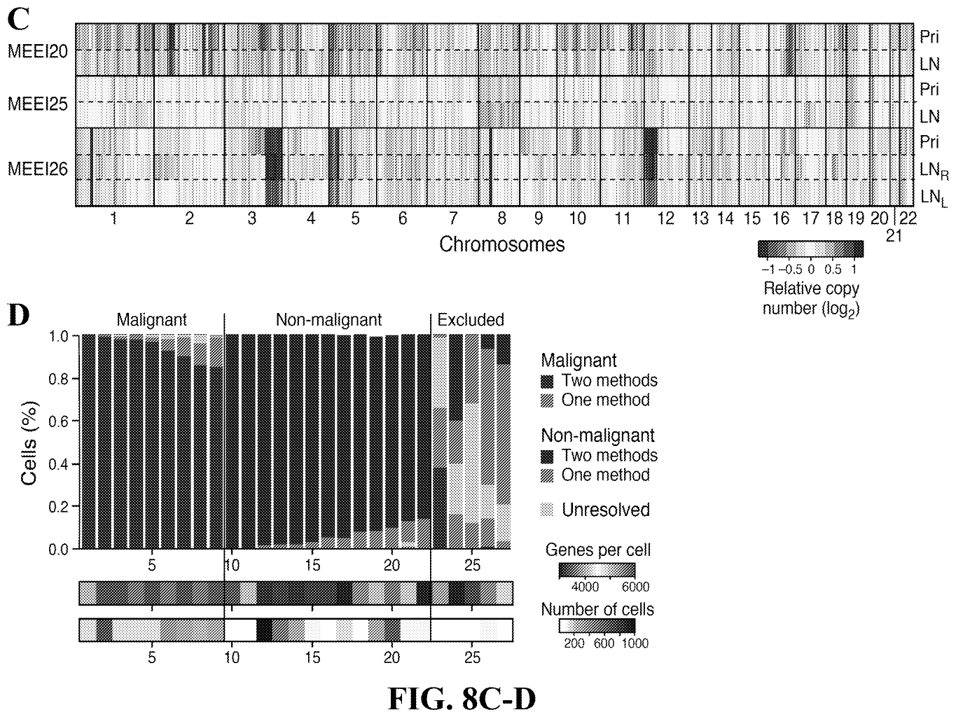

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8), which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ. I.D. No. 20)
M D P I R S R T P S P A R E L L S G P Q P D G V Q P T A D R G V S P
P A G G P L D G L P A R R T M S R T R L P S P P A P S P A F S A D S
F S D L L R Q F D P S L F N T S L F D S L P P F G A H H T E A A T G
E W D E V Q S G L R A A D A P P P T M R V A V T A A R P P R A K P A
P R R R A A Q P S D A S P A A Q V D L R T L G Y S Q Q Q Q E K I K P
K V R S T V A Q H H E A L V G H G F T H A H I V A L S Q H P A A L G
T V A V K Y Q D M I A A L P E A T H E A I V G V G K Q W S G A R A L
E A L L T V A G E L R G P P L Q L D T G Q L L K I A K R G G V T A V
E A V H A W R N A L T G A P L N

An exemplary amino acid sequence of a C-terminal capping region is:

(SEQ. I.D. No. 21)
G R P A L D A V K K G L P H A P A L I K R T N R R I P E R T S H R
V A D H A Q V V R V L G F F Q C H S H P A Q A F D D A M T Q F G M
S R H G L L Q L F R R V G V T E L E A R S G T L P P A S Q R W D R
I L Q A S G M K R A K P S P T S T Q T P D Q A S L H A F A D S L E
R D L D A P S P M H E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In advantageous embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### ZN-Finger Nucleases

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

As disclosed herein editing can be made by way of meganucleases, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain other aspects, the invention is directed to kits incorporating the disclosed herein. The kits may further comprise the reagents necessary to carry out the various enzymatic reactions and assays that may be used in conjunction with the methods disclosed herein.

The present invention advantageously provides for novel tools and methods for the treatment and prognosis of epithelial tumors. Applicants have used single cell RNA-seq to reveal novel expression programs of malignant, stromal and immune cells in the HNSCC tumor ecosystem. Malignant cells varied in expression of programs related to stress, hypoxia and epithelial differentiation. A partial EMT-like program (p-EMT) was discovered and shown to correlate highly with negative pathologies in HNSCC. Applicants also discovered that cells comprising the p-EMT signature resided at the leading edge of tumors and that metastases are dynamically regulated by the TME. Applicants also developed a computational modeling approach to refine TCGA subtypes that allows analysis of malignant cells in bulk sequencing samples. Finally, Applicants unexpectedly linked the p-EMT state to metastasis and adverse clinical features that may be used to direct treatment of epithelial cancers (e.g., HNSCC).

The invention is further described in the following examples, which do not limit the scope of the invention described in the claims.

### EXAMPLES

### Example 1—a Single-Cell Expression Atlas of HNSCC Primary Tumors and Metastases

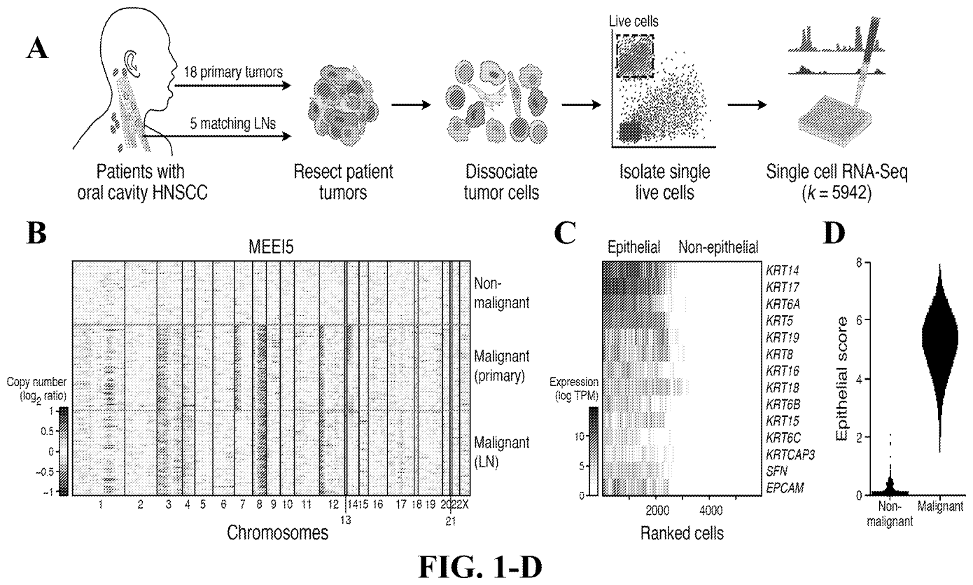

To explore the cellular diversity within and across HNSCC tumors, Applicants focused on oral cavity tumors, which represent the most common subsite of HNSCC. Resection of advanced oral cavity tumors is often accompanied by removal of locoregional LNs, providing an opportunity to obtain primary tumors with matched LN metastases. Applicants profiled single cells from 18 treatment-naïve patients with oral cavity cancer, five of whom had one or more matching LN metastasis analyzed (FIG. 1; Tables S1 and S2). Applicants dissociated freshly resected specimens and generated full-length scRNA-seq profiles (FIG. 1A; Materials and Methods. Whole exome sequencing (WES) and targeted genotyping (SNaPshot) of these tumors demonstrated a range of putative driver mutations and chromosomal aberrations (FIG. 8B; Tables S3 and S4), consistent with established HNSCC genetics (Agrawal et al., 2011; Cancer Genome Atlas, 2015; Stransky et al., 2011).

Applicants retained single-cell transcriptomes for 5,902 cells from 18 patients after initial quality controls (FIG. 8A). Applicants confidently distinguished 2,215 malignant and 3,363 non-malignant cells by three complementary approaches. First, Applicants inferred patterns of large-scale chromosomal copy-number variations (CNVs) in each single cell based on averaged expression profiles across chromosomal intervals (100 genes per interval) (Muller et al., 2016; Patel et al., 2014; Tirosh et al., 2016b). These inferred CNVs, which were consistent with WES (FIGS. 1B, 8B, and 8C), allowed us to distinguish malignant cells from non-malignant cells with normal karyotypes. Second, Applicants independently distinguished malignant cells by their epithelial origin, which differs from stromal and immune cells in the TME (FIG. 1C). Applicants found remarkable concordance between cells with epithelial marker expression and those with aberrant karyotypes (FIG. 1D). Finally, Applicants partitioned the cells to preliminary clusters by their global gene expression patterns. The vast majority of cells were part of clusters with concordant malignant or non-malignant classification, based on CNV and epithelial marker analyses (FIG. 8D; Materials and Methods). The remaining 324 cells were associated with lower data quality and were excluded from further analyses (FIG. 8D).

### Example 2—Landscape of Expression Heterogeneity in Head and Neck Cancer

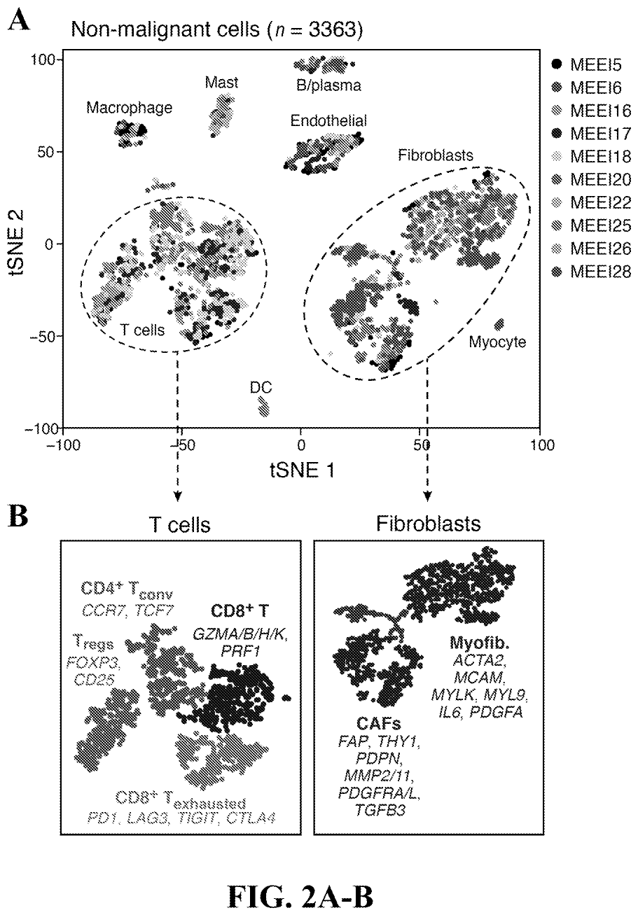

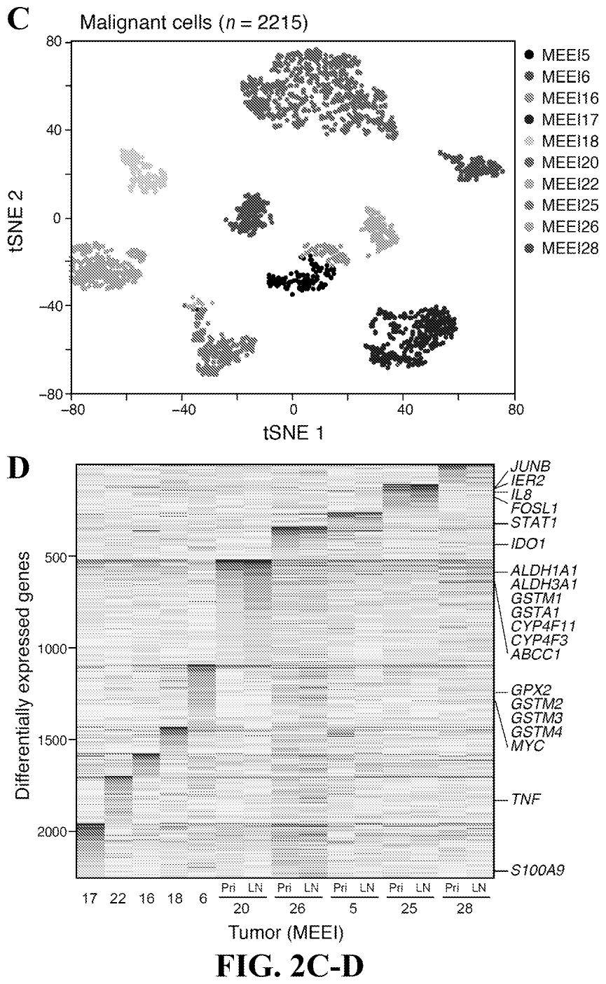

The single-cell profiles of non-malignant cells highlighted the composition of the TME. Applicants partitioned the 3,363 non-malignant cells to eight main clusters by their expression states (FIGS. 2A, 9A, 9B, and 9J). Applicants annotated clusters by the expression of known marker genes as T-cells, B/plasma cells, macrophages, dendritic cells, mast cells, endothelial cells, fibroblasts, and myocytes (FIG. 9B). Notably, each of the clusters contained cells from different patients, indicating that cell types and expression states in the TME are relatively consistent across HNSCC tumors and do not represent patient-specific subpopulations or batch effects, though they do vary in their proportions across patients.

Applicants found additional diversity within both T-cells and fibroblasts through finer clustering, powered by their relatively large numbers in the dataset (FIG. 2B). The main T-cell cluster (˜1,000 T-cells) can be further partitioned into four smaller sub-clusters (FIGS. 2B and 9C; Materials and Methods). Applicants annotated these sub-clusters by the expression of marker genes as regulatory T-cells (T_{regs}), conventional CD4+ T-helper cells (CD4+ T_{conv}), and two cytotoxic CD8+ T-cell populations (CD8+ T and CD8+ T_{exhausted}). The cytotoxic subsets differed in their expression of co-inhibitory receptors (e.g. PD1 and CTLA4) and other genes associated with T-cell dysfunction and exhaustion (Tirosh et al., 2016a), allowing us to define a putative HNSCC-specific program of T-cell exhaustion (FIGS. 2B and 9C). The proportions of exhausted CD8+ T-cells varied significantly among patients in this cohort (FIG. 9D). These T-cell expression states may inform future efforts to understand and predict responses to checkpoint immunotherapies (Mellman et al., 2011), which were recently approved for HNSCC.

Applicants also found substantial diversity among fibroblasts. Despite significant interest, the regulatory states and diversity of fibroblasts in human tumors remain obscure. The ˜1,500 fibroblasts in this dataset partitioned into two main subsets (FIG. 2B, black and blue), and a third minor subset (FIGS. 2B, brown, 9E and 9F). One subset expressed classical markers of myofibroblasts, including alpha smooth muscle actin (ACTA2) and myosin light chain proteins (MYLK, MYL9). Such myofibroblasts are an established component of the TME and have been linked to wound healing and contracture (Rockey et al., 2013). A second subset expressed many receptors, ligands, and extracellular matrix (ECM) genes, including fibroblast activation protein (FAP), podoplanin (PDPN), and connective tissue growth factor (CTGF), that have been associated with classical CAFs (Madar et al., 2013). The third subset was depleted for markers of myofibroblasts and CAFs and may represent resting fibroblasts. These diverse fibroblast expression states were reproducibly detected across primary tumors, suggesting they represent common features of the HNSCC TME.

Although the cellular identity and origin of CAFs has been ascribed to various lineages (Madar et al., 2013), the subpopulations that Applicants detect in HNSCC are highly consistent with a fibroblast identity. Further analysis partitioned these CAFs into two subsets (CAF1 and CAF2) with differential expression of immediate early response genes (e.g. JUN, FOS), mesenchymal markers (e.g. VIM, THY1), ligands and receptors (e.g. FGF7, TGFBR2/3), and ECM proteins (e.g. MMP11, CAV1) (FIGS. 9F and 9G; Table S5). This intra-tumoral fibroblast heterogeneity is consistent with current views that CAFs are involved in complex structural and paracrine interactions within the TME, a feature that Applicants examine in the following sections.

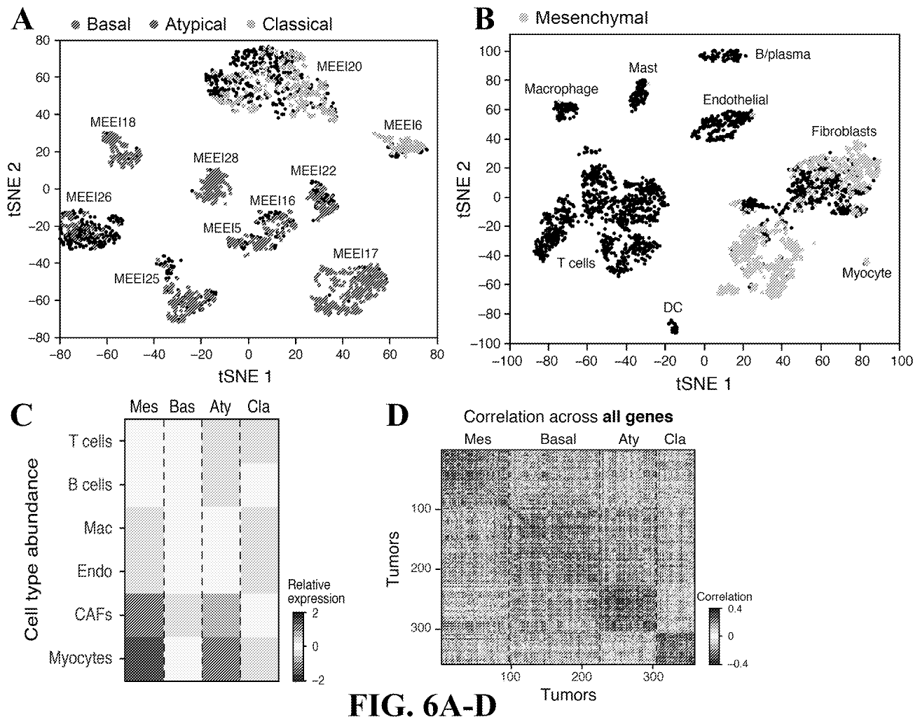

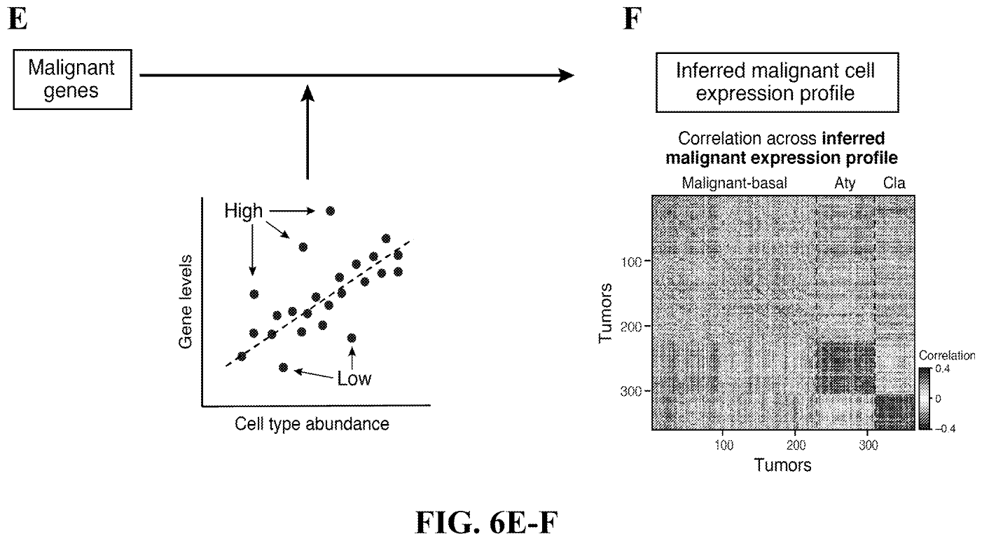

In stark contrast to non-malignant cells, the 2,215 malignant cells in this dataset clustered according to the tumor from which they were derived (FIGS. 2C and 9J). Over 2,000 genes were preferentially expressed in individual tumors (FIG. 2D). Differentially-expressed genes are enriched within CNVs that vary between tumors (FIGS. 9H and 9I), accounting for ˜25% of inter-tumoral heterogeneity. Other differences relate to tumor subtypes (see FIG. 6A and ‘HNSCC subtypes . . . ’ below). For example, genes associated with detoxification and drug metabolism (e.g. GPX2, GSTMs, CYPs, ABCC1) are preferentially expressed by the two classical subtype tumors in this cohort (MEEI6 and MEEI20; FIG. 2D). Finally, other, differentially expressed genes related to stress (e.g. JUNB, FOSL1) or immune activation (e.g. IDO1, STAT1, TNF), potentially in response to varied TMEs. Thus, inter-tumoral malignant cell expression heterogeneity likely reflects differences in genetics, expression subtypes, and TME between tumors in this cohort.

### Example 3—Intra-Tumoral Expression Heterogeneity of the Malignant Compartment

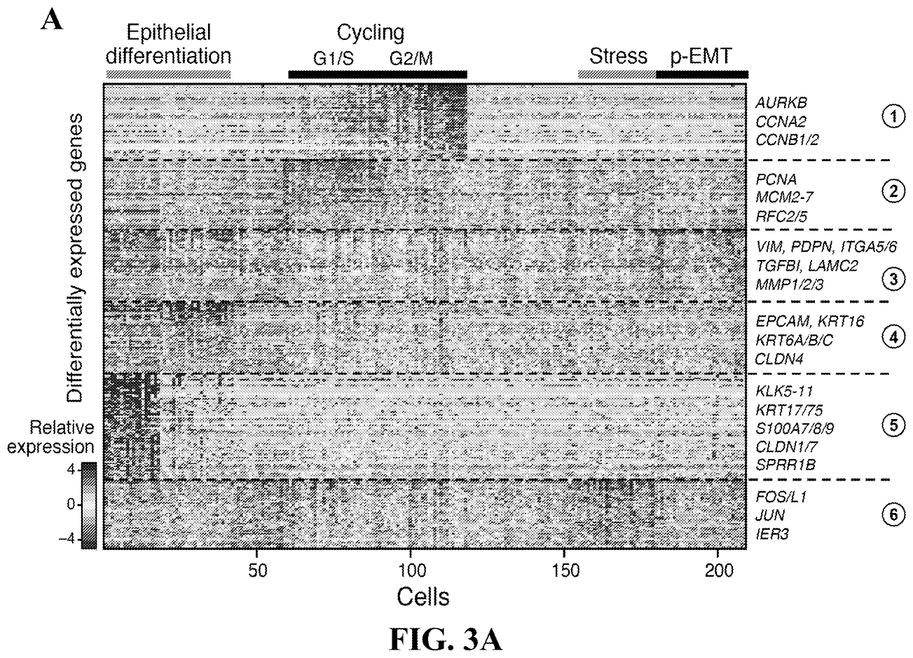

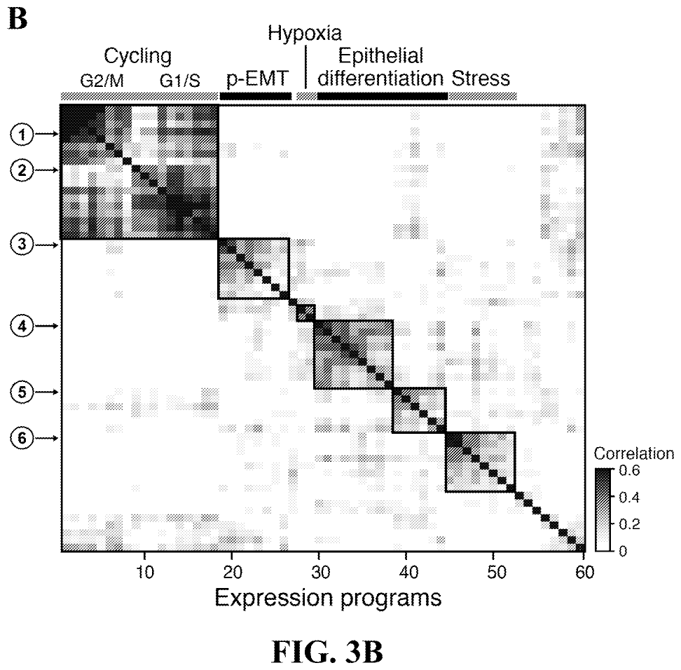

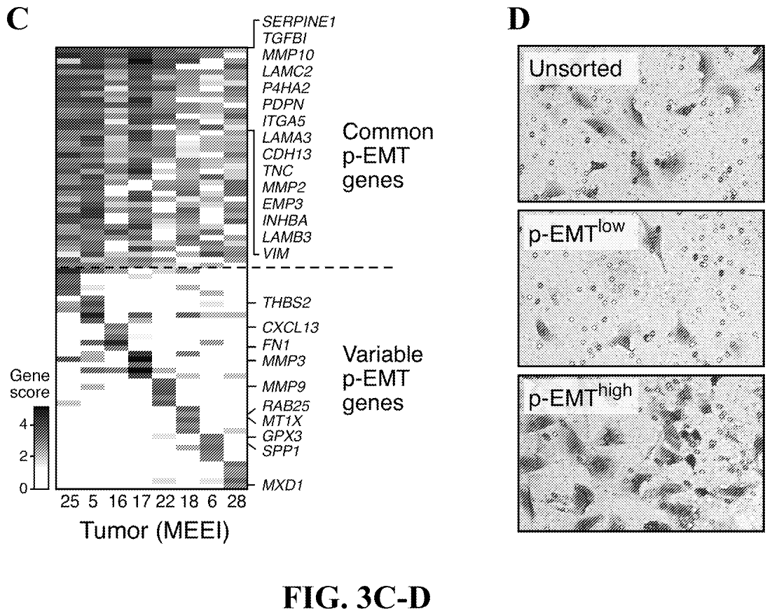

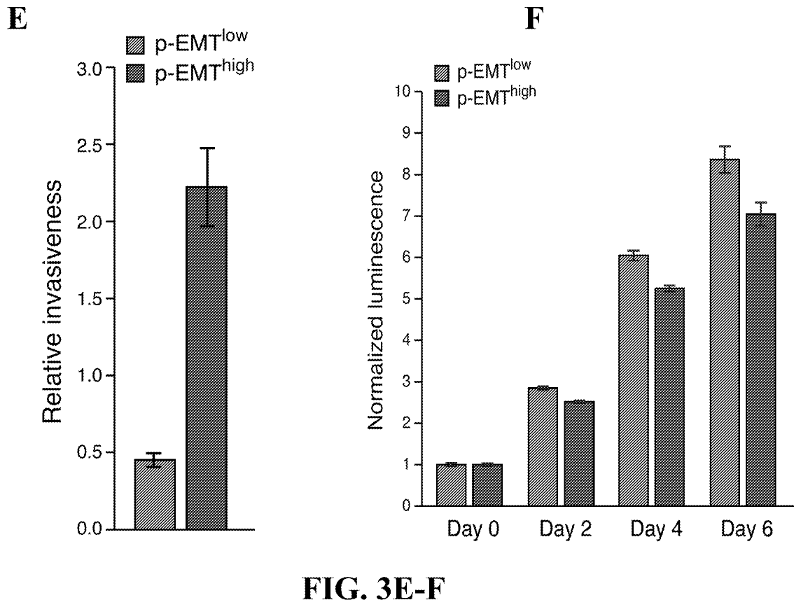

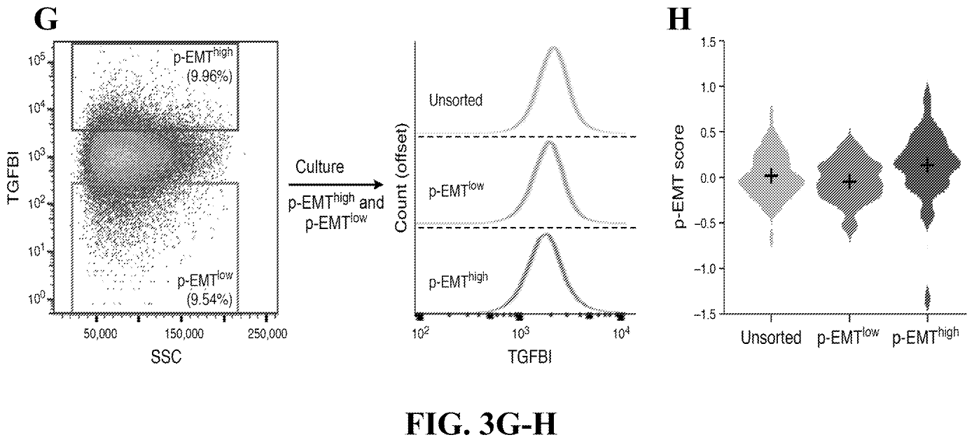

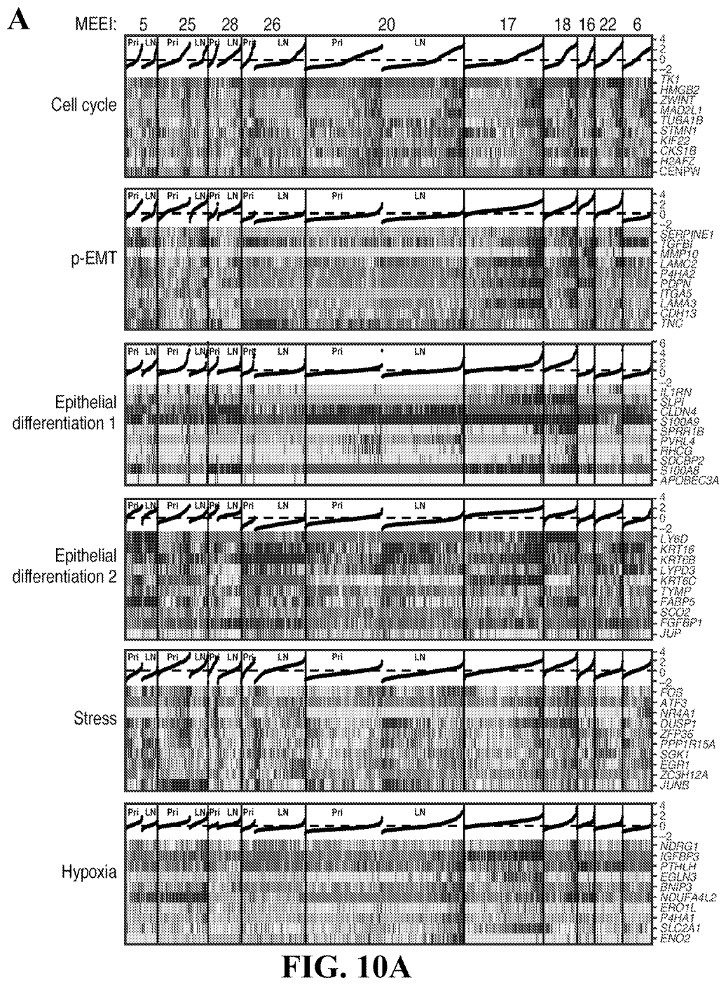

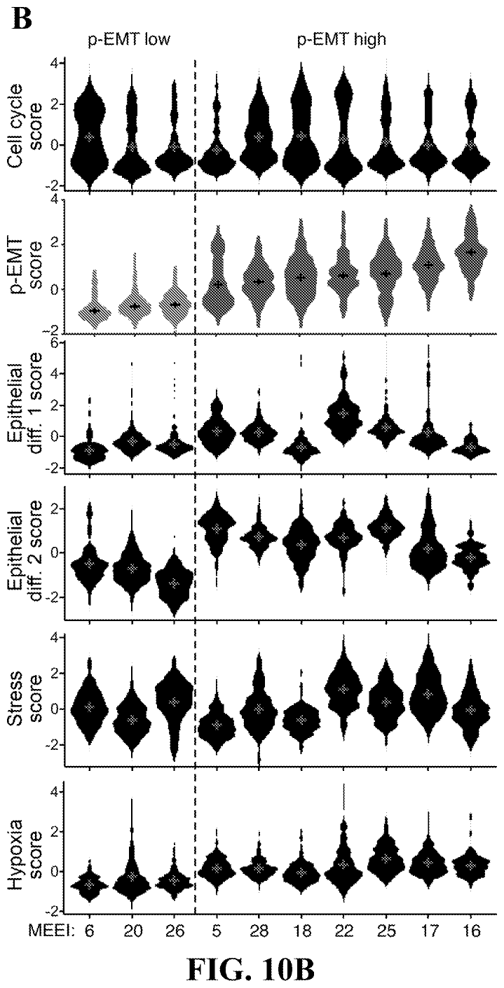

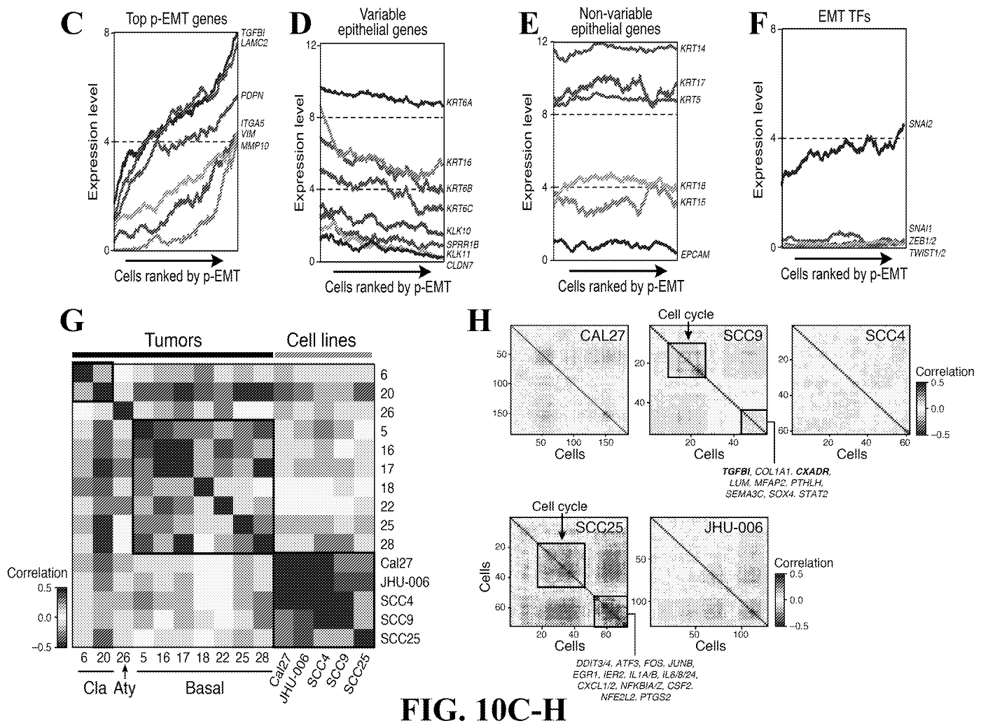

Applicants next explored how expression states varied among different malignant cells within the same tumor, focusing on the 10 tumors from which the largest numbers of malignant cell transcriptomes were acquired (Materials and Methods). Applicants used non-negative matrix factorization to uncover coherent sets of genes (“gene signatures”) that were preferentially co-expressed by subsets of malignant cells in a tumor (Materials and Methods). For example, Applicants defined six gene signatures that vary among malignant cells of MEEI25 (FIGS. 3A and 9K; Table S6). Applying the approach to each of the 10 HNSCC tumors defined a total of 60 gene signatures that coherently vary across individual cells in at least one tumor (Table S6). Next, Applicants used hierarchical clustering to distill these 60 signatures into meta-signatures that reflect common expression programs that vary within multiple tumors (FIGS. 3B, 10A, and 10B; Table S6 and S7; Materials and Methods). The high concordance between signatures from different tumors suggests that they reflect common patterns of intra-tumoral expression heterogeneity in HNSCC.

Seven expression programs were preferentially expressed by subsets of malignant cells in at least two tumors. Two programs (clusters 1, 2 in FIG. 3A and corresponding rows in FIG. 3B) reflected the G1/S and G2/M phases of the cell cycle and allowed us to identify cells in each tumor that were presumed to be cycling (14-40% of cells in the different tumors) (FIG. 10A; Table S7). A third program (cluster 6 in FIG. 3A and corresponding rows in FIG. 3B) consisted of JUN, FOS, and other immediate early genes implicated in cellular activation and stress responses (FIG. 10A; Table S7). A fourth program was enriched for hypoxia-related genes and increased in HNSCC cells cultured in hypoxic conditions (FIGS. 3B, 10A, and 12Q; Table S7).

Two additional programs (clusters 4, 5 in FIG. 3A and corresponding rows in FIG. 3B) consisted primarily of epithelial genes, such as EPCAM, cytokeratins (e.g. KRT6, 16, 17 and 75), and kallikreins (KLK5-11) (FIG. 10A; Table S7). While all malignant HNSCC cells expressed epithelial markers, many of which were largely uniform across malignant cells (FIGS. 1C, 1D, and 10E), the expression levels of these particular epithelial genes varied coherently across malignant cells (FIG. 10D; Materials and Methods) and may reflect the pattern and degree of epithelial differentiation. A final expression program (cluster 3 in FIG. 3A and corresponding rows in FIG. 3B) contained genes associated with the ECM and had features of EMT (FIG. 10A; Table S7). This program was evident in subsets of the cells in seven of the ten tumors examined (FIG. 10B).

### Example 4—A Partial EMT Program in HNSCC

Although EMT programs have been widely considered as potential drivers of drug resistance, invasion, and metastasis, their patterns and significance in human epithelial tumors in vivo remains unclear (Nieto et al., 2016; Thiery et al., 2009; Ye and Weinberg, 2015). Applicants therefore closely examined the ECM program for features of EMT. In addition to ECM genes such as matrix metalloproteinases, laminins and integrins, this program included the EMT markers vimentin (VIM) and integrin α-5 (ITGA5) (FIGS. 3A, 3C, 10A, and 10C; Table S7). Moreover, one of the top scoring genes in this program was TGFβ-induced (TGFBI), thus implicating the classic EMT regulator TGFβ (FIG. 10C).

While the program had key features of classical EMT, it lacked other hallmarks, suggesting it may be a partial EMT program. First, although the EMT signature was accompanied by reduced expression of certain epithelial genes, the overall expression of epithelial markers was clearly maintained (FIGS. 10D and 10E). Second, Applicants did not detect expression of the classical EMT TFs, ZEB1/2, TWIST1/2 and SNAIL1. Only SNAIL2 was detected (in 70% of HNSCC cells), and while its expression correlated with the program across tumors, it did not correlate with the program across individual cells within a tumor (FIG. 10F). Recent work suggests that SNAIL2 peaks earlier than other EMT TFs as cells undergo EMT (van Dijk et al., Pre-print, 2017); SNAIL2 is also implicated in controlling a partial EMT response in the context of wound healing (Savagner et al., 2005). Applicants note that EMT is recognized to be a continuous and variable process (Hong et al., 2015; Lambert et al., 2017; Lundgren et al., 2009; Nieto et al., 2016), and moreover, remains poorly defined in vivo. Applicants therefore suggest that the in vivo program identified here reflects a partial EMT-like state or ‘p-EMT’. Several additional analyses demonstrate that that this p-EMT program is distinct from full EMT programs derived from cell lines and tumor models, as well as from “Mesenchymal” signatures derived from bulk tumor expression profiles (Figures S4A-D) (Cancer Genome Atlas, 2015; Tan et al., 2014). Example 5—In vitro p-EMT cells are highly dynamic and invasive

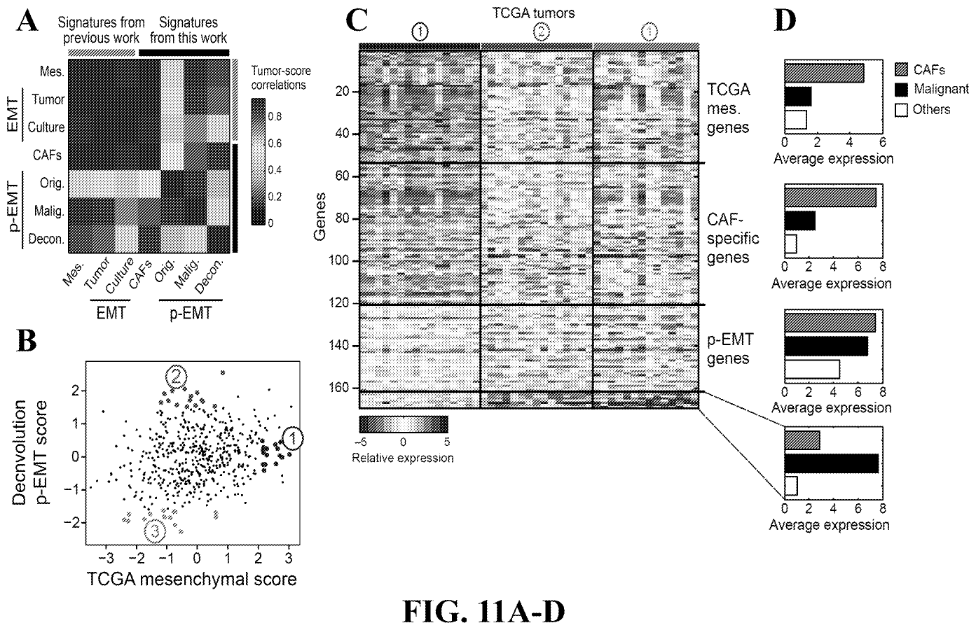

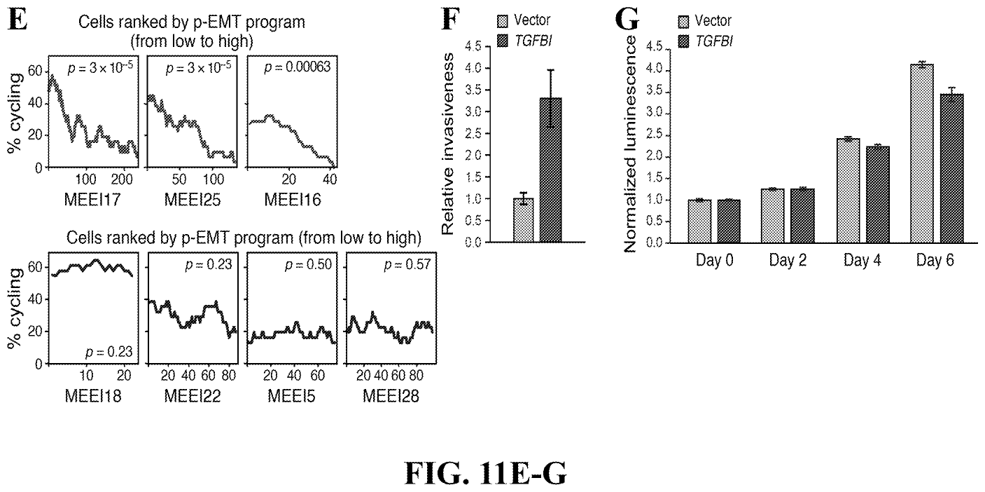

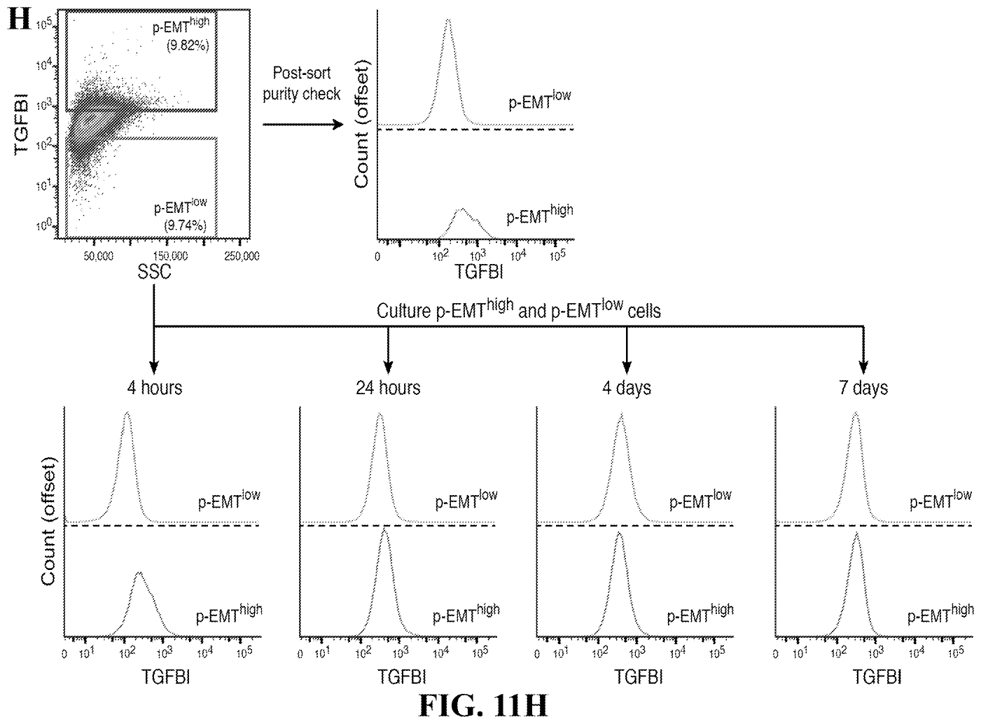

Applicants investigated the functional significance of the p-EMT program across five commonly studied HNSCC cell lines. Expression profiles of 501 cells from these five lines were largely distinct from human tumors (FIG. 10G). However, a subset of cells in SCC9, an oral cavity-derived cell line, partially recapitulated the in vivo p-EMT program (FIG. 1011). These p-EMT^{high} cells were isolated by flow cytometry using two distinct p-EMT markers (TGFBI and CXADR) and demonstrated increased invasiveness in a matrigel transwell assay (FIGS. 3D and 3E). p-EMT^{high} cells also had a decreased proliferation rate (FIG. 3F), consistent with the scRNA-seq analysis of patient samples (FIG. 11E) and prior EMT studies (Nieto et al., 2016; Ye and Weinberg, 2015).

Prior studies have suggested that early stages of EMT may be transitional or metastable (Hong et al., 2015; Lambert et al., 2017; Lundgren et al., 2009; Nieto et al., 2016). Applicants therefore considered whether the p-EMT state might reflect a transient state in dynamic equilibrium with more epithelial HNSCC subpopulations. To test this, Applicants sorted p-EMT^{high} and p-EMT^{low} cells from SCC9, cultured them in vitro, and re-assessed marker expression. The p-EMT^{high} and p-EMT^{low} populations remained distinct 4 hours and 24 hours after sorting (t-test, p<0.0001; FIG. 11H) but became largely indistinguishable after 4 days of culture, with both cultures recapitulating the distribution of marker expression across unsorted SCC9 cells (FIGS. 3G, 3H, and 11H). The dynamic nature of the p-EMT-like program in vitro raises the possibility that the in vivo p-EMT program may also represent a transient state (see Discussion).

### Example 6—p-EMT Cells Localize to the Leading Edge in Proximity to CAFs

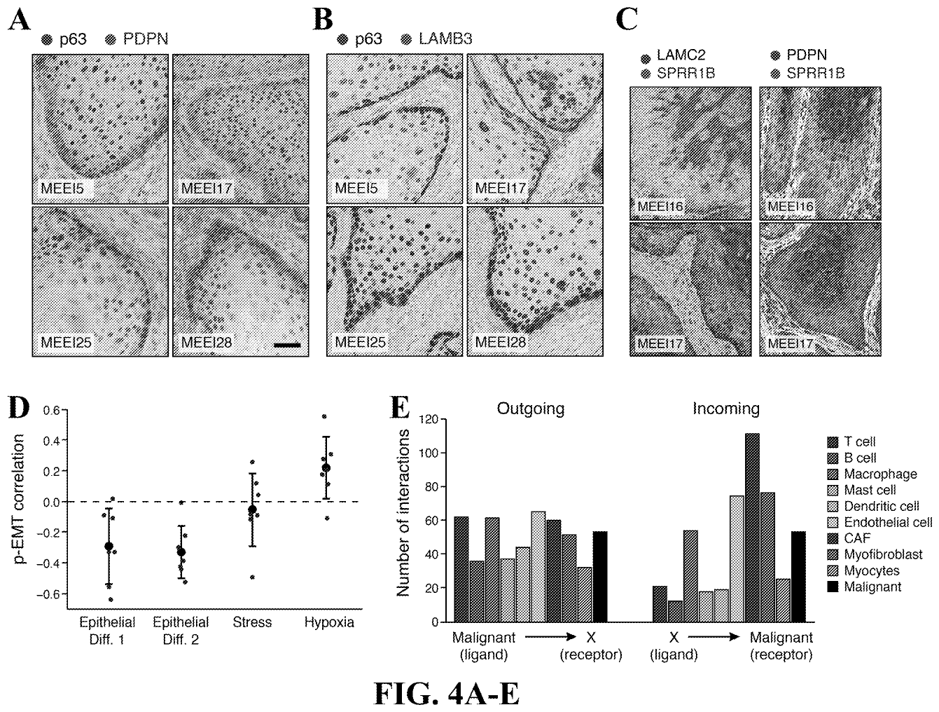

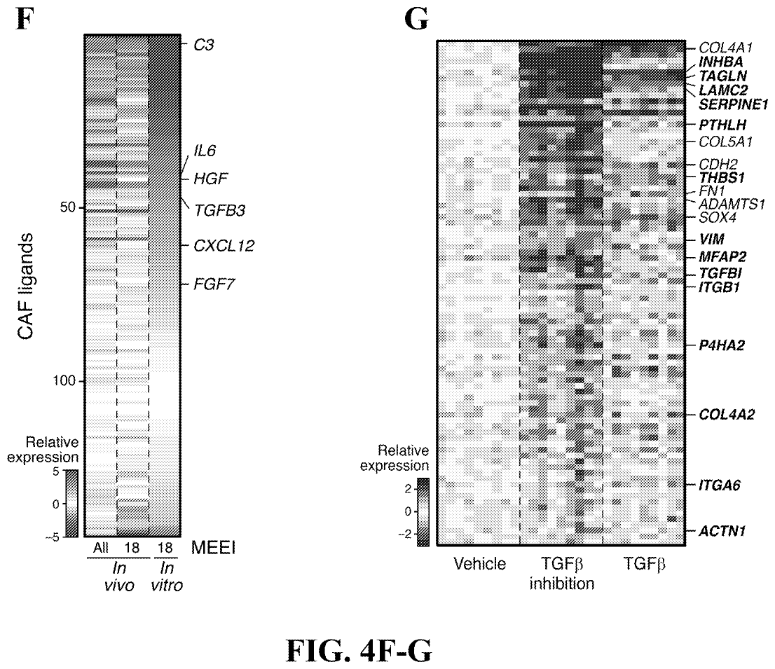

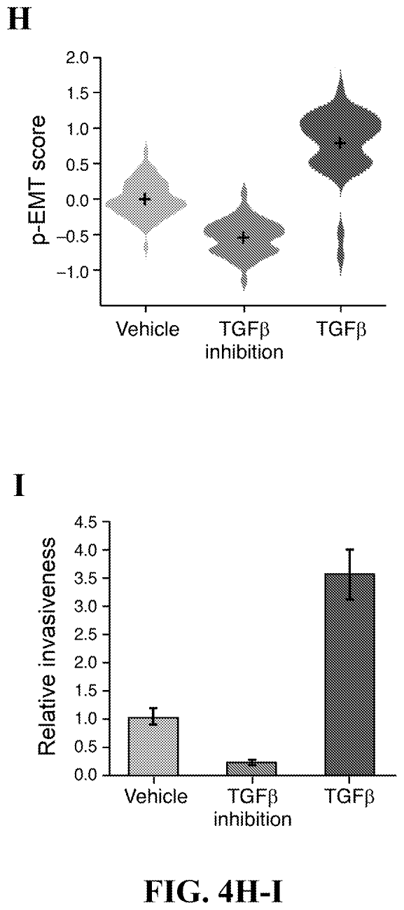

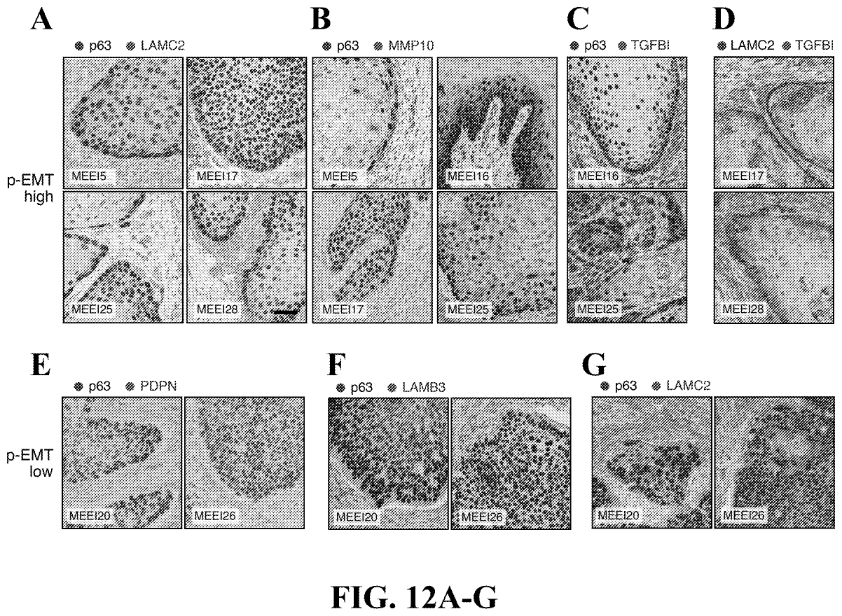

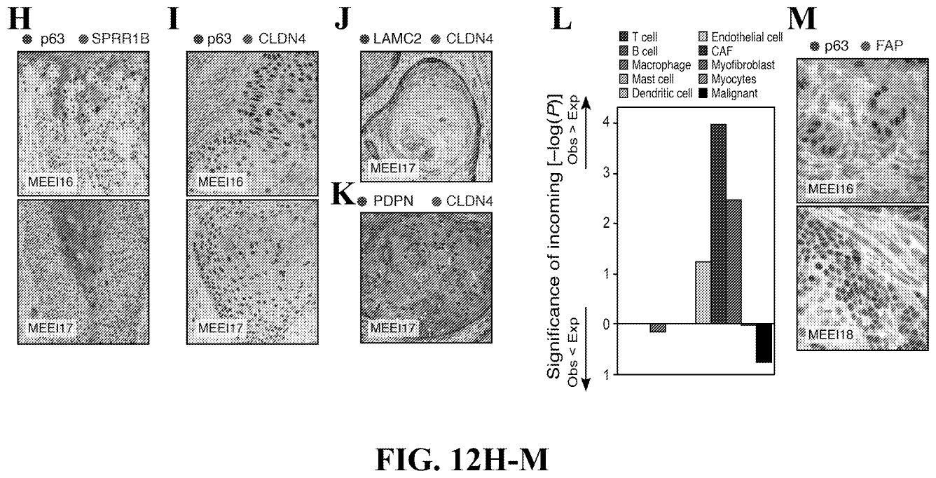

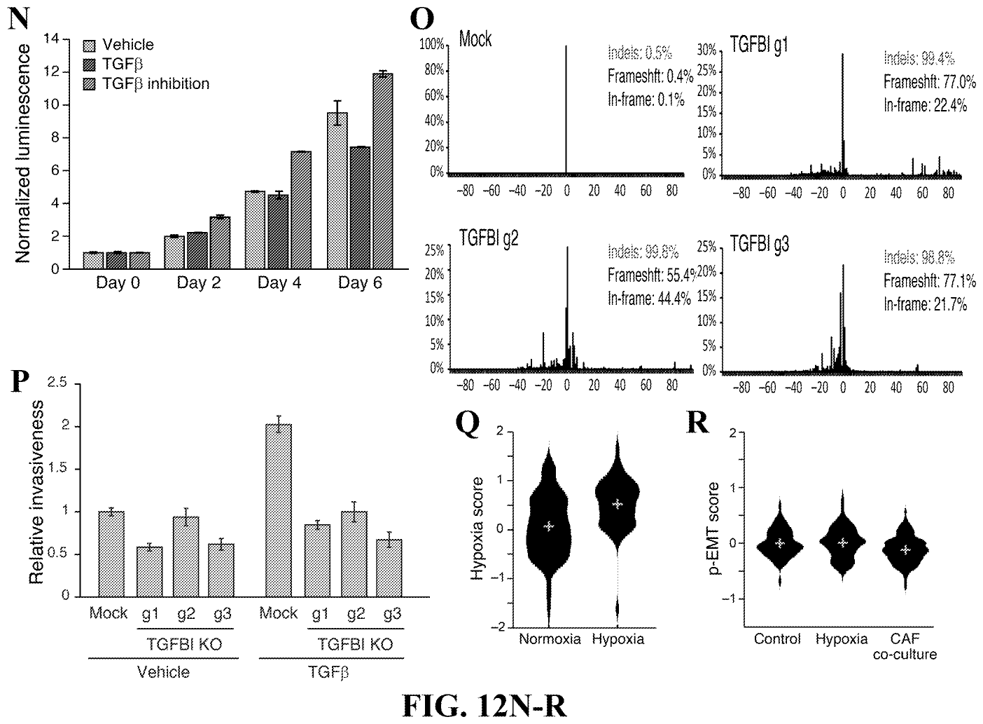

Taken together, the in vivo profiles and in vitro functional data suggest that the p-EMT program is dynamic, invasive, and potentially responsive to TME cues. This led us to investigate the in situ spatial localization of cells expressing this program within HNSCC tumors. Applicants used immunohistochemistry (IHC) to stain a collection of tumors for six of the top genes in the p-EMT program (PDPN, LAMC2, LAMB3, MMP10, TGFBI and ITGA5), along with the HNSCC marker p63 (FIGS. 4A, 4B, and 12A-D).

These experiments revealed a population of malignant cells that co-stain for the p-EMT markers and localize to the leading edge of tumors in close apposition to surrounding stroma. Tumors without cells expressing the p-EMT program in the scRNA-seq data did not stain for these markers (FIGS. 12E-G). In contrast to the p-EMT markers, epithelial differentiation markers (SPRR1B, CLDN4) stained a distinct set of cells at the core of the tumors (FIGS. 4C and 12H-K), consistent with the negative correlation between these programs across individual cells in the scRNA-seq data (FIG. 4D).

The localization of the p-EMT program to the leading edge prompted us to consider interactions with the TME, such as ligand-receptor signaling. Applicants inferred putative tumor-stromal interactions based on high expression of a ligand by one cell type and a corresponding receptor by another cell type (Ramilowski et al., 2015). This analysis predicted “outgoing” signals from malignant cells to the various TME cell types in similar proportions (FIG. 4E). Conversely, when Applicants considered “incoming” signals to malignant cells, Applicants found that CAFs expressed significantly higher numbers of ligands, compared to other cell types, that correspond to receptors expressed by the malignant cells of the corresponding tumor (hypergeometric test, p<0.05; FIGS. 4E and 12L). These included several interactions that may promote EMT, such as TGFB3-TGFBR2, FGF7-FGFR2 and CXCL12-CXCR7 (FIG. 4F) (Moustakas and Heldin, 2016; Ranieri et al., 2016; Yao et al., 2016). Accordingly, when Applicants stained HNSCC tumors for CAF markers (FAP, PDPN), Applicants found that CAF-like cells were present near the p-EMT malignant cells at the leading edge (FIGS. 4C and 12M).

To evaluate the functional significance of the ligand-receptor interactions, Applicants treated SCC9 cells with TGFβ. Four hours of exposure was sufficient to induce a p-EMT-like program, which was repressed upon inhibition of TGFβ (t-test, p<10¹⁶; FIGS. 4G and 411). TGFβ exposure also increased invasiveness and reduced proliferation, while inhibition had opposite effects (ANOVA, p<0.0001; FIGS. 4I and 12N). In addition, overexpression of TGFBI, a known target of TGFβ and the top p-EMT gene, led to similar effects on invasiveness and proliferation (t-test, p<0.005 and ANOVA, p<0.0001, respectively; FIGS. 11F and 11G). Conversely, genetic inactivation of TGFBI abrogated the TGFβ response (ANOVA, p<0.0001; FIGS. 12O and 12P). Although Applicants sought to test CAFs from primary HNSCC tumors in co-culture, Applicants found that cultured fibroblasts lost expression of characteristic activation markers and ligands (FIG. 4F) and failed to induce a p-EMT response in co-cultured cancer cells (FIG. 12R). Taken together, these data suggest that paracrine interactions between CAFs and malignant cells promote a p-EMT program at the leading edge of HNSCC tumors with potential roles in tumor invasion and spread.

### Example 7—Intra-Tumoral HNSCC Heterogeneity Recapitulated in Locoregional Metastases

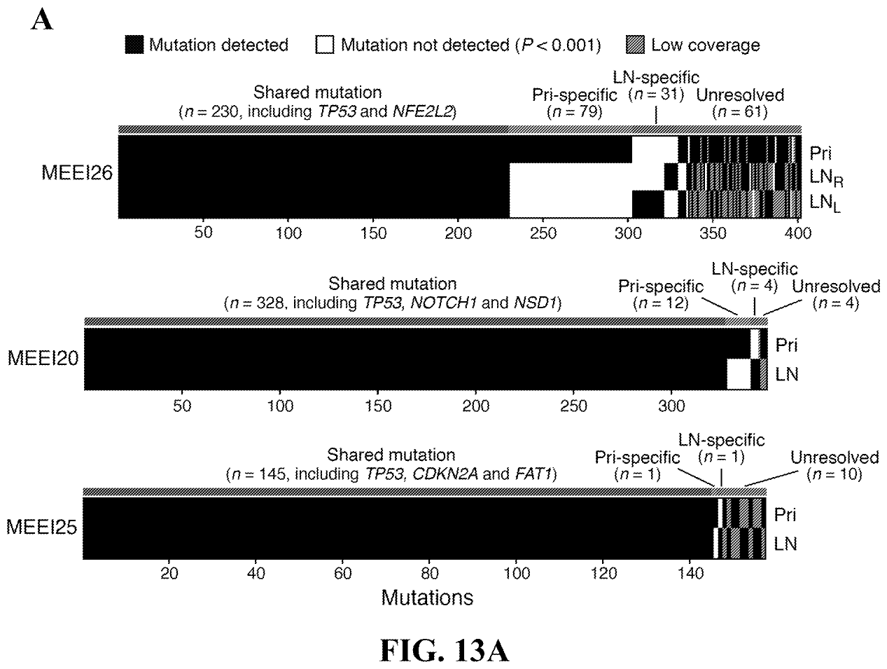

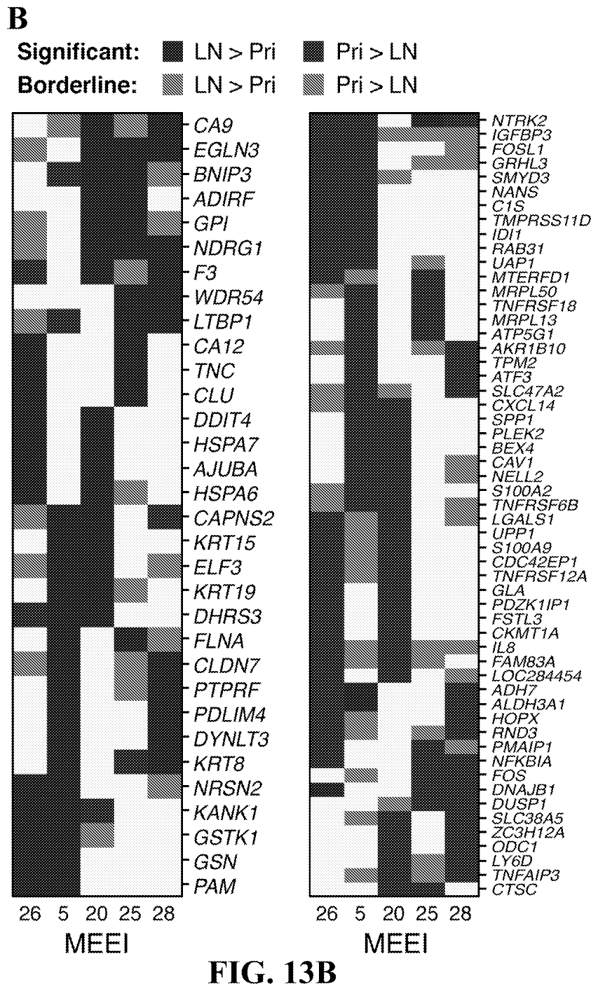

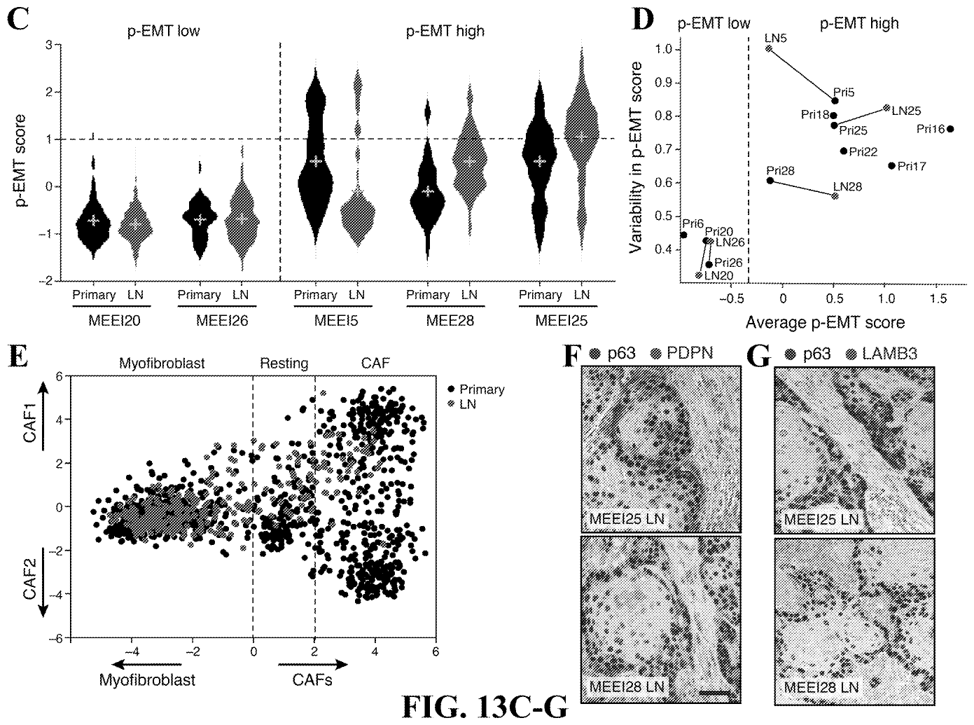

To gain further insight into potential determinants of HNSCC spread, Applicants compared data for five LNs against corresponding primary tumors. Applicants first examined genetic differences between tumor sites. Although inferred CNVs and whole exome sequencing revealed some differences between primary and matched LN samples, they did not identify any distinctions that were consistent across individuals, possibly due to the small number of individuals studied (FIGS. 8B, 8C, and 13A).

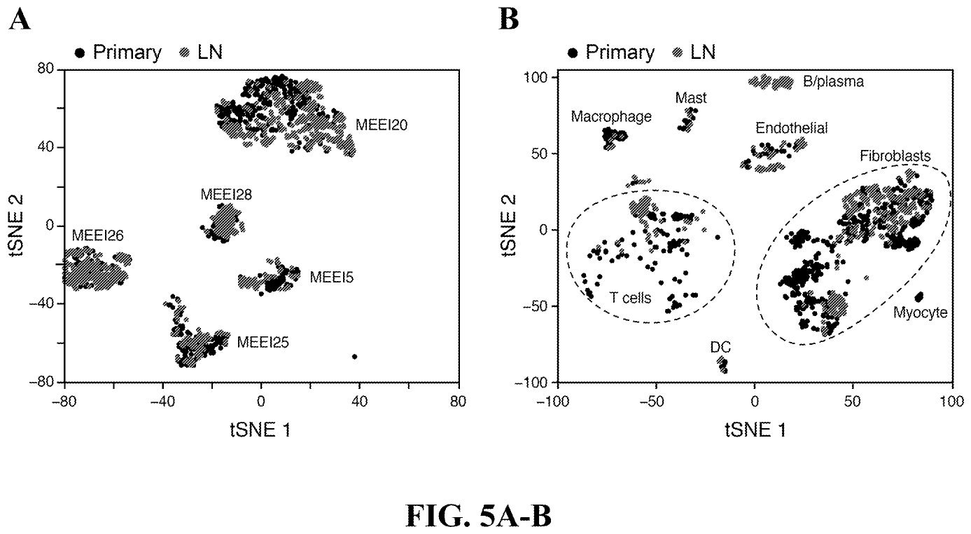

The expression profiles of malignant cells in LNs also largely matched the corresponding primary tumors (FIG. 5A). Few differentially expressed genes were evident for each matched pair, yet they were largely patient-specific and Applicants did not detect any consistent genes that may reflect a signature of LN metastasis (FIG. 13B). The existence of p-EMT high and low subpopulations was consistent between primary tumors and LNs of all patients, but the prevalence of these subpopulations differed between sites (FIGS. 13C and 13D), consistent with the possibility of p-EMT dynamics. While the sample sizes are limited, these findings raise the possibility that programs required for LN metastasis are dynamic and hence undetected in comparisons of primary tumors and LNs. Accordingly, prior studies have also failed to detect consistent genetic or transcriptional distinctions between tumors and locoregional metastases (Colella et al., 2008; Roepman et al., 2006).

Applicants also observed an overall concordance in the identity and representation of stromal and immune cell states in LNs and matched primary tumors, albeit with some important distinctions. Multiple clusters (macrophages, endothelial cells, mast cells, and dendritic cells) contained cells from both sites (FIG. 5B). However, myocytes were observed only in primary tumors, while B/plasma cells were found only in LNs (FIG. 5B). Fibroblast subsets were also differentially represented: LN fibroblasts were enriched for myofibroblasts and the CAF1 subtype (hypergeometric test; p<0.05), and preferentially expressed certain receptors and ligands (e.g. IL1R1, MMP11, SPARC) (FIGS. 5B, 9G, and 13E; Table S8). These differences support an altered signaling environment in the LN, but suggest that the TME remains largely stable upon locoregional metastasis.

These findings prompted Applicants to examine the histology of LN specimens by IHC, using the markers described above. Applicants found largely intact epithelial structures or ‘nests’ of malignant cells (FIGS. 13F and 13G) with p-EMT markers at their periphery, surrounded by CAFs and other TME components. These observations are consistent with a ‘collective migration’ model (Clark and Vignjevic, 2015; Lambert et al., 2017), wherein malignant and stromal cells move in clusters to spread lymphatogenously and form locoregional metastases. Alternatively, individual cells may disseminate and engraft at the same site (‘single-cell dissemination’), thereby recapitulating primary tumor compositional heterogeneity within LN metastases.

### Example 8—HNSCC Subtypes Refined by Deconvolution of Bulk Expression Data

Applicants next considered the generality and prognostic significance of the malignant and stromal expression programs identified from the scRNA-seq data. A recent TCGA study analyzed expression profiles for hundreds of HNSCC tumors, and classified them into four subtypes: basal, mesenchymal, classical, and atypical (Cancer Genome Atlas, 2015). Although the TCGA profiles were acquired from bulk tumors, Applicants reasoned that expression programs of the individual cellular components might enable us to extract additional insights from these data (Tirosh et al., 2016a). In particular, Applicants asked whether molecular subtypes defined from these bulk data reflect differences in malignant programs, malignant cell composition, and/or TME composition.

To address these questions, Applicants first determined the TCGA expression subtypes of the ten HNSCC tumors. Applicants scored malignant cells from each tumor for their correspondence to the TCGA subtype expression signatures. Strikingly, each tumor clearly mapped to just one of three subtypes: basal (n=7), classical (n=2), or atypical (n=1) (FIG. 6A). None of the malignant cells mapped to the mesenchymal subtype, even though it is the second most frequent subtype among oral cavity tumors (Cancer Genome Atlas, 2015). However, when Applicants expanded the analysis to include stromal and immune cells, Applicants found that hundreds of CAFs, myofibroblasts, and myocytes mapped to the mesenchymal subtype (FIG. 6B). This finding raised the possibility that the mesenchymal TCGA subtype reflects high stromal representation in the bulk samples, rather than a distinct malignant cell program. Indeed, analysis of TCGA samples confirmed that mesenchymal subtype tumors highly expressed genes specific to CAFs and myocytes (FIG. 6C). Furthermore, when Applicants directly examined histology sections for HNSCC tumors from the TCGA (Cancer Genome Atlas, 2015), Applicants confirmed that mesenchymal tumors had roughly 2.7-fold more fibroblasts than basal tumors (t-test, p<0.0001; FIGS. 14A-D).

To investigate the influence of TME composition on TCGA classifications further, Applicants devised a computational approach to subtract the effect of non-malignant cells from the TCGA profiles (Materials and Methods). Applicants first restricted the analysis to genes expressed by malignant cells. Since most of these genes were also expressed by non-malignant cells, Applicants then normalized the expression of these genes to remove the expected contribution of non-malignant cells. To this end, Applicants used cell type-specific gene signatures to estimate the relative abundance of each cell type in each tumor and then, for each gene, Applicants inferred a linear relationship between its bulk expression across tumors and the relative abundance of each cell type using multiple linear regression (FIG. 6E). By using the residual of this regression model, Applicants removed the influence of cell type frequencies, including malignant cell frequency (i.e. purity), and inferred a malignant cell-specific intrinsic expression profile for each TCGA tumor (Materials and Methods).

Remarkably, while standard analysis of TCGA tumors recovered all four subtypes (FIG. 6D), analysis of inferred malignant cell-specific expression completely eliminated the mesenchymal subtype, while maintaining the other three subtypes (FIG. 6F). Tumors previously classified as mesenchymal were found to be part of the previously described basal subtype (now referred to as ‘malignant-base’). Importantly, Applicants validated that TCGA mesenchymal scores reflect genes primarily expressed by CAFs and do not correlate with the malignant cell-specific p-EMT program (FIG. 11B-D). Applicants therefore suggest that HNSCC tumors may be refined into three subtypes of malignant cells (malignant-basal, classical, and atypical), with the previously described mesenchymal subtype reflecting malignant-basal tumors with a large stromal component. The combined malignant-basal subtype would be particularly prevalent, comprising >70% of oral cavity tumors in TCGA, consistent with the classification of seven out of ten tumors in the cohort.

### Example 9—p-EMT Predicts Nodal Metastasis and Adverse Pathological Features

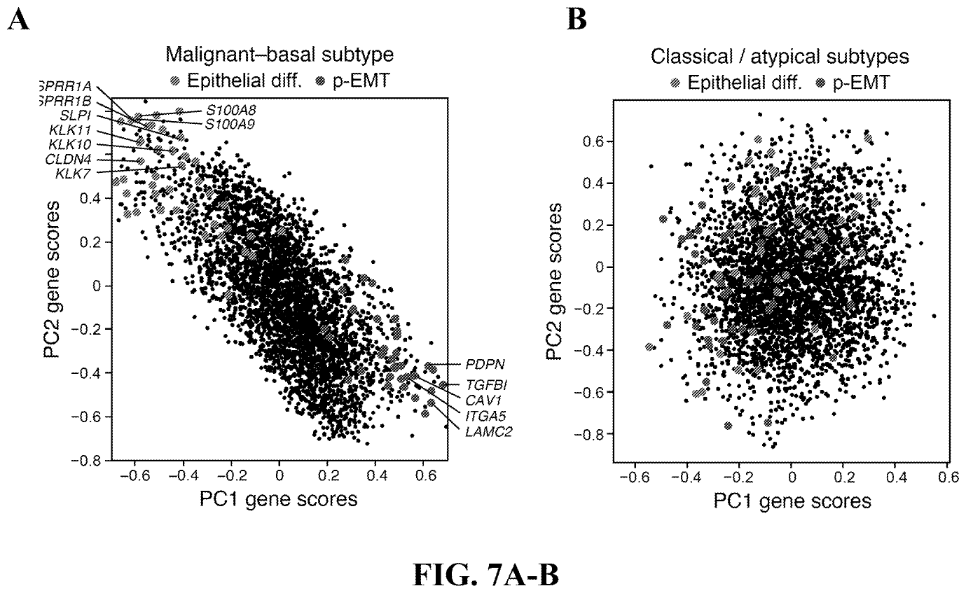

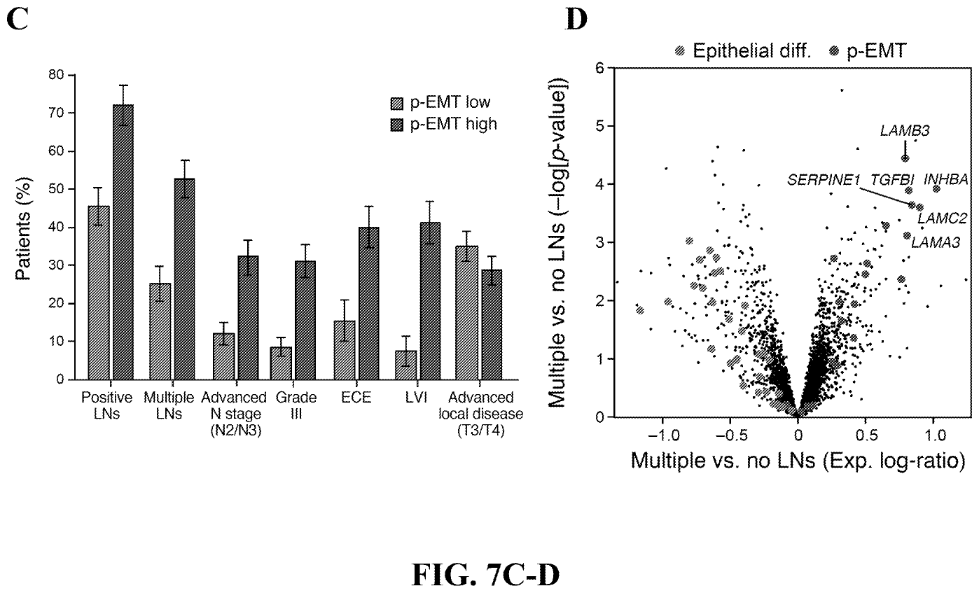

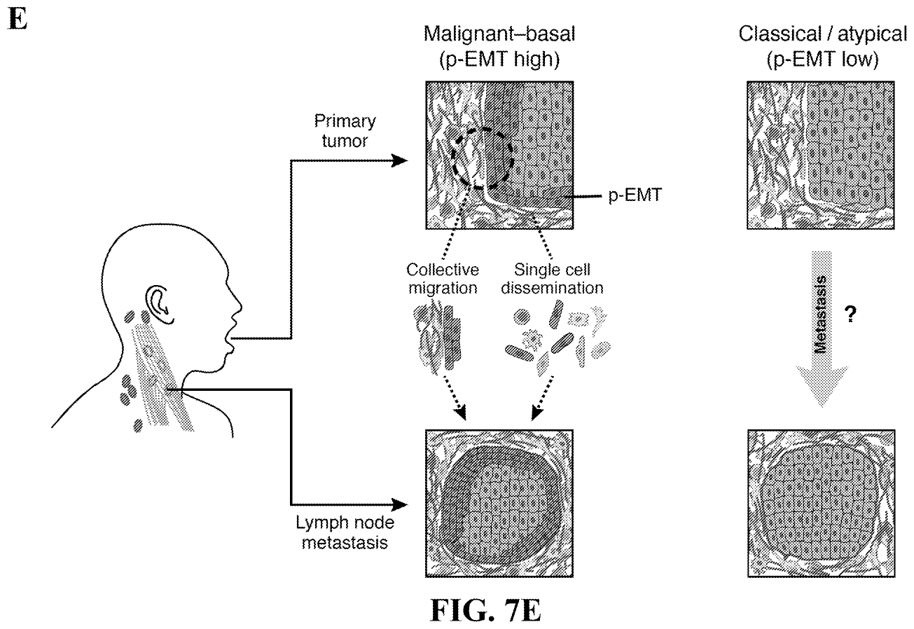

Incorporation of TCGA data gave Applicants an opportunity to examine the prevalence and significance of the p-EMT program across a larger cohort. In the smaller cohort, the p-EMT program was evident in cells from seven of ten tumors (FIG. 10B), which exactly correspond to the seven tumors that mapped to the malignant-basal subtype (FIG. 6A). Consistent with the smaller cohort, p-EMT levels were highest in malignant-basal tumors in TCGA (originally classified as basal or mesenchymal; FIG. 14E). Furthermore, principal component analysis (PCA) of malignant-basal TCGA tumors, but not of atypical and classical tumors, revealed that the first two components (PC1 and PC2) were associated with expression of p-EMT genes, and were inversely correlated with expression of epithelial differentiation genes (FIGS. 7A, 7B, 14F, and 14G). Remarkably, the p-EMT programs defined from these unbiased analyses of bulk expression data were highly consistent with those defined by the scRNA-seq analyses (FIG. 7A). They independently confirmed the absence of expression of classical EMT TFs, except for SNAIL2 (FIG. 14L), and therefore further support an in vivo p-EMT state in human tumors. Thus, by controlling for confounding effects of TME composition, Applicants demonstrate that differences in the expression of the p-EMT program represent a predominant source of inter-tumoral variability in HNSCC tumors.

Lymphatogenous spread of HNSCC tumors to form LN metastases is a major source of disease burden and mortality. Accordingly, resection of advanced oral cavity tumors is typically accompanied by neck dissection (lymphadenectomy) to remove the first echelon of draining LNs, a procedure associated with patient morbidity. In addition, tumors with poor prognostic features such as extracapsular extension or lymphovascular invasion receive adjuvant therapy (radiation with or without chemotherapy). Applicants therefore tested whether the in vivo p-EMT signature might predict unfavorable pathological features or disease outcome. Applicants partitioned malignant-basal tumors into high and low p-EMT subsets, which Applicants evaluated for major pathological and clinical features.

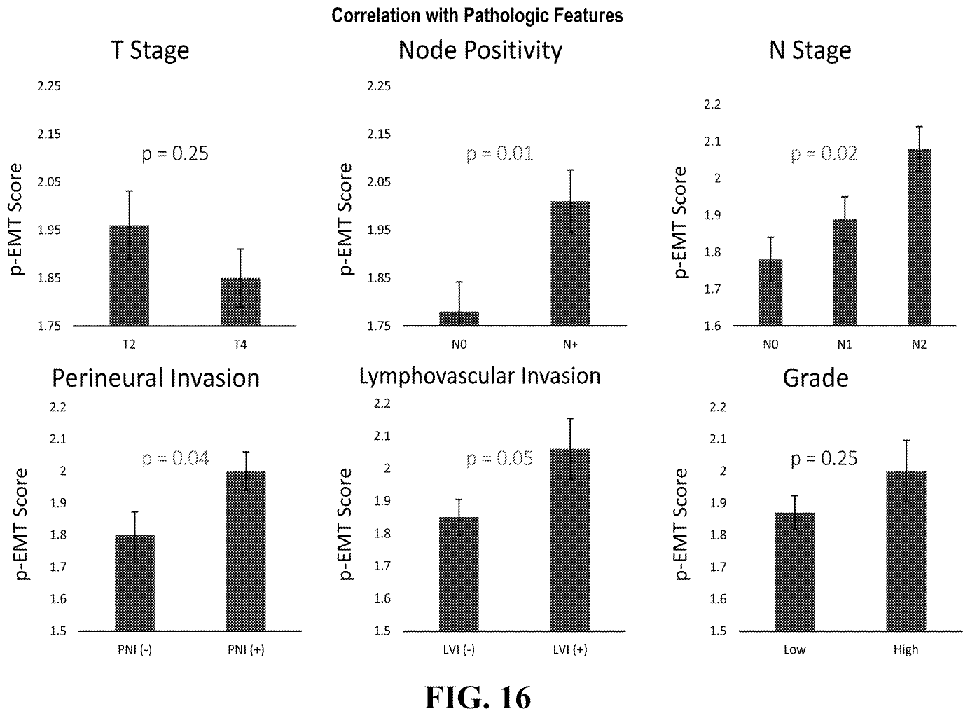

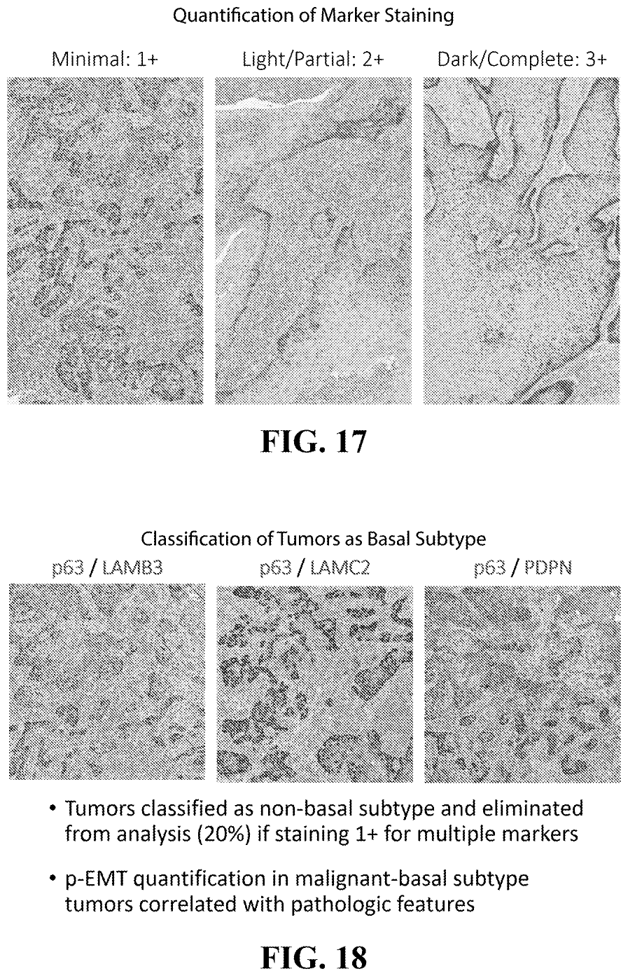

Applicants found that high p-EMT scores were associated with the existence and number of LN metastases and with higher pathological nodal (N) stage (hypergeometric test; p<0.05; FIG. 7C). Applicants also found an association with higher tumor grade, offering a potential explanation for the aggressiveness of poorly differentiated tumors. High p-EMT scores were similarly associated with adverse pathological characteristics, including extracapsular extension and lymphovascular invasion (FIG. 7C), for which no reliable biomarkers are currently known. Interestingly, p-EMT was not associated with primary tumor size (FIG. 7C), suggesting a direct association with invasion and metastasis but not with tumor growth. Overall, p-EMT genes were among the top correlated genes with these clinical features, while other programs such as cell cycle or hypoxia did not correlate nearly as strongly with any of these measures (FIGS. 7D and 14H). In contrast, the epithelial differentiation program was negatively associated with metastasis (FIG. 14H), consistent with the prior observation of an inverse correlation between p-EMT and epithelial differentiation. Importantly, the p-EMT program is a stronger predictor of nodal metastasis and local invasion (FIG. 14I) than either the TCGA mesenchymal program or conventional EMT signatures collated from literature, both of which primarily reflect CAF frequency (FIGS. 11A and 14I) (Cancer Genome Atlas, 2015; Tan et al., 2014).Current clinical practice relies on imperfect predictors of nodal metastasis, such as tumor thickness and size, resulting in a high rate (˜80%) of unnecessary neck dissections (Monroe and Gross, 2012). The p-EMT score could help predict nodal metastasis and thus spare patient morbidity associated with unnecessary neck dissections (FIG. 14J). Applicants further validated the association of p-EMT with adverse pathologic features in an independent MEET cohort of patients by IHC (FIG. 16-18).

### Example 10—Discussion

Intra-tumoral heterogeneity represents a major challenge in oncology. Among various emerging technologies, scRNA-seq has facilitated the identification of developmental hierarchies, drug resistance programs, and patterns of immune infiltration relevant to tumor biology, diagnosis, and therapy (Giustacchini et al., 2017; Kim et al., 2016; Li et al., 2017; Patel et al., 2014; Tirosh et al., 2016a; Tirosh et al., 2016b; Venteicher et al., 2017). Here, Applicants applied the approach to characterize primary HNSCC tumors and matched LN metastases. This analysis highlights a complex cellular ecosystem with active cross-talk between malignant and non-malignant cells, and an in vivo p-EMT program associated with metastasis. This study represents an important step towards understanding intra-tumoral expression heterogeneity in epithelial tumors, which encompass most solid malignancies, and identifies cell states and programs relevant to invasion and metastasis (FIG. 7E).

Among the key findings is the identification of a p-EMT program in malignant cells in vivo. This program involves upregulation of certain mesenchymal genes and moderation of epithelial programs. Although reminiscent of an EMT-like process, the program lacks classical TFs thought to drive EMT (ZEB1/2, TWIST1/2, SNAIL1) (Nieto et al., 2016; Thiery et al., 2009; Ye and Weinberg, 2015). The tumors do, however, express SNAIL2, another implicated TF. SNAIL2 levels do not correlate with the p-EMT program across individual cells in a tumor, but do correlate with the p-EMT program across tumors, both in the small cohort and in TCGA tumors (FIGS. 14K and 14L), hinting at post-transcriptional regulation. Prior studies have linked SNAIL2 to EMT-like changes required for wound healing (Savagner et al., 2005), raising the possibility that such physiologic responses are co-opted by malignant epithelial cells, especially at the invasive edge.

Given the absence of some classical regulatory programs, the retention of epithelial markers, and the likely transience of this expression state, Applicants speculate that the p-EMT program reflects a ‘metastable’ state that recapitulates certain aspects of EMT, but may be fundamentally different from those defined previously in vitro (Hong et al., 2015; Lambert et al., 2017; Lundgren et al., 2009; Nieto et al., 2016). Indeed, although Applicants describe an isolated EMT-like program, the molecular description of EMT is currently being re-evaluated with increasing evidence for a continuum of states. It has also been hypothesized that a dynamic, partial EMT state confers invasive properties without losing tumor initiation capacity (Lambert et al., 2017). It remains unclear whether a full EMT state exists in HNSCC, or if the spectrum extends only to p-EMT. Regardless, the unbiased definition of an in vivo partial EMT-like program in patients can guide future studies of this process as it relates to human cancers and metastases.

Several observations suggest that the p-EMT program may promote local invasion and LN metastasis. First, IHC analyses clearly showed that the program localizes to the leading edge of primary tumors, potentially enabling the collective migration of cohorts of cells and their locoregional or distant dissemination (FIG. 7E) (Clark and Vignjevic, 2015; Lambert et al., 2017). Interestingly, p-EMT cells are in close proximity to CAFs in the surrounding TME, consistent with ligand-receptor analyses supporting regulatory cross-talk between these populations. Second, p-EMT^{high} HNSCC cells have increased invasive potential in vitro. Third, deconvolution of bulk expression profiles for hundreds of HNSCC tumors identified the p-EMT program as a leading source of variability between patients that is strongly predictive of nodal metastases, lymphovascular invasion, and extranodal extension. Importantly, although CAF abundance did not independently predict nodal metastasis and invasion, tumors with both high CAF scores and high p-EMT scores had a particularly high propensity for metastasis, consistent with a cooperative effect (FIG. 14I). This could potentially reflect a role for paracrine signaling between CAFs and malignant cells in promoting nodal disease.

At the same time, other observations temper the conclusions. First, an important caveat is the limited size of this study—only 10 tumors were deeply characterized. Analysis of more tumors may reveal additional stromal, immune and malignant cell states, potentially including malignant cells that have further progressed towards a mesenchymal state. Second, the p-EMT program is largely absent from classical and atypical HNSCC tumors, which nonetheless metastasize at similar rates (Cancer Genome Atlas, 2015). Thus, p-EMT may be relevant in some subtypes but not others, potentially explaining discordance in prior studies regarding the importance of EMT in tumor biology (Nieto et al., 2016; Thiery et al., 2009; Ye and Weinberg, 2015). Third, although the data implies that the p-EMT state may be responsive to CAF signals, the program might simply be a function of increased TME interactions due to disrupted tumor borders and hence increased capacity for metastasis rather than a cause. Thus, although the study establishes robust associations, it does not define the precise mechanisms by which p-EMT and/or corresponding stromal interactions drive HNSCC metastasis.

Subtype classification schemes have been applied to several tumor types based on ‘bulk’ samples, which cannot effectively distinguish true malignant classes from population mixtures (Patel et al., 2014) or differences in stromal cell abundance. Here, knowledge of the expression states of the malignant, stromal, and immune cell types in HNSCC tumors enabled us to deconvolve bulk TCGA data and infer malignant cell-specific expression profiles. This analysis suggested that the mesenchymal subtype reflects the TME, namely the fraction of CAFs and myocytes within a tumor. Indeed, no malignant cells mapped to the mesenchymal subtype described by TCGA. Thus, the mesenchymal subtype may reflect stromal composition and should be re-evaluated in future studies. In contrast, Applicants find strong support for the other three HNSCC subtypes (classical, atypical, and basal) in that malignant cells from each tumor map exclusively to one of those subtypes and these subtypes remain stable when controlling for TME. Nonetheless, the potential of stromal components to offer orthogonal prognostic insight (FIG. 14I) suggests that future classification systems may ultimately need to integrate detailed information on both malignant states and non-malignant components in a tumor.

In summary, this work provides important insights into HNSCC tumor biology and an atlas of diverse malignant, stromal, and immune cells that should prove relevant to other epithelial malignancies (i.e. carcinomas). The computational approach for inferring malignant cell-specific profiles from bulk expression data refines malignant subtypes in HNSCC, and offers a powerful strategy to extract information from the large universe of existing expression profiles. Finally, the definition of a p-EMT program helps relate a large body of EMT data to the in vivo biology of a human tumor. Although further studies are can be performed, the association of this p-EMT program to unfavorable clinical features can guide diagnostic strategies and treatment algorithms.

### Example 11—Tumor Resistance Programs

Applicants additionally found that CAFs in cold tumors overexpressed genes upregulated by TGFB1 (P=1.70*10⁻⁷, hypergeometric test) and that these CAFs were associated with T cell exclusion. The genes included BHLHE40, CRYAB, ELL2, ETS2, NTF3, PDGFA, RHOB, RRAD, SMTN, TAGLN and C3. As used herein “cold tumors” refer to tumors that do not respond to immunotherapy (e.g., checkpoint blockade therapy). Therefore, CAFs that over-express TGFβ genes are also more likely to reside in “cold” HNSCC tumors. These results are consistent with the hypothesis that in HNSCC, CAFs secrete TGFβ and induce the p-EMT response. Indeed, TGFB1 and TGFB signaling has been recently shown to be highly associated with lack of response to anti-PD-L1 treatment in urothelial cancer patients (Mariathasan et al., 2018 TGFβ attenuates tumour response to PD-L1 blockade by contributing to exclusion of T cells. Nature 554, 544-548). Moreover, co-administration of TGFβ-blocking and anti-PD-L1 has been shown to modulate the tumor CAFs, which in turn facilitated T cell infiltration and tumor regression in mouse models (Mariathasan et al., 2018). Thus, TGFβ inhibition can block CAFs from inducing the p-EMT signature resulting in increased responsiveness to immunotherapy and induction of T cell infiltration.

### Example 12—Materials and Methods

### Experimental Model and Subject Details

Human Tumor Specimens. Patients at the Massachusetts Eye and Ear Infirmary (MEEI) (Table 51) were consented preoperatively to take part in the study following Institutional Review Board approval (Protocol #11-024H). Fresh biopsies of oral cavity head and neck squamous cell carcinoma (HNSCC) were collected at the time of surgical resection, either from the primary tumor or lymph node (LN) dissection. A small fragment was snap frozen for bulk whole exome sequencing and the remainder of the provided tissue was processed for single-cell RNA-seq (scRNA-seq).

The MGH Cancer Registry was used to select an independent MEEI cohort of MEEI patients for p-EMT Markers (FIGS. 16-18). The MGH cancer registry provides well documented TNM staging, type of surgery, margin status, adjuvant therapy, recurrence, and survival. Clinical and pathologic information was available for 99 patients treated surgically for primary oral cavity HNSCC between 1995-2015 (47 T2 tumors, 52 T4 tumors, and ˜50% node positive in each condition). Tissue microarrays (TMAs) were created from paraffin blocks. H&E slides were reviewed for each patient and areas of tumor were marked. Five 2 mm cores from at least 3 paraffin blocks for each primary tumor and up to four 2 mm cores for each lymph node were collected. Double IHC staining was performed for the tumor marker p63 and each marker in the p-EMT marker panel. Quantification of marker staining was performed as 1+, 2+, or 3+.

Cell Lines. Oral cavity HNSCC cell lines (Cal-27, SCC9, SCC4, SCC25, and JHU-006; all derived from male patients) were generously provided by Dr. James Rocco and colleagues after confirmation by short tandem repeat (STR) analysis (data not shown). They were cultured as follows: JHU-006 cells were grown in RPMI 1640 media (ThermoFisher Scientific), while others cells were grown in 3:1 Ham's F12 (ThermoFisher Scientific):DMEM (ThermoFisher Scientific). 10% fetal bovine serum (FBS; Peak Serum, Fort Collins, Colo.) and 1× penicillin-streptomycin-glutamine (PSG; ThermoFisher Scientific) were added to all growth media.

### Method Details

Tumor Dissociation. Fresh biopsy samples of oral cavity HNSCC were minced, washed with phosphate buffered saline (PBS; ThermoFisher Scientific, Waltham, Mass.), and dissociated using a Human Tumor Dissociation Kit (Miltenyi Biotec, Bergisch Gladbach, Germany) per manufacturer guidelines. Viability was confirmed to be >90% in all samples using trypan blue (ThermoFisher Scientific) exclusion. Cell suspensions were filtered using a 70 μm filter (ThermoFisher Scientific), and dissociated cells were pelleted and re-suspended in PBS with 1% bovine serum albumin (BSA; Sigma-Aldrich, St. Louis, Mo.). Cells were stained with CD45-vioblue (Miltenyi Biotec), along with either the combination of CD9O-PE (BD Biosciences, Franklin Lakes, N.J.) and CD31-PE-cy7 (BD Biosciences) or CD3-PE-cy7 (ThermoFisher Scientific), then washed with cold PBS, and re-suspended for flow cytometry analyses.

Sorting of Patient Samples. Cells were stained for viability with 1 μM calcein AM (ThermoFisher Scientific) and 0.33 μM TO-PRO-3 iodide (ThermoFisher Scientific) immediately prior to sorting. Fluorescence-activated cell sorting (FACS) was performed on FACSAria Fusion Special Order System (BD Biosciences) using 488 nm (calcein AM, 530/30 filter), 640 nm (TO-PRO-3, 670/14 filter), 405 nm (Vioblue, 450/50 filter), 561 nm (PE, 586/15 filter; PE-Cy7, 780/60 filter) lasers. Standard forward scatter height versus area criteria were used to discard doublets and capture singlets. Viable cells were identified as calcein^{high} and TO-PRO^{low} and additional gates were used to enrich or deplete specific cell types in each plate. For each tumor, plates were sorted containing CD45− cells (to deplete immune cells), CD45−/CD90−/CD31− cells (to further deplete fibroblasts and endothelium and enrich for malignant cells), CD45+ cells (to enrich for immune cells), and CD45+/CD3+ cells (to enrich specifically for T-cells). Single cells were sorted into 96-well plates containing TCL buffer (Qiagen, Hilden, Germany) with 1% β-mercaptoethanol. Plates were briefly centrifuged, snap frozen, and stored at −80° C. before cDNA synthesis and library construction. For each tumor sample, at least one CD45− and one CD45+ plate was sequenced.

cDNA Synthesis and Library Construction. Libraries for isolated single cells were generated based on the SMART-Seq2 protocol (Picelli et al., 2014) with the following modifications: RNA was purified using Agencourt RNAClean XP beads (Beckman Coulter, Brea, Calif.), prior to reverse transcription with Superscript II (ThermoFisher Scientific) or Maxima (ThermoFisher Scientific) reverse transcriptase and whole transcriptome amplification using KAPA HiFi HotStart ReadyMix (KAPA Biosystems, Wilmington, Mass.). Full length cDNA libraries were tagmented using the Nextera XT Library Prep Kit (Illumina, San Diego, Calif.). 384 samples were pooled and sequenced as paired-end 38 base reads on a NextSeq 500 instrument (Illumina).

Whole Exome and Targeted Sequencing. Snap frozen fresh biopsy and matched whole blood samples were processed by the Genomics Platform at the Broad Institute. Whole exome sequencing was performed per standard protocols using Illumina technology (Illumina). Briefly, library construction was performed as previously described (Fisher et al., 2011). Subsequently, hybridization and capture were performed using the Rapid Capture Exome Kit (Illumina) per manufacturer protocol. After post-capture enrichment, library pools were quantified using an automated qPCR assay on the Agilent Bravo (Agilent Technologies, Santa Clara, Calif.). Cluster amplification of denatured templates was performed per manufacturer's protocol using HiSeq 4000 cluster chemistry and HiSeq 4000 flowcells (Illumina). Flowcells were sequenced using v1 Sequencing-by-Synthesis chemistry for HiSeq 4000 flowcells. The flowcells were then analyzed using RTA v.1.18.64 or later (Illumina). In addition, SnAPShot next generation sequencing v2 assay was performed on FFPE samples at the MGH Center for Integrated Diagnostics per standard protocols as previously described (Zheng et al., 2014). Sequencing was performed on an Illumina NextSeq (Illumina). Novoalign (Novocraft Technologies, Selangor, Malaysia) was used to align reads to the hg19 human genome reference. Single nucleotide and indel variants were detected using MuTectl (Cibulskis et al., 2013), LoFreq (Wilm et al., 2012), and GATK (DePristo et al., 2011; McKenna et al., 2010; Van der Auwera et al., 2013). Exons from 91 gene targets were sequenced.

RNA-seq of Cell Lines. For scRNA-seq, cells were harvested, stained for viability, and sorted into 96-well plates, as described above. cDNA synthesis, library construction, and sequencing were also performed as described. For bulk RNA, RNA was isolated from 1,000 pooled cells using RNEasy Micro Kit (Qiagen).

Flow Cytometry and Sorting of Cell Lines. Sorting of SCC9 cells was performed using TGFBI antibody (LifeSpan Biosciences, Seattle, Wash.) conjugated to PE using the R-PE IgG labeling kit (ThermoFisher Scientific) per manufacturer specifications. Cells were sorted as described above. For stained samples, cells were considered marker-positive if marker signal was at least as high as the top ˜2% of cells in the unstained control. For repopulation experiments, 10⁵ TGFBI^{high} TGFBI^{low}, and bulk sorted cells were plated and propagated. Cells were harvested after 4 hours, 24 hours, 4 days, and 7 days, stained with TGFBI-PE as described, and re-analyzed by FACS. Cells harvested at 4 hours were not re-stained prior to FACS analysis. Final analysis was performed in FlowJo version 10.2 (TreeStar, Ashland, Oreg.). In addition, single cells in each condition at the 7 day time point were sorted into 96-well plates for scRNA-seq.

Modification of Culture Conditions. For hypoxia cultures, SCC9 cells were grown for seven days in a Galaxy 48R CO₂ incubator (Eppendorf, Hamburg, Germany), with 2% 02, 5% CO₂. Cells were then harvested and FACS sorted for scRNA-seq. For co-culture experiments, a tumor biopsy from MEEI18 was used to derive CAFs by the Broad Institute Cancer Cell Line Factory. Briefly, the tissue was washed with PBS (ThermoFisher Scientific) and minced using a scalpel. It was digested in 5 mL media with 1 mL 10X collagenase-hyaluronidase (StemCell Technologies, Vancouver, Canada) and 1 mL dispase (StemCell Technologies) for one hour at 37° C. Cells were then centrifuged at 1000 rpm for 5 minutes, followed by RBC lysis with a 5 minute incubation in ACK lysis buffer (ThermoFisher Scientific), followed by 3 minutes in 1 mL media with 1:6 DNase I (StemCell Technologies). Cells were then washed and plated for propagation in ACL4 media (RPMI with L-glutamine (ThermoFisher Scientific) with 5% FBS (Sigma-Aldrich), 0.5% BSA (Rockland Immunochemicals, Limerick, Pa.), 10 mM HEPES (Sigma-Aldrich), 0.5 mM sodium pyruvate (Sigma-Aldrich), 0.02 mg/mL insulin (Sigma-Aldrich), 0.01 mg/mL transferrin (Sigma-Aldrich), 25 nM sodium selenite (Sigma-Aldrich), 50 nM hydrocortisone (Sigma-Aldrich), and 1 ng/mL epidermal growth factor (Sigma-Aldrich)). Growth of a pure population of fibroblasts was confirmed by a PCR-based targeted sequencing assay using the TruSeq Custom Amplicon platform (Illumina). These tumor-derived fibroblasts were initially plated at a 1:3 ratio with SCC9 cells, and cells were harvested after 48 hours when the ratio of tumor-derived fibroblasts to SCC9 cells was approximately 1:1.

TGFβ Treatment and TGFBI Overexpression. For drug treatment experiments, SCC9 cells were grown in vehicle (4 μM HCl with 1 μg/mL BSA), TGFβ, or TGFβ-inhibitor. For TGFβ-treated cells, 10 ng/mL recombinant TGFβ1 (R&D Systems, Minneapolis, Minn.) or TGFβ3 (R&D systems) was applied. Cells in the TGFβ-inhibitor condition were either grown in 3:1 F12:DMEM (ThermoFisher Scientific) with 1 μM A-83-01 (Tocris Bioscience, Bristol, UK) or small airway basal medium (Lonza, Basel, Switzerland) with four inhibitors of the TGFβ pathway: 1 μM DMH-1, 1 μM A-83-01, 1 μM CHIR99021 (Tocris Bioscience), and 10 μM Y-27632 (Selleck Chemicals, Houston, Tex.). For scRNA-seq, cells in each condition were harvested 4 hours after treatment. For bulk RNA-seq, cells were harvested 2, 4, or 6 days after treatment and titrated for analysis. For matrigel invasion assay and cell proliferation assays, cells were maintained in the given conditions for the duration of the experiment.

For TGFBI overexpression, TGFBI was PCR-amplified from pDNR-Dual-TGFBI (Harvard Plasmid Consortium, Cambridge, Mass.) using the following primers (Integrated DNA Technologies, Coralville, Iowa): For: 5′-CAC CAT GGC GCT CTT CGT GCG G-3′ (SEQ. I.D. No. 3) and Rev: 5′-CTA ATG CTT CAT CCT CTC-3′ (SEQ. I.D. No. 4). The PCR product was then cloned into pMAL (van Galen et al., 2014) using the pENTR/D-TOPO Cloning Kit (ThermoFisher Scientific) and the Gateway LR Clonase protocol (ThermoFisher Scientific). SCC9 cells at 50-70% confluence were transfected with pMAL-TGFBI or pMAL-Luc (van Galen et al., 2014) using the FuGENE HD transfection reagent (Promega, Madison, Wis.) per manufacturer protocol. Transfection with pMAX-GFP (van Galen et al., 2014) in parallel conditions confirmed adequate transfection efficiency. Cells were harvested 24 hours after transfection.

TGFBI Knockout Using CRISPR-Cas9. CRISPR sgRNAs were subcloned into lentiCRISPRv2 (Addgene, Cambridge, Mass.) using primers listed in the Key Resources Table. The target sequences were: sgRNA1 (exon 1 CDS, antisense): 5′-AGC TGG TAG GGC GAC TTG GC-3′ (SEQ. I.D. No. 5); sgRNA2 (exon 1 CDS, antisense): 5′-CGA CTT GGC GGG ACC CGC CA-3′ (SEQ. I.D. No. 6); and sgRNA3 (exon 8 CDS, sense): 5′-CAT GCT CAC TAT CAA CGG GA-3′ (SEQ. I.D. No. 7). A non-targeting control (“mock”) plasmid (BRDN0001478216, Broad Genetic Perturbation Platform, Broad Institute, Cambridge Mass.) was used for comparison. CRISPR plasmids were co-transfected into 293T cells with GAG/POL and VSVG plasmids, per the Addgene third generation lentiviral system, using the FuGENE HD transfection reagent (Promega) per manufacturer's protocol. At 36 hours post-transfection, the supernatant was collected and concentrated using Lenti-X Concentrator (Clontech), per manufacturer's protocol. SCC9 cells at 70% confluence (approximately 2.5×10⁴ cells) in 24-well plates were infected with concentrated virus for 36 hours, allowed to recover for multiple passages, and selected with 1 μg/mL puromycin (Life Technologies) for 48 hours, prior to harvesting for matrigel and sequencing assays. Genomic DNA was isolated from 3×10⁶ cells using QIAamp DNA Blood Mini Kit (Qiagen). A ˜200 bp fragment surrounding the CRISPR cut site of each sample was PCR amplified (PCR Supermix, ThermoFisher Scientific) using TGFBI NGS primers listed in the Key Resources Table. Efficient genome editing was confirmed with next generation sequencing of PCR products at the Massachusetts General Hospital (MGH) Center for Computational & Integrative Biology (CCIB) DNA Core per standard core protocols. Briefly, this entailed Illumina adapter ligation, low-cycle PCR amplification, and sequencing on the Illumina MiSeq (Illumina). Results were analyzed using the CRISPResso software pipeline (Pinello et al., 2016).

Matrigel Invasion Assay. Matrigel invasion assay was performed as previously described (Puram et al., 2012). Preformed matrigel invasion chambers (Corning, Corning, N.Y.) were prepared per manufacturer protocol. Serum-containing media was placed below the invasion chambers and 2.5×10⁴ cells suspended in 500 μL serum-free media were placed above the invasion chambers and incubated for 24 hours. Cells on the lower surface of the membrane were fixed with methanol, stained with crystal violet, and counted in a blinded manner. Cells in serum-containing media were used as a negative control.

Cell Proliferation Assay. CellTiter-Glo (CTG) proliferation assay were performed per manufacturer protocol. Cells were plated in 96-well plates in 6-9 replicates per condition at 1,000 cells per well. Cells were lysed on days 2, 4, and 6 by adding CTG reagent (Promega), and point luminescence was measured via the BioTek Synergy HTX Platereader (BioTek, Winooski, Vt.). For all experiments, a proportional sampling of cells were also lysed at 1 hour after initial plating to ensure that equal numbers were plated across conditions. For cells lysed on day 6, fresh media was added on day 3. CTG luminescence values for individual wells were normalized by subtracting background luminescence (mean luminescence values for wells containing PBS, with CTG reagent added), adjusting for 2 μM adenosine triphosphate (ATP) luminescence measured on the same 96-well plate, and normalizing by numbers of plated cells in each condition (as measured by T₀ luminescence).

Staining of Tissue Sections. Sectioning and immunohistochemical (IHC) staining of formalin fixed, paraffin-embedded (FFPE) HNSCC specimens was performed by the MGH Histopathology Core per standard protocols. All sections were 5 μm thick. Briefly, antigen retrieval was performed in a decloaker (Biocare Medical) using citrate buffer at pH 6.0. Sections were deparaffinized through xylenes and graded ethanol. Primary antibodies were visualized with HRP- or AP-linked secondary antibodies, followed by diaminobenzidine (DAB; Dako, Glostrup, Denmark) or AP-red (Dako) chromogens, respectively. Sections were counterstained with hematoxylin (ThermoFisher Scientific). Human papillomavirus (HPV) in situ hybridization (ISH) was performed per Advanced Cell Diagnostics RNAscope DAB ISH protocol (Advanced Cell Diagnostics, Newark, Calif.), with dewaxing followed by a 95-minute target retrieval step, incubation with the RNAscope enzyme, and a 6-hour hybridization. Stained sections were visualized using a Nikon Eclipse 90i microscope with a Nikon DS-Fi1 high definition color camera and NIS-Elements Advanced Research version 3.10 software (Nikon, Melville, N.Y.). Images were captured with a 20× objective and were reviewed by a dedicated head and neck pathologist.

TCGA Stromal Quantification. Digital hematoxylin and eosin stained slides for TCGA tumors were downloaded and entire sections were examined in a blinded manner. Working with a dedicated head and neck pathologist (W.C.F.), the stromal content of each basal and mesenchymal tumor was quantified by percent and scored as 0 (<10% stromal content), 1+ (10% to <20%), 2+ (20% to <30%), 3+ (30% to <50%), or 4+ (≥50%).

### Quantification and Statistical Analysis

Statistical analyses were performed with GraphPad Prism version 7. (GraphPad Software, La Jolla, Calif.) or MatLab version 2014b (MathWorks, Natick, Mass.). Parameters such as sample size, the number of replicates, the number of independent experiments, measures of center, dispersion, and precision (mean±SD or SEM), and statistical significance are reported in Figures and Figure Legends. Results were considered statistically significant when p<0.05, or a lower threshold when indicated, by the appropriate test (ANOVA, t-test, Pearson correlation). The Student's t-test, permutation test, and hypergeometric test were utilized for comparisons in experiments with two sample groups. In experiments with more than two sample groups, analysis of variance (ANOVA) was performed followed by Bonferroni's post-hoc test.

Single-Cell RNA-seq Data Processing. Expression levels were quantified as E_{i,j}=log₂(TPM_{i,j}/10+1), where TPM_{i,j} refers to transcript-per-million for gene i in sample j, as calculated by RSEM (Li and Dewey, 2011). TPM values are then divided by 10 since Applicants estimate the complexity of single-cell libraries to be on the order of 100,000 transcripts and would like to avoid counting each transcript ˜10 times, as would be the case with TPM, which may inflate the difference between the expression level of a gene in cells in which the gene is detected and those in which it is not detected. This modification has a minimal influence on the expression values (Spearman correlation of 1, Pearson correlation of 0.98), but decreases the difference between the expression values of undetected genes (i.e. zero) and that of detected genes (data not shown), thereby reducing the impact of dropouts on downstream analysis. Applicants note that the SMART-Seq2 protocol cannot incorporate unique molecular identifiers (UMI) and therefore Applicants cannot directly identify duplicate reads.

For each cell, Applicants quantified two quality measures: (i) the number of genes for which at least one read was mapped, which is indicative of library complexity and (ii) the average expression level (E) of a curated list of housekeeping genes (Tirosh et al., 2016a), which is meant to verify that genes which are expected to be expressed highly, regardless of cell type, are indeed detected as highly expressed. Scatter plot analyses of all profiled cells separated low and high quality cells based on these two measures (data not shown), and Applicants therefore conservatively excluded all cells with either fewer than 2,000 detected genes or an average housekeeping expression level (E) below 2.5, as done in previous studies (Patel et al., 2014; Tirosh et al., 2016a). For cells passing these quality controls, the median number of reads were 1.34 million per cell, with a 52.2% transcriptome mapping rate and 3,880 detected genes.

Applicants used the remaining cells (k=5,902) to identify genes that are expressed at high or intermediate levels by calculating the aggregate expression of each gene i across the k cells, as Eₐ(i)=log₂(average(TPM(i)_{1 . . . k})+1), and excluded genes with Eₐ<4. For the remaining cells and genes, Applicants defined relative expression by centering the expression levels, Er_{i,j}=E_{i,j}−average[E_{i, 1 . . . k}]. The relative expression levels, across the remaining subset of cells and genes, were used for downstream analysis. Although normalization approaches can potentially introduce bias into initial clustering, relative expression levels, as defined above and as defined with an alternative normalization method (Bacher et al., 2017) were highly similar. The use of alternative normalization had a limited influence on downstream results such as the distribution of p-EMT scores.

To test for batch effects, Applicants performed preliminary clustering of all cells using t-SNE with perplexity of 30 followed by density clustering (DBscan with parameters epsilon=5 and MinPoints=15). The resulting clusters showed limited impact of sequencing batches but an apparent batch effect linked to the enzyme used for reverse transcription (Superscript II or Maxima; data not shown). Since these batch effects have a different impact on the transcriptomes of distinct cell types, Applicants corrected the effect in two steps. First, of the 27 clusters identified in the preliminary clustering described below (see Classification to Malignant and Non-malignant Cells and FIG. 8D), Applicants identified seven pairs of clusters that differed by the enzyme used but otherwise were highly similar (as defined by an average Pearson correlation above 0.9); each of these pairs of clusters were then merged, thereby reducing the impact of enzyme usage on cluster assignment. Applicants then normalized the data within each cluster to correct for within-cluster differences that may be linked to enzyme usage. In each cluster, Applicants calculated, for each gene, the average expression among cells processed with Superscript II, the average expression among cells processed with Maxima, and the difference between those. Applicants then subtracted the difference from all cells processed with Maxima in order to correct for the average differences between the two subsets of cells, and make all data comparable to that generated by Superscript II.

Annotation of t-SNE clusters (as in FIGS. 2A and 2C) by the reverse transcription enzyme revealed that all non-malignant clusters and most malignant clusters contained cells processed with both enzymes (data not shown), suggesting that the choice of enzymes has a minimal effect on the final clustering pattern. Five malignant clusters (each corresponding to all malignant cells from a specific tumor) included cells processed only with Superscript II or only with Maxima. Four of these clusters included only cells processed by Superscript II; since the normalization was done to make all data comparable to Superscript II (by only correcting the Maxima-generated data) these clusters should remain comparable to all other clusters. One malignant cluster contained only cells processed by Maxima, corresponding to all malignant cells of MEEI28, which could theoretically introduce variability between MEEI28 and other malignant clusters; however, this tumor had few differentially expressed genes compared to other tumors (FIG. 2D), indicating that batch effects are unlikely to explain the differences between tumors. Importantly, variability of the p-EMT and epithelial differentiation programs was not influenced by the enzyme used for reverse transcription (data not shown).

Epithelial Classification. Applicants defined a set of potential epithelial markers consisting of all cytokeratins, EPCAM, and SFN. Applicants excluded potential markers that were lowly expressed (Eₐ<4) or not co-regulated with the other markers across all single cells (Pearson R<0.4 with the average of all other markers). The average expression (E) of the 14 remaining genes was used to quantify an epithelial score, which was bimodally distributed (FIG. 1C). Epithelial and non-epithelial cells were defined as those with epithelial scores above 3 and below 1.5, respectively, and the remaining cells (with intermediate scores) were unresolved.

CNV Estimation. Initial CNVs (CNV₀) were estimated by sorting the analyzed genes by their chromosomal location and applying a moving average to the relative expression values, with a sliding window of 100 genes within each chromosome, as previously described (Patel et al., 2014; Tirosh et al., 2016a). To avoid considerable impact of any particular gene on the moving average, Applicants limited the relative expression values to [−3,3] by replacing all values above 3 by a ceiling of 3, and replacing values below −3 by a floor of −3. This was performed only in the context of CNV estimation. Applicants scored each cell for the extent of CNV signal, defined as the mean of squares of CNV₀ values across the genome, and for the correlation between the CNV₀ profile of each cell with the average CNV₀ profile of all cells from the corresponding tumor. Putative malignant cells were then defined as those with CNV signal above 0.05 and CNV correlation above 0.5, putative non-malignant cells as those below the two cutoffs, and unresolved cells as those above only one of the thresholds. This initial analysis was based on the average CNV₀ of all cells as a reference, which is biased due to the inclusion of many malignant cells. Applicants thus redefined CNV estimations, the CNV signal, and CNV correlations values using the average patterns of non-malignant cells as a reference. Non-malignant cells were separated into distinct clusters based on t-SNE as described below. For each cluster Applicants defined a baseline reflecting the average CNV₀ estimates of all cells in that cluster, and based on these distinct baselines Applicants defined the maximal (BaseMax) and minimal (BaseMin) baseline at each window. The final CNV estimate of cell i at position j was defined as:

$${CNV}_{f} ( i , j ) = \{ \begin{matrix}{CNV}_{0} ( i , j ) = BaseMax ( j ) , if {CNV}_{0} ( i , j ) > BaseMax ( j ) + 0.2 \\ {CNV}_{0} ( i , j ) = BaseMin ( j ) , if {CNV}_{0} ( i , j ) < BaseMin ( j ) - 0.2 \\ 0 , if BaseMin ( j ) - 0.2 < {CNV}_{0} ( i , j ) < BaseMax ( j ) + 0.2\end{matrix}$$

Classification to Malignant and Non-malignant Cells. Epithelial and CNV-based classifications were highly concordant and enabled robust assignment of single cells as malignant or non-malignant. To further support these classifications, Applicants reasoned that global similarity of gene expression programs should also distinguish between malignant and non-malignant cells. Applicants examined 27 clusters as defined by the preliminary clustering described above. Most clusters contained exclusively malignant or non-malignant cells by the above two criteria. Five clusters of smaller sizes were associated primarily with cells that had unresolved or inconsistent assignments by the above two criteria. These clusters were also associated with low complexity (number of genes detected in each cell) and low expression of housekeeping genes, leading us to suspect that they reflect low-quality data. Exclusion of these cells was therefore useful both in order to maintain confidence in malignant classifications and to remove cells of low quality for which the global expression profile and associated clustering may be highly affected by their low data quality.

Identification of Differentially Expressed Genes. To identify differentially expressed genes between different clusters, including comparisons of non-malignant clusters and of malignant clusters, Applicants combined three criteria: (i) an average fold-change of 2, (ii) a t-test p-value below 10⁻¹⁰, and (iii) a permutation test p-value below 0.001. The latter criterion was defined by shuffling the assignments of cells to clusters 10,000 times and counting the fraction of times where an equal or larger difference was obtained between the average expression of each cluster and that of the remaining clusters. The cutoff in the second criterion ensures the control for multiple testing (a stringent Bonferroni correction would result in a corrected p-value of 6.5×10⁻⁶, as there are at most 10×6,465 tests in the family of hypotheses for differential expression).

Classifying Non-malignant Cells. t-SNE analysis of all non-malignant cells using perplexity of 30 was followed by DBscan clustering (with parameters 5 and 15) to identify eight major clusters. Clustering using this approach was highly consistent with an alternative approach (FIG. 9A) (Bacher et al., 2017). Furthermore, additional t-SNE analyses with multiple perplexity parameters (15, 20, 25, 30 and 35) and six instances for each perplexity parameter confirmed the robustness of the clustering patterns (data not shown). For each original cluster, Applicants quantified its robustness in each alternative t-SNE instance by the fraction of cells for which the five nearest neighbors (in the alternative t-SNE) are all assigned to the same cluster as the cells being examined. This analysis demonstrated an average rate (across the 30 alternative t-SNE analyses) of consistent clustering larger than 99.6% for each of the clusters. Inspection of the top differentially expressed genes revealed classical cell type markers; for each cluster, Applicants thus defined a set of marker genes, which were both identified as differentially expressed and previously associated with a specific cell type. The average expression profiles of those gene-sets were indeed highly specific to the corresponding clusters (FIG. 9), supporting the cell type classifications.

To further identify subtypes Applicants focused on the two cell types with the largest numbers of cells: T-cells and fibroblasts. Applicants used refined DBscan clustering of the t-SNE analysis (with parameters Epsilon=3, and MinPoints=5) to separate each of those clusters to sub-clusters, and further examined the results with multiple t-SNE analyses to evaluate the robustness of cluster assignments.

The T-cell cluster was subdivided into four subtypes, which were annotated based on the differential expression of T cell markers (FIG. 9C). This clustering was not strict as variability among T cells was continuous, yet the four clusters were used to represent the main patterns of variability that Applicants observed among T cells (exhausted, CD4, CD8, Tregs).

For fibroblasts, Applicants first observed two robust sub-clusters (myofibroblasts and CAFs, each with more than 98% consistent clustering as defined above) and a third intermediate sub-cluster which was less robust (89% consistent clustering, data not shown). In subsequent analysis, Applicants explored further the diversity of fibroblasts using a focused PCA (FIG. 9F). This analysis was restricted to fibroblasts and to genes that are preferentially expressed by fibroblasts (defined as Eₐ of fibroblast higher than Eₐ of all other non-malignant cells combined). It recapitulated the three sub-clusters defined above, but also demonstrated that CAFs may be further separated into two subtypes (CAF1 and CAF2) that differ in the expression of many ligands, receptors, and other fibroblast-related genes (FIG. 9G).

Expression Programs of Intra-tumoral Heterogeneity. For each of the 10 tumors, non-negative matrix factorization (as implemented by the Matlab nnmf function, with the number of factors set to 10) was used to identify variable expression programs. NNMF was applied to the relative expression values (Er), by transforming all negative values to zero. Notably, undetected genes include many drop-out events (genes that are expressed but are not detected in particular cells due to the incomplete transcriptome coverage), which introduce challenges for normalization of single-cell RNA-seq; since NNMF avoids the exact normalized values of undetected genes (as they are all zero), it may be beneficial in analysis of single-cell RNA-seq (data not shown). Applicants retained only programs for which the standard deviation in cell scores within the respective tumor was larger than 0.8, which resulted in a total of 60 programs across the 10 tumors. The 60 programs were compared by hierarchical clustering (data not shown), using one minus the Pearson correlation coefficient over all gene scores as a distance metric. Six clusters of programs were identified manually (FIG. 3B) and used to define meta-signatures. For each cluster, NNMF gene scores were log₂-transformed and then averaged across the programs in the cluster, and genes were ranked by their average scores (see Table S6 for the top 50 genes in each cluster). The top 30 genes for each cluster were defined as the meta-signature that was used to define cell scores (see Table S7); each of those genes had average scores above 1 and a t-test p-value below 0.05, based on their scores across the individual programs in the cluster. Since the number of programs in a cluster was small this analysis was not powered to correct for multiple testing and thus Applicants refer to an uncorrected p-value and selected the top ranked genes. However, while confidence is difficult to establish for individual genes in each meta-program, each gene-set defined as a meta-program is highly significant in its co-variation in tumors. For each of the meta-programs, and within each of the tumors included in those meta-programs (2-8 tumors for each meta-program), the average Pearson correlation between all pairs of genes included in the gene-set (calculated across single malignant cells from the respective tumor) was higher than that obtained for 10,000 control gene-sets, which were selected to reproduce the overall distribution of expression levels of the meta-program genes (see also Defining Cell and Sample Scores).

To show the robustness of the NNMF-derived programs with regards to the number of NNMF factors in the dataset, Applicants repeated the NNMF analysis with the number of factors between 5 and 15 (data not shown). Applicants then compared the resulting NNMF programs to the meta-programs defined in the original analysis, with a threshold of global Pearson correlation (across all genes) of 0.2. This threshold is highly significant as it was never observed among 10,000 permutation analyses, in which Applicants permuted the centered expression data of each cell and repeated the analysis. Each of the six meta-programs was identified with each of the NNMF parameters.

Defining Cell and Sample Scores. Applicants used cell scores in order to evaluate the degree to which individual cells express a certain pre-defined expression program. These are initially based on the average expression of the genes from the pre-defined program in the respective cell: Given an input set of genes (Gⱼ), Applicants define a score, SCⱼ(i), for each cell i, as the average relative expression (Er) of the genes in Gⱼ. However, such initial scores may be confounded by cell complexity, as cells with higher complexity have more genes detected (i.e. less zeros) and consequently would be expected to have higher cell scores for any gene-set. To control for this effect Applicants also add a control gene-set (Gⱼ^{cont}); Applicants calculate a similar cell score with the control gene-set and subtract it from the initial cell scores: SCⱼ(i)=average[Er(Gⱼ,i)]−average[Er(Gⱼ^{cont},i)]. The control gene-set is selected in a way that ensures similar properties (distribution of expression levels) to that of the input gene-set to properly control for the effect of complexity. First, all analyzed genes are binned into 25 bins of equal size based on their aggregate expression levels (Ea). Next, for each gene in the given gene-set, Applicants randomly select 100 genes from the same expression bin. In this way, the control gene-set has a comparable distribution of expression levels to that of the considered gene-set, and is 100-fold larger, such that its average expression is analogous to averaging over 100 randomly-selected gene-sets of the same size as the considered gene-set. A similar approach was used to define bulk sample scores from TCGA.

Flow Cytometry and Sorting of Cell Lines. Applicants performed n=3 independent experiments for TGFBI staining. For stained samples, cells were considered marker-positive if marker signal was at least as high as the top ˜2% of cells in the unstained control.

Matrigel Invasion Assay. Applicants performed n=3 independent experiments per condition, and n=4-6 replicates per independent experiment. Invaded cells in each well were counted in a blinded manner across four distinct high powered fields and averaged. Error was calculated as SEM for a representative experiment.

Cell Proliferation Assay. Applicants performed n=3-4 independent experiments per condition, and n=6-9 replicates per independent experiment. CTG luminescence values for individual wells were normalized by subtracting background luminescence (mean luminescence values for wells containing PBS, with CTG reagent added), adjusting for 2 μM adenosine triphosphate (ATP) luminescence measured on the same 96-well plate, and normalizing by numbers of plated cells in each condition (as measured by T₀ luminescence). Error was calculated as SEM for a representative experiment.

Putative Interactions Between Cell Types. Applicants identified putative interactions between any pair of cell types based on expression of a receptor by one cell type and expression of an interacting ligand by the other cell type: whenever a ligand transcript is “expressed” by cell type A and the interacting receptor transcript is “expressed” by cell type B, Applicants define it as a potential interaction between A and B. If the malignant cells express the receptor or the ligand, then the corresponding interaction was defined as incoming or outgoing, respectively. This analysis required two additional definitions. First, the set of potential receptor-ligand interactions were obtained from Ramilowski et al. (Nature Communications, 2015). Second, a ligand or receptor transcript was defined as “expressed” by a given cell type if its average expression in that cell type was above the threshold of 4 (in values of log₂(TPM+1)).

TCGA Subtype Analysis. Bulk RNA-seq data of HNSCC tumors (rnaseqv2-RSEM_genes_normalized) was downloaded from the Broad Firehose website (gdac.broadinstitute.org/), along with additional tumor and clinical annotations. Expression data was log₂-transformed, filtered to include only the top 10,000 genes (based on average expression), centered for each gene, and compared between subtypes. Applicants identified all genes preferentially expressed in each of the four subtypes (fold-change >2 and p<0.01 by t-test, when comparing a given subtype to each of the other three subtypes) and scored single cells by the four subtype gene-sets (FIGS. 6A and 6B). To further examine the classification of TCGA samples, Applicants first calculated the average Pearson correlation of each sample with all samples classified by TCGA into a given subtype; samples with an average correlation above 0.1 to one (and only one) subtype were retained for further analysis (FIGS. 6C-F), while samples with lower correlations for all four subtypes or higher correlation to more than one subtype were excluded.

Inferring Cancer-cell Specific Expression. Applicants first excluded all genes that are not expressed by the malignant cells (i.e., are only expressed by the TME) based on the single-cell data. Applicants retained cells with Eₐ above 3 (as calculated only over the malignant cells). While this step reduces the influence of TME on bulk expression profiles, it is not sufficient to control for the effect of TME because most genes expressed by malignant cells are also expressed at comparable levels by additional cell types in the TME. Applicants thus aimed to remove this influence using regression analysis. For each of the cell types (t) (both TME and malignant cells) Applicants used the average expression of cell type-specific genes to estimate the relative abundance of the cell type (Frₜ) across all bulk tumors. These estimates were then used for a multiple linear regression seeking to approximate Ex(i,g), the (log-transformed and centered) expression level of gene g in bulk tumor i, by the sum of Frₜ(i), the estimated relative cell type frequencies of tumor i, multiplied by gene-specific and cell type-specific scaling factors Xₜ(g):

Ex(i,g)=Σ_{t∈T}_{_{g}}(Frₜ(i)*Xₜ(g))+R(i,g)

T_{g} includes all the cell types for which the average expression of gene g is lower than that of the malignant cells by at most 2-fold; note that this definition includes also the malignant cell as a cell type, which enables the regression to account for purity. This regression defines the scaling factors Xₜ(g) that minimize the sum of squares of the residuals, R(i,g), which reflect the component of expression level that is not accounted by the expression of cell types T_{g} based on the assumption of linear relationship between cell type abundances and total expression level; Applicants define the residuals as the inferred cancer-cell specific expression.

p-EMT Stratification of TCGA samples. Since p-EMT and epithelial differentiation scores were a prominent source of variability in malignant-basal tumors, but not in classical and atypical, Applicants classified only those tumors into p-EMT high and p-EMT low. Applicants defined sample scores (see Defining Cell and Sample Scores) for all malignant-basal tumors based on the inferred cancer-cell specific expression of the p-EMT and epithelial differentiation (Epi. Diff. 2) signatures; only the subset of genes from these signatures which were included in the inferred cancer-cell specific expression were used for these scores. Applicants then ranked the tumors based on their p-EMT score minus the epithelial differentiation, and defined the highest 40% as p-EMT high and the lowest 40% as p-EMT low, while excluding the remaining 20% of tumors with intermediate scores.

Prognostic analysis of p-EMT and CAF scores. To evaluate the effect of p-EMT on seven clinical features (FIG. 7C), Applicants compared the fractions of patients with that feature between p-EMT high and p-EMT low tumors, and evaluated the significance of enrichments with a hypergeometric test. To further evaluate the effect of p-EMT while also taking CAF frequency (which is highly consistent with TCGA mesenchymal scores) into account, Applicants used a binomial logistic regression model as implemented by MATLAB fitglm function, with binomial distribution and included interactions. These models fit a logistic regression of two effects (p-EMT scores and CAF frequency scores) and their interactions, in order to predict the clinical features, with a separate model for each feature. The p-values from these models are shown in the bottom panel of FIG. 14I.

### Data and Software Availability

Raw expression and WES data is available through dbGAP (study ID 26106). Processed expression data is available through the Gene Expression Omnibus (www.ncbi.nlm.nih.gov/geo/) with accession number GSE103322. Matlab scripts for analyses are available through the Trinity Cancer Transcriptome Analysis Toolkit (github.com/NCIP/Trinity_CTAT/wiki).

TABLE S1
Patients and samples included in dataset, Related to FIG.1.

| | | | Lymph Node(s) | Pathologic | | | | |
|---|---|---|---|---|---|---|---|---|
| Designation | Age/Sex | Primary Site | Collected | Stage | Grade | PNI | LVI | ECE |
| MEEI5 | 69/F | Left lateral tongue | Left level 2 | T2N1 | 2 | Present | Present | Absent |
| MEEI6 | 88/F | Right floor of mouth | Left level 2 | T4aN2c | 1 | Present | Present | Present |
| MEEI7 | 71/F | Right floor of mouth | — | T1N2b | 3 | Absent | Present | Present |
| MEEI8 | 82/F | Right hard palate | — | T4aN0 | 1 | Absent | Absent | — |
| MEEI9 | 77/F | Right lateral tongue | — | T1N0 | 2 | Absent | Absent | — |
| MEEI10 | 76/M | Right retromolar trigone | — | T4aN2b | 2 | Absent | Absent | Present |
| MEEI12 | 80/M | Left retromolar trigone | — | T4aN0 | 2 | Present | Present | — |
| MEEI13 | 52/F | Left lateral tongue | — | T3N1 | 2 | Present | Present | Absent |
| MEEI16 | 63/F | Left lateral tongue | — | T2N0 | 1 | Absent | Absent | — |
| MEEI17 | 59/M | Right alveolar ridge | — | T4aN0 | 2 | Present | Absent | — |
| MEEI19 | 41/M | Left lateral tongue | — | T3N1 | 2 | Present | Present | Absent |
| MEEI20 | 53/M | Right floor of mouth | Right level 3 | T4aN2c | 2 | Present | Present | Present |
| MEEI22 | 77/M | Left buccal mucosa | — | T1N0 | 2 | Absent | Absent | — |
| MEEI23 | 56/M | Right retromolar trigone | — | T3N1 | 2 | Absent | Present | Absent |
| MEEI24 | 78/F | Right alveolar ridge | — | T4aN2c | 2 | Absent | Absent | Absent |
| MEEI25 | 76/F | Left lateral tongue | Left level 2 | T3N1 | 2 | Present | Present | Present |
| MEEI26 | 51/M | Left floor of mouth | Right and left level 1 | T4aN2c | 3 | Present | Present | Absent |
| MEEI28 | 58/M | Right lateral tongue | Left level 2 | T2N2c | 1 | Present | Present | Absent |

PNI = perineural invasion;
LVI = lymphovascular invasion;
ECE = extracapsular extension

TABLE S2
Clinical and pathologic features of deeply sequenced samples,
Related to FIG. 1. p16 immunohistochemistry and HPV in situ hybridization were negative for all samples.

| Desig- | | Size | | | | | | | | P16 | HPV |
|---|---|---|---|---|---|---|---|---|---|---|---|
| nation | Primary Site | (cm) | Description | LN Involved | Stage | Grade | PNI | LVI | ECE | IHC | PCR |
| MEEI 5 | L lateral tongue | 3.6 | invasive SCC, keratinizing | 1/21. L level 2. <1.0 cm | T2N1 | 2 | Present | Present | Absent | (−) | (−) |
| MEEI 6 | R floor of mouth | 3.9 | invasiveSCC, keratinizing, | 5/34. bilateral, | T4aN2c | 1 | Present | Present | Present | (−) | (−) |
| | | | involving bone | largest 1.8 cm | | | | | | | |
| MEEI 16 | L lateral tongue | 2.2 | invasive SCC, keratinizing | 0/23 | T2N0 | 1 | Absent | Absent | — | (−) | (−) |
| MEEI 17 | R alveolar ridge | 3.8 | invasive SCC, keratinizing, | 0/30 | T4aN0 | 2 | Present | Absent | — | (−) | (−) |
| | | | involving bone | | | | | | | | |
| MEEI 18 | L lateral tongue | 5.5 | invasive SCC, keratinizing | 1/23. L level 1, 0.4 cm | T3N1 | 2 | Present | Present | Absent | (−) | (−) |
| MEEI 20 | R floor of mouth | 5.2 | invasive SCC, | 5/50. bilateral, | T4aN2c | 2 | Present | Present | Present | (−) | (−) |
| | | | non-keratinizing, | largest 3.0 cm | | | | | | | |
| | | | basaloid, necrotic | | | | | | | | |
| MEEI 22 | L buccal mucesa | 2.0 | invasive SCC, keratinizing | Not examined | T1N0 | 2 | Absent | Absent | — | (−) | (−) |
| MEEI 25 | L floor of mouth | 6.2 | invasive SCC, keratinzing | 1/9. L level 2, 2.5 cm | T3N1 | 2 | Present | Present | Present | (−) | (−) |
| MEEI 28 | L floor of mouth | 3.6 | invasive SCC, involving | 2/72. bilateral, | T4aN2c | 3 | Present | Present | Absent | (−) | (−) |
| | | | bone and muscle | largest 1.6 cm | | | | | | | |
| MEEI 28 | R lateral tongue | 3.0 | invasive SCC, keratinzing | 4/20. bilateral, | T2N2c | 1 | Present | Present | Absent | (−) | (−) |
| | | | | largest 2.1 cm | | | | | | | |

L = left;
R = right;
SCC = squamous cell carcinoma;
PNI = perineural invasion;
LVI = lymphovascular invasion;
ECE = extracapsular extension;
IHC = immunohistochemistry;
HPV = human papillomavirus;
PCR = polymerase chain reaction

TABLE S3
Mutations and copy number variations detected in profiled primary tumors, Related to FIG. 1.
Common mutations evaluated by whole exome sequencing of a subset of samples and SNaPshot next generation sequencing
assay of all samples include the top 5 mutations in TCGA HNSCC tumors, as well as mutations in TERT promoter.
CNVs evaluated include top 4 abnormalities noted in TCGA HNSCC tumors.

| | | Reported | MEEI5 | MEEI6 | MEEI16 | MEEI17 | MEEI18 | MEEI20 | MEEI22 | MEEI25 | MEEI26 | MEEI28 |
|---|---|---|---|---|---|---|---|---|---|---|---|---|
| Common | TP53 | 72%^{a} | Mut^{1,2} | Mut² | ND | ND | Mut² | Mut^{1,2} | Mut² | Mut^{1,2} | Mut^{1,2} | ND |
| Mutations | TERT promoter | 30%^{b} | Mut² | ND | Mut² | Mut² | ND | ND | Mut² | Mut² | ND | Mut2 |
| | FAT1 | 23%^{a} | ND | ND | ND | ND | ND | ND | ND | Mut¹ | ND | ND |
| | CDKN2A | 22%^{a} | ND | ND | ND | ND | ND | Mut^{1,2} | Mut² | Mut¹ | ND | ND |
| | PIK-3CA | 21%^{a} | Mut^{1,2} | ND | ND | ND | Mut² | ND | ND | ND | ND | ND |
| | NOTCH1 | 19%^{a} | ND | ND | ND | ND | ND | Mut¹ | ND | ND | ND | Mut¹ |
| Other | — | — | TSC2² | FSXW7² | — | AJUBA¹, | MET², | NSD1¹ | — | — | NFE2L2¹ | AJUBA¹, |
| | | | | | | FBXW7² | BRCA2² | | | | | TGFER2¹, |
| | | | | | | | | | | | | SMARCB1², |
| | | | | | | | | | | | | CIC² |
| CRVs | 3p loss | 57% | ++ | + | ND | ND | ++ | ++ | ++ | ND | ND | ND |
| | 3q gain | 44% | ++ | + | ++ | ND | ++ | ++ | ++ | ND | ++ | ND |
| | 8p loss | 29% | ++ | ND | ND | ND | ++ | + | + | ++ | ND | + |
| | 8q gain | 56% | ++ | + | ND | ND | ++ | ++ | ++ | ++ | ND | ND |

^{a}Reported in TCGA;
^{b}reported in Morris et al. (2016);
Mut = mutation detected;
ND = mutation not detected;
¹mutation detected by whole exome sequencing;
²mutation detected by SNaPshot;
CNV = copy number variation

TABLE S4
Mutations detected by whole exome sequencing, Related to FIG. 1.
Mutations are sorted by patient number, within patient by primary tumor followed by lymph node,
and within sample by location within the genome.

| | | | | Alleles | | Protein | Count | |
|---|---|---|---|---|---|---|---|---|
| Sample | Gene | Genome Position | Mutation Type | Ref | Alt | Change | Ref | Alt |
| MEEI5 Primary | LRRC47 | chr1:3700584 | Missense | C | T | R429Q | 42 | 5 |
| MEEI5 Primary | HDAC1 | chr1:32796249 | Missense | C | G | S267C | 64 | 6 |
| MEEI5 Primary | COL24A1 | chr1:86252048 | Nonsense | G | A | Q1350* | 53 | 5 |
| MEEI5 Primary | GSTM5 | chr1:110255922 | 5′UTR | C | G | | 26 | 3 |
| MEEI5 Primary | ST7L | chr1:113153504 | Missense | G | C | S137W | 39 | 7 |
| MEEI5 Primary | AQP10 | chr1:154293718 | Silent | G | A | L29L | 33 | 4 |
| MEEI5 Primary | GEN1 | chr2:17962979 | Missense | G | C | E834Q | 70 | 7 |
| MEEI5 Primary | GCKR | chr2:27730108 | Missense | G | C | R358P | 115 | 6 |
| MEEI5 Primary | CLIP4 | chr2:29355024 | Missense | A | C | K94Q | 42 | 7 |
| MEEI5 Primary | RALB | chr2:121047157 | Splice Site | G | A | E109_splice | 42 | 4 |
| MEEI5 Primary | RALB | chr2:121047174 | Silent | G | A | V114V | 45 | 4 |
| MEEI5 Primary | MAP2 | chr2:210559379 | Missense | G | T | A829S | 55 | 8 |
| MEEI5 Primary | PIK3CA | chr3:178952085 | Missense | A | G | H1047R | 79 | 5 |
| MEEI5 Primary | AHSG | chr3:186338633 | Missense | C | T | R340C | 107 | 17 |
| MEEI5 Primary | MB21D2 | chr3:192516720 | Missense | G | C | Q311E | 59 | 8 |
| MEEI5 Primary | TACC3 | chr4:1746508 | Silent | G | A | L800L | 98 | 9 |
| MEEI5 Primary | CPZ | chr4:8608565 | Silent | G | A | A199A | 73 | 5 |
| MEEI5 Primary | WDR36 | chr5:110461326 | Missense | C | A | L847M | 110 | 10 |
| MEEI5 Primary | PCDHA12 | chr5:140255349 | Missense | C | T | R98W | 222 | 27 |
| MEEI5 Primary | PCDHB4 | chr5:140503140 | Silent | C | T | Y520Y | 244 | 13 |
| MEEI5 Primary | FAM71B | chr5:156589623 | Silent | G | A | V551V | 97 | 6 |
| MEEI5 Primary | SOX30 | chr5:157073806 | Missense | C | T | R409H | 49 | 6 |
| MEEI5 Primary | ID4 | chr6:19838204 | Silent | C | T | S73S | 87 | 5 |
| MEEI5 Primary | HIST1H2AH | chr6:27115181 | Missense | G | A | E92K | 143 | 12 |
| MEEI5 Primary | HIST1H2AK | chr6:27805934 | Nonsense | C | A | E62* | 89 | 8 |
| MEEI5 Primary | PLAGL1 | chr6:144262931 | Missense | G | A | 5341L | 103 | 5 |
| MEEI5 Primary | SYNE1 | chr6:152665335 | Nonsense | G | A | Q4036* | 131 | 12 |
| MEEI5 Primary | DGKB | chr7:14216421 | 3′UTR | C | A | | 18 | 5 |
| MEEI5 Primary | MPDZ | chr9:13247678 | Missense | G | C | Q47E | 76 | 5 |
| MEEI5 Primary | SFMBT2 | chr10:7239547 | Missense | G | A | S554L | 64 | 6 |
| MEEI5 Primary | SLC29A3 | chr10:73115934 | Missense | C | T | T236M | 113 | 8 |
| MEEI5 Primary | OR52N1 | chr11:5809606 | Silent | C | G | G147G | 174 | 7 |
| MEEI5 Primary | LDHA | chr11:18428720 | Missense | G | C | Q297H | 60 | 5 |
| MEEI5 Primary | HEPHL1 | chr11:93779040 | Silent | C | T | Y124Y | 72 | 9 |
| MEEI5 Primary | BCO2 | chr11:112086986 | Missense | T | C | V520A | 77 | 8 |
| MEEI5 Primary | ZBTB16 | chr11:114112938 | Silent | G | A | A501A | 142 | 8 |
| MEEI5 Primary | SLC38A1 | chr12:46594880 | Splice Site | C | T | | 64 | 6 |
| MEEI5 Primary | MARS | chr12:57892344 | Missense | C | G | I343M | 172 | 11 |
| MEEI5 Primary | MARS | chr12:57905609 | Silent | C | G | L499L | 113 | 5 |
| MEEI5 Primary | NAV3 | chr12:78574810 | Missense | G | A | E1893K | 41 | 5 |
| MEEI5 Primary | NAV3 | chr12:78574816 | Missense | G | A | V1895I | 36 | 6 |
| MEEI5 Primary | MYBPC1 | chr12:102053525 | Missense | G | A | E603K | 237 | 18 |
| MEEI5 Primary | TMEM132C | chr12:128899409 | Missense | G | A | R73Q | 69 | 6 |
| MEEI5 Primary | TMEM132D | chr12:129694156 | Missense | G | A | P451L | 54 | 6 |
| MEEI5 Primary | COL4A2 | chr13:111164485 | Missense | G | A | G16965 | 41 | 5 |
| MEEI5 Primary | MYH7 | chr14:23884311 | Missense | G | A | R1818W | 183 | 15 |
| MEEI5 Primary | SIX4 | chr14:61187006 | Missense | T | C | I34W | 72 | 7 |
| MEEI5 Primary | AHNAK2 | chr14:105416973 | Silent | C | G | L1605L | 171 | 5 |
| MEEI5 Primary | CHST14 | chr15:40764072 | Silent | C | G | L220L | 251 | 15 |
| MEEI5 Primary | MAP1A | chr15:43814174 | Missense | G | A | R406H | 53 | 11 |
| MEEI5 Primary | GRIN2A | chr16:9858247 | Missense | G | T | L1052I | 185 | 10 |
| MEEI5 Primary | CACNG3 | chr16:24268008 | 5′UTR | G | T | | 20 | 3 |
| MEEI5 Primary | SRCAP | chr16:30740437 | Missense | G | A | G1937S | 43 | 6 |
| MEEI5 Primary | TAF1C | chr16:84216925 | Silent | G | C | L111L | 77 | 4 |
| MEEI5 Primary | ANKRD11 | chr16:89353559 | Intron | G | C | | 30 | 3 |
| MEEI5 Primary | ELP5 | chr17:7163048 | 3′UTR | G | A | | 22 | 4 |
| MEEI5 Primary | TP53 | chr17:7577509 | Missense | C | T | E258K | 66 | 5 |
| MEEI5 Primary | TVP23B | chr17:18692727 | Missense | G | C | R25T | 65 | 5 |
| MEEI5 Primary | VTN | chr17:26694761 | Missense | G | C | I433M | 47 | 4 |
| MEEI5 Primary | LINC00482 | chr17:79278543 | lincRNA | C | T | | 123 | 9 |
| MEEI5 Primary | LAMA3 | chr18:21426363 | Silent | C | T | T1274T | 115 | 7 |
| MEEI5 Primary | DSG2 | chr18:29126415 | Silent | C | T | F1022F | 63 | 5 |
| MEEI5 Primary | PGPEP1 | chr19:18474211 | Missense | G | A | D150N | 34 | 4 |
| MEEI5 Primary | CRTC1 | chr19:18879591 | Silent | C | T | I452I | 20 | 3 |
| MEEI5 Primary | ATP1A3 | chr19:42479866 | Silent | G | A | I739I | 138 | 7 |
| MEEI5 Primary | ERCC2 | chr19:45860963 | Intron | G | C | | 109 | 9 |
| MEEI5 Primary | ZNF331 | chr19:54074873 | Missense | G | A | A9T | 28 | 3 |
| MEEI5 Primary | PHEX | chrX:22239861 | Splice Site | G | A | | 103 | 10 |
| MEEI5 Primary | FAM47C | chrX:37027654 | Missense | C | T | R391C | 155 | 16 |
| MEEI5 Primary | SYP | chrX:49055471 | Missense | G | A | R20W | 139 | 6 |
| MEEI5 Primary | SASH3 | chrX:128927086 | Missense | C | T | T308M | 85 | 5 |
| MEEI5 Primary | UTP14A | chrX:129055570 | 3′UTR | T | C | | 31 | 6 |
| MEEI5 Primary | Unknown | chrGL000205.1:117244 | IGR | G | A | | 50 | 11 |
| MEEI17 Primary | PEX14 | chr1:10683298 | Intron | G | A | | 85 | 12 |
| MEEI17 Primary | NBPF1 | chr1:16892261 | Silent | G | C | V977V | 3107 | 35 |
| MEEI17 Primary | KIAA0754 | chr1:39876883 | Missense | G | A | E180K | 161 | 16 |
| MEEI17 Primary | PABPC4 | chr1:40038188 | Missense | C | G | Q88H | 233 | 18 |
| MEEI17 Primary | PPIE | chr1:40205848 | Missense | G | A | E15K | 91 | 17 |
| MEEI17 Primary | PTPRF | chr1:44063417 | Splice Site | A | C | | 85 | 15 |
| MEEI17 Primary | HPDL | chr1:45793070 | Missense | G | A | D84N | 171 | 17 |
| MEEI17 Primary | ZYG11B | chr1:53255680 | Silent | G | A | Q425Q | 56 | 7 |
| MEEI17 Primary | C1orf177 | chr1:55277792 | Missense | C | T | T231I | 436 | 38 |
| MEEI17 Primary | TMEM61 | chr1:55457693 | Missense | C | G | L184V | 341 | 24 |
| MEEI17 Primary | ELTD1 | chr1:79392609 | Missense | C | G | E349Q | 139 | 14 |
| MEEI17 Primary | NTNG1 | chr1:107691206 | 5′UTR | G | C | | 98 | 13 |
| MEEI17 Primary | POGZ | chr1:151377986 | Silent | G | A | F1175F | 221 | 26 |
| MEEI17 Primary | GATAD2B | chr1:153800799 | Missense | G | C | L9V | 107 | 18 |
| MEEI17 Primary | TPM3 | chr1:154155674 | 5′UTR | C | T | | 142 | 19 |
| MEEI17 Primary | ASH1L | chr1:155308026 | Missense | G | C | A2891G | 318 | 41 |
| MEEI17 Primary | NES | chr1:156641146 | Missense | G | C | 5945C | 285 | 14 |
| MEEI17 Primary | SH2D2A | chr1:156777079 | Missense | A | G | L364P | 99 | 14 |
| MEEI17 Primary | AIM2 | chr1:159035839 | Missense | C | T | R226H | 276 | 5 |
| MEEI17 Primary | KIAA0040 | chr1:175130027 | Silent | G | T | I41I | 257 | 25 |
| MEEI17 Primary | PTPN7 | chr1:202119391 | 5′UTR | G | C | | 141 | 4 |
| MEEI17 Primary | RPS6KC1 | chr1:213445971 | Missense | G | A | M1065I | 196 | 21 |
| MEEI17 Primary | RAB3GAP2 | chr1:220363471 | Missense | C | T | R550Q | 182 | 41 |
| MEEI17 Primary | RYR2 | chr1:237608761 | Missense | G | A | E411K | 205 | 37 |
| MEEI17 Primary | ATAD2B | chr2:24055197 | 5′UTR | G | C | | 16 | 3 |
| MEEI17 Primary | CAD | chr2:27460930 | Missense | G | A | E1579K | 145 | 16 |
| MEEI17 Primary | ElF2B4 | chr2:27587233 | 3′UTR | G | T | | 252 | 22 |
| MEEI17 Primary | FEZ2 | chr2:36805813 | Missense | G | T | A277E | 136 | 11 |
| MEEI17 Primary | PRKD3 | chr2:37516575 | Missense | T | A | N214I | 144 | 12 |
| MEEI17 Primary | EML4 | chr2:42544613 | Silent | C | G | L701L | 73 | 4 |
| MEEI17 Primary | THADA | chr2:43455169 | Intron | A | G | | 22 | 4 |
| MEEI17 Primary | AC096579.13 | chr2:89160209 | RNA | T | A | | 34 | 5 |
| MEEI17 Primary | IGKV1-5 | chr2:89246970 | RNA | T | C | | 539 | 6 |
| MEEI17 Primary | MAP4K4 | chr2:102460728 | Missense | G | C | Q396H | 260 | 27 |
| MEEI17 Primary | MARCO | chr2:119752005 | Missense | C | T | T413M | 170 | 8 |
| MEEI17 Primary | TTN | chr2:179669354 | Missense | G | A | P6S | 334 | 5 |
| MEEI17 Primary | GULP1 | chr2:189393848 | Missense | G | A | E63K | 93 | 10 |
| MEEI17 Primary | STK11IP | chr2:220474016 | Intron | G | A | | 91 | 15 |
| MEEI17 Primary | NYAP2 | chr2:226447625 | Missense | G | T | A498S | 459 | 51 |
| MEEI17 Primary | BHLHE40 | chr3:5022064 | Missense | G | C | D77H | 175 | 23 |
| MEEI17 Primary | TRIM71 | chr3:32932472 | Silent | G | A | P592P | 111 | 8 |
| MEEI17 Primary | LAMB2 | chr3:49159238 | Missense | G | A | A1660V | 483 | 40 |
| MEEI17 Primary | ABHD6 | chr3:58253021 | Silent | C | T | L75L | 178 | 13 |
| MEEI17 Primary | FRMD4B | chr3:69230594 | Missense | C | G | Q715H | 193 | 13 |
| MEEI17 Primary | CCDC14 | chr3:123633715 | Missense | C | T | E725K | 83 | 10 |
| MEEI17 Primary | COL6A6 | chr3:130293368 | Splice Site | C | A | S1182_splice | 74 | 9 |
| MEEI17 Primary | GFM1 | chr3:158402347 | Missense | C | T | S600F | 275 | 6 |
| MEEI17 Primary | SI | chr3:164712194 | Splice Site | C | T | | 171 | 17 |
| MEEI17 Primary | PARL | chr3:183585696 | Missense | T | C | Y93C | 204 | 16 |
| MEEI17 Primary | PIGG | chr4:502620 | Silent | G | A | E254E | 118 | 9 |
| MEEI17 Primary | CRIPAK | chr4:1388985 | Missense | C | T | P229L | 840 | 14 |
| MEEI17 Primary | UGT2B15 | chr4:69513036 | Missense | C | T | R460Q | 293 | 32 |
| MEEI17 Primary | MTHFD2L | chr4:75107460 | 3′UTR | G | C | | 15 | 3 |
| MEEI17 Primary | FRAS1 | chr4:79385249 | Missense | G | C | E2280Q | 70 | 4 |
| MEEI17 Primary | AGPAT9 | chr4:84457805 | Silent | C | T | I10I | 63 | 8 |
| MEEI17 Primary | SLC9B2 | chr4:103949943 | Missense | T | C | I451M | 77 | 15 |
| MEEI17 Primary | FBXW7 | chr4:153253852 | Nonsense | G | C | S294* | 185 | 21 |
| MEEI17 Primary | SLC6A3 | chr5:1406366 | Silent | G | T | T512T | 279 | 6 |
| MEEI17 Primary | NSUN2 | chr5:6623348 | Silent | G | C | L172L | 154 | 12 |
| MEEI17 Primary | TARS | chr5:33441163 | 5′UTR | C | A | | 173 | 17 |
| MEEI17 Primary | ZSWIM6 | chr5:60831313 | Missense | G | A | G750R | 116 | 12 |
| MEEI17 Primary | KIAA0825 | chr5:93820533 | Missense | C | T | G358D | 88 | 9 |
| MEEI17 Primary | APC | chr5:112090679 | Missense | C | T | S31F | 278 | 25 |
| MEEI17 Primary | CD14 | chr5:140012193 | Missense | G | T | L126I | 249 | 20 |
| MEEI17 Primary | PCDHA3 | chr5:140180885 | Missense | G | A | V35I | 364 | 40 |
| MEEI17 Primary | PCDHB18 | chr5:140615818 | RNA | G | A | | 938 | 116 |
| MEEI17 Primary | DOCK2 | chr5:169472889 | Missense | G | C | E1316Q | 299 | 7 |
| MEEI17 Primary | TLX3 | chr5:170736340 | 5′UTR | A | C | | 164 | 27 |
| MEEI17 Primary | TLX3 | chr5:170736345 | 5′UTR | A | C | | 174 | 26 |
| MEEI17 Primary | FGF18 | chr5:170847102 | 5′UTR | G | A | | 81 | 5 |
| MEEI17 Primary | DBN1 | chr5:176885263 | Silent | G | A | G526G | 267 | 34 |
| MEEI17 Primary | ADAMTS2 | chr5:178770992 | Missense | C | T | E104K | 230 | 19 |
| MEEI17 Primary | TFAP2A | chr6:10404765 | Missense | G | A | S249L | 148 | 40 |
| MEEI17 Primary | ERVFRD-1 | chr6:11105298 | Silent | C | G | L82L | 235 | 33 |
| MEEI17 Primary | E2F3 | chr6:20402610 | Silent | C | T | A49A | 419 | 7 |
| MEEI17 Primary | HIST1H2BE | chr6:26184237 | Missense | G | C | E72Q | 399 | 61 |
| MEEI17 Primary | POM121L2 | chr6:27279625 | Missense | G | C | R109G | 298 | 24 |
| MEEI17 Primary | DDAH2 | chr6:31696886 | Missense | C | G | G18A | 153 | 22 |
| MEEI17 Primary | COL11A2 | chr6:33156150 | Missense | C | T | E199K | 677 | 60 |
| MEEI17 Primary | ANKS1A | chr6:34985604 | Missense | G | A | G593D | 346 | 40 |
| MEEI17 Primary | DNAH8 | chr6:38905891 | Missense | C | A | A3685D | 299 | 24 |
| MEEI17 Primary | TFAP2B | chr6:50796332 | Splice Site | T | A | S190_splice | 184 | 16 |
| MEEI17 Primary | BAI3 | chr6:70070810 | Silent | A | G | T1215T | 67 | 9 |
| MEEI17 Primary | ME1 | chr6:83933629 | Silent | G | A | G433G | 130 | 12 |
| MEEI17 Primary | RARS2 | chr6:88234357 | Missense | C | T | V298I | 361 | 6 |
| MEEI17 Primary | HSF2 | chr6:122734769 | Missense | G | A | E144K | 152 | 12 |
| MEEI17 Primary | THEMIS | chr6:128150822 | Missense | C | G | E91Q | 114 | 14 |
| MEEI17 Primary | SMLR1 | chr6:131156091 | Missense | C | T | T107M | 208 | 17 |
| MEEI17 Primary | SYNE1 | chr6:152671854 | Missense | C | G | E3878Q | 275 | 31 |
| MEEI17 Primary | RPA3 | chr7:7676696 | Missense | C | T | E101K | 151 | 16 |
| MEEI17 Primary | DNAH11 | chr7:21778423 | Missense | G | A | D2591N | 84 | 8 |
| MEEI17 Primary | HOXA3 | chr7:27150260 | 5′UTR | C | T | | 247 | 32 |
| MEEI17 Primary | HOXA11 | chr7:27224189 | Missense | G | A | S192L | 508 | 59 |
| MEEI17 Primary | FKBP9 | chr7:33042311 | Missense | G | A | V466I | 246 | 6 |
| MEEI17 Primary | ABCA13 | chr7:48312377 | Missense | C | G | N1038K | 210 | 19 |
| MEEI17 Primary | BAZ1B | chr7:72912953 | Missense | C | T | E149K | 253 | 28 |
| MEEI17 Primary | SLC25A13 | chr7:95951304 | 5′UTR | T | C | | 256 | 24 |
| MEEI17 Primary | ZKSCAN5 | chr7:99130932 | 3′UTR | A | C | | 30 | 5 |
| MEEI17 Primary | ZNF655 | chr7:99161523 | Missense | T | G | S129R | 118 | 8 |
| MEEI17 Primary | KCP | chr7:128525206 | RNA | G | A | | 381 | 38 |
| MEEI17 Primary | CTAGE6 | chr7:143453626 | Missense | C | T | E376K | 1050 | 27 |
| MEEI17 Primary | Unknown | chr7:143533805 | IGR | G | A | | 484 | 17 |
| MEEI17 Primary | CUL1 | chr7:148451132 | Missense | C | T | P69S | 139 | 12 |
| MEEI17 Primary | ZNF467 | chr7:149463038 | Missense | C | T | E185K | 259 | 25 |
| MEEI17 Primary | CSMD1 | chr8:2832046 | Missense | C | G | E2890D | 180 | 17 |
| MEEI17 Primary | FAM66D | chr8:11986534 | RNA | C | G | | 1785 | 73 |
| MEEI17 Primary | PSD3 | chr8:18413851 | Silent | C | T | L934L | 73 | 8 |
| MEEI17 Primary | RAB11FIP1 | chr8:37732638 | Missense | G | T | S339R | 261 | 30 |
| MEEI17 Primary | ANK1 | chr8:41525881 | Silent | G | A | P1766P | 292 | 5 |
| MEEI17 Primary | KAT6A | chr8:41791645 | Missense | C | G | D1365H | 117 | 13 |
| MEEI17 Primary | KAT6A | chr8:41791897 | Missense | C | T | E1281K | 298 | 31 |
| MEEI17 Primary | XKR4 | chr8:56015555 | Silent | C | T | H169H | 104 | 7 |
| MEEI17 Primary | CSMD3 | chr8:113326163 | Missense | G | T | N2556K | 178 | 9 |
| MEEI17 Primary | GPIHBP1 | chr8:144296937 | Nonsense | C | A | C77* | 272 | 33 |
| MEEI17 Primary | TOP1MT | chr8:144406198 | Missense | G | A | R213W | 166 | 16 |
| MEEI17 Primary | KIAA2026 | chr9:5922709 | Missense | C | G | G1096A | 210 | 24 |
| MEEI17 Primary | KIAA2026 | chr9:5922996 | Silent | C | T | Q1000Q | 210 | 19 |
| MEEI17 Primary | GLDC | chr9:6550845 | Nonsense | G | A | R843* | 432 | 43 |
| MEEI17 Primary | Unknown | chr9:68414845 | IGR | C | T | | 45 | 8 |
| MEEI17 Primary | KIF27 | chr9:86514596 | Missense | G | T | Q528K | 115 | 7 |
| MEEI17 Primary | FBP2 | chr9:97333806 | Missense | C | T | G169S | 86 | 10 |
| MEEI17 Primary | ZNF189 | chr9:104171650 | Missense | C | T | H520Y | 161 | 13 |
| MEEI17 Primary | MAPKAP1 | chr9:128268606 | Missense | C | A | C350F | 369 | 32 |
| MEEI17 Primary | C9orf50 | chr9:132375404 | Silent | C | T | P390P | 356 | 30 |
| MEEI17 Primary | RXRA | chr9:137314322 | Intron | A | C | | 36 | 8 |
| MEEI17 Primary | NOTCH1 | chr9:139409069 | Silent | G | A | F700F | 766 | 84 |
| MEEI17 Primary | ID12 | chr10:1065637 | Silent | C | T | T168T | 350 | 28 |
| MEEI17 Primary | ARMC3 | chr10:23244738 | Missense | G | A | E57K | 124 | 14 |
| MEEI17 Primary | GPR158 | chr10:25883146 | 3′UTR | G | C | | 115 | 8 |
| MEEI17 Primary | PDE6C | chr10:95425205 | 3′UTR | T | A | | 149 | 14 |
| MEEI17 Primary | PNLIPRP2 | chr10:118385470 | RNA | C | T | | 176 | 28 |
| MEEI17 Primary | STK33 | chr11:8486305 | Missense | T | C | D135G | 152 | 10 |
| MEEI17 Primary | CDC42BPG | chr11:64600406 | Silent | G | A | T919T | 126 | 12 |
| MEEI17 Primary | PCNXL3 | chr11:65404235 | Splice Site | A | C | | 71 | 16 |
| MEEI17 Primary | PACS1 | chr11:65985029 | Intron | C | T | | 114 | 18 |
| MEEI17 Primary | PACS1 | chr11:65985165 | Intron | C | G | | 28 | 4 |
| MEEI17 Primary | PACS1 | chr11:65985172 | Intron | C | G | | 29 | 4 |
| MEEI17 Primary | LRTOMT | chr11:71799427 | Splice Site | G | A | | 101 | 6 |
| MEEI17 Primary | ALG8 | chr11:77838434 | Silent | G | A | H48H | 268 | 25 |
| MEEI17 Primary | FAT3 | chr11:92577460 | Missense | G | A | E3643K | 205 | 23 |
| MEEI17 Primary | BIRC2 | chr11:102221135 | Missense | G | A | E184K | 245 | 27 |
| MEEI17 Primary | DDX25 | chr11:125781365 | Missense | A | G | H261R | 70 | 8 |
| MEEI17 Primary | IGSF9B | chr11:133790271 | Missense | G | A | P1117S | 73 | 4 |
| MEEI17 Primary | KCNA6 | chr12:4919528 | Silent | G | A | R107R | 219 | 23 |
| MEEI17 Primary | CD163L1 | chr12:7559220 | Missense | G | A | S332F | 107 | 11 |
| MEEI17 Primary | LRRK2 | chr12:40629475 | Missense | C | T | S132L | 215 | 26 |
| MEEI17 Primary | PDZRN4 | chr12:41966269 | Missense | G | A | R303Q | 376 | 35 |
| MEEI17 Primary | AQP2 | chr12:50347966 | Missense | C | T | A130V | 382 | 47 |
| MEEI17 Primary | SLC11A2 | chr12:51386092 | Missense | C | A | A439S | 175 | 27 |
| MEEI17 Primary | HOXC5 | chr12:54428086 | Missense | C | A | T160N | 392 | 44 |
| MEEI17 Primary | NFE2 | chr12:54686625 | Missense | G | A | R219W | 150 | 26 |
| MEEI17 Primary | BAZ2A | chr12:56997411 | Missense | G | A | R1008W | 185 | 21 |
| MEEI17 Primary | EID3 | chr12:104698639 | Missense | G | C | L309F | 86 | 13 |
| MEEI17 Primary | DDX54 | chr12:113605757 | Intron | C | T | | 81 | 5 |
| MEEI17 Primary | SIRT4 | chr12:120741514 | Silent | C | T | F50F | 258 | 31 |
| MEEI17 Primary | PUS1 | chr12:132425971 | Missense | G | A | E174K | 288 | 25 |
| MEEI17 Primary | NOC4L | chr12:132631927 | Silent | C | T | L149L | 84 | 7 |
| MEEI17 Primary | KLHL1 | chr13:70293649 | Missense | G | A | H623Y | 310 | 16 |
| MEEI17 Primary | ABCC4 | chr13:95858804 | Silent | G | A | V381V | 164 | 19 |
| MEEI17 Primary | TUBGCP3 | chr13:113200091 | Silent | G | C | L419L | 261 | 19 |
| MEEI17 Primary | GRTP1 | chr13:114018221 | Missense | C | G | D13H | 184 | 29 |
| MEEI17 Primary | CHD8 | chr14:21870222 | Missense | T | C | D1319G | 97 | 12 |
| MEEI17 Primary | AJUBA | chr14:23444289 | Missense | A | T | Y422N | 55 | 4 |
| MEEI17 Primary | DICER1 | chr14:95572381 | Missense | G | A | S995L | 248 | 19 |
| MEEI17 Primary | BDKRB2 | chr14:96707400 | Missense | G | A | M218I | 106 | 14 |
| MEEI17 Primary | BDKRB2 | chr14:96707584 | Missense | G | A | E280K | 100 | 11 |
| MEEI17 Primary | DYNC1H1 | chr14:102466705 | Missense | A | C | E1348A | 393 | 35 |
| MEEI17 Primary | Unknown | chr14:103576752 | IGR | C | T | | 211 | 21 |
| MEEI17 Primary | IGHV4-4 | chr14:106478158 | RNA | C | G | | 620 | 9 |
| MEEI17 Primary | B2M | chr15:45003745 | Start Codon | A | T | M1L | 486 | 53 |
| MEEI17 Primary | LDHAL6B | chr15:59499247 | Silent | C | T | I36I | 251 | 32 |
| MEEI17 Primary | HERC1 | chr15:63972295 | Missense | C | A | S2177I | 129 | 11 |
| MEEI17 Primary | RHOT2 | chr16:723520 | Missense | C | T | L591F | 92 | 13 |
| MEEI17 Primary | CHTF18 | chr16:845741 | Missense | C | G | I953M | 101 | 13 |
| MEEI17 Primary | SPSB3 | chr16:1827212 | Silent | G | A | S318S | 345 | 7 |
| MEEI17 Primary | SEC14L5 | chr16:5038249 | Missense | C | T | R105C | 194 | 25 |
| MEEI17 Primary | SMG1 | chr16:18937327 | Missense | C | T | G135 | 276 | 6 |
| MEEI17 Primary | ZNF768 | chr16:30536461 | Missense | G | A | R334C | 287 | 33 |
| MEEI17 Primary | RP11-17M15.2 | chr16:32321755 | RNA | A | G | | 25 | 3 |
| MEEI17 Primary | Unknown | chr16:34404240 | IGR | A | G | | 138 | 15 |
| MEEI17 Primary | ZNF23 | chr16:71487240 | Silent | G | C | L16L | 156 | 21 |
| MEEI17 Primary | ZNF276 | chr16:89805829 | 3′UTR | G | A | | 218 | 30 |
| MEEI17 Primary | MC1R | chr16:89986035 | Silent | C | T | I123I | 211 | 25 |
| MEEI17 Primary | ARHGEF15 | chr17:8215998 | Intron | C | T | | 316 | 6 |
| MEEI17 Primary | GPR179 | chr17:36487329 | Missense | C | G | R708P | 91 | 5 |
| MEEI17 Primary | AXIN2 | chr17:63545769 | Missense | C | G | K275N | 397 | 7 |
| MEEI17 Primary | FBF1 | chr17:73910089 | Missense | C | T | E997K | 122 | 16 |
| MEEI17 Primary | UBE2O | chr17:74398194 | Missense | G | A | T234M | 102 | 10 |
| MEEI17 Primary | TMC6 | chr17:76117146 | Missense | C | T | A495T | 384 | 52 |
| MEEI17 Primary | RNF213 | chr17:78324190 | Missense | C | T | S3393L | 441 | 8 |
| MEEI17 Primary | CDH20 | chr18:59221757 | Silent | C | T | L745L | 444 | 56 |
| MEEI17 Primary | CDH20 | chr18:59221904 | Silent | G | A | S794S | 109 | 11 |
| MEEI17 Primary | ZNF407 | chr18:72343597 | Missense | C | T | H208Y | 164 | 23 |
| MEEI17 Primary | DAZAP1 | chr19:1432548 | Nonsense | C | T | Q303* | 285 | 37 |
| MEEI17 Primary | PLIN5 | chr19:4523796 | Missense | G | A | A379V | 115 | 10 |
| MEEI17 Primary | SAFB | chr19:5667147 | Nonsense | G | T | E809* | 179 | 15 |
| MEEI17 Primary | KHSRP | chr19:6413540 | 3′UTR | C | G | | 59 | 15 |
| MEEI17 Primary | SLC25A41 | chr19:6433594 | Silent | A | G | P37P | 192 | 22 |
| MEEI17 Primary | MUC16 | chr19:9088981 | Missense | G | A | T945M | 312 | 6 |
| MEEI17 Primary | NOTCH3 | chr19:15278185 | Missense | T | C | D1746G | 242 | 23 |
| MEEI17 Primary | ZNF181 | chr19:35232275 | Missense | T | C | F374S | 185 | 4 |
| MEEI17 Primary | ZNF780B | chr19:40541132 | Missense | G | C | S545C | 232 | 18 |
| MEEI17 Primary | CNTD2 | chr19:40732369 | Silent | C | T | A60A | 252 | 25 |
| MEEI17 Primary | SNRPA | chr19:41268873 | Missense | C | T | P165L | 206 | 22 |
| MEEI17 Primary | IRGC | chr19:44223234 | Missense | C | A | A175E | 53 | 4 |
| MEEI17 Primary | ZC3H4 | chr19:47584789 | Missense | G | A | T474M | 268 | 31 |
| MEEI17 Primary | GRIN2D | chr19:48908274 | Missense | G | A | R250Q | 151 | 18 |
| MEEI17 Primary | IRF3 | chr19:50166455 | Missense | G | C | S133C | 146 | 18 |
| MEEI17 Primary | ADM5 | chr19:50193112 | Silent | C | T | L6L | 249 | 24 |
| MEEI17 Primary | ZNF761 | chr19:53958345 | RNA | A | G | | 329 | 30 |
| MEEI17 Primary | CNOT3 | chr19:54646887 | Missense | G | A | E20K | 288 | 22 |
| MEEI17 Primary | LILRB5 | chr19:54754876 | Missense | C | G | E587Q | 281 | 20 |
| MEEI17 Primary | EPN1 | chr19:56200721 | Missense | G | A | R221Q | 129 | 10 |
| MEEI17 Primary | ZNF304 | chr19:57867695 | Missense | C | T | S153L | 192 | 14 |
| MEEI17 Primary | ZNF550 | chr19:58058874 | Silent | G | A | L214L | 227 | 23 |
| MEEI17 Primary | CDH4 | chr20:60448935 | Silent | G | T | V343V | 98 | 15 |
| MEEI17 Primary | ANKRD30BP2 | chr21:14414914 | RNA | G | A | | 146 | 7 |
| MEEI17 Primary | OLIG2 | chr21:34399428 | Silent | G | A | T86T | 370 | 38 |
| MEEI17 Primary | OLIG1 | chr21:34443201 | Missense | G | A | A217T | 112 | 19 |
| MEEI17 Primary | U2AF1 | chr21:44513125 | 3′UTR | G | T | | 68 | 10 |
| MEEI17 Primary | RRP1B | chr21:45106735 | Missense | G | A | D354N | 236 | 9 |
| MEEI17 Primary | TRPM2 | chr21:45789172 | Silent | C | T | I239I | 149 | 12 |
| MEEI17 Primary | COL18A1 | chr21:46897813 | Silent | C | T | V800V | 138 | 20 |
| MEEI17 Primary | AC008103.5 | chr22:18843018 | RNA | C | G | | 845 | 35 |
| MEEI17 Primary | SCARF2 | chr22:20791909 | Missense | C | G | E45Q | 446 | 45 |
| MEEI17 Primary | NF2 | chr22:30090901 | 3′UTR | G | A | | 136 | 12 |
| MEEI17 Primary | MYH9 | chr22:36689445 | Missense | C | G | R1342P | 340 | 43 |
| MEEI17 Primary | PVALB | chr22:37213063 | 5′UTR | G | C | | 135 | 6 |
| MEEI17 Primary | SH3BP1 | chr22:38051320 | Missense | C | G | Q579E | 85 | 9 |
| MEEI17 Primary | ENTHD1 | chr22:40217051 | Missense | G | C | S260C | 230 | 19 |
| MEEI17 Primary | ACO2 | chr22:41919943 | Missense | G | C | E519Q | 258 | 33 |
| MEEI17 Primary | PKDREJ | chr22:46654106 | Missense | C | T | R1705K | 128 | 13 |
| MEEI17 Primary | PKDREJ | chr22:46654777 | Missense | C | G | L1481F | 126 | 14 |
| MEEI17 Primary | PKDREJ | chr22:46656431 | Missense | C | T | G930E | 184 | 18 |
| MEEI17 Primary | PKDREJ | chr22:46656903 | Missense | C | T | A773T | 127 | 13 |
| MEEI17 Primary | CTA-299D3.8 | chr22:48940680 | Silent | G | A | F43F | 214 | 18 |
| MEEI17 Primary | ZNF674 | chrX:46360510 | Missense | C | T | D172N | 80 | 23 |
| MEEI17 Primary | CXorf57 | chrX:105875923 | Missense | G | C | E350Q | 34 | 11 |
| MEEI17 Primary | ATP2B3 | chrX:152818528 | Missense | G | A | R606Q | 102 | 7 |
| MEEI17 Primary | Unknown | chrGL000205.1:118335 | IGR | G | A | | 153 | 16 |
| MEEI17 Primary | Unknown | chrGL000195.1:138011 | IGR | C | T | | 9 | 3 |
| MEEI17 Primary | Unknown | chrGL000194.1:58626 | IGR | T | C | | 64 | 6 |
| MEEI20 Primary | NOC2L | chr1:894240 | Intron | G | A | | 67 | 40 |
| MEEI20 Primary | TARDBP | chr1:11073956 | Missense | G | C | E58Q | 177 | 102 |
| MEEI20 Primary | MACF1 | chr1:39797507 | Silent | C | T | F1749F | 167 | 56 |
| MEEI20 Primary | ZZZ3 | chr1:78097765 | Missense | T | A | Q425H | 26 | 18 |
| MEEI20 Primary | KIAA1107 | chr1:92647732 | Missense | G | A | E1115K | 61 | 31 |
| MEEI20 Primary | GSTM5 | chr1:110255418 | 5′UTR | G | C | | 67 | 44 |
| MEEI20 Primary | GABPB2 | chr1:151062895 | Missense | C | T | P41L | 71 | 16 |
| MEEI20 Primary | S100A16 | chr1:153579813 | 3′UTR | G | C | | 6 | 3 |
| MEEI20 Primary | FCRL5 | chr1:157494259 | Silent | C | T | L683L | 184 | 44 |
| MEEI20 Primary | RCSD1 | chr1:167666670 | Missense | A | G | E270G | 255 | 55 |
| MEEI20 Primary | RABGAP1L | chr1:174769546 | Missense | G | A | D26N | 132 | 33 |
| MEEI20 Primary | COLGALT2 | chr1:183899415 | Nonsense | G | C | S538* | 253 | 60 |
| MEEI20 Primary | COLGALT2 | chr1:183933253 | 5′UTR | G | C | | 19 | 4 |
| MEEI20 Primary | ASPM | chr1:197070960 | Missense | T | C | K2474R | 784 | 110 |
| MEEI20 Primary | IL20 | chr1:207039175 | 5′UTR | G | C | | 204 | 28 |
| MEEI20 Primary | RD3 | chr1:211652607 | Missense | G | A | S120L | 111 | 34 |
| MEEI20 Primary | PTPN14 | chr1:214625204 | Missense | G | T | F96L | 125 | 25 |
| MEEI20 Primary | MIA3 | chr1:222824217 | Missense | G | C | R1294T | 115 | 40 |
| MEEI20 Primary | SUSD4 | chr1:223400943 | Missense | G | A | P352S | 67 | 97 |
| MEEI20 Primary | DNAH14 | chr1:225506368 | Silent | C | T | V3015V | 115 | 14 |
| MEEI20 Primary | DNAH14 | chr1:225562549 | Missense | G | A | E4067K | 223 | 53 |
| MEEI20 Primary | OBSCN | chr1:228459730 | Missense | G | C | E1932Q | 598 | 108 |
| MEEI20 Primary | OBSCN | chr1:228505340 | Silent | C | T | P5536P | 570 | 288 |
| MEEI20 Primary | TRIM67 | chr1:231299118 | Missense | C | G | P135A | 107 | 34 |
| MEEI20 Primary | TBCE | chr1:235612150 | 3′UTR | G | A | | 122 | 32 |
| MEEI20 Primary | FMN2 | chr1:240458138 | Silent | T | C | D1390D | 204 | 41 |
| MEEI20 Primary | AHCTF1 | chr1:247014172 | Silent | T | C | K1747K | 178 | 195 |
| MEEI20 Primary | OR2C3 | chr1:247695215 | Missense | A | G | M200T | 206 | 42 |
| MEEI20 Primary | NBAS | chr2:15307376 | Silent | G | C | V2304V | 121 | 65 |
| MEEI20 Primary | SDC1 | chr2:20403986 | Missense | G | A | T72M | 93 | 21 |
| MEEI20 Primary | APOB | chr2:21225265 | Nonsense | A | T | Y4343* | 55 | 18 |
| MEEI20 Primary | ATAD2B | chr2:24009067 | Missense | G | A | L935F | 66 | 45 |
| MEEI20 Primary | SPAST | chr2:32340842 | Missense | G | C | L314F | 217 | 44 |
| MEEI20 Primary | CCT4 | chr2:62099620 | Missense | C | G | C410S | 243 | 29 |
| MEEI20 Primary | TET3 | chr2:74274322 | Silent | A | T | P291P | 343 | 438 |
| MEEI20 Primary | INO80B | chr2:74683244 | Missense | C | G | L129V | 45 | 26 |
| MEEI20 Primary | POLR1A | chr2:86332979 | 5′UTR | C | T | | 24 | 9 |
| MEEI20 Primary | POLR1A | chr2:86333207 | 5′UTR | G | A | | 41 | 8 |
| MEEI20 Primary | RNF103 | chr2:86831783 | Missense | G | C | S414C | 157 | 45 |
| MEEI20 Primary | ANKRD36C | chr2:96557426 | Missense | T | G | E948D | 148 | 48 |
| MEEI20 Primary | POLR1B | chr2:113326347 | Missense | G | A | E648K | 58 | 16 |
| MEEI20 Primary | TMEM177 | chr2:120439062 | Silent | A | G | A211A | 164 | 36 |
| MEEI20 Primary | CNTNAP5 | chr2:125232387 | Silent | C | T | I330I | 75 | 17 |
| MEEI20 Primary | PLEKHB2 | chr2:131890577 | Missense | C | G | L146V | 113 | 37 |
| MEEI20 Primary | MGAT5 | chr2:135028001 | Missense | C | A | R965 | 87 | 41 |
| MEEI20 Primary | KIF5C | chr2:149847540 | Missense | G | C | G578A | 72 | 34 |
| MEEI20 Primary | NEB | chr2:152359330 | Missense | G | A | R7969C | 124 | 36 |
| MEEI20 Primary | UBR3 | chr2:170850928 | Missense | G | A | D1294N | 343 | 64 |
| MEEI20 Primary | GORASP2 | chr2:171822706 | 3′UTR | C | G | | 102 | 23 |
| MEEI20 Primary | TTN | chr2:179497043 | Silent | T | C | R14526R | 284 | 35 |
| MEEI20 Primary | CCDC141 | chr2:179770213 | Missense | C | G | G370R | 179 | 21 |
| MEEI20 Primary | PGAP1 | chr2:197755602 | Missense | C | G | E375Q | 241 | 28 |
| MEEI20 Primary | INO80D | chr2:206869892 | Missense | C | T | V762I | 371 | 66 |
| MEEI20 Primary | PTH2R | chr2:209345831 | Silent | C | T | L340L | 95 | 45 |
| MEEI20 Primary | MAP2 | chr2:210595014 | Nonsense | C | T | R1793* | 462 | 61 |
| MEEI20 Primary | COL4A3 | chr2:228128601 | Missense | C | A | S419Y | 102 | 36 |
| MEEI20 Primary | SLC16A14 | chr2:230910914 | Missense | G | C | L310V | 44 | 43 |
| MEEI20 Primary | COL6A3 | chr2:238263762 | Intron | A | G | | 16 | 8 |
| MEEI20 Primary | TRAF3IP1 | chr2:239256102 | Missense | C | A | S423Y | 133 | 46 |
| MEEI20 Primary | EOMES | chr3:27759160 | Missense | G | A | R488C | 53 | 113 |
| MEEI20 Primary | XIRP1 | chr3:39227759 | Missense | T | A | M1060L | 23 | 62 |
| MEEI20 Primary | ZNF852 | chr3:44541164 | Missense | G | C | Q369E | 95 | 37 |
| MEEI20 Primary | CYB561D2 | chr3:50388837 | 5′UTR | C | T | | 32 | 8 |
| MEEI20 Primary | KIAA2018 | chr3:113379729 | Missense | T | C | Q267R | 104 | 32 |
| MEEI20 Primary | CASR | chr3:121980379 | Missense | G | A | S166N | 327 | 72 |
| MEEI20 Primary | MUC13 | chr3:124631993 | Silent | G | A | P392P | 133 | 22 |
| MEEI20 Primary | CHST13 | chr3:126260591 | Silent | C | T | L66L | 63 | 11 |
| MEEI20 Primary | ATP2C1 | chr3:130682805 | Intron | T | A | | 321 | 68 |
| MEEI20 Primary | EPHB1 | chr3:134825342 | Silent | C | T | H286H | 475 | 63 |
| MEEI20 Primary | SLC35G2 | chr3:136574442 | Silent | C | T | I380I | 131 | 20 |
| MEEI20 Primary | TERC | chr3:169482512 | lincRNA | C | T | | 140 | 35 |
| MEEI20 Primary | LRRC31 | chr3:169557996 | Missense | C | T | R478Q | 303 | 73 |
| MEEI20 Primary | OPA1 | chr3:193355029 | Missense | G | T | D332Y | 144 | 97 |
| MEEI20 Primary | PPARGC1A | chr4:23830201 | Silent | C | G | A193A | 234 | 33 |
| MEEI20 Primary | SMR3B | chr4:71255799 | 3′UTR | C | A | | 113 | 45 |
| MEEI20 Primary | NPFFR2 | chr4:72897735 | Silent | C | T | R39R | 74 | 11 |
| MEEI20 Primary | MTTP | chr4:100522763 | Splice Site | G | C | | 224 | 68 |
| MEEI20 Primary | SLC9B1 | chr4:103870473 | Missense | C | T | G108E | 43 | 10 |
| MEEI20 Primary | TBCK | chr4:107154194 | Nonsense | G | A | Q514* | 129 | 30 |
| MEEI20 Primary | PHF17 | chr4:129793144 | Silent | G | C | R752R | 215 | 50 |
| MEEI20 Primary | LRBA | chr4:151509256 | Missense | C | T | E2092K | 54 | 24 |
| MEEI20 Primary | KLHL2 | chr4:166199071 | Intron | A | T | | 111 | 77 |
| MEEI20 Primary | IRX1 | chr5:3599530 | Silent | C | T | T156T | 732 | 143 |
| MEEI20 Primary | DNAH5 | chr5:13919404 | Missense | C | T | E286K | 292 | 67 |
| MEEI20 Primary | FAM105A | chr5:14581990 | 5′UTR | G | T | | 156 | 34 |
| MEEI20 Primary | FAM105A | chr5:14581991 | 5′UTR | C | T | | 154 | 35 |
| MEEI20 Primary | RAD1 | chr5:34914899 | Silent | A | G | A33A | 354 | 67 |
| MEEI20 Primary | PPWD1 | chr5:64859267 | Missense | G | A | E44K | 87 | 44 |
| MEEI20 Primary | ZNF366 | chr5:71739493 | 3′UTR | G | A | | 3 | 8 |
| MEEI20 Primary | PCDHA13 | chr5:140263972 | Missense | G | A | V707M | 178 | 82 |
| MEEI20 Primary | PCDHB3 | chr5:140481075 | Missense | T | G | F281C | 68 | 31 |
| MEEI20 Primary | PCDHI2 | chr5:141336I58 | Missense | C | T | R420K | 109 | 68 |
| MEEI20 Primary | KIF4B | chr5:154394844 | Silent | G | A | Q475Q | 163 | 69 |
| MEEI20 Primary | FAM196B | chr5:169310735 | Silent | G | C | V56V | 53 | 20 |
| MEEI20 Primary | NSD1 | chr5:1766386I4 | Nonsense | C | T | R1072* | 81 | 42 |
| MEEI20 Primary | RGS14 | chr5:176795865 | Missense | G | A | E333K | 123 | 68 |
| MEEI20 Primary | GMDS | chr6:1961111 | Silent | G | A | I145I | 75 | 28 |
| MEEI20 Primary | LRRC16A | chr6:25495409 | Missense | C | A | L431I | 80 | 25 |
| MEEI20 Primary | SLC17A3 | chr6:25862664 | Missense | A | T | L34I | 221 | 68 |
| MEEI20 Primary | HIST1H2AG | chr6:27100950 | Silent | C | T | L34L | 58 | 177 |
| MEEI20 Primary | ZNRD1-AS1 | chr6:29976003 | RNA | G | A | | 63 | 27 |
| MEEI20 Primary | AGPAT1 | chr6:32I36868 | 3′UTR | G | T | | 1 | 6 |
| MEEI20 Primary | BRD2 | chr6:32947795 | Missense | G | C | D713H | 238 | 89 |
| MEEI20 Primary | DNAH8 | chr6:38821105 | Silent | C | T | L1688L | 58 | 17 |
| MEEI20 Primary | UBR2 | chr6:42629988 | Missense | A | G | H1170R | 78 | 20 |
| MEEI20 Primary | MAD2LIBP | chr6:43597287 | 5′UTR | A | C | | 36 | 17 |
| MEEI20 Primary | SLC25A27 | chr6:46623757 | Missense | T | C | I95T | 65 | 20 |
| MEEI20 Primary | GPR110 | chr6:46979830 | Silent | C | G | G343G | 134 | 43 |
| MEEI20 Primary | EYS | chr6:65767552 | Missense | C | A | D698Y | 135 | 43 |
| MEEI20 Primary | LAMA4 | chr6:112575392 | 5′UTR | G | T | | 82 | 49 |
| MEEI20 Primary | CH5T12 | chr7:2473426 | Silent | C | A | I384I | 99 | 88 |
| MEEI20 Primary | TNRC18 | chr7:5428096 | Silent | G | A | S453S | 40 | 14 |
| MEEI20 Primary | TNRC18 | chr7:5428097 | Missense | G | A | S453F | 39 | 15 |
| MEEI20 Primary | USP42 | chr7:6I93522 | Silent | C | T | R779R | 144 | 32 |
| MEEI20 Primary | ZNF853 | chr7:6661136 | Missense | C | A | QI72K | 196 | 75 |
| MEEI20 Primary | GARS | chr7:30662062 | Silent | C | T | L533L | 239 | 89 |
| MEEI20 Primary | BBS9 | chr7:33427715 | Missense | C | G | L692V | 207 | 76 |
| MEEI20 Primary | AMPH | chr7:38469134 | Intron | T | G | | 69 | 16 |
| MEEI20 Primary | AEBPI | chr7:44152354 | Silent | G | A | E805E | 120 | 63 |
| MEEI20 Primary | NPCIL1 | chr7:44571753 | Missense | C | G | E825Q | 195 | 86 |
| MEEI20 Primary | NPCIL1 | chr7:44578713 | Missense | C | T | G428E | 220 | 67 |
| MEEI20 Primary | OGDH | chr7:44714050 | Missense | C | T | R277C | 115 | 44 |
| MEEI20 Primary | ZNF736 | chr7:63808578 | Missense | T | A | LI13I | 230 | 63 |
| MEEI20 Primary | AUTS2 | chr7:70252291 | Missense | C | T | S802L | 180 | 71 |
| MEEI20 Primary | MDH2 | chr7:75687389 | Missense | C | T | A14IV | 68 | 77 |
| MEEI20 Primary | GNAI1 | chr7:79840299 | Missense | G | T | G202V | 67 | 68 |
| MEEI20 Primary | RNF133 | chr7:122338664 | Silent | T | C | A103A | 197 | 59 |
| MEEI20 Primary | LMOD2 | chr7:123296256 | Missense | T | G | L80R | 120 | 45 |
| MEEI20 Primary | GRM8 | chr7:126882896 | Missense | C | A | Q121H | 209 | 57 |
| MEEI20 Primary | KIAA1549 | chr7:138522791 | Missense | C | G | GI889R | 85 | 107 |
| MEEI20 Primary | PRSS3P2 | chr7:14248I377 | RNA | G | C | | 315 | 135 |
| MEEI20 Primary | RP11-61L23.2 | chr7:143510092 | RNA | G | A | | 440 | 137 |
| MEEI20 Primary | CUL1 | chr7:148454131 | Silent | A | G | E124E | 63 | 22 |
| MEEI20 Primary | ZNF282 | chr7:148921263 | Missense | C | T | R514C | 33 | 41 |
| MEEI20 Primary | ANGPT2 | chr8:6366411 | Missense | T | G | M457L | 51 | 22 |
| MEEI20 Primary | BLK | chr8:114I2293 | Missense | G | C | E172Q | 215 | 124 |
| MEEI20 Primary | PENK | chr8:57354065 | Missense | C | T | M190I | 169 | 60 |
| MEEI20 Primary | CHD7 | chr8:61769121 | Nonsense | C | T | R2428* | 249 | 67 |
| MEEI20 Primary | ZFHX4 | chr8:77775863 | Missense | C | G | Q3305E | 121 | 43 |
| MEEI20 Primary | DECR1 | chr8:91033274 | Silent | A | G | KI76K | 28 | 11 |
| MEEI20 Primary | OXR1 | chr8:107705027 | Silent | A | T | VI99V | 109 | 62 |
| MEEI20 Primary | PKHD1L1 | chr8:110439314 | Missense | G | C | E977Q | 218 | 51 |
| MEEI20 Primary | ENPP2 | chr8:120629779 | Missense | G | C | I164M | 87 | 21 |
| MEEI20 Primary | CDKN2A | chr9:21971000 | Nonsense | C | A | E120* | 103 | 401 |
| MEEI20 Primary | MELK | chr9:36599467 | Nonsense | C | G | S152* | 61 | 23 |
| MEEI20 Primary | GNA14 | chr9:80262831 | 5′UTR | G | A | | 16 | 6 |
| MEEI20 Primary | ECM2 | chr9:95263289 | Missense | G | A | P551S | 171 | 61 |
| MEEI20 Primary | PHF2 | chr9:96429442 | Missense | G | T | E756D | 42 | 156 |
| MEEI20 Primary | DBC1 | chr9:122004494 | Splice Site | C | G | G137_splice | 53 | 46 |
| MEEI20 Primary | SPTAN1 | chr9:131374009 | Missense | A | T | H1597L | 429 | 142 |
| MEEI20 Primary | NOTCH1 | chr9:139409769 | Nonsense | C | A | E663* | 89 | 321 |
| MEEI20 Primary | PTPLA | chr10:17632310 | 3′UTR | G | C | | 14 | 8 |
| MEEI20 Primary | LYZL1 | chr10:29577994 | 5′UTR | G | C | | 28 | 20 |
| MEEI20 Primary | Unknown | chr10:38737632 | IGR | C | A | | 266 | 120 |
| MEEI20 Primary | RSU1P2 | chr10:45602410 | RNA | A | C | | 18 | 5 |
| MEEI20 Primary | EGR2 | chr10:64573084 | Silent | G | C | P438P | 14 | 11 |
| MEEI20 Primary | LRRTM3 | chr10:68686197 | 5′UTR | C | T | | 12 | 8 |
| MEEI20 Primary | CDH23 | chr10:73565590 | Missense | G | A | E2639K | 57 | 28 |
| MEEI20 Primary | TNKS2 | chr10:93619394 | Silent | C | T | Y1090Y | 34 | 21 |
| MEEI20 Primary | C10orf120 | chr10:124458877 | Nonsense | G | C | Y76* | 62 | 29 |
| MEEI20 Primary | HMX2 | chr10:124909443 | Missense | C | T | S209L | 93 | 62 |
| MEEI20 Primary | DHX32 | chr10:127527692 | Missense | T | A | I587F | 67 | 36 |
| MEEI20 Primary | DHX32 | chr10:127527693 | Silent | A | T | V586V | 68 | 36 |
| MEEI20 Primary | KCNQ1 | chr11:2869097 | Missense | G | MEEI | R632K | 69 | 42 |
| | | | | | 20 | | | |
| | | | | | Primary | | | |
| MEEI20 Primary | OR51M1 | chr11:5411257 | Missense | A | MEEI | Y210F | 79 | 30 |
| | | | | | 20 | | | |
| | | | | | Primary | | | |
| MEEI20 Primary | PAX6 | chr11:31811444 | 3′UTR | C | T | | 46 | 30 |
| MEEI20 Primary | OR5L1 | chr11:55579155 | Missense | C | G | F71L | 128 | 55 |
| MEEI20 Primary | SLC15A3 | chr11:60718567 | Missense | G | T | P153T | 83 | 26 |
| MEEI20 Primary | C11orf48 | chr11:62435057 | Missense | G | C | S185C | 227 | 87 |
| MEEI20 Primary | CCDC88B | chr11:64124704 | Missense | C | T | S628F | 172 | 55 |
| MEEI20 Primary | MAP3K11 | chr11:65367093 | Missense | C | T | D660N | 141 | 57 |
| MEEI20 Primary | CATSPER1 | chr11:65793245 | Silent | G | C | L202L | 68 | 32 |
| MEEI20 Primary | CCDC87 | chr11:66360235 | Missense | G | C | I84M | 58 | 26 |
| MEEI20 Primary | LRFN4 | chr11:66625234 | Missense | C | G | L7V | 78 | 30 |
| MEEI20 Primary | RELT | chr11:73103469 | Missense | C | T | T194M | 292 | 94 |
| MEEI20 Primary | P4HA3 | chr11:74013514 | Missense | C | T | R156Q | 213 | 79 |
| MEEI20 Primary | CACNA1C | chr12:2690884 | Missense | G | A | G675E | 139 | 76 |
| MEEI20 Primary | CACNA1C | chr12:2786826 | Intron | C | T | | 46 | 9 |
| MEEI20 Primary | CACNA1C | chr12:2797785 | Missense | G | C | S1986T | 408 | 100 |
| MEEI20 Primary | PARP11 | chr12:3921231 | 3′UTR | C | A | | 21 | 7 |
| MEEI20 Primary | VAMP1 | chr12:6575058 | Missense | C | T | E80K | 171 | 40 |
| MEEI20 Primary | KLRG1 | chr12:9144826 | Missense | C | T | S36F | 132 | 33 |
| MEEI20 Primary | CLEC12A | chr12:10133239 | Silent | C | T | F146F | 81 | 58 |
| MEEI20 Primary | PRICKLE1 | chr12:42854296 | Missense | G | T | P604Q | 116 | 36 |
| MEEI20 Primary | TENC1 | chr12:53452958 | Silent | C | T | S511S | 36 | 12 |
| MEEI20 Primary | AAAS | chr12:53701437 | Missense | G | A | R493C | 101 | 57 |
| MEEI20 Primary | DCD | chr12:55039031 | Missense | C | G | G72A | 197 | 82 |
| MEEI20 Primary | PA2G4 | chr12:56498234 | 5′UTR | T | A | | 94 | 37 |
| MEEI20 Primary | BTBD11 | chr12:107914298 | Missense | C | G | H390Q | 180 | 54 |
| MEEI20 Primary | CUX2 | chr12:111786131 | 3′UTR | G | A | | 36 | 7 |
| MEEI20 Primary | PRKAB1 | chr12:120118120 | Missense | A | T | K268M | 127 | 40 |
| MEEI20 Primary | HIP1R | chr12:123341234 | Missense | G | C | E527D | 135 | 31 |
| MEEI20 Primary | SBNO1 | chr12:123795625 | Missense | G | A | S1091L | 82 | 48 |
| MEEI20 Primary | CHFR | chr12:133423633 | Silent | C | A | L588L | 127 | 104 |
| MEEI20 Primary | OLFM4 | chr13:53624619 | Missense | C | A | Q416K | 99 | 22 |
| MEEI20 Primary | PCDH17 | chr13:58207963 | Missense | G | A | R428H | 16 | 70 |
| MEEI20 Primary | HS6ST3 | chr13:97484833 | Missense | C | A | T266N | 23 | 116 |
| MEEI20 Primary | TRAV9-1 | chr14:22279905 | RNA | C | T | | 111 | 40 |
| MEEI20 Primary | PSMB5 | chr14:23495401 | Missense | C | G | G230A | 226 | 91 |
| MEEI20 Primary | PSME1 | chr14:24605422 | 5′UTR | C | T | | 29 | 45 |
| MEEI20 Primary | FKBP3 | chr14:45590794 | Silent | C | T | L116L | 143 | 27 |
| MEEI20 Primary | TBPL2 | chr14:55890788 | Intron | C | T | | 20 | 30 |
| MEEI20 Primary | OTX2 | chr14:57268852 | Silent | G | A | I165I | 186 | 47 |
| MEEI20 Primary | PCNX | chr14:71413729 | Missense | C | G | A84G | 114 | 141 |
| MEEI20 Primary | IGHD2-21 | chr14:106354434 | RNA | G | C | | 109 | 20 |
| MEEI20 Primary | NPAP1 | chr15:24923689 | Missense | A | G | N8925 | 180 | 53 |
| MEEI20 Primary | ATP10A | chr15:25940172 | Missense | G | T | S961Y | 188 | 184 |
| MEEI20 Primary | OTUD7A | chr15:31941891 | Intron | C | T | | 36 | 18 |
| MEEI20 Primary | SLCI2A1 | chr15:48524954 | Missense | A | T | I336F | 43 | 192 |
| MEEI20 Primary | THAP10 | chr15:71174825 | Missense | T | C | M248V | 26 | 76 |
| MEEI20 Primary | TTC23 | chr15:99696417 | Missense | C | T | R360Q | 86 | 222 |
| MEEI20 Primary | CRAMP1L | chr16:1723984 | Missense | C | T | R1250C | 197 | 69 |
| MEEI20 Primary | CREBBP | chr16:3781810 | Missense | C | G | Q1619H | 313 | 102 |
| MEEI20 Primary | PDXDC1 | chr16:15068872 | 5′UTR | C | T | | 162 | 34 |
| MEEI20 Primary | TMC5 | chr16:19451967 | Missense | G | A | G203R | 197 | 62 |
| MEEI20 Primary | GP2 | chr16:20322603 | Splice Site | C | T | G516_splice | 141 | 38 |
| MEEI20 Primary | SLC5A11 | chr16:24909423 | Missense | C | A | L201I | 55 | 21 |
| MEEI20 Primary | PRRT2 | chr16:29825108 | Missense | C | T | R245C | 15 | 4 |
| MEEI20 Primary | ZNF688 | chr16:30583569 | Silent | G | A | L10L | 26 | 12 |
| MEEI20 Primary | CDH5 | chr16:66426087 | Missense | A | T | T340S | 297 | 168 |
| MEEI20 Primary | CDH5 | chr16:66432000 | Silent | C | A | V492V | 320 | 59 |
| MEEI20 Primary | CCDC79 | chr16:66803918 | Missense | G | C | Q523E | 108 | 56 |
| MEEI20 Primary | CCDC79 | chr16:66822087 | Missense | G | C | LI29V | 26 | 22 |
| MEEI20 Primary | RLTPR | chr16:67690171 | Silent | G | C | SI261S | 176 | 30 |
| MEEI20 Primary | ACD | chr16:67693674 | Silent | G | T | RI72R | 112 | 29 |
| MEEI20 Primary | ENKD1 | chr16:67700094 | Missense | G | T | P54T | 171 | 31 |
| MEEI20 Primary | DPEP3 | chr16:68011617 | Nonsense | G | C | S3I6* | 180 | 25 |
| MEEI20 Primary | ZNF821 | chr16:71898890 | Missense | C | G | E76D | 96 | 80 |
| MEEI20 Primary | CNTNAP4 | chr16:76501302 | Missense | C | G | Q512E | 375 | 60 |
| MEEI20 Primary | KIAA0513 | chr16:85100869 | Silent | G | A | P64P | 143 | 104 |
| MEEI20 Primary | DPH1 | chr17:1946416 | 3′UTR | C | G | | 48 | 9 |
| MEEI20 Primary | KIF1C | chr17:4925757 | Missense | A | G | D794G | 31 | 52 |
| MEEI20 Primary | TP53 | chr17:7578493 | Nonsense | C | T | W146* | 42 | 154 |
| MEEI20 Primary | PIK3R5 | chr17:8789859 | Missense | C | T | D657N | 42 | 17 |
| MEEI20 Primary | ARHGAP44 | chr17:12855898 | Splice Site | G | C | R379_splice | 73 | 30 |
| MEEI20 Primary | RAI1 | chr17:17714169 | Missense | C | A | S1633Y | 7 | 58 |
| MEEI20 Primary | TOP3A | chr17:18181234 | Missense | G | A | A86IV | 278 | 32 |
| MEEI20 Primary | SMCR8 | chr17:18226425 | 3′UTR | G | A | | 28 | 13 |
| MEEI20 Primary | VTN | chr17:26697213 | Silent | C | G | L4L | 73 | 34 |
| MEEI20 Primary | PEX12 | chr17:33904475 | Missense | C | T | G88S | 405 | 55 |
| MEEI20 Primary | CUEDC1 | chr17:55962593 | Silent | C | T | P111P | 17 | 5 |
| MEEI20 Primary | KCNH6 | chr17:61620952 | Missense | C | T | H722Y | 113 | 34 |
| MEEI20 Primary | SMIM5 | chr17:73636395 | Silent | C | T | I38I | 82 | 28 |
| MEEI20 Primary | SMIM5 | chr17:73636398 | Silent | C | T | I39I | 79 | 28 |
| MEEI20 Primary | SMIM5 | chr17:73636953 | Silent | C | G | V70V | 206 | 50 |
| MEEI20 Primary | TNRC6C | chr17:76089705 | Silent | C | T | F1386F | 25 | 114 |
| MEEI20 Primary | YES1 | chr18:724504 | Missense | T | G | T518P | 241 | 73 |
| MEEI20 Primary | ASXL3 | chr18:31325043 | Missense | A | T | E1744V | 43 | 31 |
| MEEI20 Primary | NFATC1 | chr18:77227465 | Missense | G | C | E659Q | 33 | 19 |
| MEEI20 Primary | ELANE | chr19:852350 | Missense | G | C | A8P | 122 | 62 |
| MEEI20 Primary | EIF3G | chr19:10230155 | Intron | G | C | | 50 | 29 |
| MEEI20 Primary | ASF1B | chr19:14247259 | Missense | C | G | V4L | 47 | 36 |
| MEEI20 Primary | DNAJB1 | chr19:14629164 | 5′UTR | C | A | | 33 | 7 |
| MEEI20 Primary | NOTCH3 | chr19:15292590 | Silent | C | T | S863S | 54 | 33 |
| MEEI20 Primary | ZNF100 | chr19:21910494 | Missense | C | A | C207F | 75 | 14 |
| MEEI20 Primary | ZNF676 | chr19:22362665 | 3′UTR | C | G | | 48 | 20 |
| MEEI20 Primary | ZNF676 | chr19:22363123 | Missense | T | C | K466E | 173 | 25 |
| MEEI20 Primary | TSHZ3 | chr19:31769178 | Silent | C | T | L507L | 239 | 74 |
| MEEI20 Primary | GAPDHS | chr19:36027753 | Missense | G | A | V36I | 94 | 34 |
| MEEI20 Primary | RYR1 | chr19:39051859 | Missense | G | A | R4125H | 132 | 41 |
| MEEI20 Primary | CYP2A7 | chr19:41386441 | Missense | C | G | E146Q | 134 | 29 |
| MEEI20 Primary | CEACAM5 | chr19:42212617 | 5′UTR | T | G | | 216 | 65 |
| MEEI20 Primary | MEGF8 | chr19:42853692 | Silent | G | A | T713T | 124 | 28 |
| MEEI20 Primary | PLAUR | chr19:44159686 | Missense | T | C | Y171C | 43 | 121 |
| MEEI20 Primary | PVR | chr19:45153109 | Silent | G | A | Q152Q | 132 | 35 |
| MEEI20 Primary | BCAM | chr19:45323063 | 3′UTR | C | G | | 5 | 14 |
| MEEI20 Primary | EHD2 | chr19:48239756 | Missense | C | G | S349C | 31 | 12 |
| MEEI20 Primary | MIR520E | chr19:54179008 | RNA | G | C | | 90 | 30 |
| MEEI20 Primary | LILRB5 | chr19:54756147 | Intron | G | T | | 19 | 11 |
| MEEI20 Primary | LILRB5 | chr19:54756783 | Silent | G | A | L466L | 51 | 160 |
| MEEI20 Primary | NLRP7 | chr19:55452842 | Missense | G | C | L108V | 262 | 101 |
| MEEI20 Primary | ZNF787 | chr19:56614225 | Missense | C | T | G121D | 25 | 45 |
| MEEI20 Primary | ADAM33 | chr20:3653920 | 5′UTR | G | C | | 262 | 59 |
| MEEI20 Primary | GPCPD1 | chr20:5559164 | Silent | G | A | S189S | 272 | 62 |
| MEEI20 Primary | RALGAPA2 | chr20:20586024 | Silent | G | C | L611L | 233 | 63 |
| MEEI20 Primary | FAM182B | chr20:25755811 | Nonsense | G | A | Q49* | 701 | 119 |
| MEEI20 Primary | SYS1 | chr20:43992331 | Missense | G | C | E54Q | 63 | 32 |
| MEEI20 Primary | ZMYND8 | chr20:45938888 | Missense | G | A | S29F | 166 | 45 |
| MEEI20 Primary | ZNF831 | chr20:57768943 | Missense | C | T | P957S | 62 | 25 |
| MEEI20 Primary | COL20A1 | chr20:61936807 | Missense | C | T | P78S | 20 | 10 |
| MEEI20 Primary | LSS | chr21:47642593 | Missense | T | A | I127F | 65 | 36 |
| MEEI20 Primary | USP18 | chr22:18644562 | Missense | C | T | T87M | 68 | 21 |
| MEEI20 Primary | HIRA | chr22:19349315 | Missense | C | G | E639Q | 286 | 53 |
| MEEI20 Primary | SMTN | chr22:31478961 | Intron | G | T | | 124 | 38 |
| MEEI20 Primary | PANX2 | chr22:50615649 | Missense | G | A | E170K | 172 | 53 |
| MEEI20 Primary | STS | chrX:7243411 | Silent | C | T | I376I | 48 | 97 |
| MEEI20 Primary | CNKSR2 | chrX:21670494 | Missense | A | T | Q957L | 17 | 60 |
| MEEI20 Primary | ZNF674 | chrX:46384812 | Intron | C | T | | 7 | 19 |
| MEEI20 Primary | Unknown | chrX:47695443 | IGR | C | T | | 2 | 7 |
| MEEI20 Primary | MAGIX | chrX:49022757 | 3′UTR | G | T | | 2 | 9 |
| MEEI20 Primary | AR | chrX:66909477 | 3′UTR | T | C | | 16 | 24 |
| MEEI20 Primary | KLHL4 | chrX:86924344 | Silent | C | T | L705L | 126 | 101 |
| MEEI20 LN | NOC2L | chr1:894240 | Intron | G | A | | 100 | 31 |
| MEEI20 LN | TARDBP | chr1:11073956 | Missense | G | C | E58Q | 280 | 83 |
| MEEI20 LN | MACF1 | chr1:39797507 | Silent | C | T | F1749F | 153 | 49 |
| MEEI20 LN | ZZZ3 | chr1:78097765 | Missense | T | A | Q425H | 48 | 16 |
| MEEI20 LN | KIAA1107 | chr1:92647732 | Missense | G | A | E1115K | 67 | 25 |
| MEEI20 LN | GSTM5 | chr1:110255418 | 5′UTR | G | C | | 74 | 30 |
| MEEI20 LN | GABPB2 | chr1:151062895 | Missense | C | T | P41L | 73 | 12 |
| MEEI20 LN | S100A16 | chr1:153579813 | 3′UTR | G | C | | 7 | 3 |
| MEEI20 LN | RCSD1 | chr1:167666670 | Missense | A | G | E270G | 228 | 46 |
| MEEI20 LN | RABGAP1L | chr1:174769546 | Missense | G | A | D26N | 136 | 25 |
| MEEI20 LN | TNR | chr1:175299356 | Missense | T | C | H1216R | 94 | 19 |
| MEEI20 LN | COLGALT2 | chr1:183899415 | Nonsense | G | C | S538* | 237 | 39 |
| MEEI20 LN | COLGALT2 | chr1:183933253 | 5′UTR | G | C | | 24 | 5 |
| MEEI20 LN | ASPM | chr1:197070960 | Missense | T | C | K2474R | 710 | 97 |
| MEEI20 LN | IL20 | chr1:207039175 | 5′UTR | G | C | | 181 | 28 |
| MEEI20 LN | RD3 | chr1:211652607 | Missense | G | A | S120L | 107 | 15 |
| MEEI20 LN | PTPN14 | chr1:214625204 | Missense | G | T | F96L | 118 | 17 |
| MEEI20 LN | MIA3 | chr1:222824217 | Missense | G | C | R1294T | 120 | 41 |
| MEEI20 LN | SUSD4 | chr1:223400943 | Missense | G | A | P352S | 76 | 55 |
| MEEI20 LN | DNAH14 | chr1:225562549 | Missense | G | A | E4067K | 199 | 34 |
| MEEI20 LN | OBSCN | chr1:228459730 | Missense | G | C | E1932Q | 575 | 92 |
| MEEI20 LN | OBSCN | chr1:228505340 | Silent | C | T | P5536P | 602 | 200 |
| MEEI20 LN | TRIM67 | chr1:231299118 | Missense | C | G | P135A | 137 | 23 |
| MEEI20 LN | FMN2 | chr1:240256314 | Missense | C | T | S302F | 40 | 7 |
| MEEI20 LN | FMN2 | chr1:240458138 | Silent | T | C | D1390D | 196 | 34 |
| MEEI20 LN | AHCTF1 | chr1:247014172 | Silent | T | C | K1747K | 186 | 163 |
| MEEI20 LN | OR2C3 | chr1:247695215 | Missense | A | G | M200T | 176 | 26 |
| MEEI20 LN | NBAS | chr2:15307376 | Silent | G | C | V2304V | 171 | 55 |
| MEEI20 LN | SDC1 | chr2:20403986 | Missense | G | A | T72M | 85 | 18 |
| MEEI20 LN | APOB | chr2:21225265 | Nonsense | A | T | Y4343* | 49 | 9 |
| MEEI20 LN | ATAD2B | chr2:24009067 | Missense | G | A | L935F | 60 | 34 |
| MEEI20 LN | SPAST | chr2:32340842 | Missense | G | C | L314F | 214 | 28 |
| MEEI20 LN | CCT4 | chr2:62099620 | Missense | C | G | C4105 | 233 | 31 |
| MEEI20 LN | TET3 | chr2:74274322 | Silent | A | T | P291P | 373 | 341 |
| MEEI20 LN | INO80B | chr2:74683244 | Missense | C | G | L129V | 40 | 21 |
| MEEI20 LN | POLR1A | chr2:86332979 | 5′UTR | C | T | | 20 | 13 |
| MEEI20 LN | POLR1A | chr2:86333207 | 5′UTR | G | A | | 37 | 9 |
| MEEI20 LN | RNF103 | chr2:86831783 | Missense | G | C | S414C | 153 | 17 |
| MEEI20 LN | ANKRD36C | chr2:96557426 | Missense | T | G | E948D | 144 | 33 |
| MEEI20 LN | POLR1B | chr2:113326347 | Missense | G | A | E648K | 89 | 17 |
| MEEI20 LN | TMEM177 | chr2:120439062 | Silent | A | G | A211A | 136 | 29 |
| MEEI20 LN | MGAT5 | chr2:135028001 | Missense | C | A | R965 | 111 | 23 |
| MEEI20 LN | KIF5C | chr2:149847540 | Missense | G | C | G578A | 71 | 18 |
| MEEI20 LN | NEB | chr2:152359330 | Missense | G | A | R7969C | 117 | 20 |
| MEEI20 LN | UBR3 | chr2:170850928 | Missense | G | A | D1294N | 289 | 58 |
| MEEI20 LN | GORASP2 | chr2:171822706 | 3′UTR | C | G | | 98 | 23 |
| MEEI20 LN | TTN | chr2:179497043 | Silent | T | C | R14526R | 247 | 27 |
| MEEI20 LN | CCDC141 | chr2:179770213 | Missense | C | G | G370R | 160 | 15 |
| MEEI20 LN | INO80D | chr2:206869892 | Missense | C | T | V762I | 379 | 70 |
| MEEI20 LN | PTH2R | chr2:209345831 | Silent | C | T | L340L | 90 | 38 |
| MEEI20 LN | MAP2 | chr2:210595014 | Nonsense | C | T | R1793* | 409 | 47 |
| MEEI20 LN | SLC16A14 | chr2:230910914 | Missense | G | C | L310V | 68 | 23 |
| MEEI20 LN | TRAF3IP1 | chr2:239256102 | Missense | C | A | S423Y | 150 | 33 |
| MEEI20 LN | MRPS25 | chr3:15091199 | 3′UTR | C | T | | 4 | 3 |
| MEEI20 LN | EOMES | chr3:27759160 | Missense | G | A | R488C | 97 | 74 |
| MEEI20 LN | XIRP1 | chr3:39227759 | Missense | T | A | M1060L | 42 | 47 |
| MEEI20 LN | ZNF852 | chr3:44541164 | Missense | G | C | Q369E | 119 | 39 |
| MEEI20 LN | CYB561D2 | chr3:50388837 | 5′UTR | C | T | | 29 | 12 |
| MEEI20 LN | KIAA2018 | chr3:113379729 | Missense | T | C | Q267R | 119 | 29 |
| MEEI20 LN | CASR | chr3:121980379 | Missense | G | A | S166N | 335 | 66 |
| MEEI20 LN | MUC13 | chr3:124631993 | Silent | G | A | P392P | 110 | 13 |
| MEEI20 LN | ATP2C1 | chr3:130682805 | Intron | T | A | | 249 | 41 |
| MEEI20 LN | AMOTL2 | chr3:134077549 | Missense | G | A | P763L | 92 | 28 |
| MEEI20 LN | SLC35G2 | chr3:136574442 | Silent | C | T | I380I | 122 | 27 |
| MEEI20 LN | TERC | chr3:169482512 | lincRNA | C | T | | 144 | 33 |
| MEEI20 LN | LRRC31 | chr3:169557996 | Missense | C | T | R478Q | 305 | 44 |
| MEEI20 LN | OPA1 | chr3:193355029 | Missense | G | T | D332Y | 131 | 52 |
| MEEI20 LN | DLG1 | chr3:196792240 | Missense | T | A | K793N | 67 | 15 |
| MEEI20 LN | PPARGC1A | chr4:23830201 | Silent | C | G | A193A | 223 | 79 |
| MEEI20 LN | SMR3B | chr4:71255799 | 3′UTR | C | A | | 182 | 34 |
| MEEI20 LN | NPFFR2 | chr4:72897735 | Silent | C | T | R39R | 68 | 22 |
| MEEI20 LN | AGPAT9 | chr4:84502751 | Missense | G | T | G82V | 69 | 46 |
| MEEI20 LN | PTPN13 | chr4:87622858 | Missense | G | A | E367K | 139 | 54 |
| MEEI20 LN | MTTP | chr4:100522763 | Splice Site | G | C | | 207 | 35 |
| MEEI20 LN | SLC9B1 | chr4:103870473 | Missense | C | T | G108E | 36 | 6 |
| MEEI20 LN | TBCK | chr4:107154194 | Nonsense | G | A | Q514* | 99 | 20 |
| MEEI20 LN | PHF17 | chr4:129793144 | Silent | G | C | R752R | 177 | 40 |
| MEEI20 LN | LRBA | chr4:151509256 | Missense | C | T | E2092K | 36 | 16 |
| MEEI20 LN | KLHL2 | chr4:166199071 | Intron | A | T | | 131 | 41 |
| MEEI20 LN | IRX1 | chr5:3599530 | Silent | C | T | T156T | 756 | 62 |
| MEEI20 LN | DNAH5 | chr5:13919404 | Missense | C | T | E286K | 269 | 33 |
| MEEI20 LN | FAM105A | chr5:14581990 | 5′UTR | G | T | | 161 | 24 |
| MEEI20 LN | FAM105A | chr5:14581991 | 5′UTR | C | T | | 163 | 24 |
| MEEI20 LN | PRDM9 | chr5:23527712 | Silent | C | A | R839R | 948 | 94 |
| MEEI20 LN | RAD1 | chr5:34914899 | Silent | A | G | A33A | 364 | 41 |
| MEEI20 LN | PPWD1 | chr5:64859267 | Missense | G | A | E44K | 97 | 18 |
| MEEI20 LN | PCDHA13 | chr5:140263972 | Missense | G | A | V707M | 230 | 61 |
| MEEI20 LN | PCDHB3 | chr5:140481075 | Missense | T | G | F281C | 83 | 23 |
| MEEI20 LN | PCDH12 | chr5:141336158 | Missense | C | T | R420K | 158 | 64 |
| MEEI20 LN | KIF4B | chr5:154394844 | Silent | G | A | Q475Q | 203 | 54 |
| MEEI20 LN | NSD1 | chr5:176638614 | Nonsense | C | T | R1072* | 117 | 28 |
| MEEI20 LN | RGS14 | chr5:176795865 | Missense | G | A | E333K | 166 | 51 |
| MEEI20 LN | GMDS | chr6:1961111 | Silent | G | A | I145I | 119 | 24 |
| MEEI20 LN | LRRC16A | chr6:25495409 | Missense | C | A | L431I | 102 | 15 |
| MEEI20 LN | SLC17A3 | chr6:25862664 | Missense | A | T | L34I | 209 | 50 |
| MEEI20 LN | HIST1H2AG | chr6:27100950 | Silent | C | T | L34L | 103 | 113 |
| MEEI20 LN | ZNRD1-AS1 | chr6:29976003 | RNA | G | A | | 101 | 13 |
| MEEI20 LN | BRD2 | chr6:32947795 | Missense | G | C | D713H | 263 | 64 |
| MEEI20 LN | DNAH8 | chr6:38821105 | Silent | C | T | L1688L | 73 | 11 |
| MEEI20 LN | UBR2 | chr6:42629988 | Missense | A | G | H1170R | 78 | 23 |
| MEEI20 LN | MAD2L1BP | chr6:43597287 | 5′UTR | A | C | | 59 | 8 |
| MEEI20 LN | SLC25A27 | chr6:46623757 | Missense | T | C | I95T | 58 | 16 |
| MEEI20 LN | GPR110 | chr6:46979830 | Silent | C | G | G343G | 172 | 38 |
| MEEI20 LN | EYS | chr6:65767552 | Missense | C | A | D698Y | 140 | 29 |
| MEEI20 LN | MDN1 | chr6:90458976 | Missense | C | T | C1243Y | 84 | 26 |
| MEEI20 LN | LAMA4 | chr6:112575392 | 5′UTR | G | T | | 109 | 32 |
| MEEI20 LN | CHST12 | chr7:2473426 | Silent | C | A | I384I | 105 | 53 |
| MEEI20 LN | TNRC18 | chr7:5428096 | Silent | G | A | S453S | 56 | 16 |
| MEEI20 LN | TNRC18 | chr7:5428097 | Missense | G | A | S453F | 57 | 16 |
| MEEI20 LN | USP42 | chr7:6193522 | Silent | C | T | R779R | 141 | 22 |
| MEEI20 LN | ZNF853 | chr7:6661136 | Missense | C | A | Q172K | 219 | 46 |
| MEEI20 LN | GARS | chr7:30662062 | Silent | C | T | L533L | 264 | 72 |
| MEEI20 LN | BBS9 | chr7:33427715 | Missense | C | G | L692V | 223 | 48 |
| MEEI20 LN | AMPH | chr7:38469134 | Intron | T | G | | 62 | 10 |
| MEEI20 LN | AEBP1 | chr7:44152354 | Silent | G | A | E805E | 135 | 38 |
| MEEI20 LN | NPC1L1 | chr7:44571753 | Missense | C | G | E825Q | 213 | 49 |
| MEEI20 LN | NPC1L1 | chr7:44578713 | Missense | C | T | G428E | 240 | 60 |
| MEEI20 LN | OGDH | chr7:44714050 | Missense | C | T | R277C | 147 | 25 |
| MEEI20 LN | ZNF736 | chr7:63808578 | Missense | T | A | L113I | 177 | 35 |
| MEEI20 LN | AUTS2 | chr7:70252291 | Missense | C | T | S802L | 154 | 53 |
| MEEI20 LN | MDH2 | chr7:75687389 | Missense | C | T | A141V | 53 | 60 |
| MEEI20 LN | GNAI1 | chr7:79840299 | Missense | G | T | G202V | 62 | 60 |
| MEEI20 LN | RNF133 | chr7:122338664 | Silent | T | C | A103A | 215 | 50 |
| MEEI20 LN | LMOD2 | chr7:123296256 | Missense | T | G | L80R | 104 | 40 |
| MEEI20 LN | KIAA1549 | chr7:138522791 | Missense | C | G | G1889R | 95 | 66 |
| MEEI20 LN | PRSS3P2 | chr7:142481377 | RNA | G | C | | 247 | 89 |
| MEEI20 LN | ZNF282 | chr7:148921263 | Missense | C | T | R514C | 35 | 30 |
| MEEI20 LN | ANGPT2 | chr8:6366411 | Missense | T | G | M457L | 67 | 22 |
| MEEI20 LN | BLK | chr8:11412293 | Missense | G | C | E172Q | 271 | 84 |
| MEEI20 LN | PENK | chr8:57354065 | Missense | C | T | M190I | 199 | 51 |
| MEEI20 LN | CHD7 | chr8:61769121 | Nonsense | C | T | R2428* | 248 | 46 |
| MEEI20 LN | ZFHX4 | chr8:77775863 | Missense | C | G | Q3305E | 126 | 33 |
| MEEI20 LN | DECR1 | chr8:91033274 | Silent | A | G | K176K | 37 | 7 |
| MEEI20 LN | OXR1 | chr8:107705027 | Silent | A | T | V199V | 120 | 38 |
| MEEI20 LN | PKHD1L1 | chr8:110439314 | Missense | G | C | E977Q | 226 | 31 |
| MEEI20 LN | ENPP2 | chr8:120629779 | Missense | G | C | I164M | 109 | 18 |
| MEEI20 LN | CDKN2A | chr9:21971000 | Nonsense | C | A | E120* | 186 | 340 |
| MEEI20 LN | MELK | chr9:36599467 | Nonsense | C | G | S152* | 102 | 21 |
| MEEI20 LN | ECM2 | chr9:95263289 | Missense | G | A | P551S | 197 | 51 |
| MEEI20 LN | PHF2 | chr9:96429442 | Missense | G | T | E756D | 97 | 117 |
| MEEI20 LN | DBC1 | chr9:122004494 | Splice Site | C | G | G137_splice | 62 | 32 |
| MEEI20 LN | TRAF1 | chr9:123675635 | Missense | G | A | R226C | 91 | 17 |
| MEEI20 LN | SPTAN1 | chr9:131374009 | Missense | A | T | H1597L | 513 | 115 |
| MEEI20 LN | NOTCH1 | chr9:139409769 | Nonsense | C | A | E663* | 147 | 237 |
| MEEI20 LN | LYZL1 | chr10:29577994 | 5′UTR | G | C | | 44 | 10 |
| MEEI20 LN | Unknown | chr10:38737632 | IGR | C | A | | 364 | 76 |
| MEEI20 LN | RSU1P2 | chr10:45602410 | RNA | A | C | | 20 | 11 |
| MEEI20 LN | EGR2 | chr10:64573084 | Silent | G | C | P438P | 22 | 7 |
| MEEI20 LN | CDH23 | chr10:73565590 | Missense | G | A | E2639K | 72 | 26 |
| MEEI20 LN | TNKS2 | chr10:93619394 | Silent | C | T | Y1090Y | 43 | 8 |
| MEEI20 LN | C10orf120 | chr10:124458877 | Nonsense | G | C | Y76* | 90 | 22 |
| MEEI20 LN | HMX2 | chr10:124909443 | Missense | C | T | S209L | 149 | 32 |
| MEEI20 LN | DHX32 | chr10:127527692 | Missense | T | A | I587F | 89 | 28 |
| MEEI20 LN | DHX32 | chr10:127527693 | Silent | A | T | V586V | 89 | 28 |
| MEEI20 LN | TTC40 | chr10:134622392 | Missense | G | C | L2561V | 27 | 8 |
| MEEI20 LN | KCNQ1 | chr11:2869097 | Missense | G | A | R632K | 94 | 50 |
| MEEI20 LN | PAX6 | chr11:31811444 | 3′UTR | C | T | | 89 | 29 |
| MEEI20 LN | SPI1 | chr11:47379894 | 3′UTR | C | T | | 0 | 2 |
| MEEI20 LN | OR5L1 | chr11:55579155 | Missense | C | G | F71L | 158 | 38 |
| MEEI20 LN | SLC15A3 | chr11:60718567 | Missense | G | T | P153T | 101 | 16 |
| MEEI20 LN | C11orf48 | chr11:62435057 | Missense | G | C | S185C | 290 | 71 |
| MEEI20 LN | CCDC88B | chr11:64124704 | Missense | C | T | S628F | 194 | 49 |
| MEEI20 LN | MAP3K11 | chr11:65367093 | Missense | C | T | D660N | 138 | 38 |
| MEEI20 LN | CATSPER1 | chr11:65793245 | Silent | G | C | L202L | 74 | 22 |
| MEEI20 LN | CCDC87 | chr11:66360235 | Missense | G | C | I84M | 84 | 14 |
| MEEI20 LN | LRFN4 | chr11:66625234 | Missense | C | G | L7V | 86 | 19 |
| MEEI20 LN | RELT | chr11:73103469 | Missense | C | T | T194M | 338 | 86 |
| MEEI20 LN | P4HA3 | chr11:74013514 | Missense | C | T | R156Q | 219 | 56 |
| MEEI20 LN | KMT2A | chr11:118376301 | Nonsense | C | T | R3232* | 169 | 46 |
| MEEI20 LN | CACNA1C | chr12:2690884 | Missense | G | A | G675E | 161 | 83 |
| MEEI20 LN | CACNA1C | chr12:2786826 | Intron | C | T | | 69 | 10 |
| MEEI20 LN | CACNA1C | chr12:2797785 | Missense | G | C | S1986T | 425 | 67 |
| MEEI20 LN | VAMP1 | chr12:6575058 | Missense | C | T | E80K | 237 | 32 |
| MEEI20 LN | KLRG1 | chr12:9144826 | Missense | C | T | S36F | 181 | 23 |
| MEEI20 LN | CLEC12A | chr12:10133239 | Silent | C | T | F146F | 111 | 64 |
| MEEI20 LN | PRICKLE1 | chr12:42854296 | Missense | G | T | P604Q | 132 | 29 |
| MEEI20 LN | TENC1 | chr12:53452958 | Silent | C | T | S511S | 35 | 14 |
| MEEI20 LN | AAAS | chr12:53701437 | Missense | G | A | R493C | 120 | 38 |
| MEEI20 LN | DCD | chr12:55039031 | Missense | C | G | G72A | 247 | 49 |
| MEEI20 LN | PA2G4 | chr12:56498234 | 5′UTR | T | A | | 119 | 21 |
| MEEI20 LN | BTBD11 | chr12:107914298 | Missense | C | G | H390Q | 224 | 36 |
| MEEI20 LN | CUX2 | chr12:111786131 | 3′UTR | G | A | | 47 | 10 |
| MEEI20 LN | PRKAB1 | chr12:120118120 | Missense | A | T | K268M | 129 | 21 |
| MEEI20 LN | HIP1R | chr12:123341234 | Missense | G | C | E527D | 179 | 22 |
| MEEI20 LN | SBNO1 | chr12:123795625 | Missense | G | A | S1091L | 97 | 43 |
| MEEI20 LN | CHFR | chr12:133423633 | Silent | C | A | L588L | 146 | 70 |
| MEEI20 LN | OLFM4 | chr13:53624619 | Missense | C | A | Q416K | 100 | 13 |
| MEEI20 LN | PCDH17 | chr13:58207963 | Missense | G | A | R428H | 45 | 51 |
| MEEI20 LN | HS6ST3 | chr13:97484833 | Missense | C | A | T266N | 67 | 83 |
| MEEI20 LN | TRAV9-1 | chr14:22279905 | RNA | C | T | | 125 | 38 |
| MEEI20 LN | PSMB5 | chr14:23495401 | Missense | C | G | G230A | 304 | 65 |
| MEEI20 LN | PSME1 | chr14:24605422 | 5′UTR | C | T | | 35 | 24 |
| MEEI20 LN | FKBP3 | chr14:45590794 | Silent | C | T | L116L | 147 | 24 |
| MEEI20 LN | TBPL2 | chr14:55890788 | Intron | C | T | | 15 | 10 |
| MEEI20 LN | OTX2 | chr14:57268852 | Silent | G | A | I165I | 198 | 41 |
| MEEI20 LN | PCNX | chr14:71413729 | Missense | C | G | A84G | 178 | 102 |
| MEEI20 LN | IGHD2-21 | chr14:106354434 | RNA | G | C | | 95 | 19 |
| MEEI20 LN | NPAPI | chr15:24923689 | Missense | A | G | N892S | 206 | 39 |
| MEEI20 LN | ATP10A | chr15:25940172 | Missense | G | T | S961Y | 253 | 146 |
| MEEI20 LN | SLCI2A1 | chr15:48524954 | Missense | A | T | I336F | 98 | 113 |
| MEEI20 LN | THAP10 | chr15:71174825 | Missense | T | C | M248V | 43 | 71 |
| MEEI20 LN | TTC23 | chr15:99696417 | Missense | C | T | R360Q | 158 | 173 |
| MEEI20 LN | CRAMP1L | chr16:1723984 | Missense | C | T | R1250C | 184 | 56 |
| MEEI20 LN | CREBBP | chr16:3781810 | Missense | C | G | QI619H | 395 | 74 |
| MEEI20 LN | PDXDC1 | chr16:15068872 | 5′UTR | C | T | | 163 | 39 |
| MEEI20 LN | TMC5 | chr16:19451967 | Missense | G | A | G203R | 243 | 57 |
| MEEI20 LN | GP2 | chr16:20322603 | Splice Site | C | T | G516_splice | 193 | 28 |
| MEEI20 LN | SLC5A11 | chr16:24909423 | Missense | C | A | L201I | 51 | 15 |
| MEEI20 LN | ZNF688 | chr16:30583569 | Silent | G | A | L10L | 34 | 13 |
| MEEI20 LN | CDH5 | chr16:66426087 | Missense | A | T | T340S | 411 | 144 |
| MEEI20 LN | CDH5 | chr16:66432000 | Silent | C | A | V492V | 297 | 53 |
| MEEI20 LN | CCDC79 | chr16:66803918 | Missense | G | C | Q523E | 112 | 77 |
| MEEI20 LN | CCDC79 | chr16:66822087 | Missense | G | C | LI29V | 39 | 26 |
| MEEI20 LN | RLTPR | chr16:67690171 | Silent | G | C | SI261S | 203 | 20 |
| MEEI20 LN | ACD | chr16:67693674 | Silent | G | T | RI72R | 119 | 20 |
| MEEI20 LN | ENKD1 | chr16:67700094 | Missense | G | T | P54T | 233 | 31 |
| MEEI20 LN | DPEP3 | chr16:68011617 | Nonsense | G | C | S3I6* | 192 | 24 |
| MEEI20 LN | ZNF821 | chr16:71898890 | Missense | C | G | E76D | 113 | 75 |
| MEEI20 LN | WDR59 | chr16:74926451 | Missense | A | T | N67IK | 321 | 25 |
| MEEI20 LN | CNTNAP4 | chr16:76501302 | Missense | C | G | Q5I2E | 378 | 48 |
| MEEI20 LN | KIAA0513 | chr16:85100869 | Silent | G | A | P64P | 144 | 86 |
| MEEI20 LN | DPHI | chr17:1946416 | 3′UTR | C | G | | 36 | 13 |
| MEEI20 LN | KIFIC | chr17:4925757 | Missense | A | G | D794G | 69 | 38 |
| MEEI20 LN | TP53 | chr17:7578493 | Nonsense | C | T | WI46* | 103 | 120 |
| MEEI20 LN | PIK3R5 | chr17:8789859 | Missense | C | T | D657N | 62 | 11 |
| MEEI20 LN | ARHGAP44 | chr17:12855898 | Splice Site | G | C | R379_splice | 82 | 23 |
| MEEI20 LN | RAI1 | chr17:17714169 | Missense | C | A | S1633Y | 33 | 67 |
| MEEI20 LN | SMCR8 | chr17:18226425 | 3′UTR | G | A | | 42 | 10 |
| MEEI20 LN | VTN | chr17:26697213 | Silent | C | G | L4L | 81 | 30 |
| MEEI20 LN | PEXI2 | chr17:33904475 | Missense | C | T | G88S | 423 | 54 |
| MEEI20 LN | STAT5A | chr17:40456393 | Silent | C | T | C401C | 291 | 24 |
| MEEI20 LN | CUEDC1 | chr17:55962593 | Silent | C | T | P111P | 22 | 7 |
| MEEI20 LN | KCNH6 | chr17:61620952 | Missense | C | T | H722Y | 131 | 28 |
| MEEI20 LN | ABCA9 | chr17:67022525 | Missense | G | A | H712Y | 62 | 19 |
| MEEI20 LN | RECQL5 | chr17:73625440 | Missense | C | A | G688V | 8 | 3 |
| MEEI20 LN | SMIM5 | chr17:73636395 | Silent | C | T | I38I | 73 | 16 |
| MEEI20 LN | SMIM5 | chr17:73636398 | Silent | C | T | I39I | 72 | 17 |
| MEEI20 LN | SMIM5 | chr17:73636953 | Silent | C | G | V70V | 208 | 47 |
| MEEI20 LN | TNRC6C | chr17:76089705 | Silent | C | T | FI386F | 50 | 83 |
| MEEI20 LN | YES1 | chr18:724504 | Missense | T | G | T5I8P | 254 | 70 |
| MEEI20 LN | NFATC1 | chr18:77227465 | Missense | G | C | E659Q | 35 | 12 |
| MEEI20 LN | ELANE | chr19:852350 | Missense | G | C | A8P | 146 | 49 |
| MEEI20 LN | EIF3G | chr19:10230155 | Intron | G | C | | 66 | 21 |
| MEEI20 LN | ASF1B | chr19:14247259 | Missense | C | G | V4L | 67 | 27 |
| MEEI20 LN | NOTCH3 | chr19:15292590 | Silent | C | T | S863S | 91 | 27 |
| MEEI20 LN | ZNF676 | chr19:22362665 | 3′UTR | C | G | | 39 | 7 |
| MEEI20 LN | TSHZ3 | chr19:31769178 | Silent | C | T | L507L | 230 | 62 |
| MEEI20 LN | GAPDHS | chr19:36027753 | Missense | G | A | V36I | 123 | 26 |
| MEEI20 LN | RYR1 | chr19:39051859 | Missense | G | A | R4I25H | 133 | 33 |
| MEEI20 LN | CYP2A7 | chr19:41386441 | Missense | C | G | E146Q | 132 | 33 |
| MEEI20 LN | CEACAM5 | chr19:42212617 | 5′UTR | T | G | | 226 | 56 |
| MEEI20 LN | PLAUR | chr19:44159686 | Missense | T | C | Y171C | 64 | 95 |
| MEEI20 LN | PVR | chr19:45153109 | Silent | G | A | Q152Q | 161 | 33 |
| MEEI20 LN | MIR520E | chr19:54179008 | RNA | G | C | | 112 | 20 |
| MEEI20 LN | LILRB5 | chr19:54756783 | Silent | G | A | L466L | 95 | 95 |
| MEEI20 LN | NLRP7 | chr19:55452842 | Missense | G | C | L108V | 338 | 77 |
| MEEI20 LN | ZNF787 | chr19:56614225 | Missense | C | T | G121D | 33 | 15 |
| MEEI20 LN | ADAM33 | chr20:3653920 | 5′UTR | G | C | | 286 | 54 |
| MEEI20 LN | GPCPD1 | chr20:5559164 | Silent | G | A | S189S | 287 | 51 |
| MEEI20 LN | RALGAPA2 | chr20:20586024 | Silent | G | C | L611L | 247 | 39 |
| MEEI20 LN | FAM182B | chr20:25755811 | Nonsense | G | A | Q49* | 730 | 81 |
| MEEI20 LN | SYS1 | chr20:43992331 | Missense | G | C | E54Q | 97 | 34 |
| MEEI20 LN | ZMYND8 | chr20:45938888 | Missense | G | A | S29F | 155 | 45 |
| MEEI20 LN | ZNF831 | chr20:57768943 | Missense | C | T | P957S | 75 | 16 |
| MEEI20 LN | COL20A1 | chr20:61936807 | Missense | C | T | P78S | 35 | 6 |
| MEEI20 LN | LSS | chr21:47642593 | Missense | T | A | I127F | 85 | 33 |
| MEEI20 LN | USP18 | chr22:18644562 | Missense | C | T | T87M | 74 | 12 |
| MEEI20 LN | HIRA | chr22:19349315 | Missense | C | G | E639Q | 280 | 44 |
| MEEI20 LN | OSBP2 | chr22:31289541 | Silent | C | T | L694L | 218 | 19 |
| MEEI20 LN | SMTN | chr22:31478961 | Intron | G | T | | 117 | 37 |
| MEEI20 LN | PANX2 | chr22:50615649 | Missense | G | A | E170K | 178 | 37 |
| MEEI20 LN | STS | chrX:7243411 | Silent | C | T | I376I | 68 | 92 |
| MEEI20 LN | CNKSR2 | chrX:21670494 | Missense | A | T | Q957L | 37 | 35 |
| MEEI20 LN | ZNF674 | chrX:46384812 | Intron | C | T | | 18 | 16 |
| MEEI20 LN | Unknown | chrX:47695443 | IGR | C | T | | 5 | 5 |
| MEEI20 LN | MAGIX | chrX:49022757 | 3′UTR | G | T | | 3 | 6 |
| MEEI20 LN | AR | chrX:66909477 | 3′UTR | T | C | | 22 | 12 |
| MEEI20 LN | KLHL4 | chrX:86924344 | Silent | C | T | L705L | 122 | 34 |
| MEEI20 LN | CSTF2 | chrX:100077337 | Missense | G | A | G79R | 121 | 37 |
| MEEI25 Primary | ACAP3 | chr1:1229477 | Missense | G | C | R748G | 52 | 19 |
| MEEI25 Primary | MYOM3 | chr1:24432519 | Missense | C | T | G1525 | 44 | 22 |
| MEEI25 Primary | KCNQ4 | chr1:41285897 | Missense | G | A | E336K | 180 | 54 |
| MEEI25 Primary | PLK3 | chr1:45271337 | Missense | G | A | R643H | 66 | 9 |
| MEEI25 Primary | CYP4B1 | chr1:47279982 | Missense | G | T | A292S | 36 | 15 |
| MEEI25 Primary | DMRTA2 | chr1:50886810 | Silent | G | A | Y133Y | 64 | 19 |
| MEEI25 Primary | BARHL2 | chr1:91182528 | Silent | C | T | P75P | 191 | 4 |
| MEEI25 Primary | SPRR3 | chr1:152975978 | Missense | C | T | P161L | 60 | 25 |
| MEEI25 Primary | PVRL4 | chr1:161047432 | Missense | C | T | V181M | 79 | 44 |
| MEEI25 Primary | DESI2 | chr1:244868941 | Silent | C | T | L145L | 140 | 14 |
| MEEI25 Primary | SNTG2 | chr2:1241770 | Missense | G | A | R277K | 61 | 5 |
| MEEI25 Primary | TPO | chr2:1481327 | Missense | C | T | A430V | 91 | 21 |
| MEEI25 Primary | MERTK | chr2:112786057 | Silent | C | T | Y872Y | 119 | 32 |
| MEEI25 Primary | SCN1A | chr2:166901591 | Silent | G | T | R542R | 97 | 29 |
| MEEI25 Primary | HOXD3 | chr2:177036714 | Missense | T | A | H337Q | 83 | 13 |
| MEEI25 Primary | TTN | chr2:179452446 | Missense | A | G | I21197T | 40 | 11 |
| MEEI25 Primary | ZNF804A | chr2:185801932 | Silent | T | C | C603C | 39 | 4 |
| MEEI25 Primary | DCLK3 | chr3:36756703 | 3′UTR | C | G | | 19 | 6 |
| MEEI25 Primary | DOCK3 | chr3:51392331 | Missense | C | T | R1376C | 27 | 5 |
| MEEI25 Primary | ALAS1 | chr3:52245399 | Silent | G | A | L477L | 169 | 28 |
| MEEI25 Primary | FBXO40 | chr3:121345790 | 3′UTR | G | T | | 33 | 6 |
| MEEI25 Primary | DNAJC13 | chr3:132224254 | Nonsense | A | T | K1665* | 50 | 6 |
| MEEI25 Primary | XRN1 | chr3:142075870 | Missense | T | C | T1186A | 152 | 26 |
| MEEI25 Primary | NAALADL2 | chr3:174951824 | Missense | T | C | Y217H | 86 | 28 |
| MEEI25 Primary | GABRA4 | chr4:46979448 | Missense | C | G | G158A | 47 | 19 |
| MEEI25 Primary | THAP9 | chr4:83825939 | Missense | G | A | R44H | 109 | 32 |
| MEEI25 Primary | PITX2 | chr4:111539425 | Silent | C | T | P270P | 200 | 7 |
| MEEI25 Primary | C4orf46 | chr4:159590626 | 3′UTR | G | A | | 5 | 3 |
| MEEI25 Primary | TRIM60 | chr4:165961938 | Silent | G | A | E238E | 79 | 16 |
| MEEI25 Primary | FAT1 | chr4:187584759 | Nonsense | C | A | E1092* | 45 | 14 |
| MEEI25 Primary | CTD-2031P19.3 | chr5:55297590 | RNA | G | A | | 54 | 16 |
| MEEI25 Primary | COL4A3BP | chr5:74754984 | Missense | C | A | R213L | 47 | 11 |
| MEEI25 Primary | PCDHGB7 | chr5:140799533 | Missense | T | A | F703I | 81 | 11 |
| MEEI25 Primary | MAML1 | chr5:179193271 | Silent | G | A | P420P | 48 | 8 |
| MEEI25 Primary | MEP1A | chr6:46803277 | Missense | C | T | A692V | 138 | 50 |
| MEEI25 Primary | GFRAL | chr6:55216206 | Nonsense | C | T | Q176* | 99 | 11 |
| MEEI25 Primary | OOEP | chr6:74079365 | Missense | C | T | V51M | 105 | 30 |
| MEEI25 Primary | C7orf10 | chr7:40488949 | Missense | G | A | V301I | 58 | 15 |
| MEEI25 Primary | FZD9 | chr7:72849451 | Missense | G | A | V372I | 45 | 20 |
| MEEI25 Primary | PTCD1 | chr7:99021422 | Silent | G | A | A632A | 104 | 14 |
| MEEI25 Primary | TMEM213 | chr7:138487647 | Missense | G | A | V53M | 28 | 13 |
| MEEI25 Primary | DPP6 | chr7:154681234 | Silent | C | T | S751S | 42 | 10 |
| MEEI25 Primary | NCAPG2 | chr7:158448079 | Silent | C | T | P819P | 106 | 38 |
| MEEI25 Primary | ADRA1A | chr8:26722037 | Silent | G | A | V150V | 83 | 15 |
| MEEI25 Primary | HTRA4 | chr8:38831702 | 5′UTR | C | T | | 56 | 24 |
| MEEI25 Primary | PKHD1L1 | chr8:110476761 | Missense | C | T | P2567L | 109 | 27 |
| MEEI25 Primary | COL22A1 | chr8:139890558 | Splice Site | A | G | G31_splice | 16 | 15 |
| MEEI25 Primary | MAFA | chr8:144511604 | Missense | T | A | S325C | 90 | 51 |
| MEEI25 Primary | CDKN2A | chr9:21971028 | Nonsense | C | T | W110* | 120 | 49 |
| MEEI25 Primary | MUSK | chr9:113547110 | Missense | C | T | T459M | 42 | 6 |
| MEEI25 Primary | FAM178A | chr10:102685854 | Nonsense | T | A | L707* | 49 | 21 |
| MEEI25 Primary | POLL | chr10:103347271 | 5′UTR | C | G | | 40 | 11 |
| MEEI25 Primary | INSC | chr11:15243032 | Missense | G | A | V324I | 14 | 4 |
| MEEI25 Primary | OR5M11 | chr11:56310097 | Missense | C | T | V213I | 66 | 21 |
| MEEI25 Primary | ZBTB16 | chr11:113934180 | Missense | C | T | A53V | 176 | 24 |
| MEEI25 Primary | LRP1 | chr12:57592404 | Silent | C | T | R3209R | 107 | 49 |
| MEEI25 Primary | TCTN1 | chr12:111064196 | Missense | C | A | A124E | 89 | 44 |
| MEEI25 Primary | P2RX7 | chr12:121615201 | Silent | C | T | N210N | 126 | 19 |
| MEEI25 Primary | COG3 | chr13:46093117 | Missense | A | G | H681R | 50 | 8 |
| MEEI25 Primary | MIR381HG | chr14:101513704 | lincRNA | G | A | | 172 | 44 |
| MEEI25 Primary | JAG2 | chr14:105617241 | Silent | G | A | N463N | 218 | 107 |
| MEEI25 Primary | MKRN3 | chr15:23855804 | 3′UTR | C | T | | 47 | 11 |
| MEEI25 Primary | NDNL2 | chr15:29561099 | Missense | G | T | H271N | 115 | 28 |
| MEEI25 Primary | CATSPER2 | chr15:43927945 | Silent | G | A | F367F | 278 | 56 |
| MEEI25 Primary | TRPM7 | chr15:50870862 | Splice Site | C | T | | 62 | 16 |
| MEEI25 Primary | CYP1A1 | chr15:75015124 | Silent | G | T | G105G | 93 | 9 |
| MEEI25 Primary | CAPN15 | chr16:602155 | Missense | C | T | A817V | 42 | 14 |
| MEEI25 Primary | GNPTG | chr16:1412218 | Silent | G | A | A141A | 154 | 39 |
| MEEI25 Primary | IGFALS | chr16:1842429 | Missense | G | A | P35L | 9 | 5 |
| MEEI25 Primary | PHKB | chr16:47495232 | 5′UTR | G | A | | 53 | 9 |
| MEEI25 Primary | CDH11 | chr16:65026854 | Missense | C | T | E203K | 102 | 22 |
| MEEI25 Primary | NFATC3 | chr16:68156527 | Silent | C | T | S247S | 81 | 17 |
| MEEI25 Primary | DLG4 | chr17:7120905 | Intron | C | A | | 4 | 8 |
| MEEI25 Primary | TP53 | chr17:7578268 | Missense | A | C | L194R | 177 | 156 |
| MEEI25 Primary | MYO15A | chr17:18025358 | Nonsense | G | T | G1082* | 131 | 14 |
| MEEI25 Primary | CUEDC1 | chr17:55962701 | Silent | G | A | G75G | 112 | 21 |
| MEEI25 Primary | ACE | chr17:61557729 | Silent | C | T | S229S | 194 | 52 |
| MEEI25 Primary | GAREM | chr18:29867120 | Silent | C | T | Q480Q | 77 | 15 |
| MEEI25 Primary | DAPK3 | chr19:3959459 | Silent | G | A | A335A | 48 | 14 |
| MEEI25 Primary | DKFZP761J1410 | chr19:11472074 | Nonsense | C | A | C19I* | 344 | 18 |
| MEEI25 Primary | NPHS1 | chr19:36335333 | Silent | C | T | V653V | 55 | 18 |
| MEEI25 Primary | LILRB5 | chr19:54757928 | Missense | C | A | G428V | 48 | 15 |
| MEEI25 Primary | PCSK2 | chr20:17207904 | 5′UTR | C | T | | 22 | 8 |
| MEEI25 Primary | PYGB | chr20:25273152 | Missense | G | T | D694Y | 167 | 44 |
| MEEI25 Primary | KCNQ2 | chr20:62073807 | Silent | C | T | G256G | 275 | 71 |
| MEEI25 Primary | NCAM2 | chr21:22658612 | Missense | G | A | V121I | 52 | 15 |
| MEEI25 Primary | RUNX1 | chr21:36164220 | 3′UTR | G | A | | 16 | 6 |
| MEEI25 Primary | Unknown | chr21:43720714 | IGR | T | C | | 66 | 13 |
| MEEI25 Primary | CCT8L2 | chr22:17072961 | Missense | A | T | D160E | 89 | 17 |
| MEEI25 Primary | MN1 | chr22:28194843 | Silent | C | T | S563S | 107 | 29 |
| MEEI25 Primary | GRAP2 | chr22:40356136 | Missense | G | A | R83Q | 77 | 21 |
| MEEI25 Primary | EFCAB6 | chr22:44107464 | Missense | C | T | A308T | 95 | 16 |
| MEEI25 Primary | WWC3 | chrX:10106972 | Missense | G | A | R1027Q | 29 | 5 |
| MEEI25 Primary | TLR8 | chrX:12938470 | Silent | C | T | T437T | 55 | 18 |
| MEEI25 Primary | CXorf30 | chrX:36254193 | 5′UTR | C | T | | 18 | 6 |
| MEEI25 Primary | SYN1 | chrX:47479178 | 5′UTR | G | A | | 20 | 12 |
| MEEI25 Primary | CACNA1F | chrX:49081329 | Missense | A | G | C602R | 170 | 52 |
| MEEI25 Primary | SATL1 | chrX:84362518 | Missense | G | C | P486R | 146 | 10 |
| MEEI25 Primary | ZNF711 | chrX:84502438 | 5′UTR | G | A | | 15 | 4 |
| MEEI25 Primary | DRP2 | chrX:100513469 | Silent | G | A | Q854Q | 178 | 6 |
| MEEI25 Primary | ZCCHC12 | chrX:117959874 | Missense | G | T | D223Y | 234 | 65 |
| MEEI25 Primary | SMARCA1 | chrX:128602861 | Nonsense | G | A | R863* | 48 | 5 |
| MEEI25 Primary | GABRE | chrX:151123498 | Missense | C | T | R399H | 30 | 11 |
| MEEI25 Primary | Unknown | chrGL000237.1:734 | IGR | C | T | | 84 | 4 |
| MEEI25 LN | ACAP3 | chr1:1229477 | Missense | G | C | R748G | 38 | 16 |
| MEEI25 LN | NPHP4 | chr1:5934954 | Missense | C | A | E1008D | 56 | 7 |
| MEEI25 LN | MYOM3 | chr1:24432519 | Missense | C | T | G152S | 33 | 20 |
| MEEI25 LN | NIPAL3 | chr1:24795615 | Silent | C | T | H305H | 69 | 26 |
| MEEI25 LN | KCNQ4 | chr1:41285897 | Missense | G | A | E336K | 141 | 60 |
| MEEI25 LN | PLK3 | chr1:45271337 | Missense | G | A | R643H | 54 | 11 |
| MEEI25 LN | CYP4B1 | chr1:47279982 | Missense | G | T | A2925 | 29 | 12 |
| MEEI25 LN | DMRTA2 | chr1:50886810 | Silent | G | A | Y133Y | 43 | 22 |
| MEEI25 LN | SPRR3 | chr1:152975978 | Missense | C | T | P161L | 37 | 26 |
| MEEI25 LN | PVRL4 | chr1:161047432 | Missense | C | T | V181M | 77 | 42 |
| MEEI25 LN | DESI2 | chr1:244868941 | Silent | C | T | L145L | 115 | 19 |
| MEEI25 LN | SNTG2 | chr2:1241770 | Missense | G | A | R277K | 64 | 13 |
| MEEI25 LN | TPO | chr2:1481327 | Missense | C | T | A430V | 74 | 30 |
| MEEI25 LN | MERTK | chr2:112786057 | Silent | C | T | Y872Y | 85 | 23 |
| MEEI25 LN | SCN1A | chr2:166901591 | Silent | G | T | R542R | 60 | 40 |
| MEEI25 LN | HOXD3 | chr2:177036714 | Missense | T | A | H337Q | 45 | 23 |
| MEEI25 LN | TTN | chr2:179452446 | Missense | A | G | I21197T | 40 | 19 |
| MEEI25 LN | ZNF804A | chr2:185801932 | Silent | T | C | C603C | 27 | 20 |
| MEEI25 LN | DCLK3 | chr3:36756703 | 3′UTR | C | G | | 20 | 3 |
| MEEI25 LN | DOCK3 | chr3:51392331 | Missense | C | T | R1376C | 22 | 4 |
| MEEI25 LN | IQCF6 | chr3:51812868 | Missense | C | A | R32L | 82 | 14 |
| MEEI25 LN | ALAS1 | chr3:52245399 | Silent | G | A | L477L | 113 | 22 |
| MEEI25 LN | FBX040 | chr3:121345790 | 3′UTR | G | T | | 46 | 9 |
| MEEI25 LN | DNAJC13 | chr3:132224254 | Nonsense | A | T | K1665* | 37 | 15 |
| MEEI25 LN | XRN1 | chr3:142075870 | Missense | T | C | T1186A | 117 | 46 |
| MEEI25 LN | NAALADL2 | chr3:174951824 | Missense | T | C | Y217H | 64 | 28 |
| MEEI25 LN | GABRA4 | chr4:46979448 | Missense | C | G | G158A | 36 | 20 |
| MEEI25 LN | THAP9 | chr4:83825939 | Missense | G | A | R44H | 73 | 32 |
| MEEI25 LN | TRIM60 | chr4:165961938 | Silent | G | A | E238E | 69 | 14 |
| MEEI25 LN | FAT1 | chr4:187584759 | Nonsense | C | A | E1092* | 32 | 16 |
| MEEI25 LN | CTD-2031P19.3 | chr5:55297590 | RNA | G | A | | 39 | 15 |
| MEEI25 LN | COL4A3BP | chr5:74754984 | Missense | C | A | R213L | 44 | 18 |
| MEEI25 LN | PCDHGB7 | chr5:140799533 | Missense | T | A | F703I | 68 | 13 |
| MEEI25 LN | MAML1 | chr5:179193271 | Silent | G | A | P420P | 40 | 12 |
| MEEI25 LN | MEP1A | chr6:46803277 | Missense | C | T | A692V | 115 | 58 |
| MEEI25 LN | GFRAL | chr6:55216206 | Nonsense | C | T | Q176* | 103 | 20 |
| MEEI25 LN | OOEP | chr6:74079365 | Missense | C | T | V51M | 57 | 37 |
| MEEI25 LN | C7orf10 | chr7:40488949 | Missense | G | A | V301I | 48 | 9 |
| MEEI25 LN | FZD9 | chr7:72849451 | Missense | G | A | V372I | 38 | 23 |
| MEEI25 LN | PTCD1 | chr7:99021422 | Silent | G | A | A632A | 77 | 17 |
| MEEI25 LN | TMEM213 | chr7:138487647 | Missense | G | A | V53M | 24 | 7 |
| MEEI25 LN | DPP6 | chr7:154681234 | Silent | C | T | S751S | 41 | 15 |
| MEEI25 LN | NCAPG2 | chr7:158448079 | Silent | C | T | P819P | 107 | 43 |
| MEEI25 LN | ADRA1A | chr8:26722037 | Silent | G | A | V150V | 64 | 18 |
| MEEI25 LN | HTRA4 | chr8:38831702 | 5′UTR | C | T | | 45 | 30 |
| MEEI25 LN | PKHD1L1 | chr8:110476761 | Missense | C | T | P2567L | 93 | 23 |
| MEEI25 LN | COL22A1 | chr8:139890558 | Splice Site | A | G | G31_splice | 10 | 10 |
| MEEI25 LN | MAFA | chr8:144511604 | Missense | T | A | S325C | 67 | 62 |
| MEEI25 LN | CDKN2A | chr9:21971028 | Nonsense | C | T | W110* | 64 | 50 |
| MEEI25 LN | MUSK | chr9:113547110 | Missense | C | T | T459M | 31 | 4 |
| MEEI25 LN | FAM178A | chr10:102685854 | Nonsense | T | A | L707* | 45 | 21 |
| MEEI25 LN | INSC | chr11:15243032 | Missense | G | A | V324I | 11 | 4 |
| MEEI25 LN | OR5M11 | chr11:56310097 | Missense | C | T | V213I | 38 | 18 |
| MEEI25 LN | ZBTB16 | chr11:113934180 | Missense | C | T | A53V | 139 | 29 |
| MEEI25 LN | LRP1 | chr12:57592404 | Silent | C | T | R3209R | 89 | 31 |
| MEEI25 LN | TCTN1 | chr12:111064196 | Missense | C | A | A124E | 103 | 32 |
| MEEI25 LN | P2RX7 | chr12:121615201 | Silent | C | T | N210N | 127 | 25 |
| MEEI25 LN | COG3 | chr13:46093117 | Missense | A | G | H681R | 55 | 14 |
| MEEI25 LN | NOVA1 | chr14:27064643 | Silent | G | A | L85L | 35 | 13 |
| MEEI25 LN | MIR381HG | chr14:101513704 | lincRNA | G | A | | 124 | 42 |
| MEEI25 LN | JAG2 | chr14:105617241 | Silent | G | A | N463N | 198 | 79 |
| MEEI25 LN | MKRN3 | chr15:23855804 | 3′UTR | C | T | | 47 | 15 |
| MEEI25 LN | NDNL2 | chr15:29561099 | Missense | G | T | H271N | 96 | 33 |
| MEEI25 LN | CATSPER2 | chr15:43927945 | Silent | G | A | F367F | 241 | 47 |
| MEEI25 LN | TRPM7 | chr15:50870862 | Splice Site | C | T | | 49 | 15 |
| MEEI25 LN | CYP1A1 | chr15:75015124 | Silent | G | T | G105G | 113 | 17 |
| MEEI25 LN | CAPN15 | chr16:602155 | Missense | C | T | A817V | 40 | 20 |
| MEEI25 LN | GNPTG | chr16:1412218 | Silent | G | A | A141A | 107 | 37 |
| MEEI25 LN | PHKB | chr16:47495232 | 5′UTR | G | A | | 38 | 17 |
| MEEI25 LN | CDH11 | chr16:65026854 | Missense | C | T | E203K | 63 | 22 |
| MEEI25 LN | NFATC3 | chr16:68156527 | Silent | C | T | S247S | 67 | 28 |
| MEEI25 LN | DLG4 | chr17:7120905 | Intron | C | A | | 7 | 4 |
| MEEI25 LN | TP53 | chr17:7578268 | Missense | A | C | L194R | 96 | 182 |
| MEEI25 LN | MYO15A | chr17:18025358 | Nonsense | G | T | G1082* | 112 | 21 |
| MEEI25 LN | GOSR1 | chr17:28850935 | 3′UTR | C | T | | 5 | 12 |
| MEEI25 LN | CUEDC1 | chr17:55962701 | Silent | G | A | G75G | 68 | 16 |
| MEEI25 LN | ACE | chr17:61557729 | Silent | C | T | S229S | 110 | 55 |
| MEEI25 LN | DAPK3 | chr19:3959459 | Silent | G | A | A335A | 59 | 17 |
| MEEI25 LN | AKAP8L | chr19:15511228 | Intron | A | C | | 25 | 8 |
| MEEI25 LN | NPHS1 | chr19:36335333 | Silent | C | T | V653V | 58 | 19 |
| MEEI25 LN | SYMPK | chr19:46318775 | 3′UTR | A | C | | 8 | 7 |
| MEEI25 LN | LILRB5 | chr19:54757928 | Missense | C | A | G428V | 44 | 14 |
| MEEI25 LN | PCSK2 | chr20:17207904 | 5′UTR | C | T | | 21 | 5 |
| MEEI25 LN | PYGB | chr20:25273152 | Missense | G | T | D694Y | 110 | 40 |
| MEEI25 LN | KCNQ2 | chr20:62073807 | Silent | C | T | G256G | 206 | 80 |
| MEEI25 LN | NCAM2 | chr21:22658612 | Missense | G | A | V121I | 48 | 24 |
| MEEI25 LN | RUNX1 | chr21:36164220 | 3′UTR | G | A | | 8 | 4 |
| MEEI25 LN | Unknown | chr21:43720714 | IGR | T | C | | 37 | 13 |
| MEEI25 LN | CCT8L2 | chr22:17072961 | Missense | A | T | D160E | 66 | 13 |
| MEEI25 LN | MN1 | chr22:28194843 | Silent | C | T | S563S | 65 | 35 |
| MEEI25 LN | GRAP2 | chr22:40356136 | Missense | G | A | R83Q | 67 | 32 |
| MEEI25 LN | EFCAB6 | chr22:44107464 | Missense | C | T | A308T | 71 | 19 |
| MEEI25 LN | WWC3 | chrX:10106972 | Missense | G | A | R1027Q | 30 | 7 |
| MEEI25 LN | TLR8 | chrX:12938470 | Silent | C | T | T437T | 31 | 14 |
| MEEI25 LN | MAGEB2 | chrX:30236816 | Missense | G | T | C40F | 81 | 14 |
| MEEI25 LN | CXorf30 | chrX:36254193 | 5′UTR | C | T | | 13 | 8 |
| MEEI25 LN | SYN1 | chrX:47479178 | 5′UTR | G | A | | 14 | 12 |
| MEEI25 LN | CACNA1F | chrX:49081329 | Missense | A | G | C602R | 133 | 55 |
| MEEI25 LN | SATL1 | chrX:84362518 | Missense | G | C | P486R | 157 | 29 |
| MEEI25 LN | ZNF711 | chrX:84502438 | 5′UTR | G | A | | 7 | 8 |
| MEEI25 LN | ZCCHC12 | chrX:117959874 | Missense | G | T | D223Y | 207 | 72 |
| MEEI25 LN | SMARCA1 | chrX:128602861 | Nonsense | G | A | R863* | 41 | 5 |
| MEEI25 LN | GABRE | chrX:151123498 | Missense | C | T | R399H | 18 | 20 |
| MEEI26 Primary | FBXO44 | chr1:11718910 | | | | | | |
| MEEI26 Primary | CROCC | chr1:17257001 | Missense | G | C | S254T | 225 | 18 |
| MEEI26 Primary | LDLRAD2 | chr1:22150218 | 3′UTR | G | A | | 195 | 36 |
| MEEI26 Primary | ASAP3 | chr1:23756197 | 3′UTR | C | T | | 2 | 3 |
| MEEI26 Primary | SFN | chr1:27189974 | Missense | G | A | E91K | 72 | 20 |
| MEEI26 Primary | AHDC1 | chr1:27874924 | Missense | G | A | R1235W | 39 | 31 |
| MEEI26 Primary | SESN2 | chr1:28599209 | Missense | G | C | E219Q | 105 | 31 |
| MEEI26 Primary | KIF2C | chr1:45226006 | Missense | C | G | F420L | 481 | 44 |
| MEEI26 Primary | PLK3 | chr1:45271335 | Missense | C | G | D642E | 203 | 20 |
| MEEI26 Primary | PTCH2 | chr1:45293992 | Missense | A | T | L562H | 96 | 488 |
| MEEI26 Primary | CMPK1 | chr1:47799713 | Silent | C | T | L32L | 175 | 23 |
| MEEI26 Primary | PTGER3 | chr1:71513167 | Missense | C | T | E32K | 64 | 17 |
| MEEI26 Primary | MSH4 | chr1:76333241 | Missense | A | G | K425E | 42 | 43 |
| MEEI26 Primary | TRMT13 | chr1:100613532 | Missense | G | C | K300N | 67 | 21 |
| MEEI26 Primary | MOV10 | chr1:113231481 | 5′UTR | G | A | | 25 | 20 |
| MEEI26 Primary | PTPN22 | chr1:114380455 | Missense | G | C | L523V | 162 | 42 |
| MEEI26 Primary | RNF115 | chr1:145688126 | Missense | G | C | G274A | 110 | 14 |
| MEEI26 Primary | LCE1E | chr1:152760033 | Silent | C | T | H86H | 120 | 55 |
| MEEI26 Primary | ARHGAP30 | chr1:161021288 | Missense | G | C | I412M | 108 | 18 |
| MEEI26 Primary | GPA33 | chr1:167042740 | Missense | G | A | P27L | 47 | 22 |
| MEEI26 Primary | NME7 | chr1:169293653 | Missense | G | A | P30L | 89 | 22 |
| MEEI26 Primary | FAM5C | chr1:190067408 | Missense | G | A | R681W | 129 | 25 |
| MEEI26 Primary | IGFN1 | chr1:201186533 | Missense | G | C | E3238D | 115 | 10 |
| MEEI26 Primary | C4BPB | chr1:207265155 | Silent | C | A | I133I | 75 | 17 |
| MEEI26 Primary | INTS7 | chr1:212156145 | Silent | C | G | V335V | 75 | 21 |
| MEEI26 Primary | TGFB2 | chr1:218536680 | Silent | C | G | V117V | 108 | 27 |
| MEEI26 Primary | CEP170 | chr1:243354534 | Silent | C | T | V298V | 118 | 38 |
| MEEI26 Primary | ZNF124 | chr1:247320212 | Missense | C | G | E176Q | 98 | 14 |
| MEEI26 Primary | OR2T6 | chr1:248551248 | Missense | C | A | F113L | 32 | 12 |
| MEEI26 Primary | MATN3 | chr2:20192865 | 3′UTR | G | C | | 164 | 21 |
| MEEI26 Primary | TTC27 | chr2:32889484 | Missense | A | G | D252G | 139 | 18 |
| MEEI26 Primary | SLC8A1 | chr2:40342502 | Missense | C | T | G902D | 203 | 34 |
| MEEI26 Primary | ACYP2 | chr2:54342872 | Missense | G | A | E41K | 61 | 20 |
| MEEI26 Primary | DNAH6 | chr2:85014299 | Missense | G | T | K3704N | 42 | 51 |
| MEEI26 Primary | CHMP3 | chr2:86734629 | Missense | C | T | D166N | 73 | 11 |
| MEEI26 Primary | IGKV1-12 | chr2:89339971 | RNA | G | A | | 452 | 98 |
| MEEI26 Primary | TSGA10 | chr2:99685371 | Nonsense | T | A | K400* | 36 | 8 |
| MEEI26 Primary | ZC3H8 | chr2:113012612 | 5′UTR | G | C | | 41 | 7 |
| MEEI26 Primary | EPC2 | chr2:149542400 | Silent | G | C | L727L | 71 | 19 |
| MEEI26 Primary | WDSUB1 | chr2:160132150 | Splice Site | C | T | | 49 | 7 |
| MEEI26 Primary | GRB14 | chr2:165349590 | Missense | G | C | L527V | 66 | 40 |
| MEEI26 Primary | DLX2 | chr2:172965392 | Missense | G | C | S289C | 46 | 11 |
| MEEI26 Primary | DLX2 | chr2:172965576 | Missense | G | A | P228S | 276 | 57 |
| MEEI26 Primary | NFE2L2 | chr2:178098864 | Missense | G | C | Q61E | 231 | 49 |
| MEEI26 Primary | TTN | chr2:179476137 | Missense | G | C | S16940C | 301 | 71 |
| MEEI26 Primary | TTN | chr2:179590563 | Missense | C | G | G6829A | 45 | 28 |
| MEEI26 Primary | CCDC141 | chr2:179701747 | Missense | C | A | S1400I | 53 | 114 |
| MEEI26 Primary | PLCL1 | chr2:198950305 | Silent | C | T | N688N | 58 | 128 |
| MEEI26 Primary | ABCA12 | chr2:215855472 | Missense | A | G | L1193P | 65 | 122 |
| MEEI26 Primary | PTPRN | chr2:220155600 | Silent | C | G | G914G | 104 | 14 |
| MEEI26 Primary | NGEF | chr2:233839670 | 5′UTR | G | C | | 30 | 5 |
| MEEI26 Primary | ZCWPW2 | chr3:28476708 | Nonsense | C | G | S147* | 103 | 26 |
| MEEI26 Primary | CCDC71 | chr3:49200688 | Silent | G | C | V318V | 272 | 66 |
| MEEI26 Primary | CCDC71 | chr3:49201377 | Missense | G | C | R89G | 151 | 51 |
| MEEI26 Primary | ST3GAL6 | chr3:98475259 | 5′UTR | C | G | | 180 | 13 |
| MEEI26 Primary | STAG1 | chr3:136323193 | Silent | G | C | V85V | 108 | 17 |
| MEEI26 Primary | IL20RB | chr3:136701125 | Silent | C | G | V113V | 83 | 10 |
| MEEI26 Primary | ZBBX | chr3:167023514 | Missense | G | C | Q548E | 129 | 13 |
| MEEI26 Primary | NAALADL2 | chr3:175455162 | Silent | T | C | A655A | 272 | 47 |
| MEEI26 Primary | PSMD2 | chr3:184019386 | Missense | T | G | L140R | 253 | 39 |
| MEEI26 Primary | ACO24560.3 | chr3:197354702 | RNA | G | A | | 16 | 11 |
| MEEI26 Primary | FAM184B | chr4:17636663 | Missense | G | C | P953R | 74 | 9 |
| MEEI26 Primary | NCAPG | chr4:17812743 | Missense | C | T | R15W | 37 | 14 |
| MEEI26 Primary | KIAA1239 | chr4:37446024 | Missense | C | T | S805F | 91 | 110 |
| MEEI26 Primary | CNGA1 | chr4:47942814 | Silent | G | C | V279V | 95 | 32 |
| MEEI26 Primary | ENAM | chr4:71508072 | Missense | G | A | R310K | 211 | 27 |
| MEEI26 Primary | NPFFR2 | chr4:73013177 | Missense | C | T | P406L | 234 | 31 |
| MEEI26 Primary | COPS4 | chr4:83989658 | Missense | G | A | G357E | 96 | 23 |
| MEEI26 Primary | FAM13A | chr4:89670923 | Splice Site | C | G | R693_splice | 31 | 10 |
| MEEI26 Primary | CCSER1 | chr4:91389411 | Missense | G | T | V544F | 75 | 30 |
| MEEI26 Primary | ANK2 | chr4:114279576 | Missense | G | A | D3268N | 104 | 22 |
| MEEI26 Primary | NDNF | chr4:121961107 | Silent | C | T | E97E | 101 | 35 |
| MEEI26 Primary | SH3RF1 | chr4:170038681 | Silent | C | T | V590V | 115 | 14 |
| MEEI26 Primary | NUP155 | chr5:37364401 | Silent | G | T | I81I | 472 | 45 |
| MEEI26 Primary | TMEM174 | chr5:72469365 | Missense | C | A | Q99K | 222 | 82 |
| MEEI26 Primary | ANKRD34B | chr5:79854712 | Nonsense | G | C | S376* | 119 | 44 |
| MEEI26 Primary | VCAN | chr5:82816437 | Missense | C | G | T771R | 190 | 61 |
| MEEI26 Primary | VCAN | chr5:82835979 | Missense | C | G | S2386C | 173 | 42 |
| MEEI26 Primary | MEF2C | chr5:88057020 | Silent | G | T | I128I | 84 | 70 |
| MEEI26 Primary | MEF2C | chr5:88057021 | Missense | A | G | I128T | 83 | 69 |
| MEEI26 Primary | PCDHA2 | chr5:140175104 | Missense | T | G | N185K | 144 | 32 |
| MEEI26 Primary | PCDHA5 | chr5:140203362 | Silent | C | T | L668L | 98 | 132 |
| MEEI26 Primary | PCDHA11 | chr5:140249252 | Missense | G | C | Q188H | 106 | 30 |
| MEEI26 Primary | PCDHB13 | chr5:140595303 | Silent | C | T | H536H | 493 | 129 |
| MEEI26 Primary | FAT2 | chr5:150945456 | Missense | G | A | L1013F | 99 | 29 |
| MEEI26 Primary | SLIT3 | chr5:168098289 | Silent | C | T | E1347E | 176 | 53 |
| MEEI26 Primary | ERGIC1 | chr5:172342676 | 3′UTR | G | C | | 48 | 19 |
| MEEI26 Primary | MBOAT1 | chr6:20115562 | Missense | A | G | Y196H | 108 | 20 |
| MEEI26 Primary | PPP1R18 | chr6:30653375 | Missense | C | G | E141Q | 92 | 19 |
| MEEI26 Primary | MDC1 | chr6:30675692 | Silent | C | T | E888E | 267 | 34 |
| MEEI26 Primary | MED20 | chr6:41874850 | Missense | A | G | I200T | 121 | 20 |
| MEEI26 Primary | B3GAT2 | chr6:71571595 | Missense | C | G | D275H | 242 | 44 |
| MEEI26 Primary | FILIP1 | chr6:76023690 | Missense | C | G | E620Q | 11 | 7 |
| MEEI26 Primary | PREP | chr6:105726245 | Missense | A | G | L636P | 149 | 86 |
| MEEI26 Primary | REV3L | chr6:111696409 | Nonsense | G | C | S972* | 78 | 8 |
| MEEI26 Primary | MED23 | chr6:131948597 | Missense | T | A | E33V | 156 | 71 |
| MEEI26 Primary | GRM1 | chr6:146351152 | Missense | G | T | V167L | 244 | 41 |
| MEEI26 Primary | STXBP5 | chr6:147636860 | Missense | G | A | E538K | 85 | 39 |
| MEEI26 Primary | QKI | chr6:163991728 | Missense | G | C | A338P | 98 | 22 |
| MEEI26 Primary | INTS1 | chr7:1519275 | Missense | G | A | R1573C | 34 | 10 |
| MEEI26 Primary | NEUROD6 | chr7:31378079 | Missense | A | C | N268K | 81 | 78 |
| MEEI26 Primary | NEUROD6 | chr7:31378245 | Missense | C | G | S213T | 79 | 71 |
| MEEI26 Primary | DDX56 | chr7:44613461 | Missense | T | C | M12V | 112 | 51 |
| MEEI26 Primary | POM121 | chr7:72419917 | 3′UTR | G | C | | 135 | 29 |
| MEEI26 Primary | LRRN3 | chr7:110762939 | Missense | C | G | I37M | 128 | 25 |
| MEEI26 Primary | CAPZA2 | chr7:116546356 | Missense | G | T | A156S | 96 | 19 |
| MEEI26 Primary | KCND2 | chr7:120385840 | Missense | G | A | E492K | 82 | 67 |
| MEEI26 Primary | FLNC | chr7:128496664 | Silent | G | T | S2448S | 178 | 24 |
| MEEI26 Primary | CREB3L2 | chr7:137597805 | Missense | G | C | T1725 | 78 | 21 |
| MEEI26 Primary | ZNF777 | chr7:149128876 | Silent | C | T | T829T | 43 | 36 |
| MEEI26 Primary | ZNF467 | chr7:149462099 | Missense | G | A | R498C | 29 | 37 |
| MEEI26 Primary | KAT6A | chr8:41800387 | Nonsense | G | C | S787* | 545 | 67 |
| MEEI26 Primary | Unknown | chr8:74171716 | IGR | G | C | | 50 | 11 |
| MEEI26 Primary | ZNF572 | chr8:125990012 | Missense | G | C | G501A | 73 | 20 |
| MEEI26 Primary | FAM135B | chr8:139379643 | Intron | C | G | | 98 | 16 |
| MEEI26 Primary | SPATA6L | chr9:4626364 | Intron | G | A | | 17 | 5 |
| MEEI26 Primary | TLN1 | chr9:35704047 | Missense | G | A | L2058F | 21 | 30 |
| MEEI26 Primary | GOLM1 | chr9:88661396 | Missense | G | C | F152L | 82 | 33 |
| MEEI26 Primary | SECISBP2 | chr9:91943769 | Missense | G | C | E257Q | 97 | 18 |
| MEEI26 Primary | ECM2 | chr9:95263009 | Splice Site | C | T | R644_splice | 29 | 9 |
| MEEI26 Primary | ZNF782 | chr9:99581281 | Missense | G | A | P342S | 28 | 29 |
| MEEI26 Primary | ZNF483 | chr9:114289875 | Missense | G | A | R67K | 54 | 64 |
| MEEI26 Primary | NUP214 | chr9:134106098 | Missense | G | C | Q2052H | 144 | 32 |
| MEEI26 Primary | SFMBT2 | chr10:7214545 | Missense | G | T | P688H | 71 | 30 |
| MEEI26 Primary | UNC5B | chr10:73045106 | Missense | G | C | E158Q | 145 | 54 |
| MEEI26 Primary | CDH23 | chr10:73538055 | Missense | C | G | S1731C | 51 | 21 |
| MEEI26 Primary | ECD | chr10:74912177 | Silent | G | C | V262V | 89 | 16 |
| MEEI26 Primary | DNAJC9 | chr10:75007269 | 5′UTR | G | C | | 99 | 39 |
| MEEI26 Primary | Unknown | chr10:75181975 | IGR | C | T | | 17 | 6 |
| MEEI26 Primary | VCL | chr10:75757905 | 5′UTR | C | T | | 95 | 14 |
| MEEI26 Primary | 42799 | chr10:94070940 | Silent | A | G | E28E | 132 | 51 |
| MEEI26 Primary | FBXW4 | chr10:103433327 | Missense | G | A | R154W | 131 | 37 |
| MEEI26 Primary | TACC2 | chr10:123976245 | Missense | G | A | C2483Y | 218 | 78 |
| MEEI26 Primary | PLEKHA1 | chr10:124187945 | Intron | A | G | | 30 | 5 |
| MEEI26 Primary | HMX3 | chr10:124896826 | Missense | G | A | S218N | 100 | 33 |
| MEEI26 Primary | TUBGCP2 | chr10:135116312 | Missense | G | A | S45F | 87 | 14 |
| MEEI26 Primary | NUP98 | chill :3784133 | Splice Site | G | A | S362_splice | 70 | 15 |
| MEEI26 Primary | OR52A4 | chr11:5142484 | RNA | G | A | | 62 | 37 |
| MEEI26 Primary | HPX | chr11:6458607 | Intron | G | C | | 51 | 17 |
| MEEI26 Primary | ZNF214 | chr11:7022705 | Missense | C | A | W70L | 93 | 23 |
| MEEI26 Primary | PPFIBP2 | chr11:7654148 | Silent | G | A | Q373Q | 132 | 21 |
| MEEI26 Primary | BDNF | chr11:27679676 | Missense | C | T | E146K | 319 | 59 |
| MEEI26 Primary | MAPK8IP1 | chr11:45921932 | Missense | G | A | G1415 | 120 | 43 |
| MEEI26 Primary | FNBP4 | chr11:47739036 | Missense | C | T | E998K | 162 | 21 |
| MEEI26 Primary | LGALS12 | chr11:63283143 | Silent | G | A | L213L | 105 | 23 |
| MEEI26 Primary | SLC22A12 | chr11:64367916 | Missense | T | A | Y455N | 78 | 18 |
| MEEI26 Primary | TBC1D10C | chr11:67177099 | Silent | G | A | P405P | 188 | 26 |
| MEEI26 Primary | UVRAG | chr11:75563030 | Missense | G | A | E74K | 64 | 12 |
| MEEI26 Primary | RAB30 | chr11:82693313 | Missense | C | T | R169Q | 213 | 43 |
| MEEI26 Primary | MMP27 | chr11:102562489 | 3′UTR | C | G | | 23 | 7 |
| MEEI26 Primary | USP2-AS1 | chr11:119369466 | RNA | T | A | | 54 | 86 |
| MEEI26 Primary | Unknown | chr11:124135484 | IGR | C | A | | 83 | 83 |
| MEEI26 Primary | DDX25 | chr11:125780292 | Missense | C | G | L181V | 81 | 26 |
| MEEI26 Primary | CACNA2D4 | chr12:2024097 | Missense | T | G | K78Q | 219 | 15 |
| MEEI26 Primary | TULP3 | chr12:3029941 | Missense | G | A | E36K | 281 | 50 |
| MEEI26 Primary | ATN1 | chr12:7045469 | Missense | G | C | E347Q | 124 | 85 |
| MEEI26 Primary | Unknown | chr12:7052373 | IGR | G | A | | 93 | 9 |
| MEEI26 Primary | Unknown | chr12:9462490 | IGR | C | G | | 54 | 12 |
| MEEI26 Primary | CLEC2D | chr12:9847456 | Missense | G | C | D188H | 168 | 131 |
| MEEI26 Primary | PPFIBP1 | chr12:27832883 | Nonsense | C | G | S601* | 246 | 20 |
| MEEI26 Primary | CNTN1 | chr12:41327586 | Silent | C | T | I297I | 234 | 29 |
| MEEI26 Primary | RAPGEF3 | chr12:48133006 | Missense | G | A | S752L | 188 | 40 |
| MEEI26 Primary | PRKAG1 | chr12:49412516 | Splice Site | G | T | T3_splice | 92 | 33 |
| MEEI26 Primary | SLC4A8 | chr12:51844497 | Intron | C | G | | 29 | 4 |
| MEEI26 Primary | SLC4A8 | chr12:51845970 | Missense | C | T | H114Y | 227 | 31 |
| MEEI26 Primary | TESPA1 | chr12:55357536 | Silent | G | C | L77L | 145 | 12 |
| MEEI26 Primary | GLI1 | chr12:57864433 | Missense | G | A | R637Q | 63 | 12 |
| MEEI26 Primary | LRIG3 | chr12:59308053 | Silent | G | T | R101R | 98 | 17 |
| MEEI26 Primary | PWP1 | chr12:108079654 | 5′UTR | G | C | | 86 | 9 |
| MEEI26 Primary | SDSL | chr12:113865895 | Silent | C | G | L36L | 238 | 51 |
| MEEI26 Primary | CLIP1 | chr12:122861975 | Silent | C | T | K206K | 128 | 14 |
| MEEI26 Primary | CCDC92 | chr12:124428789 | 5′UTR | C | G | | 114 | 13 |
| MEEI26 Primary | ULK1 | chr12:132399728 | Missense | A | G | R492G | 22 | 16 |
| MEEI26 Primary | Unknown | chr13:19425963 | IGR | G | C | | 193 | 28 |
| MEEI26 Primary | SACS | chr13:23914642 | Silent | G | A | L1125L | 43 | 11 |
| MEEI26 Primary | SACS | chr13:23928706 | De_novo_Start_OutOfFrame | G | C | | 71 | 37 |
| MEEI26 Primary | PABPC3 | chr13:25671891 | Missense | C | T | R519C | 159 | 44 |
| MEEI26 Primary | RNF6 | chr13:26789563 | Silent | T | C | E152E | 31 | 61 |
| MEEI26 Primary | FLT1 | chr13:29004202 | Missense | G | A | S364L | 155 | 28 |
| MEEI26 Primary | MTUS2 | chr13:29599440 | Missense | C | T | S212F | 59 | 52 |
| MEEI26 Primary | MTUS2 | chr13:29599441 | Silent | C | T | S212S | 59 | 53 |
| MEEI26 Primary | BRCA2 | chr13:32914045 | Silent | C | T | I1851I | 19 | 13 |
| MEEI26 Primary | NBEA | chr13:35745535 | Missense | G | C | E1457Q | 70 | 24 |
| MEEI26 Primary | SLITRK5 | chr13:88329176 | Missense | C | G | F511L | 122 | 35 |
| MEEI26 Primary | ARHGEF40 | chr14:21553067 | Silent | C | T | Y1315Y | 59 | 36 |
| MEEI26 Primary | KIAA0391 | chr14:35592848 | Nonsense | G | T | E133* | 120 | 27 |
| MEEI26 Primary | FBX033 | chr14:39870794 | Missense | C | T | D328N | 138 | 20 |
| MEEI26 Primary | SIX4 | chr14:61190475 | Silent | C | T | L106L | 46 | 13 |
| MEEI26 Primary | VASH1 | chr14:77229338 | Silent | C | T | V58V | 31 | 34 |
| MEEI26 Primary | MARK3 | chr14:103933461 | Missense | C | G | S348C | 22 | 22 |
| MEEI26 Primary | ADS5L1 | chr14:105211189 | Missense | G | A | E415K | 117 | 29 |
| MEEI26 Primary | IGHV1-46 | chr14:106967503 | RNA | C | T | | 73 | 25 |
| MEEI26 Primary | OR4N4 | chr15:22383022 | Missense | G | C | V184L | 369 | 52 |
| MEEI26 Primary | MAPKBP1 | chr15:42114509 | Missense | G | C | E1040Q | 102 | 21 |
| MEEI26 Primary | SPG11 | chr15:44941065 | Splice Site | T | A | E534_splice | 129 | 43 |
| MEEI26 Primary | SPATA5L1 | chr15:45709498 | Missense | G | C | M623I | 69 | 24 |
| MEEI26 Primary | MY05C | chr15:52571749 | Silent | G | C | L87L | 81 | 33 |
| MEEI26 Primary | BBS4 | chr15:73023750 | Silent | C | T | L272L | 294 | 77 |
| MEEI26 Primary | SH3GL3 | chr15:84286981 | Missense | G | A | G337E | 120 | 42 |
| MEEI26 Primary | Unknown | chr15:84946969 | IGR | C | A | | 124 | 41 |
| MEEI26 Primary | TICRR | chr15:90167446 | Missense | C | T | S1302F | 139 | 28 |
| MEEI26 Primary | RGS11 | chr16:318695 | 3′UTR | G | A | | 43 | 11 |
| MEEI26 Primary | CCDC78 | chr16:774678 | Intron | C | G | | 99 | 17 |
| MEEI26 Primary | PRR25 | chr16:855561 | Missense | G | A | R40Q | 115 | 15 |
| MEEI26 Primary | PDXDC1 | chr16:15083846 | Intron | C | T | | 45 | 78 |
| MEEI26 Primary | KIAA0430 | chr16:15690543 | 3′UTR | A | G | | 31 | 5 |
| MEEI26 Primary | AMFR | chr16:56443377 | Missense | G | T | L158M | 45 | 10 |
| MEEI26 Primary | AMFR | chr16:56443450 | Silent | G | A | I133I | 59 | 12 |
| MEEI26 Primary | AMFR | chr16:56443453 | Silent | G | A | F132F | 60 | 12 |
| MEEI26 Primary | CBFA2T3 | chr16:88964489 | Missense | G | A | L126F | 162 | 26 |
| MEEI26 Primary | SPG7 | chr16:89623334 | Missense | G | C | E741Q | 315 | 59 |
| MEEI26 Primary | CRK | chr17:1359482 | 5′UTR | C | A | | 14 | 11 |
| MEEI26 Primary | WDR81 | chr17:1633734 | Missense | G | A | R1243H | 47 | 22 |
| MEEI26 Primary | DHX33 | chr17:5372104 | Missense | A | C | F26V | 48 | 18 |
| MEEI26 Primary | TP53 | chr17:7577106 | Missense | G | A | P278S | 181 | 277 |
| MEEI26 Primary | KDM6B | chr17:7755300 | Silent | C | T | V1399V | 381 | 92 |
| MEEI26 Primary | KRT12 | chr17:39020047 | Missense | C | G | D293H | 109 | 21 |
| MEEI26 Primary | LINC00671 | chr17:41031777 | lincRNA | C | G | | 54 | 19 |
| MEEI26 Primary | EFTUD2 | chr17:42931994 | Missense | C | T | R730H | 70 | 16 |
| MEEI26 Primary | PLEKHM1P | chr17:62825358 | RNA | C | G | | 214 | 42 |
| MEEI26 Primary | MY015B | chr17:73612814 | 3′UTR | G | A | | 216 | 47 |
| MEEI26 Primary | TBCD | chr17:80866292 | Intron | C | G | | 74 | 25 |
| MEEI26 Primary | PPP4R1 | chr18:9559507 | Silent | T | A | A646A | 98 | 19 |
| MEEI26 Primary | PIGN | chr18:59781831 | Missense | G | C | A405G | 109 | 30 |
| MEEI26 Primary | MKNK2 | chr19:2043138 | Missense | C | G | E160Q | 41 | 7 |
| MEEI26 Primary | MAP2K2 | chr19:4095428 | Missense | T | C | N335S | 45 | 29 |
| MEEI26 Primary | GTF2F1 | chr19:6393087 | 5′UTR | G | A | | 52 | 17 |
| MEEI26 Primary | FBN3 | chr19:8193958 | Silent | G | A | S750S | 65 | 15 |
| MEEI26 Primary | CERS4 | chr19:8320565 | Silent | G | C | R71R | 59 | 11 |
| MEEI26 Primary | MUC16 | chr19:9074580 | Missense | G | A | S4289L | 87 | 19 |
| MEEI26 Primary | ZNF878 | chr19:12155729 | Missense | T | C | R210G | 176 | 48 |
| MEEI26 Primary | C19orf60 | chr19:18699953 | Intron | C | T | | 19 | 9 |
| MEEI26 Primary | ZNF567 | chr19:37185723 | 5′UTR | G | C | | 38 | 11 |
| MEEI26 Primary | ACTN4 | chr19:39216475 | Missense | G | C | E708Q | 186 | 28 |
| MEEI26 Primary | HIPK4 | chr19:40889765 | Missense | G | C | F249L | 67 | 24 |
| MEEI26 Primary | ZNF284 | chr19:44590583 | Missense | G | A | D318N | 47 | 77 |
| MEEI26 Primary | ZNF808 | chr19:53056414 | Missense | G | C | R82T | 58 | 8 |
| MEEI26 Primary | ZNF808 | chr19:53056639 | Missense | G | C | G157A | 187 | 30 |
| MEEI26 Primary | ZNF808 | chr19:53056710 | Missense | G | C | E181Q | 171 | 26 |
| MEEI26 Primary | ZNF331 | chr19:54080516 | Missense | G | C | Q234H | 118 | 21 |
| MEEI26 Primary | NLRP11 | chr19:56320236 | Silent | G | C | V580V | 86 | 24 |
| MEEI26 Primary | ZSCAN18 | chr19:58600079 | Nonsense | C | A | E177* | 72 | 18 |
| MEEI26 Primary | ZNF497 | chr19:58870272 | Intron | G | C | | 46 | 12 |
| MEEI26 Primary | ZNF497 | chr19:58870844 | Intron | G | C | | 116 | 16 |
| MEEI26 Primary | N0P56 | chr20:2633801 | Intron | G | A | | 21 | 6 |
| MEEI26 Primary | PANK2 | chr20:3870037 | Missense | C | G | S97C | 101 | 16 |
| MEEI26 Primary | NINL | chr20:25507072 | Missense | G | A | T51M | 83 | 9 |
| MEEI26 Primary | MMP9 | chr20:44642100 | Missense | G | A | D513N | 158 | 67 |
| MEEI26 Primary | VAPB | chr20:57024667 | 3′UTR | G | C | | 147 | 12 |
| MEEI26 Primary | LAMA5 | chr20:60906095 | Missense | G | A | P1215S | 44 | 29 |
| MEEI26 Primary | LAMA5 | chr20:60922001 | Missense | G | A | P347L | 108 | 39 |
| MEEI26 Primary | STMN3 | chr20:62275172 | Silent | C | T | K76K | 78 | 17 |
| MEEI26 Primary | ITSN1 | chr21:35186316 | Missense | G | C | Q889H | 75 | 23 |
| MEEI26 Primary | COL6A1 | chr21:47404298 | Missense | A | C | S115R | 163 | 216 |
| MEEI26 Primary | RTN4R | chr22:20229380 | Missense | G | C | R426G | 200 | 26 |
| MEEI26 Primary | TOP3B | chr22:22318356 | Silent | G | T | G381G | 31 | 19 |
| MEEI26 Primary | BCR | chr22:23523874 | Missense | G | A | D243N | 92 | 23 |
| MEEI26 Primary | Unknown | chr22:25041592 | IGR | C | T | | 725 | 59 |
| MEEI26 Primary | APOL2 | chr22:36623531 | Missense | C | G | K311N | 64 | 16 |
| MEEI26 Primary | RRP7A | chr22:42910266 | Missense | C | A | E201D | 87 | 25 |
| MEEI26 Primary | FLJ27365 | chr22:46501575 | Missense | G | A | G165E | 182 | 26 |
| MEEI26 Primary | TRABD | chr22:50631520 | Missense | G | A | E11K | 98 | 17 |
| MEEI26 Primary | TYMP | chr22:50968008 | Missense | C | G | R44P | 242 | 41 |
| MEEI26 Primary | ATP7A | chrX:77245113 | Missense | G | C | R332T | 78 | 39 |
| MEEI26 L LN | FBX044 | chr1:11718910 | Nonsense | C | T | R161* | 65 | 21 |
| MEEI26 L LN | AHDC1 | chr1:27874924 | Missense | G | A | R1235W | 46 | 21 |
| MEEI26 L LN | KIF2C | chr1:45226006 | Missense | C | G | F420L | 587 | 35 |
| MEEI26 L LN | PLK3 | chr1:45271335 | Missense | C | G | D642E | 236 | 22 |
| MEEI26 L LN | PTCH2 | chr1:45293992 | Missense | A | T | L562H | 151 | 855 |
| MEEI26 L LN | CMPK1 | chr1:47799713 | Silent | C | T | L32L | 164 | 20 |
| MEEI26 L LN | PTGER3 | chr1:71513167 | Missense | C | T | E32K | 77 | 16 |
| MEEI26 L LN | MSH4 | chr1:76333241 | Missense | A | G | K425E | 47 | 39 |
| MEEI26 L LN | DPYD | chr1:97771782 | Silent | C | T | L710L | 225 | 43 |
| MEEI26 L LN | MOV10 | chr1:113231481 | 5′UTR | G | A | | 45 | 22 |
| MEEI26 L LN | LCE1E | chr1:152760033 | Silent | C | T | H86H | 180 | 33 |
| MEEI26 L LN | NUP210L | chr1:153973447 | Silent | G | C | V1757V | 164 | 34 |
| MEEI26 L LN | UHMK1 | chr1:162492298 | Silent | G | A | L406L | 136 | 20 |
| MEEI26 L LN | UAP1 | chr1:162557310 | Missense | C | G | R294G | 102 | 11 |
| MEEI26 L LN | GPA33 | chr1:167042740 | Missense | G | A | P27L | 57 | 13 |
| MEEI26 L LN | GORAB | chr1:170508677 | Missense | G | A | D155N | 61 | 23 |
| MEEI26 L LN | ZC3H11A | chr1:203821384 | Missense | A | C | K764Q | 322 | 47 |
| MEEI26 L LN | DNAH14 | chr1:225458485 | Missense | G | A | D2524N | 112 | 16 |
| MEEI26 L LN | CEP170 | chr1:243354534 | Silent | C | T | V298V | 138 | 32 |
| MEEI26 L LN | OR2AK2 | chr1:248129574 | Missense | G | A | G314E | 116 | 14 |
| MEEI26 L LN | ACYP2 | chr2:54342872 | Missense | G | A | E41K | 89 | 9 |
| MEEI26 L LN | DNAH6 | chr2:85014299 | Missense | G | T | K3704N | 56 | 55 |
| MEEI26 L LN | IGKV1-12 | chr2:89339971 | RNA | G | A | | 437 | 88 |
| MEEI26 L LN | TSGA10 | chr2:99685371 | Nonsense | T | A | K400* | 18 | 5 |
| MEEI26 L LN | TMEM182 | chr2:103414430 | Missense | A | C | K147T | 132 | 26 |
| MEEI26 L LN | EPC2 | chr2:149542400 | Silent | G | C | L727L | 79 | 13 |
| MEEI26 L LN | BAZ2B | chr2:160239253 | Silent | C | T | L1274L | 217 | 42 |
| MEEI26 L LN | GRB14 | chr2:165349590 | Missense | G | C | L527V | 72 | 45 |
| MEEI26 L LN | NFE2L2 | chr2:178098864 | Missense | G | C | Q61E | 272 | 47 |
| MEEI26 L LN | TTN | chr2:179476137 | Missense | G | C | S16940C | 324 | 54 |
| MEEI26 L LN | TTN | chr2:179590563 | Missense | C | G | G6829A | 43 | 25 |
| MEEI26 L LN | CCDC141 | chr2:179701747 | Missense | C | A | S1400I | 87 | 86 |
| MEEI26 L LN | PLCL1 | chr2:198950305 | Silent | C | T | N688N | 106 | 107 |
| MEEI26 L LN | ABCA12 | chr2:215855472 | Missense | A | G | L1193P | 121 | 90 |
| MEEI26 L LN | PTPRN | chr2:220155600 | Silent | C | G | G914G | 119 | 20 |
| MEEI26 L LN | SNED1 | chr2:242011668 | Intron | A | C | | 99 | 14 |
| MEEI26 L LN | CNTN4 | chr3:3072590 | Missense | G | C | G572R | 277 | 50 |
| MEEI26 L LN | ZCWPW2 | chr3:28476708 | Nonsense | C | G | S147* | 141 | 34 |
| MEEI26 L LN | SHOX2 | chr3:157823807 | Missense | C | G | E3Q | 478 | 20 |
| MEEI26 L LN | ZBBX | chr3:167023514 | Missense | G | C | Q548E | 104 | 30 |
| MEEI26 L LN | NAALADL2 | chr3:175455162 | Silent | T | C | A655A | 245 | 64 |
| MEEI26 L LN | PSMD2 | chr3:184019386 | Missense | T | G | L140R | 236 | 20 |
| MEEI26 L LN | ACO24560.3 | chr3:197354702 | RNA | G | A | | 16 | 5 |
| MEEI26 L LN | FAM184B | chr4:17636663 | Missense | G | C | P953R | 83 | 9 |
| MEEI26 L LN | NCAPG | chr4:17812743 | Missense | C | T | R15W | 44 | 12 |
| MEEI26 L LN | KIAA1239 | chr4:37446024 | Missense | C | T | S805F | 149 | 90 |
| MEEI26 L LN | CNGA1 | chr4:47942814 | Silent | G | C | V279V | 68 | 20 |
| MEEI26 L LN | FAM13A | chr4:89670923 | Splice Site | C | G | R693_splice | 57 | 11 |
| MEEI26 L LN | CCSER1 | chr4:91389411 | Missense | G | T | V544F | 118 | 25 |
| MEEI26 L LN | NDNF | chr4:121961107 | Silent | C | T | E97E | 142 | 29 |
| MEEI26 L LN | SH3RF1 | chr4:170038681 | Silent | C | T | V590V | 108 | 31 |
| MEEI26 L LN | NUP155 | chr5:37364401 | Silent | G | T | I81I | 475 | 49 |
| MEEI26 L LN | MROH2B | chr5:41033158 | Silent | C | T | L782L | 252 | 19 |
| MEEI26 L LN | TMEM174 | chr5:72469365 | Missense | C | A | Q99K | 268 | 71 |
| MEEI26 L LN | ANKRD34B | chr5:79854712 | Nonsense | G | C | S376* | 173 | 48 |
| MEEI26 L LN | VCAN | chr5:82816437 | Missense | C | G | T771R | 279 | 56 |
| MEEI26 L LN | MEF2C | chr5:88057020 | Silent | G | T | I128I | 112 | 45 |
| MEEI26 L LN | MEF2C | chr5:88057021 | Missense | A | G | I128T | 111 | 45 |
| MEEI26 L LN | PCDHA2 | chr5:140175104 | Missense | T | G | N185K | 146 | 46 |
| MEEI26 L LN | PCDHA5 | chr5:140203362 | Silent | C | T | L668L | 143 | 96 |
| MEEI26 L LN | PCDHB13 | chr5:140595303 | Silent | C | T | H536H | 553 | 90 |
| MEEI26 L LN | RARS | chr5:167944899 | Missense | G | A | E569K | 151 | 28 |
| MEEI26 L LN | SLIT3 | chr5:168098289 | Silent | C | T | E1347E | 193 | 39 |
| MEEI26 L LN | ERGIC1 | chr5:172342676 | 3′UTR | G | C | | 61 | 20 |
| MEEI26 L LN | KIF13A | chr6:17873656 | Missense | C | G | D58H | 75 | 12 |
| MEEI26 L LN | PPP1R18 | chr6:30653375 | Missense | C | G | E141Q | 98 | 20 |
| MEEI26 L LN | MED20 | chr6:41874850 | Missense | A | G | I200T | 152 | 15 |
| MEEI26 L LN | DST | chr6:56350020 | Intron | G | A | | 35 | 9 |
| MEEI26 L LN | B3GAT2 | chr6:71571595 | Missense | C | G | D275H | 239 | 38 |
| MEEI26 L LN | FILIP1 | chr6:76023690 | Missense | C | G | E620Q | 21 | 11 |
| MEEI26 L LN | PREP | chr6:105726245 | Missense | A | G | L636P | 166 | 84 |
| MEEI26 L LN | MED23 | chr6:131948597 | Missense | T | A | E33V | 169 | 56 |
| MEEI26 L LN | BCLAF1 | chr6:136589490 | Intron | G | A | | 69 | 5 |
| MEEI26 L LN | KIAA1244 | chr6:138584700 | Missense | G | C | E694Q | 112 | 7 |
| MEEI26 L LN | GRM1 | chr6:146351152 | Missense | G | T | V167L | 307 | 37 |
| MEEI26 L LN | STXBP5 | chr6:147636860 | Missense | G | A | E538K | 103 | 42 |
| MEEI26 L LN | GRID2IP | chr7:6548741 | Missense | T | G | T659P | 22 | 8 |
| MEEI26 L LN | NEUROD6 | chr7:31378079 | Missense | A | C | N268K | 102 | 65 |
| MEEI26 L LN | NEUROD6 | chr7:31378245 | Missense | C | G | S213T | 114 | 69 |
| MEEI26 L LN | DDX56 | chr7:44613461 | Missense | T | C | M12V | 149 | 35 |
| MEEI26 L LN | PKD1L1 | chr7:47835746 | Silent | C | T | L2732L | 75 | 19 |
| MEEI26 L LN | AP1S1 | chr7:100799961 | Missense | G | C | K30N | 82 | 5 |
| MEEI26 L LN | KCND2 | chr7:120385840 | Missense | G | A | E492K | 130 | 70 |
| MEEI26 L LN | CREB3L2 | chr7:137597805 | Missense | G | C | T172S | 106 | 20 |
| MEEI26 L LN | ZNF777 | chr7:149128876 | Silent | C | T | T829T | 52 | 35 |
| MEEI26 L LN | ZNF467 | chr7:149462099 | Missense | G | A | R498C | 46 | 23 |
| MEEI26 L LN | KAT6A | chr8:41800387 | Nonsense | G | C | S787* | 616 | 92 |
| MEEI26 L LN | POLB | chr8:42206560 | Missense | A | C | E71D | 179 | 33 |
| MEEI26 L LN | RP1 | chr8:55541307 | Missense | A | G | E1622G | 159 | 29 |
| MEEI26 L LN | Unknown | chr8:74171716 | IGR | G | C | | 42 | 9 |
| MEEI26 L LN | TRHR | chr8:110099987 | Missense | C | G | N82K | 244 | 51 |
| MEEI26 L LN | ZNF572 | chr8:125990012 | Missense | G | C | G501A | 70 | 20 |
| MEEI26 L LN | FAM135B | chr8:139379643 | Intron | C | G | | 116 | 29 |
| MEEI26 L LN | SPATA6L | chr9:4626364 | Intron | G | A | | 24 | 7 |
| MEEI26 L LN | TLN1 | chr9:35704047 | Missense | G | A | L2058F | 59 | 37 |
| MEEI26 L LN | GOLM1 | chr9:88661396 | Missense | G | C | F152L | 88 | 29 |
| MEEI26 L LN | CENPP | chr9:95094452 | Splice Site | A | C | Q36_splice | 25 | 4 |
| MEEI26 L LN | ECM2 | chr9:95263009 | Splice Site | C | T | R644_splice | 49 | 13 |
| MEEI26 L LN | ZNF782 | chr9:99581281 | Missense | G | A | P342S | 33 | 24 |
| MEEI26 L LN | ZNF483 | chr9:114289875 | Missense | G | A | R67K | 77 | 45 |
| MEEI26 L LN | ARRDC1 | chr9:140500156 | 5′UTR | G | T | | 24 | 5 |
| MEEI26 L LN | UNC5B | chr10:73045106 | Missense | G | C | E158Q | 178 | 33 |
| MEEI26 L LN | CDH23 | chr10:73538055 | Missense | C | G | S1731C | 69 | 14 |
| MEEI26 L LN | DNAJC9 | chr10:75007269 | 5′UTR | G | C | | 92 | 24 |
| MEEI26 L LN | 42799 | chr10:94070940 | Silent | A | G | E28E | 179 | 42 |
| MEEI26 L LN | SLIT1 | chr10:98763835 | Silent | G | A | L1285L | 29 | 12 |
| MEEI26 L LN | FBXW4 | chr10:103433327 | Missense | G | A | R154W | 123 | 29 |
| MEEI26 L LN | TACC2 | chr10:123976245 | Missense | G | A | C2483Y | 259 | 60 |
| MEEI26 L LN | PLEKHA1 | chr10:124187945 | Intron | A | G | | 19 | 6 |
| MEEI26 L LN | HMX3 | chr10:124896826 | Missense | G | A | S218N | 118 | 20 |
| MEEI26 L LN | NUP98 | chr11:3784133 | Splice Site | G | A | S362_splice | 72 | 22 |
| MEEI26 L LN | OR52A4 | chr11:5142484 | RNA | G | A | | 60 | 27 |
| MEEI26 L LN | TRIM6 | chr11:5624790 | Missense | A | T | E111V | 228 | 52 |
| MEEI26 L LN | ZNF214 | chr11:7022705 | Missense | C | A | W70L | 129 | 25 |
| MEEI26 L LN | KIF18A | chr11:28058202 | Nonsense | G | C | S653* | 88 | 16 |
| MEEI26 L LN | MAPK8IP1 | chr11:45921932 | Missense | G | A | G141S | 147 | 27 |
| MEEI26 L LN | LGALS12 | chr11:63283143 | Silent | G | A | L213L | 125 | 17 |
| MEEI26 L LN | SLC22A12 | chr11:64367916 | Missense | T | A | Y455N | 79 | 14 |
| MEEI26 L LN | GRIA4 | chr11:105795133 | Missense | G | C | E495D | 32 | 5 |
| MEEI26 L LN | USP2-AS1 | chr11:119369466 | RNA | T | A | | 98 | 63 |
| MEEI26 L LN | Unknown | chr11:124135484 | IGR | C | A | | 121 | 63 |
| MEEI26 L LN | TULP3 | chr12:3029941 | Missense | G | A | E36K | 163 | 36 |
| MEEI26 L LN | ATN1 | chr12:7045469 | Missense | G | C | E347Q | 122 | 61 |
| MEEI26 L LN | Unknown | chr12:9462490 | IGR | C | G | | 34 | 6 |
| MEEI26 L LN | CLEC2D | chr12:9847456 | Missense | G | C | D188H | 155 | 60 |
| MEEI26 L LN | RP11-967K21.1 | chr12:28336700 | RNA | C | T | | 237 | 20 |
| MEEI26 L LN | RAPGEF3 | chr12:48133006 | Missense | G | A | S752L | 180 | 27 |
| MEEI26 L LN | PRKAG1 | chr12:49412516 | Splice Site | G | T | T3_splice | 113 | 28 |
| MEEI26 L LN | TESPA1 | chr12:55357536 | Silent | G | C | L77L | 143 | 15 |
| MEEI26 L LN | PWP1 | chr12:108079654 | 5′UTR | G | C | | 92 | 14 |
| MEEI26 L LN | SDSL | chr12:113865895 | Silent | C | G | L36L | 307 | 30 |
| MEEI26 L LN | IL31 | chr12:122658717 | Start_Codon_SNP | C | T | M1I | 173 | 24 |
| MEEI26 L LN | CLIP1 | chr12:122861975 | Silent | C | T | K206K | 145 | 19 |
| MEEI26 L LN | HCAR3 | chr12:123200711 | Missense | T | C | M192V | 271 | 45 |
| MEEI26 L LN | ULK1 | chr12:132399728 | Missense | A | G | R492G | 41 | 6 |
| MEEI26 L LN | SACS | chr13:23928706 | De_novo_Start_OutOfFrame | G | C | | 101 | 21 |
| MEEI26 L LN | PABPC3 | chr13:25671891 | Missense | C | T | R519C | 171 | 43 |
| MEEI26 L LN | RNF6 | chr13:26789563 | Silent | T | C | E152E | 85 | 50 |
| MEEI26 L LN | MTUS2 | chr13:29599440 | Missense | C | T | S212F | 84 | 48 |
| MEEI26 L LN | MTUS2 | chr13:29599441 | Silent | C | T | S212S | 84 | 49 |
| MEEI26 L LN | BRCA2 | chr13:32914045 | Silent | C | T | I1851I | 35 | 11 |
| MEEI26 L LN | SLITRK5 | chr13:88329176 | Missense | C | G | F511L | 201 | 37 |
| MEEI26 L LN | ARHGEF40 | chr14:21553067 | Silent | C | T | Y1315Y | 85 | 19 |
| MEEI26 L LN | HECTD1 | chr14:31602821 | Silent | C | G | V1213V | 96 | 21 |
| MEEI26 L LN | VASH1 | chr14:77229338 | Silent | C | T | V58V | 55 | 27 |
| MEEI26 L LN | MARK3 | chr14:103933461 | Missense | C | G | S348C | 15 | 14 |
| MEEI26 L LN | OR4N4 | chr15:22383022 | Missense | G | C | V184L | 456 | 39 |
| MEEI26 L LN | EXD1 | chr15:41483741 | Missense | G | A | L197F | 78 | 19 |
| MEEI26 L LN | SPG11 | chr15:44941065 | Splice Site | T | A | E534_splice | 177 | 36 |
| MEEI26 L LN | SPATA5L1 | chr15:45709498 | Missense | G | C | M623I | 63 | 18 |
| MEEI26 L LN | MYO5C | chr15:52571749 | Silent | G | C | L87L | 87 | 29 |
| MEEI26 L LN | SH3GL3 | chr15:84286981 | Missense | G | A | G337E | 157 | 32 |
| MEEI26 L LN | Unknown | chr15:84946969 | IGR | C | A | | 139 | 30 |
| MEEI26 L LN | PRSS41 | chr16:2848552 | RNA | G | A | | 42 | 17 |
| MEEI26 L LN | PDXDC1 | chr16:15083846 | Intron | C | T | | 63 | 48 |
| MEEI26 L LN | KIAA0430 | chr16:15690543 | 3′UTR | A | G | | 24 | 13 |
| MEEI26 L LN | CRK | chr17:1359482 | 5′UTR | C | A | | 12 | 7 |
| MEEI26 L LN | WDR81 | chr17:1633734 | Missense | G | A | R1243H | 74 | 17 |
| MEEI26 L LN | RABEP1 | chr17:5264669 | Missense | C | T | A421V | 337 | 57 |
| MEEI26 L LN | DHX33 | chr17:5372104 | Missense | A | C | F26V | 69 | 12 |
| MEEI26 L LN | TP53 | chr17:7577106 | Missense | G | A | P278S | 302 | 231 |
| MEEI26 L LN | MPRIP | chr17:17069775 | Intron | G | C | | 114 | 6 |
| MEEI26 L LN | DUSP14 | chr17:35872411 | Missense | C | T | L13F | 64 | 8 |
| MEEI26 L LN | DUSP14 | chr17:35872479 | Missense | C | G | F35L | 176 | 20 |
| MEEI26 L LN | LINC00671 | chr17:41031777 | lincRNA | C | G | | 70 | 11 |
| MEEI26 L LN | EFTUD2 | chr17:42931994 | Missense | C | T | R730H | 78 | 26 |
| MEEI26 L LN | DLX3 | chr17:48072208 | Missense | G | C | S52W | 274 | 42 |
| MEEI26 L LN | TRIM25 | chr17:54981779 | Nonsense | G | C | S25S* | 236 | 37 |
| MEEI26 L LN | CA4 | chr17:58235448 | Missense | C | G | F180L | 211 | 36 |
| MEEI26 L LN | CA4 | chr17:58235471 | Missense | C | G | S188C | 219 | 30 |
| MEEI26 L LN | PLEKHM1P | chr17:62825358 | RNA | C | G | | 272 | 27 |
| MEEI26 L LN | CYTH1 | chr17:76698687 | Splice Site | C | T | | 88 | 8 |
| MEEI26 L LN | TBCD | chr17:80866292 | Intron | C | G | | 86 | 8 |
| MEEI26 L LN | LRRC30 | chr18:7231142 | Silent | G | A | G2G | 35 | 5 |
| MEEI26 L LN | PPP4R1 | chr18:9559507 | Silent | T | A | A646A | 100 | 20 |
| MEEI26 L LN | PIGN | chr18:59781831 | Missense | G | C | A405G | 90 | 19 |
| MEEI26 L LN | MUM1 | chr19:1360306 | Missense | C | T | S61L | 36 | 11 |
| MEEI26 L LN | MUM1 | chr19:1360786 | Missense | C | T | S221L | 16 | 5 |
| MEEI26 L LN | KLF16 | chr19:1863108 | Missense | G | A | P130L | 67 | 20 |
| MEEI26 L LN | MAP2K2 | chr19:4095428 | Missense | T | C | N335S | 56 | 13 |
| MEEI26 L LN | GTF2F1 | chr19:6393087 | 5′UTR | G | A | | 83 | 18 |
| MEEI26 L LN | MUC16 | chr19:8974070 | Missense | C | G | E14201Q | 106 | 6 |
| MEEI26 L LN | ZNF878 | chr19:12155729 | Missense | T | C | R210G | 210 | 53 |
| MEEI26 L LN | ZNF383 | chr19:37726901 | Missense | C | G | Q53E | 78 | 13 |
| MEEI26 L LN | HIPK4 | chr19:40889765 | Missense | G | C | F249L | 66 | 11 |
| MEEI26 L LN | ZNF284 | chr19:44590583 | Missense | G | A | D318N | 69 | 41 |
| MEEI26 L LN | DNAAF3 | chr19:55678004 | Missense | G | C | LSV | 50 | 16 |
| MEEI26 L LN | DEFB127 | chr20:139420 | Missense | G | A | E19K | 63 | 15 |
| MEEI26 L LN | PYGB | chr20:25252062 | Silent | C | T | Y156Y | 135 | 20 |
| MEEI26 L LN | Unknown | chr20:43883206 | IGR | G | A | | 99 | 8 |
| MEEI26 L LN | MMP9 | chr20:44642100 | Missense | G | A | D513N | 194 | 26 |
| MEEI26 L LN | VAPB | chr20:57024667 | 3′UTR | G | C | | 118 | 16 |
| MEEI26 L LN | LAMA5 | chr20:60906095 | Missense | G | A | P1215S | 56 | 10 |
| MEEI26 L LN | LAMA5 | chr20:60922001 | Missense | G | A | P347L | 107 | 39 |
| MEEI26 L LN | AP000251.2 | chr21:32932394 | lincRNA | G | T | | 174 | 8 |
| MEEI26 L LN | ITSN1 | chr21:35186316 | Missense | G | C | Q889H | 89 | 12 |
| MEEI26 L LN | COL6A1 | chr21:47404298 | Missense | A | C | S115R | 268 | 157 |
| MEEI26 L LN | Unknown | chr22:25041592 | IGR | C | T | | 614 | 31 |
| MEEI26 L LN | RRP7A | chr22:42910266 | Missense | C | A | E201D | 95 | 20 |
| MEEI26 L LN | ASMTL | chrX:1537892 | Missense | G | C | S396C | 106 | 7 |
| MEEI26 L LN | TSPAN7 | chrX:38420755 | 5′UTR | G | T | | 29 | 31 |
| MEEI26 L LN | ATP7A | chrX:77245113 | Missense | G | C | R332T | 47 | 25 |
| MEEI26 L LN | COL4A5 | chrX:107865969 | Missense | G | A | G944E | 54 | 40 |
| MEEI26 R LN | FBXO44 | chr1:11718910 | Nonsense | C | T | R161* | 55 | 24 |
| MEEI26 R LN | C1orf167 | chr1:11842274 | Missense | G | C | Q987H | 25 | 6 |
| MEEI26 R LN | ASAP3 | chr1:23756197 | 3′UTR | C | T | | 7 | 4 |
| MEEI26 R LN | AHDC1 | chr1:27874924 | Missense | G | A | R1235W | 27 | 33 |
| MEEI26 R LN | OSCP1 | chr1:36883811 | Missense | C | G | E367Q | 53 | 10 |
| MEEI26 R LN | PTCH2 | chr1:45293992 | Missense | A | T | L562H | 92 | 553 |
| MEEI26 R LN | MMACHC | chr1:45973965 | Missense | G | T | A120S | 77 | 26 |
| MEEI26 R LN | CMPK1 | chr1:47799713 | Silent | C | T | L32L | 137 | 18 |
| MEEI26 R LN | PTGER3 | chr1:71513167 | Missense | C | T | E32K | 47 | 20 |
| MEEI26 R LN | MSH4 | chr1:76333241 | Missense | A | G | K425E | 28 | 22 |
| MEEI26 R LN | BCAR3 | chr1:94048170 | Silent | G | A | L458L | 159 | 57 |
| MEEI26 R LN | MOV10 | chr1:113231481 | 5′UTR | G | A | | 17 | 26 |
| MEEI26 R LN | RNF115 | chr1:145688126 | Missense | G | C | G274A | 95 | 26 |
| MEEI26 R LN | LCE1E | chr1:152760033 | Silent | C | T | H86H | 144 | 37 |
| MEEI26 R LN | NUP210L | chr1:153973447 | Silent | G | C | V1757V | 136 | 27 |
| MEEI26 R LN | UHMK1 | chr1:162492298 | Silent | G | A | L406L | 96 | 21 |
| MEEI26 R LN | UAP1 | chr1:162557310 | Missense | C | G | R294G | 52 | 19 |
| MEEI26 R LN | GPA33 | chr1:167042740 | Missense | G | A | P27L | 24 | 25 |
| MEEI26 R LN | GORAB | chr1:170508677 | Missense | G | A | D155N | 52 | 12 |
| MEEI26 R LN | FAM5C | chr1:190067408 | Missense | G | A | R681W | 100 | 26 |
| MEEI26 R LN | IGFN1 | chr1:201186533 | Missense | G | C | E3238D | 110 | 19 |
| MEEI26 R LN | ZC3H11A | chr1:203821384 | Missense | A | C | K764Q | 255 | 77 |
| MEEI26 R LN | DNAH14 | chr1:225458485 | Missense | G | A | D2524N | 112 | 24 |
| MEEI26 R LN | CEP170 | chr1:243354534 | Silent | C | T | V298V | 114 | 34 |
| MEEI26 R LN | OR2AK2 | chr1:248129574 | Missense | G | A | G314E | 72 | 21 |
| MEEI26 R LN | OR2T6 | chr1:248551248 | Missense | C | A | F113L | 19 | 11 |
| MEEI26 R LN | RMDN2 | chr2:38294125 | Splice Site | G | C | E572_splice | 97 | 24 |
| MEEI26 R LN | ACYP2 | chr2:54342872 | Missense | G | A | E41K | 61 | 20 |
| MEEI26 R LN | DNAH6 | chr2:85014299 | Missense | G | T | K3704N | 46 | 50 |
| MEEI26 R LN | IGKV1-12 | chr2:89339971 | RNA | G | A | | 371 | 76 |
| MEEI26 R LN | KIAA1211L | chr2:99438567 | Silent | C | T | P723P | 69 | 30 |
| MEEI26 R LN | EPC2 | chr2:149542400 | Silent | G | C | L727L | 69 | 11 |
| MEEI26 R LN | GRB14 | chr2:165349590 | Missense | G | C | L527V | 62 | 34 |
| MEEI26 R LN | NFE2L2 | chr2:178098864 | Missense | G | C | Q61E | 174 | 42 |
| MEEI26 R LN | TTN | chr2:179476137 | Missense | G | C | S16940C | 237 | 70 |
| MEEI26 R LN | TTN | chr2:179590563 | Missense | C | G | G6829A | 25 | 27 |
| MEEI26 R LN | CCDC141 | chr2:179701747 | Missense | C | A | S1400I | 56 | 96 |
| MEEI26 R LN | PLCL1 | chr2:198950305 | Silent | C | T | N688N | 63 | 117 |
| MEEI26 R LN | ABCA12 | chr2:215855472 | Missense | A | G | Li193P | 64 | 90 |
| MEEI26 R LN | CNTN4 | chr3:3072590 | Missense | G | C | G572R | 188 | 71 |
| MEEI26 R LN | ZCWPW2 | chr3:28476708 | Nonsense | C | G | S147* | 71 | 30 |
| MEEI26 R LN | PAQR9 | chr3:142681435 | Silent | G | A | V248V | 229 | 31 |
| MEEI26 R LN | ZBBX | chr3:167023514 | Missense | G | C | Q548E | 72 | 22 |
| MEEI26 R LN | NAALADL2 | chr3:175455162 | Silent | T | C | A655A | 215 | 48 |
| MEEI26 R LN | GRK4 | chr4:3039175 | Silent | C | G | T494T | 70 | 18 |
| MEEI26 R LN | FAM184B | chr4:17636663 | Missense | G | C | P953R | 42 | 8 |
| MEEI26 R LN | NCAPG | chr4:17812743 | Missense | C | T | R15W | 38 | 12 |
| MEEI26 R LN | KIAA1239 | chr4:37446024 | Missense | C | T | S805F | 94 | 117 |
| MEEI26 R LN | CNGA1 | chr4:47942814 | Silent | G | C | V279V | 47 | 14 |
| MEEI26 R LN | COPS4 | chr4:83989658 | Missense | G | A | G357E | 40 | 11 |
| MEEI26 R LN | FAM13A | chr4:89670923 | Splice Site | C | G | R693_splice | 21 | 5 |
| MEEI26 R LN | CCSER1 | chr4:91389411 | Missense | G | T | V544F | 63 | 17 |
| MEEI26 R LN | NDNF | chr4:121961107 | Silent | C | T | E97E | 68 | 28 |
| MEEI26 R LN | SEMA5A | chr5:9380048 | Missense | G | A | T4I | 134 | 21 |
| MEEI26 R LN | NUP155 | chr5:37364401 | Silent | G | T | I81I | 429 | 47 |
| MEEI26 R LN | MROH2B | chr5:41033158 | Silent | C | T | L782L | 229 | 30 |
| MEEI26 R LN | FCHO2 | chr5:72286666 | Missense | G | A | G157E | 37 | 15 |
| MEEI26 R LN | TMEM174 | chr5:72469365 | Missense | C | A | Q99K | 230 | 75 |
| MEEI26 R LN | ANKRD34B | chr5:79854712 | Nonsense | G | C | S376* | 114 | 50 |
| MEEI26 R LN | VCAN | chr5:82816437 | Missense | C | G | T771R | 198 | 41 |
| MEEI26 R LN | MEF2C | chr5:88057020 | Silent | G | T | I128I | 68 | 71 |
| MEEI26 R LN | MEF2C | chr5:88057021 | Missense | A | G | I128T | 67 | 70 |
| MEEI26 R LN | PCDHA2 | chr5:140175104 | Missense | T | G | N185K | 96 | 35 |
| MEEI26 R LN | PCDHA5 | chr5:140203362 | Silent | C | T | L668L | 74 | 92 |
| MEEI26 R LN | PCDHB13 | chr5:140595303 | Silent | C | T | H536H | 398 | 109 |
| MEEI26 R LN | RARS | chr5:167944899 | Missense | G | A | E569K | 114 | 25 |
| MEEI26 R LN | SLIT3 | chr5:168098289 | Silent | C | T | E1347E | 130 | 51 |
| MEEI26 R LN | ERGIC1 | chr5:172342676 | 3′UTR | G | C | | 47 | 11 |
| MEEI26 R LN | HIST1H2BL | chr6:27775601 | Silent | C | T | K28K | 306 | 54 |
| MEEI26 R LN | PPP1R18 | chr6:30653375 | Missense | C | G | E141Q | 92 | 23 |
| MEEI26 R LN | MNF1 | chr6:33665325 | 3′UTR | G | C | | 15 | 5 |
| MEEI26 R LN | MED20 | chr6:41874850 | Missense | A | G | 1200T | 131 | 21 |
| MEEI26 R LN | DST | chr6:56350020 | Intron | G | A | | 23 | 12 |
| MEEI26 R LN | LMBRD1 | chr6:70386155 | Silent | C | T | L506L | 145 | 27 |
| MEEI26 R LN | B3GAT2 | chr6:71571595 | Missense | C | G | D275H | 186 | 37 |
| MEEI26 R LN | FILIP1 | chr6:76023690 | Missense | C | G | E620Q | 16 | 9 |
| MEEI26 R LN | PREP | chr6:105726245 | Missense | A | G | L636P | 153 | 66 |
| MEEI26 R LN | MED23 | chr6:131948597 | Missense | T | A | E33V | 131 | 75 |
| MEEI26 R LN | EPM2A | chr6:146056515 | Silent | C | G | L40L | 14 | 4 |
| MEEI26 R LN | GRM1 | chr6:146351152 | Missense | G | T | V167L | 220 | 53 |
| MEEI26 R LN | STXBP5 | chr6:147636860 | Missense | G | A | E538K | 75 | 43 |
| MEEI26 R LN | QKI | chr6:163991728 | Missense | G | C | A338P | 91 | 22 |
| MEEI26 R LN | NEUROD6 | chr7:31378079 | Missense | A | C | N268K | 64 | 78 |
| MEEI26 R LN | NEUROD6 | chr7:31378245 | Missense | C | G | S213T | 62 | 76 |
| MEEI26 R LN | DDX56 | chr7:44613461 | Missense | T | C | M12V | 108 | 34 |
| MEEI26 R LN | PKD1L1 | chr7:47835746 | Silent | C | T | L2732L | 61 | 17 |
| MEEI26 R LN | WNT2 | chr7:116955138 | Missense | C | G | R192T | 66 | 28 |
| MEEI26 R LN | KCND2 | chr7:120385840 | Missense | G | A | E492K | 65 | 62 |
| MEEI26 R LN | CREB3L2 | chr7:137597805 | Missense | G | C | T172S | 55 | 25 |
| MEEI26 R LN | ZNF777 | chr7:149128876 | Silent | C | T | T829T | 28 | 43 |
| MEEI26 R LN | ZNF467 | chr7:149462099 | Missense | G | A | R498C | 28 | 25 |
| MEEI26 R LN | KAT6A | chr8:41800387 | Nonsense | G | C | S787* | 609 | 120 |
| MEEI26 R LN | RP1 | chr8:55541307 | Missense | A | G | E1622G | 112 | 31 |
| MEEI26 R LN | Unknown | chr8:74171716 | IGR | G | C | | 34 | 8 |
| MEEI26 R LN | TRHR | chr8:110099987 | Missense | C | G | N82K | 160 | 47 |
| MEEI26 R LN | ZNF572 | chr8:125990012 | Missense | G | C | G501A | 51 | 11 |
| MEEI26 R LN | FAM135B | chr8:139379643 | Intron | C | G | | 84 | 19 |
| MEEI26 R LN | SPATA6L | chr9:4626364 | Intron | G | A | | 20 | 10 |
| MEEI26 R LN | BNC2 | chr9:16738420 | Missense | C | T | E23K | 172 | 32 |
| MEEI26 R LN | TLN1 | chr9:35704047 | Missense | G | A | L2058F | 19 | 39 |
| MEEI26 R LN | GOLM1 | chr9:88661396 | Missense | G | C | F152L | 87 | 21 |
| MEEI26 R LN | SPTLC1 | chr9:94809941 | Missense | G | C | S313C | 72 | 38 |
| MEEI26 R LN | ECM2 | chr9:95263009 | Splice Site | C | T | R644_splice | 25 | 14 |
| MEEI26 R LN | ZNF782 | chr9:99581281 | Missense | G | A | P342S | 27 | 28 |
| MEEI26 R LN | ZNF483 | chr9:114289875 | Missense | G | A | R67K | 56 | 59 |
| MEEI26 R LN | ARRDC1 | chr9:140500156 | 5′UTR | G | T | | 17 | 8 |
| MEEI26 R LN | SFMBT2 | chr10:7214545 | Missense | G | T | P688H | 66 | 23 |
| MEEI26 R LN | UNC5B | chr10:73045106 | Missense | G | C | E158Q | 128 | 46 |
| MEEI26 R LN | CDH23 | chr10:73538055 | Missense | C | G | S1731C | 47 | 25 |
| MEEI26 R LN | DNAJC9 | chr10:75007269 | 5′UTR | G | C | | 85 | 34 |
| MEEI26 R LN | 42799 | chr10:94070940 | Silent | A | G | E28E | 109 | 41 |
| MEEI26 R LN | FBXW4 | chr10:103433327 | Missense | G | A | R154W | 108 | 31 |
| MEEI26 R LN | TACC2 | chr10:123976245 | Missense | G | A | C2483Y | 175 | 67 |
| MEEI26 R LN | PLEKHA1 | chr10:124187945 | Intron | A | G | | 18 | 9 |
| MEEI26 R LN | HMX3 | chr10:124896826 | Missense | G | A | S218N | 102 | 32 |
| MEEI26 R LN | NUP98 | chr11:3784133 | Splice Site | G | A | S362_splice | 64 | 18 |
| MEEI26 R LN | OR52A4 | chr11:5142484 | RNA | G | A | | 50 | 27 |
| MEEI26 R LN | ZNF214 | chr11:7022705 | Missense | C | A | W70L | 107 | 16 |
| MEEI26 R LN | LGALS12 | chr11:63283143 | Silent | G | A | L213L | 93 | 22 |
| MEEI26 R LN | SLC22A12 | chr11:64367916 | Missense | T | A | Y455N | 63 | 14 |
| MEEI26 R LN | GRIA4 | chr11:105795133 | Missense | G | C | E495D | 14 | 5 |
| MEEI26 R LN | USP2-AS1 | chr11:119369466 | RNA | T | A | | 60 | 68 |
| MEEI26 R LN | Unknown | chr11:124135484 | IGR | C | A | | 60 | 49 |
| MEEI26 R LN | TULP3 | chr12:3029941 | Missense | G | A | E36K | 155 | 52 |
| MEEI26 R LN | ATN1 | chr12:7045469 | Missense | G | C | E347Q | 116 | 53 |
| MEEI26 R LN | CLEC2D | chr12:9847456 | Missense | G | C | D188H | 120 | 63 |
| MEEI26 R LN | RAPGEF3 | chr12:48133006 | Missense | G | A | 5752L | 183 | 31 |
| MEEI26 R LN | PRKAG1 | chr12:49412516 | Splice Site | G | T | T3_splice | 83 | 36 |
| MEEI26 R LN | TESPA1 | chr12:55357536 | Silent | G | C | L77L | 127 | 33 |
| MEEI26 R LN | PWP1 | chr12:108079654 | 5′UTR | G | C | | 74 | 13 |
| MEEI26 R LN | SDSL | chr12:113865895 | Silent | C | G | L36L | 230 | 38 |
| MEEI26 R LN | IL31 | chr12:122658717 | Start_Codon_SNP | C | T | M1I | 136 | 25 |
| MEEI26 R LN | CLIP1 | chr12:122861975 | Silent | C | T | K206K | 99 | 16 |
| MEEI26 R LN | HCAR3 | chr12:123200711 | Missense | T | C | M192V | 237 | 48 |
| MEEI26 R LN | GTF2H3 | chr12:124118390 | 5′UTR | G | A | | 77 | 13 |
| MEEI26 R LN | ULK1 | chr12:132399728 | Missense | A | G | R492G | 30 | 15 |
| MEEI26 R LN | SACS | chr13:23928706 | De_novo_Start_OutOfFrame | G | C | | 68 | 21 |
| MEEI26 R LN | PABPC3 | chr13:25671891 | Missense | C | T | R519C | 102 | 35 |
| MEEI26 R LN | RNF6 | chr13:26789563 | Silent | T | C | E152E | 31 | 46 |
| MEEI26 R LN | MTUS2 | chr13:29599440 | Missense | C | T | S212F | 46 | 48 |
| MEEI26 R LN | MTUS2 | chr13:29599441 | Silent | C | T | S212S | 45 | 49 |
| MEEI26 R LN | DLEU1 | chr13:50678935 | Missense | G | C | D73H | 64 | 25 |
| MEEI26 R LN | SLITRK5 | chr13:88329176 | Missense | C | G | F511L | 103 | 38 |
| MEEI26 R LN | ARHGEF40 | chr14:21553067 | Silent | C | T | Y1315Y | 47 | 13 |
| MEEI26 R LN | VASH1 | chr14:77229338 | Silent | C | T | V58V | 34 | 29 |
| MEEI26 R LN | MARK3 | chr14:103933461 | Missense | C | G | S348C | 7 | 16 |
| MEEI26 R LN | OR4N4 | chr15:22383022 | Missense | G | C | V184L | 267 | 42 |
| MEEI26 R LN | EXD1 | chr15:41483741 | Missense | G | A | L197F | 55 | 15 |
| MEEI26 R LN | PLA2G4E | chr15:42281673 | Missense | C | T | E555K | 67 | 15 |
| MEEI26 R LN | SPG11 | chr15:44941065 | Splice Site | T | A | E534_splice | 116 | 45 |
| MEEI26 R LN | SPATA5L1 | chr15:45709498 | Missense | G | C | M623I | 42 | 22 |
| MEEI26 R LN | MYO5C | chr15:52571749 | Silent | G | C | L87L | 68 | 24 |
| MEEI26 R LN | SH3GL3 | chr15:84286981 | Missense | G | A | G337E | 101 | 46 |
| MEEI26 R LN | Unknown | chr15:84946969 | IGR | C | A | | 79 | 38 |
| MEEI26 R LN | C16orf59 | chr16:2510244 | 5′UTR | G | A | | 46 | 27 |
| MEEI26 R LN | PRSS41 | chr16:2848552 | RNA | G | A | | 51 | 15 |
| MEEI26 R LN | PDXDC1 | chr16:15083846 | Intron | C | T | | 42 | 74 |
| MEEI26 R LN | KIAA0430 | chr16:15690543 | 3′UTR | A | G | | 24 | 11 |
| MEEI26 R LN | WDR81 | chr17:1633734 | Missense | G | A | R1243H | 61 | 12 |
| MEEI26 R LN | DHX33 | chr17:5372104 | Missense | A | C | F26V | 70 | 18 |
| MEEI26 R LN | TP53 | chr17:7577106 | Missense | G | A | P278S | 182 | 281 |
| MEEI26 R LN | NF1 | chr17:29528456 | Missense | A | C | T405P | 90 | 26 |
| MEEI26 R LN | DUSP14 | chr17:35872411 | Missense | C | T | L13F | 44 | 9 |
| MEEI26 R LN | DUSP14 | chr17:35872479 | Missense | C | G | F35L | 132 | 29 |
| MEEI26 R LN | EFTUD2 | chr17:42931994 | Missense | C | T | R730H | 61 | 37 |
| MEEI26 R LN | DLX3 | chr17:48072208 | Missense | G | C | S52W | 172 | 48 |
| MEEI26 R LN | TRIM25 | chr17:54981779 | Nonsense | G | C | S255* | 190 | 31 |
| MEEI26 R LN | CA4 | chr17:58235448 | Missense | C | G | F180L | 168 | 31 |
| MEEI26 R LN | CA4 | chr17:58235471 | Missense | C | G | S188C | 159 | 29 |
| MEEI26 R LN | INTS2 | chr17:59945304 | Missense | C | G | R1112P | 35 | 13 |
| MEEI26 R LN | PLEKHM1P | chr17:62825358 | RNA | C | G | | 210 | 31 |
| MEEI26 R LN | SLC39A11 | chr17:70645009 | Missense | G | C | L295V | 123 | 32 |
| MEEI26 R LN | CYTH1 | chr17:76698687 | Splice Site | C | T | | 58 | 15 |
| MEEI26 R LN | SLC38A10 | chr17:79226448 | Missense | C | T | E498K | 43 | 15 |
| MEEI26 R LN | TBCD | chr17:80866292 | Intron | C | G | | 77 | 15 |
| MEEI26 R LN | LRRC30 | chr18:7231142 | Silent | G | A | G2G | 27 | 9 |
| MEEI26 R LN | PPP4R1 | chr18:9559507 | Silent | T | A | A646A | 99 | 23 |
| MEEI26 R LN | PIGN | chr18:59781831 | Missense | G | C | A405G | 76 | 16 |
| MEEI26 R LN | MUM1 | chr19:1360306 | Missense | C | T | S61L | 26 | 13 |
| MEEI26 R LN | KLF16 | chr19:1863108 | Missense | G | A | P130L | 41 | 15 |
| MEEI26 R LN | MAP2K2 | chr19:4095428 | Missense | T | C | N335S | 29 | 31 |
| MEEI26 R LN | GTF2F1 | chr19:6393087 | 5′UTR | G | A | | 49 | 18 |
| MEEI26 R LN | ZNF878 | chr19:12155729 | Missense | T | C | R210G | 139 | 55 |
| MEEI26 R LN | ZNF383 | chr19:37726901 | Missense | C | G | Q53E | 51 | 24 |
| MEEI26 R LN | HIPK4 | chr19:40889765 | Missense | G | C | F249L | 71 | 20 |
| MEEI26 R LN | ZNF284 | chr19:44590583 | Missense | G | A | D318N | 37 | 66 |
| MEEI26 R LN | DEFB127 | chr20:139420 | Missense | G | A | E19K | 37 | 19 |
| MEEI26 R LN | MMP9 | chr20:44642100 | Missense | G | A | D513N | 130 | 36 |
| MEEI26 R LN | VAPB | chr20:57024667 | 3′UTR | G | C | | 75 | 16 |
| MEEI26 R LN | LAMAS | chr20:60906095 | Missense | G | A | P1215S | 48 | 9 |
| MEEI26 R LN | LAMAS | chr20:60922001 | Missense | G | A | P347L | 61 | 49 |
| MEEI26 R LN | ITSN1 | chr21:35186316 | Missense | G | C | Q889H | 75 | 19 |
| MEEI26 R LN | COL6A1 | chr21:47404298 | Missense | A | C | S115R | 154 | 199 |
| MEEI26 R LN | TOP3B | chr22:22318356 | Silent | G | T | G381G | 34 | 10 |
| MEEI26 R LN | RRP7A | chr22:42910266 | Missense | C | A | E201D | 73 | 19 |
| MEEI26 R LN | TSPAN7 | chrX:38420755 | 5′UTR | G | T | | 10 | 37 |
| MEEI26 R LN | ATP7A | chrX:77245113 | Missense | G | C | R332T | 31 | 36 |
| MEEI26 R LN | COL4A5 | chrX:107865969 | Missense | G | A | G944E | 27 | 35 |
| MEEI28 LN | ZCCHC17 | chr1:31836995 | Missense | G | C | K227N | 111 | 5 |
| MEEI28 LN | Unknown | chr1:92109150 | IGR | C | T | | 192 | 8 |
| MEEI28 LN | DBT | chr1:100681693 | Silent | G | C | L206L | 235 | 6 |
| MEEI28 LN | REG4 | chr1:120342468 | Silent | G | A | Y61Y | 114 | 4 |
| MEEI28 LN | TMOD4 | chr1:151146081 | Intron | A | G | | 92 | 4 |
| MEEI28 LN | NCSTN | chr1:160313303 | Intron | C | T | | 116 | 4 |
| MEEI28 LN | KDM5B | chr1:202743794 | Missense | G | C | L118V | 153 | 9 |
| MEEI28 LN | SLC41A1 | chr1:205770130 | Missense | C | T | G144E | 213 | 10 |
| MEEI28 LN | HHIPL2 | chr1:222721212 | Missense | C | G | E59Q | 144 | 6 |
| MEEI28 LN | SMYD3 | chr1:246021855 | Missense | T | C | N2815 | 152 | 6 |
| MEEI28 LN | MSH6 | chr2:48018193 | Missense | C | G | H130D | 237 | 9 |
| MEEI28 LN | GAD1 | chr2:171678601 | Silent | C | T | Y29Y | 197 | 8 |
| MEEI28 LN | SP110 | chr2:231077706 | Missense | G | A | P118L | 156 | 5 |
| MEEI28 LN | GPR35 | chr2:241569804 | Silent | C | T | I145I | 92 | 6 |
| MEEI28 LN | ATG7 | chr3:11399971 | Missense | G | A | R455H | 65 | 4 |
| MEEI28 LN | TOP2B | chr3:25674016 | Missense | G | A | S391F | 110 | 4 |
| MEEI28 LN | TGFBR2 | chr3:30732963 | Missense | G | A | E526K | 145 | 7 |
| MEEI28 LN | ARPP21 | chr3:35835368 | Missense | C | T | S786F | 198 | 6 |
| MEEI28 LN | KLHL40 | chr3:42727776 | Missense | G | C | E222D | 38 | 3 |
| MEEI28 LN | KIF15 | chr3:44894204 | Missense | G | C | E1382Q | 84 | 4 |
| MEEI28 LN | AMT | chr3:49454524 | 3′UTR | C | T | | 353 | 14 |
| MEEI28 LN | DUSP7 | chr3:52084849 | Silent | A | G | N414N | 62 | 5 |
| MEEI28 LN | ABI3BP | chr3:100535460 | 3′UTR | T | A | | 199 | 9 |
| MEEI28 LN | SERPINI2 | chr3:167167104 | Splice Site | C | T | G351_splice | 272 | 10 |
| MEEI28 LN | PHC3 | chr3:169846740 | Missense | G | A | S495F | 112 | 5 |
| MEEI28 LN | UGDH | chr4:39512364 | Missense | C | G | E128Q | 297 | 9 |
| MEEI28 LN | CHRNA9 | chr4:40356174 | Silent | C | T | L359L | 108 | 6 |
| MEEI28 LN | UGT2B28 | chr4:70160320 | Silent | C | G | V461V | 190 | 5 |
| MEEI28 LN | ENAM | chr4:71510351 | Missense | A | G | T1070A | 67 | 4 |
| MEEI28 LN | GSTCD | chr4:106640384 | Missense | G | C | Q198H | 153 | 8 |
| MEEI28 LN | PLK4 | chr4:128819607 | Missense | G | A | E942K | 125 | 5 |
| MEEI28 LN | DCHS2 | chr4:155157203 | Missense | C | A | W2412C | 217 | 6 |
| MEEI28 LN | TLR3 | chr4:187004123 | Missense | C | G | S428C | 81 | 4 |
| MEEI28 LN | TXNDC15 | chr5:134229202 | Silent | C | T | N204N | 117 | 6 |
| MEEI28 LN | PCDHGA2 | chr5:140718711 | Missense | C | T | A58V | 128 | 4 |
| MEEI28 LN | STK32A | chr5:146752801 | Missense | G | C | D283H | 190 | 7 |
| MEEI28 LN | FAM71B | chr5:156589725 | Silent | C | T | K517K | 247 | 5 |
| MEEI28 LN | RANBP9 | chr6:13622583 | 3′UTR | G | C | | 36 | 4 |
| MEEI28 LN | TPMT | chr6:18143952 | Missense | C | T | D81N | 232 | 8 |
| MEEI28 LN | HIST1H1E | chr6:26156961 | Nonsense | G | T | E115* | 265 | 9 |
| MEEI28 LN | ZNF391 | chr6:27369053 | Missense | G | A | E302K | 111 | 6 |
| MEEI28 LN | LHFPL5 | chr6:35773543 | Silent | C | T | L32L | 329 | 12 |
| MEEI28 LN | ETV7 | chr6:36336811 | Silent | G | A | L179L | 158 | 6 |
| MEEI28 LN | FOXP4 | chr6:41562611 | Missense | G | A | A514T | 100 | 7 |
| MEEI28 LN | UBR2 | chr6:42641505 | Intron | G | T | | 37 | 7 |
| MEEI28 LN | COL21A1 | chr6:55922480 | Missense | G | A | P950L | 267 | 11 |
| MEEI28 LN | FAXC | chr6:99771386 | Missense | G | A | R253C | 244 | 5 |
| MEEI28 LN | SOBP | chr6:107827452 | Missense | C | A | S81Y | 156 | 5 |
| MEEI28 LN | T | chr6:166572027 | Missense | A | T | S362T | 33 | 4 |
| MEEI28 LN | T | chr6:166572040 | Silent | G | T | A357A | 25 | 3 |
| MEEI28 LN | GHRHR | chr7:31014627 | Missense | T | A | I221N | 243 | 13 |
| MEEI28 LN | HECW1 | chr7:43531744 | Missense | T | C | I1102T | 96 | 7 |
| MEEI28 LN | CACNA2D1 | chr7:81601167 | Missense | C | G | L689F | 174 | 7 |
| MEEI28 LN | PEX1 | chr7:92148333 | Silent | G | C | L111L | 157 | 4 |
| MEEI28 LN | ZAN | chr7:100350009 | RNA | A | C | | 206 | 10 |
| MEEI28 LN | KCP | chr7:128533485 | RNA | C | G | | 99 | 5 |
| MEEI28 LN | CUL1 | chr7:148487456 | Nonsense | C | T | R577* | 192 | 4 |
| MEEI28 LN | DCAF4L2 | chr8:88885230 | Missense | C | T | E324K | 140 | 6 |
| MEEI28 LN | PKHD1L1 | chr8:110534461 | Silent | A | G | G4026G | 169 | 6 |
| MEEI28 LN | ZC3H3 | chr8:144621326 | Missense | G | A | R71C | 144 | 5 |
| MEEI28 LN | CDKN2A | chr9:21994190 | Silent | C | T | L47L | 166 | 9 |
| MEEI28 LN | APTX | chr9:33001382 | 5′UTR | G | C | | 87 | 5 |
| MEEI28 LN | SPATA31E1 | chr9:90498010 | Silent | C | T | F68F | 212 | 6 |
| MEEI28 LN | CYLC2 | chr9:105767302 | Missense | C | T | S130L | 32 | 5 |
| MEEI28 LN | ABCA1 | chr9:107576413 | Missense | G | C | S1296C | 243 | 8 |
| MEEI28 LN | NOTCH1 | chr9:139412239 | Missense | T | C | D469G | 201 | 16 |
| MEEI28 LN | CUBN | chr10:17130165 | Missense | C | G | E649Q | 156 | 7 |
| MEEI28 LN | ARMC3 | chr10:23297828 | Silent | C | T | L671L | 92 | 8 |
| MEEI28 LN | AGAP11 | chr10:88768826 | RNA | G | A | | 331 | 14 |
| MEEI28 LN | AC129929.5 | chr11:2356907 | RNA | C | T | | 192 | 10 |
| MEEI28 LN | OR10A3 | chr11:7960941 | Missense | C | A | A43S | 104 | 5 |
| MEEI28 LN | RIC3 | chr11:8161626 | Missense | T | C | K80R | 267 | 11 |
| MEEI28 LN | ABCC8 | chr11:17428327 | Silent | G | A | T1058T | 249 | 8 |
| MEEI28 LN | KIAA1549L | chr11:33566517 | Missense | C | T | P696L | 178 | 10 |
| MEEI28 LN | OR4C46 | chr11:51516009 | Missense | C | T | T243M | 108 | 8 |
| MEEI28 LN | CTNND1 | chr11:57577589 | Nonsense | C | G | S809* | 90 | 4 |
| MEEI28 LN | M54A2 | chr11:59856269 | Missense | C | A | L11I | 165 | 4 |
| MEEI28 LN | SLC22A6 | chr11:62751890 | Silent | G | C | L91L | 265 | 13 |
| MEEI28 LN | SSH3 | chr11:67070974 | 5′UTR | G | A | | 40 | 5 |
| MEEI28 LN | OR8A1 | chr11:124440042 | Silent | G | A | V26V | 98 | 7 |
| MEEI28 LN | FGF23 | chr12:4479940 | Missense | C | T | D109N | 325 | 7 |
| MEEI28 LN | CHD4 | chr12:6697040 | Missense | C | G | E1181Q | 161 | 6 |
| MEEI28 LN | GUCY2C | chr12:14775001 | Missense | C | T | D847N | 164 | 7 |
| MEEI28 LN | MYO1A | chr12:57424929 | Silent | G | A | N793N | 168 | 7 |
| MEEI28 LN | DPY19L2 | chr12:64062086 | Missense | C | T | E30K | 167 | 5 |
| MEEI28 LN | MYF6 | chr12:81101703 | Missense | C | A | Q69K | 239 | 12 |
| MEEI28 LN | SLC17A8 | chr12:100774702 | Missense | G | A | V109I | 160 | 5 |
| MEEI28 LN | Unknown | chr13:19419905 | IGR | C | T | | 133 | 7 |
| MEEI28 LN | TRAV21 | chr14:22521115 | RNA | G | T | | 157 | 7 |
| MEEI28 LN | AJUBA | chr14:23443288 | Missense | G | A | R487W | 106 | 5 |
| MEEI28 LN | CTAGE5 | chr14:39734530 | 5′UTR | C | G | | 110 | 4 |
| MEEI28 LN | FUT8 | chr14:66082718 | Nonsense | C | T | Q76* | 86 | 5 |
| MEEI28 LN | MIR494 | chr14:101495978 | RNA | G | C | | 214 | 8 |
| MEEI28 LN | FBN1 | chr15:48717990 | Missense | G | C | H2426D | 174 | 11 |
| MEEI28 LN | RPL3L | chr16:2000933 | Missense | C | T | R138Q | 108 | 5 |
| MEEI28 LN | HS3ST4 | chr16:26147153 | Missense | C | T | R319W | 149 | 9 |
| MEEI28 LN | IRX5 | chr16:54967119 | Silent | C | T | D262D | 127 | 7 |
| MEEI28 LN | DOK4 | chr16:57513429 | 5′UTR | A | G | | 108 | 5 |
| MEEI28 LN | ESRP2 | chr16:68269602 | Missense | C | G | E88Q | 102 | 10 |
| MEEI28 LN | FAM157C | chr16:90233558 | RNA | C | A | | 564 | 11 |
| MEEI28 LN | KRTAP4-8 | chr17:39253822 | Missense | G | C | S172C | 55 | 4 |
| MEEI28 LN | CCR10 | chr17:40832003 | Silent | C | T | P219P | 127 | 4 |
| MEEI28 LN | NFE2L1 | chr17:46128488 | Missense | C | G | S3C | 136 | 6 |
| MEEI28 LN | NFE2L1 | chr17:46128846 | Silent | C | T | L122L | 165 | 5 |
| MEEI28 LN | B4GALNT2 | chr17:47230259 | Missense | C | T | P211S | 182 | 9 |
| MEEI28 LN | ITGA3 | chr17:48158748 | Silent | C | T | I965I | 100 | 5 |
| MEEI28 LN | MIR21 | chr17:57918654 | RNA | G | A | | 117 | 10 |
| MEEI28 LN | BRIP1 | chr17:59938862 | Silent | C | A | V13V | 211 | 7 |
| MEEI28 LN | 42804 | chr17:60813593 | Missense | C | G | D545H | 240 | 11 |
| MEEI28 LN | 42804 | chr17:60814545 | Silent | C | T | Q227Q | 243 | 11 |
| MEEI28 LN | RNF213 | chr17:78327779 | Intron | C | T | | 201 | 11 |
| MEEI28 LN | USP14 | chr18:163416 | Missense | C | T | A42V | 92 | 5 |
| MEEI28 LN | MAPK4 | chr18:48255680 | Missense | G | A | R407H | 92 | 4 |
| MEEI28 LN | POLR2E | chr19:1095360 | 5′UTR | G | C | | 70 | 4 |
| MEEI28 LN | ELAVL1 | chr19:8038758 | Missense | G | A | S94L | 71 | 5 |
| MEEI28 LN | UBL5 | chr19:9939283 | Silent | C | T | I24I | 138 | 7 |
| MEEI28 LN | RFX1 | chr19:14083755 | Missense | T | C | T372A | 224 | 9 |
| MEEI28 LN | WDR62 | chr19:36556891 | Missense | G | A | D122N | 172 | 4 |
| MEEI28 LN | ZNF383 | chr19:37721322 | 5′UTR | C | G | | 74 | 6 |
| MEEI28 LN | ZNF780B | chr19:40541403 | Nonsense | G | A | R455* | 211 | 11 |
| MEEI28 LN | TEX101 | chr19:43920564 | Missense | C | T | P101L | 72 | 5 |
| MEEI28 LN | NPAS1 | chr19:47548631 | Missense | C | T | R499W | 160 | 10 |
| MEEI28 LN | PRPF31 | chr19:54621832 | Missense | G | C | K58N | 176 | 5 |
| MEEI28 LN | DZANK1 | chr20:18371050 | Silent | G | C | V507V | 127 | 4 |
| MEEI28 LN | RAB22A | chr20:56918779 | Missense | C | G | S41C | 76 | 4 |
| MEEI28 LN | EEF1A2 | chr20:62126268 | Missense | C | T | V171I | 232 | 11 |
| MEEI28 LN | IL10RB | chr21:34655420 | Missense | G | C | D174H | 240 | 9 |
| MEEI28 LN | UFD1L | chr22:19442165 | Intron | G | C | | 58 | 4 |
| MEEI28 LN | SMARCB1 | chr22:24176339 | Missense | G | A | R386H | 249 | 9 |
| MEEI28 LN | NEFH | chr22:29876293 | Silent | G | A | P14P | 62 | 6 |
| MEEI28 LN | MICALL1 | chr22:38321666 | Splice Site | A | C | | 23 | 8 |
| MEEI28 LN | CSNK1E | chr22:38690390 | Missense | C | T | E346K | 82 | 6 |
| MEEI28 LN | PLXNB2 | chr22:50716562 | Missense | C | T | R1624Q | 46 | 4 |
| MEEI28 LN | PLXNB2 | chr22:50721833 | Missense | C | A | G871V | 151 | 8 |
| MEEI28 LN | PLXNB2 | chr22:50728293 | Missense | G | A | R241W | 138 | 8 |
| MEEI28 LN | IL1RAPL1 | chrX:29973610 | Silent | G | A | S588S | 53 | 11 |
| MEEI28 LN | ZMYM3 | chrX:70464238 | Missense | G | A | S1053L | 78 | 6 |
| MEEI28 LN | HNRNPH2 | chrX:100667593 | Missense | G | T | R206L | 87 | 9 |
| MEEI28 LN | IRS4 | chrX:107977583 | Silent | C | T | T664T | 148 | 11 |
| MEEI28 LN | AFF2 | chrX:148037612 | Silent | C | T | A679A | 49 | 4 |
| MEEI28 LN | F8 | chrX:154157570 | Missense | G | A | L1499F | 107 | 10 |
| MEEI28 LN | Unknown | chrGL000237.1:2589 | IGR | C | T | | 75 | 5 |
| MEEI28 LN | Unknown | chrGL000205.1:117350 | IGR | C | A | | 30 | 5 |

LN = lymph node;
L = left;
R = right;
chr = chromosome

TABLE S5
Differentially expressed genes between CAF subsets, Related to FIG. 2.
Genes are sorted from most to least significant.

| CAF1 genes | | | | | CAF2 genes | | | | |
|---|---|---|---|---|---|---|---|---|---|
| Genes | Genes | Genes | Genes | Genes | Genes | Genes | Genes | Genes | Genes |
| 1-50 | 51-100 | 101-150 | 151-200 | 201-208 | 1-50 | 51-100 | 101-150 | 151-200 | 201-241 |
| CTHRC1 | COL5A2 | CPXM1 | LOXL3 | CXCL6 | CFD | ANGPTL1 | CLU | FBLN5 | TMEM176A |
| COL1A1 | GBP1 | RGS3 | COL6A2 | FBXO32 | APOD | JUNB | LHFP | SCARA5 | ADH5 |
| POSTN | ITGB1 | MYO1B | GEM | FKBP10 | CXCL12 | RPL13A | NDRG2 | PROS1 | IL11RA |
| TPM4 | IL24 | WBP5 | TGFB1 | PTS | GPC3 | CILP | SOD3 | FOXO3 | C5orf4 |
| MFAP2 | PLAU | MYH9 | SGCB | SMIM3 | SEPP1 | MEG3 | TFPI | RECK | CYP27A1 |
| SPARC | AEBP1 | ITGB5 | PDLIM3 | TRIB2 | G5N | JUN | NFIB | SLPI | CBLB |
| WNT5A | COL1A2 | SEC23A | FKBP3 | HEPH | CXCL14 | CD34 | MTUS1 | RPL22 | PID1 |
| COL3A1 | TPM2 | HSPB1 | EDNRA | RRBP1 | MFAP4 | FOSB | TENC1 | ANKRD36BP1 | EGFR |
| LOC541471 | CLEC11A | FRMD6 | ITM2C | | GPX3 | MTRNR2L1 | E8F1 | EBF2 | TMEM159 |
| TNFRSF12A | AXL | PDLIM7 | CRISPLD2 | | MGP | VIM | STOM | ECHOC2 | MT1M |
| INHBA | ADAMTSZ | PRKCDBP | F2RL2 | | SPARCL1 | CHRDL1 | ZFA51 | AGT | PPP1R10 |
| THY1 | IGFBP3 | ILK | RCN1 | | LTBP4 | SERPINA3 | SAMHD1 | NPDC1 | ARHGAP10 |
| SERPINH1 | GREM1 | WIPI1 | IL8 | | AOH1B | TIMP3 | IGF8P5 | DDIT3 | NID1 |
| LOXL2 | MMP1 | CDH11 | RAB2A | | Z8TB18 | TGFBR3 | KLF4 | TPPP3 | SLIT3 |
| RAB31 | DUSP14 | PLAT | F2R | | ABCA8 | NTRK2 | CDO1 | PL5CR4 | CRTAP |
| GPM6B | TAGLN | SGIPI | SEC31A | | C3 | MYOC | ADAM33 | NKF1 | CA839L |
| ACTN1 | PPIC | STEAP1 | C12orf75 | | IGF1 | THBS4 | PCOHGC3 | F10 | COL42EP4 |
| IFI27 | REEP3 | PON2 | STARD13 | | PODN | CST3 | PRNP | AKR1C3 | SLC19A2 |
| LGALS1 | RAP18 | HA52 | TWSG1 | | PRELP | PLAC9 | EPB41L2 | PDGFQ | SLC4DA1 |
| SERPINE1 | TNFRSF21 | PTEN | MRPL32 | | PDK4 | PI15 | FBLN2 | CTSF | SPG20 |
| PLAUR | CTSK | FMOD | RSU1 | | FHL1 | IGFBP6 | GADD45B | FCGRT | CALCOCO1 |
| HIF1A | SPON2 | CALD1 | FBN1 | | AB13BP | IER2 | VAT1 | RARRES1 | SOC2 |
| CHN1 | RARRES2 | MMP3 | B4GALT1 | | RNASE4 | FGL2 | PLAGL1 | MATN2 | BCL6 |
| THBS2 | LEPRE1 | PHLDA2 | VOL | | PLA2G2A | RPL14 | CFH | ZNF460 | DDX39B |
| ASPN | RHOC | P4HA2 | TDO2 | | BTG2 | COL14A1 | BTF3 | CYR81 | GPMMB |
| TNC | PRSSZ3 | ECM1 | CCL2 | | TNX5 | RAMP2 | KIAA1683 | CDKN1A | MT1A |
| ARF4 | HIF0 | SNAI2 | MARCKS | | CYBRD1 | DHRS3 | ESD | INMT | BDHZ |
| CALU | PLOD2 | FNDC3B | CNIH4 | | FBLN1 | OLFML3 | EDNRB | EIF1B | ENPP2 |
| LUM | XIAA1217 | UBD | GOLM1 | | HSPB6 | ABLIM1 | MYC | SOCS3 | SYNPO2 |
| TWIST1 | C1OTNF8 | BMP1 | CNN3 | | MGST1 | CD302 | LEPR | APP | IL8ST |
| MMP11 | CPE | SDC4 | STEAP2 | | ZFP36 | CCDC60 | GAS6 | ITIH5 | SERINC1 |
| SPHK1 | IFITM1 | FAF | DKK3 | | WT | RBMS3 | DDAH2 | GABARAPL1 | TRIP10 |
| PRDM1 | MYL6 | NPTN | ANXA5 | | SFRP1 | MT1X | FIBIN | GSTM2 | PLBD1 |
| NREP | PHLDA1 | GNAIT | TMEM30A | | ADAMTS1 | FOS | C7 | RASD1 | PHF17 |
| LYBE | LTBP1 | CLIC4 | MRPS24 | | MAMDC2 | CAPN6 | SPTBN1 | NR4A1 | MT1E |
| TNFAIP8 | ANTXR1 | AQP1 | MMP19 | | PLTP | LAMA2 | RPL10A | NT5E | NFKBIZ |
| BPGM | YIF1A | ITGA1 | EPAS1 | | SRPX | HAPA1B | ITM2B | MAP1LC3C | MOB36 |
| COL5A1 | CRABP2 | LAMP5 | XCTD10 | | PDGFRL | ABCA8 | BRD2 | EPB41L4A-AS1 | SELENBP1 |
| CAV1 | ITGA5 | FADS1 | IERXP1 | | ALDH1A1 | MFAP5 | CPQ | ACVR2A | TCEA3 |
| TMEM45A | COL6A3 | MYL8 | TNFRSF8B | | EFEMP1 | FXVD8 | HSD17B11 | RPS4Y1 | AKAP12 |
| COL12A1 | TWIST2 | SELM | CD59 | | A2M | OGN | CEBPD | TNFAIP2 | PPAP2A |
| PXON | SULF1 | FN1 | ID1 | | MTRNR2L2 | LRP1 | GSTM5 | FAM13C | |
| PDPN | C1orf198 | ANGPTL2 | DDAH1 | | SMOC2 | PPP1R15A | SESN1 | CPED1 | |
| RIN3 | SRPX2 | HAPLN3 | STC1 | | MTRNR2L8 | COL15A1 | PDGFRA | TGFBR2 | |
| S100A18 | LMCO1 | SULF2 | SLC39A6 | | PTGDS | ADIRF | ANG | SCPEP1 | |
| IL1R1 | XDELR3 | FAM114A1 | PGM3 | | NFIA | ALDH2 | FMO2 | RND3 | |
| PTXZ | PKIG | ID3 | PLOD1 | | DPT | PPAP28 | CTGF | HSPB8 | |
| TPM1 | IFIT3 | TSKU | C12orf23 | | FGF7 | METTL7A | BMP4 | SGCE | |
| RCN3 | VMP1 | ACTA2 | CTTN | | ITM2A | WISP2 | ZFAND6 | ASS1 | |
| COL6A1 | RABAC1 | C5orf15 | GGT5 | | FIGF | LGALS3 | GALNT16 | LAMB2 | |

TABLE S6
Expression programs detected by NNMF in each of 10 patients,
Related to FIG. 3. Clusters are ordered as in FIG. 3B, and within each cluster the genes
are ordered from most to least significant. For each cluster, headers also indicate the patient
from which it was derived and an inferred annotation. See also online tables.

| | Inferred | Patient | | | | | | | | | | |
|---|---|---|---|---|---|---|---|---|---|---|---|---|
| Cluster | Annotation | (MEEI) | | | | | | | | | | |
| | | | | Genes 1-10 | | | | | | | | |
| 1 | Cell | 17 | CCNB1 | CDC20 | CCNB2 | CDKN3 | KIF20A | BIRC5 | CENPW | TPX2 | PLK1 | PTTG1 |
| | cycle(G2/m) | | | | | | | | | | | |
| 2 | Cell | 20 | CDC20 | CCNB2 | CCNB1 | CDKN3 | PLK1 | PTTG1 | BIRC5 | NUSAP1 | TOP2A | HMGB2 |
| | cycle(G2/m) | | | | | | | | | | | |
| 3 | Cell | 26 | CDC20 | CDCA8 | CCNB1 | BIRC5 | CCNB2 | PTTG1 | HMGB2 | PLK1 | TPX2 | TROAP |
| | cycle(G2/m) | | | | | | | | | | | |
| 4 | Cell | 25 | CDC20 | CCNB1 | CCNB2 | CDKN3 | NUSAP1 | HMGB2 | BIRC5 | TPX2 | TK1 | PTTG1 |
| | cycle(G2/m) | | | | | | | | | | | |
| 5 | Cell | 28 | CCNB1 | CDC20 | CCNB2 | CDKN3 | PTTG1 | PLK1 | TPX2 | KIF22 | BIRC5 | CENPW |
| | cycle(G2/m) | | | | | | | | | | | |
| 6 | Cell | 18 | CDC20 | TK1 | MMP9 | CCNB2 | TUBA1B | BIRC5 | CCNB1 | CDKN3 | CENPW | PTTG1 |
| | cycle(G2/m) | | | | | | | | | | | |
| 7 | Cell | 22 | BIRC5 | CDKN3 | CCNB1 | CDC20 | CCNB2 | TGFBI | KPNA2 | PLK1 | LAMC2 | RNASEH2A |
| | cycle(G2/m) | | | | | | | | | | | |
| 8 | Cell | 22 | CCNB1 | CDC20 | PLK1 | FABP4 | HMGB2 | BIRC5 | CCNB2 | TROAP | CDKN3 | PTTG1 |
| | cycle(G2/m) | | | | | | | | | | | |
| 9 | Cell cycle (G1/S) | 25 | MCM5 | GINS2 | UNG | MCM3 | MCM4 | C19orf48 | CDC6 | MCM2 | CD74 | WDR34 |
| 10 | Cell cycle (G1/S) | 22 | GINS2 | MCM3 | MCM5 | UNG | CDC6 | MCM2 | UHRF1 | MCM6 | PSMC3IP | RFC2 |
| 11 | Cell cycle (G1/S) | 28 | CDC45 | CXCL10 | MCM5 | MCM6 | MCM2 | MCM3 | PCNA | MCM7 | FEN1 | RFC4 |
| 12 | Cell cycle (G1/S) | 5 | UBE2C | ZWINT | HMGB2 | NUSAP1 | MAD2L1 | RFC4 | CCNB1 | CDC20 | MCM7 | TPX2 |
| 13 | Cell cycle (G1/S) | 17 | MCM5 | GINS2 | MCM2 | UHRF1 | FEN1 | MCM7 | MCM3 | PCNA | ZWINT | MCM6 |
| 14 | Cell cycle (G1/S) | 20 | GINS2 | TK1 | PCNA | MCM5 | MCM3 | TUBA1B | FEN1 | MCM2 | CDC45 | UBE2T |
| 15 | Cell cycle (G1/S) | 26 | TK1 | MCM5 | H2AFZ | MCM4 | MCM3 | PCNA | ZWINT | RFC4 | UBE2T | TUBA1B |
| 16 | Cell | 6 | TK1 | BIRC5 | RRM2 | HMGB2 | MAD2L1 | CCNB1 | HIST1H4C | CDK1 | ZWINT | PBK |
| | cycle(G1/S + G2/ | | | | | | | | | | | |
| | M) | | | | | | | | | | | |
| 17 | Cell | 18 | UBE2C | CDC6 | TK1 | HIST1H4C | C19orf48 | RRM2 | CENPW | ANLN | TUBA1B | MCM5 |
| | cycle(G1/S + G2/ | | | | | | | | | | | |
| | M) | | | | | | | | | | | |
| 18 | Cell | 16 | CDC20 | CENPW | TK1 | HMGB2 | CCNB1 | MAD2L1 | CCNB2 | BIRC5 | ZWINT | CDKN3 |
| | cycle(G1/S + G2/ | | | | | | | | | | | |
| | M) | | | | | | | | | | | |
| 19 | EMT-like | 25 | TGFBI | IGFBP3 | MMP1 | ITGB1 | LAMC2 | PDPN | TNC | LAMB3 | VIM | SERPINE1 |
| 20 | EMT-like | 5 | CXCL14 | LAMC2 | COL17A1 | DKK3 | TNC | SERPINE2 | SERPINHI | PRKCDBP | LAMA3 | BGN |
| 21 | EMT-like | 16 | FN1 | CLU | TGFBI | COL1A1 | TAGLN | AXL | LAMC2 | KRT8 | CTHRC1 | MMP2 |
| 22 | EMT-like | 17 | AMTN | MMP3 | MMP10 | TNC | ITGB6 | TGFBI | RBP1 | FSTL3 | IGFL1 | COL5A2 |
| 23 | EMT-like | 22 | LAMC2 | MMP10 | INHBA | LAMA3 | KRT6B | MMP9 | SERPINE1 | MMP1 | G0S2 | CDH1 |
| 24 | EMT-like | 18 | MMP10 | NDUFA4L2 | MFAP2 | GJB6 | TGFBI | GJB2 | APP | LOX | DKK3 | CXCL14 |
| 25 | EMT-like | 6 | LGALS1 | NNMT | SERPING1 | IGFBP7 | C1S | VIM | CD74 | SERPINF1 | SPARC | MEG3 |
| 26 | EMT-like | 18 | LAMC2 | CTNNAL1 | LTBP1 | SLC20A1 | ECH1 | INHBA | PTHLH | PLIN2 | BECN1 | THBS1 |
| 27 | MHC-II | 17 | HLA- | CD74 | HLA- | CXCL14 | NUPR1 | HLA- | DLK2 | HLA- | PTHLH | HLA- |
| | | | DRA | | DRB1 | | | DMA | | DRB5 | | DPA1 |
| 28 | Hypoxia | 18 | IGFBP3 | NDRG1 | PTHLH | NDUFA4L2 | EGLN3 | SERPINE1 | SLC2A1 | DHRS3 | GPNMB | TIMP3 |
| 29 | Hypoxia | 20 | ERO1L | FAM162A | BNIP3 | SLC2A3 | ENO2 | NDRG1 | PGK1 | PDK1 | LDHA | P4HA1 |
| 30 | Epi-dif | 25 | KRT6B | SBSN | KLK7 | KRTDAP | SPRR1B | CRYAB | KRT17 | CLIC3 | SULT2B1 | CALML5 |
| 31 | Epi-dif | 17 | SPRR1B | SLPI | S100A7 | PI3 | C10orf99 | FABP5 | SPRR2D | KRT16 | CLDN7 | IL1RN |
| 32 | Epi-dif | 25 | SLPI | KRT17 | IL1R2 | APOBEC3A | SDCBP2 | ERO1L | CEACAM6 | SDR16C5 | IL1RN | KRT6B |
| 33 | Epi-dif | 26 | LCN2 | S100A9 | IL1RN | PRSS8 | S100P | GPRC5A | CLIC3 | KRT23 | SLPI | SDCBP2 |
| 34 | Epi-dif | 18 | LCN2 | SPRR3 | CEACAM5 | SPRR1A | SPRR2A | KRT23 | GCNT3 | PSCA | TMPRSS11E | S100P |
| 35 | Epi-dif | 22 | S100A9 | CLDN4 | APOBEC3A | S100A8 | SPRR1B | PLAUR | IL1RN | SPRR2A | LCN2 | KRT19 |
| 36 | Epi-dif | 5 | S100A7 | S100A8 | S100A9 | CRABP2 | SERPINB3 | PDZK1IP1 | SERPINB4 | PROS1 | SPRR1B | TXNIP |
| 37 | Epi-dif | 6 | GPNMB | LYPD3 | DMKN | KRT16 | ANXA8L2 | GJB2 | FABP5 | DAPL1 | LY6D | ANXA8L1 |
| 38 | Epi-dif | 18 | SBSN | SPRR1B | S100A7 | LYPD3 | SULT2B1 | MFSD5 | SPRR1A | KLK6 | KRTDAP | KLK7 |
| 39 | Epi-dif | 25 | LY6D | FABP5 | KRT16 | FGFBP1 | IGFL1 | KRT6C | KRT6B | THBD | AKR1B10 | LYPD3 |
| 40 | Epi-dif | 22 | LY6D | KRT6C | KRT16 | KRT13 | KRT6B | CSRP2 | FABP5 | S100A9 | SERPINB3 | FABP4 |
| 41 | Epi-dif | 5 | S100A9 | S100A8 | SERPINB4 | KRT6C | SERPINB3 | LY6D | FABP4 | CA2 | AKR1B10 | MRPL23 |
| 42 | Epi-dif + MHC-II | 26 | OLFM4 | KRT15 | HCAR3 | HLA- | MOXD1 | HCAR2 | UBD | HLA- | HLA- | NCK1 |
| | | | | | | DRA | | | | DPA1 | DQA1 | |
| 43 | Epi-dif | 6 | AKR1B10 | LYPD3 | ID1 | ANXA1 | S100A16 | KLC3 | RAB38 | S100A2 | KRT16 | KRT17 |
| 44 | Epi-dif | 20 | S100A2 | KRT6B | KRT14 | ANXA8L1 | ANXA3 | TYMP | ANXA8L2 | TUBA4A | LAMC2 | LYPD3 |
| 45 | stress | 25 | CXCL2 | G0S2 | CXCL1 | CXCL3 | TNFAIP3 | NEDD9 | IL8 | ATF3 | NFKBIA | SOD2 |
| 46 | stress | 17 | ATF3 | CXCL2 | TNFAIP3 | NR4A1 | PPP1R15A | IER2 | FOS | NFKBIZ | CYR61 | NFKBIA |
| 47 | stress | 20 | ATF3 | FOS | EGR1 | DUSP1 | EGR2 | PPP1R15A | FOSB | KRT15 | KRT19 | IER2 |
| 48 | stress | 28 | KRT16 | C10orf99 | AKR1B10 | AQP3 | ATF3 | NR4A1 | LUM | SERTAD1 | LCN2 | ZC3H12A |
| 49 | stress | 26 | DCN | C1S | LUM | KRT14 | ICAM1 | DHRS3 | LOC284454 | LAMB3 | SOD2 | CCL20 |
| 50 | stress | 18 | S100A7 | HLA- | HLA- | HLA- | CD74 | HLA- | PDZK1IP1 | DUSP2 | ENTPD6 | HLA- |
| | | | | DMA | DRA | DMB | | DPA1 | | | | DRB1 |
| 51 | stress | 26 | CXCL10 | MT2A | HLA- | FDCSP | HSPA1B | DDIT4 | TYMP | UBD | CSN3 | C8orf4 |
| | | | | | DRA | | | | | | | |
| 52 | stress | 16 | MMP1 | LDLR | KEAP1 | HIST1H2AM | PILRB | SERPINB4 | PEPD | ZFP36 | FLII | SERPINB13 |
| 53 | | 5 | CTHRC1 | GABRP | SERPINA3 | KRT19 | SOSTDC1 | CAPNS2 | KRT15 | RTP4 | CALML5 | HEBP1 |
| 54 | | 5 | GABRP | MMP7 | SOSTDC1 | KRT19 | CALML5 | CAPNS2 | KRT15 | NUPR1 | SERPINA3 | CLDN7 |
| 55 | detoxification | 20 | SUSD4 | CREG1 | CALML3 | PRODH | CYP4F3 | GSTA4 | CCND3 | GCLM | UCP2 | ALDH3B2 |
| 56 | | 6 | GPC3 | ALDH1A1 | PLA2G16 | KRT19 | FAM83D | VPS25 | EPCAM | KRT8 | UNG | FAM136A |
| 57 | | 20 | KLK5 | PLD3 | S100A2 | KLK6 | SPP1 | UPP1 | KLK9 | LGALS1 | SLC7A5 | ASNS |
| 58 | cell cycle + EMT | 16 | FN1 | KRT8 | VIM | TAGLN | COL1A1 | NNMT | CDK5RAP1 | TUBA1A | TGFBI | MCM7 |
| 59 | | 28 | TAGLN | THBS1 | IL1R2 | KRT8 | ANXA3 | F3 | CD24 | LAMC2 | MFAP5 | SPINK6 |
| 60 | cell cycle + EMT | 28 | CDC20 | CCNB1 | BIRC5 | ANXA3 | CDKN3 | PLAUR | RNF6 | KLK5 | LAMC2 | HAS2 |
| | | | | Genes 11-20 | | | | | | | | |
| 1 | Cell | 17 | DLGAP5 | KIF22 | PRC1 | NUSAP1 | HMGB2 | CKS1B | PBK | KNSTRN | STMN1 | MAD2L1 |
| | cycle(G2/m) | | | | | | | | | | | |
| 2 | Cell | 20 | UBE2C | TPX2 | ARL6IP1 | SPAG5 | NCAPD2 | KIF2C | PRC1 | CKS2 | AURKB | KPNA2 |
| | cycle(G2/m) | | | | | | | | | | | |
| 3 | Cell | 26 | KIF2C | CDCA3 | NCAPD2 | CDKN3 | TOP2A | KIF20A | DLGAP5 | CCNA2 | NUSAP1 | ECT2 |
| | cycle(G2/m) | | | | | | | | | | | |
| 4 | Cell | 25 | UBE2C | CENPW | KIF22 | MELK | CCNA2 | ZWINT | PLK1 | AURKB | MAD2L1 | CKS1B |
| | cycle(G2/m) | | | | | | | | | | | |
| 5 | Cell | 28 | HMGB2 | STMN1 | CKS1B | CCNA2 | TACC3 | CKS2 | TK1 | DTYMK | AURKB | KIF20A |
| | cycle(G2/m) | | | | | | | | | | | |
| 6 | Cell | 18 | DTYMK | COTL1 | RNF26 | FSTL1 | PLAU | STMN1 | PTTG1IP | LAMC2 | RNASEH2A | CIRH1A |
| | cycle(G2/m) | | | | | | | | | | | |
| 7 | Cell | 22 | RRM2 | SMS | TMEM106C | TPX2 | NCAPD2 | TK1 | TUBA1B | PTTG1 | DDX39A | STMN1 |
| | cycle(G2/m) | | | | | | | | | | | |
| 8 | Cell | 22 | TPX2 | ECT2 | PRC1 | DDIT3 | HLA- | CHTF18 | UBE2C | IFIT1 | KAT5 | GPN1 |
| | cycle(G2/m) | | | | | | DRA | | | | | |
| 9 | Cell cycle (G1/S) | 25 | SLC43A3 | FEN1 | MTHFD1 | MCM7 | RANBP1 | SLC29A1 | DCTPP1 | GALE | CLNS1A | FBXW9 |
| 10 | Cell cycle (G1/S) | 22 | MTHFD1 | PCNA | MCM4 | DSN1 | MCM7 | TYMS | CDC45 | RFC4 | FEN1 | UBE2C |
| 11 | Cell cycle (G1/S) | 28 | WARS | ASF1B | GINS2 | GMNN | PKMYT1 | TYMS | GALE | DNAJC9 | CDC6 | MCM4 |
| 12 | Cell cycle (G1/S) | 5 | ANLN | KIF22 | MLF1IP | RNASEH2A | TUBA1B | KPNA2 | TK1 | PCNA | TUBB4B | MCM3 |
| 13 | Cell cycle (G1/S) | 17 | DNAJC9 | TYMS | TK1 | RFC2 | CDC45 | RFC4 | RRM1 | CKS1B | MELK | GMNN |
| 14 | Cell cycle (G1/S) | 20 | CDC6 | TYMS | RFC4 | DTL | MCM4 | CENPM | ZWINT | MCM7 | STRA13 | GMNN |
| 15 | Cell cycle (G1/S) | 26 | TYMS | MCM7 | KIAA0101 | CDCA5 | RRM1 | RPL39L | CDC6 | CENPW | RANBP1 | GINS2 |
| 16 | Cell | 6 | UBE2C | MELK | AURKB | NUSAP1 | ASF1B | CCNA2 | FEN1 | CKS1B | PTTG1 | TOP2A |
| | cycle(G1/S + G2/ | | | | | | | | | | | |
| | M) | | | | | | | | | | | |
| 17 | Cell | 18 | KPNA2 | CDC20 | ZWINT | MCM7 | MAD2L1 | STMN1 | ASF1B | GGCT | HMGB2 | GGH |
| | cycle(G1/S + G2/ | | | | | | | | | | | |
| | M) | | | | | | | | | | | |
| 18 | Cell | 16 | RRM2 | APOBEC3B | PRC1 | CKS1B | PTTG1 | TUBA1B | STMN1 | PBK | H2AFZ | KIF20A |
| | cycle(G1/S + G2/ | | | | | | | | | | | |
| | M) | | | | | | | | | | | |
| 19 | EMT-like | 25 | LAMA3 | ITGA6 | COL17A1 | CD99 | PTHLH | GJA1 | LTBP1 | ITGB6 | LIMA1 | CA9 |
| 20 | EMT-like | 5 | MMP10 | SLC7A8 | LAMB3 | PRSS23 | PDPN | IGFBP7 | TGFBI | SLC38A5 | SERPINE1 | EFEMP1 |
| 21 | EMT-like | 16 | TMEM45A | LEPRE1 | TPM1 | SERPINE1 | MUL1 | FRMD6 | MAGED1 | CTSL1 | GADD45B | ITGB6 |
| 22 | EMT-like | 17 | SERPINE1 | SEMA3C | LAMC2 | SLC38A5 | IL32 | PDLIM7 | PIK3IP1 | PDPN | PRKCDBP | LAMB3 |
| 23 | EMT-like | 22 | TGFBI | SPRR1B | ECM1 | CD68 | IL1R2 | ODC1 | HTRA1 | SQRDL | P4HA2 | F3 |
| 24 | EMT-like | 18 | MT1X | SLC16A3 | WFDC2 | BNIP3 | TNFRSF6B | SULF2 | FCGRT | FHL2 | RNF25 | PLD3 |
| 25 | EMT-like | 6 | CNN3 | IFITM2 | C1R | S100A4 | ANGPTL4 | COL1A1 | GPX3 | TAGLN | TGFBI | GSTA1 |
| 26 | EMT-like | 18 | PSMD13 | SMARCA1 | PDCL3 | TNFRSF6B | LEMD1 | LAMA3 | ACTR3 | VAMP3 | TSR2 | PLEK2 |
| 27 | MHC-II | 17 | SERPING1 | EGLN3 | HLA- | LUM | NDUFA4L2 | CYBRD1 | SPINK6 | IGFBP3 | IFI6 | CALCOCO1 |
| | | | | | DQB1 | | | | | | | |
| 28 | Hypoxia | 18 | PLAU | GJB6 | P4HA1 | PLD3 | PGF | BNIP3 | NUPR1 | MFAP2 | ACP5 | GORASP1 |
| 29 | Hypoxia | 20 | HK2 | EGLN3 | PTHLH | C4orf3 | IGFBP3 | NDUFA4L2 | SLC2A1 | PGF | CA9 | GPI |
| 30 | Epi-dif | 25 | KRT6C | A2ML1 | KLK10 | KLK6 | APOBEC3A | CNFN | S100P | KLK11 | TGM1 | S100A7 |
| 31 | Epi-dif | 17 | SPRR2A | IL36G | CLDN4 | DMKN | LY6D | S100A8 | KLK10 | GPRC5A | MXD1 | SDCBP2 |
| 32 | Epi-dif | 25 | ELF3 | ALDH1A3 | KLK7 | EMP1 | GRB7 | PIK3IP1 | S100A9 | KRT16 | SERPINB1 | PVRL4 |
| 33 | Epi-dif | 26 | PIM1 | PVRL4 | MALL | ALDH1A3 | ELF3 | S100A8 | SPRR1B | EMP1 | ISG15 | RHCG |
| 34 | Epi-dif | 18 | ISG15 | NCCRP1 | MUC4 | PRSS8 | SAA2 | KLK6 | CEACAM6 | KLK13 | SPRR1B | CXCL1 |
| 35 | Epi-dif | 22 | SLPI | FDCSP | PDZK1IP1 | CLDN7 | GPRC5A | ELF3 | TMPRSS4 | ANXA1 | CD55 | MXD1 |
| 36 | Epi-dif | 5 | IL1RN | TMEM79 | SLPI | RHCG | C10orf99 | DMKN | DHRS3 | PVRL4 | PPAP2A | NUPR1 |
| 37 | Epi-dif | 6 | CALML3 | KRT6B | KRTDAP | C10orf99 | GJB6 | THBD | DSG3 | RHOV | CLDN1 | CAPG |
| 38 | Epi-dif | 18 | HIST1H2AC | KRT6B | HIST1H4H | CALML5 | GAST | SPRR2D | CLIC3 | IL1RN | KLK5 | TMEM79 |
| 39 | Epi-dif | 25 | SBSN | AQP3 | ZNF750 | MAL2 | UPK3BL | CRABP2 | CLDN4 | VSNL1 | CA2 | DEGS1 |
| 40 | Epi-dif | 22 | THBD | SFXN4 | ADH7 | KLK8 | MAL2 | LYPD3 | S100A14 | CYB561 | ACAT2 | KRT6A |
| 41 | Epi-dif | 5 | WARS | NELL2 | ACOT7 | GADD45GIP1 | RHOV | C20orf24 | ADPRHL2 | MRPS17 | CFI | RPL21 |
| 42 | Epi-dif + MHC-II | 26 | CD74 | HLA- | DUSP1 | GABRP | GBP2 | HLA- | FABP7 | SNX17 | CLDN1 | MYCL1 |
| | | | | DRB1 | | | | DMA | | | | |
| 43 | Epi-dif | 6 | SDCBP2 | SERPINB5 | TYMP | CSTA | TMBIM1 | THBD | KRT6B | ALDH3B2 | FABP5 | GLTP |
| 44 | Epi-dif | 20 | KRT16 | CLDN5 | SFN | ANXA1 | RHCG | TNFRSF12A | S100A14 | SPRR1B | SQRDL | GJB3 |
| 45 | stress | 25 | ZC3H12A | HCAR2 | PMAIP1 | IER3 | DUSP1 | GEM | FOS | INHBA | NFKBIZ | RND3 |
| 46 | stress | 17 | SOD2 | EGR1 | ZFP36 | HCAR2 | IL8 | CCL20 | RND3 | HBEGF | CDKN1A | DUSP1 |
| 47 | stress | 20 | OSR2 | CYR61 | DDIT3 | JUNB | MYH11 | MYC | KLF6 | EMP1 | OSGIN1 | HERPUD1 |
| 48 | stress | 28 | ID1 | HOPX | DUSP1 | ZFP36 | IER3 | FABP5 | ALDH3A1 | AKR1C2 | NFKBIA | S100A7 |
| 49 | stress | 26 | TNFAIP3 | IDO1 | C1R | NFKBIA | SAA1 | GPNMB | KRT13 | CYP24A1 | IL32 | ZNF267 |
| 50 | stress | 18 | SOD2 | SAA2 | SLC1A3 | SAA1 | SEPP1 | ATP1A1 | HLA- | TNFSF10 | HLA- | ADRB2 |
| | | | | | | | | | DRB5 | | DPB1 | |
| 51 | stress | 26 | IFI6 | CXCL13 | ISG15 | FOS | RASD1 | JUN | DNAJB1 | PSMB10 | CD74 | PHLDA2 |
| 52 | stress | 16 | FOS | SERGEF | STAT1 | JUN | ERRFI1 | RPF2 | LAMA3 | HSPA2 | SGK1 | CDH1 |
| 53 | | 5 | HLA- | NUPR1 | CLDN4 | C11orf1 | CYB5R2 | SDR39U1 | HOPX | ERV3-1 | UNC119 | TXNIP |
| | | | DRA | | | | | | | | | |
| 54 | | 5 | CLDN1 | TXNIP | CTHRC1 | FBXO32 | LY6D | EFNA5 | CLDN4 | CCNA1 | GPX2 | DHRS3 |
| 55 | detoxification | 20 | SULT2B1 | CAPN1 | OSGIN1 | TM7SF2 | GSTA1 | KLK11 | SLC9A9 | PTDSS1 | ALDH1A1 | UGT1A7 |
| 56 | | 6 | CCDC58 | ZNF57 | IMPDH2 | MYH11 | ZNF766 | MCM7 | DDX1 | HDAC3 | C2orf47 | NDUFB5 |
| 57 | | 20 | ARPC1A | CXCR7 | MTHFD2 | GLA | SLC1A5 | LAMB3 | CTSL2 | RHBDD2 | DHCR7 | DPH2 |
| 58 | cell cycle + EMT | 16 | ANXA3 | BIRC5 | CCNB1 | DLEU1 | CDKN3 | FAM69A | IGFBP7 | DBN1 | GLIPR1 | SLC37A3 |
| 59 | | 28 | KRT17 | KLK5 | LGALS1 | TUBB3 | NNMT | CRIP2 | CDA | IL32 | COTL1 | FSTL3 |
| 60 | cell cycle + EMT | 28 | CD24 | TGFBI | IARS2 | ERLEC1 | ANLN | BUB1 | IGFL2 | VAMP7 | PSMC6 | KIF20A |
| | | | | Genes 21-30 | | | | | | | | |
| 1 | Cell | 17 | SPAG5 | TK1 | ANLN | TOP2A | ZWINT | CKS2 | KPNA2 | KIF2C | CDCA3 | CCNA2 |
| | cycle(G2/m) | | | | | | | | | | | |
| 2 | Cell | 20 | KNSTRN | TUBA1B | NUF2 | CKS1B | MAD2L1 | TK1 | DLGAP5 | DTYMK | ECT2 | CENPF |
| | cycle(G2/m) | | | | | | | | | | | |
| 3 | Cell | 26 | FAM64A | AURKB | NUF2 | TK1 | CKS2 | NEK2 | MAD2L1 | STMN1 | H2AFZ | TUBA1B |
| | cycle(G2/m) | | | | | | | | | | | |
| 4 | Cell | 25 | KPNA2 | STMN1 | TYMS | TACC3 | RRM2 | KIF2C | PRC1 | TUBA1B | CDCA3 | PKMYT1 |
| | cycle(G2/m) | | | | | | | | | | | |
| 5 | Cell | 28 | RRM2 | TOP2A | PBK | KPNA2 | NUSAP1 | NCAPD2 | CDCA3 | TRIP13 | CDCA8 | H2AFZ |
| | cycle(G2/m) | | | | | | | | | | | |
| 6 | Cell | 18 | FSTL3 | LAMB3 | C1S | DDX41 | SEPHS2 | ARL6IP1 | NMU | UNC50 | VIM | MAD2L1 |
| | cycle(G2/m) | | | | | | | | | | | |
| 7 | Cell | 22 | LTBP1 | VIM | LGALS1 | VKORC1 | KIF20A | RCC1 | MAD2L1 | EIF2B1 | NUSAP1 | CDCA3 |
| | cycle(G2/m) | | | | | | | | | | | |
| 8 | Cell | 22 | KIF2C | HIST1H4H | ARL6IP1 | KPNA2 | DKC1 | TUBA1B | TOP2A | CKS1B | MMP13 | MX1 |
| | cycle(G2/m) | | | | | | | | | | | |
| 9 | Cell | 25 | PCNA | GMNN | MCM6 | SOD2 | FGFBP1 | RFC5 | IMPDH2 | CDK4 | RFC2 | KIAA0101 |
| | cycle(G1/S) | | | | | | | | | | | |
| 10 | Cell | 22 | SLBP | C19orf48 | KIAA0101 | CDK2 | CDCA7L | DNAJC9 | DUT | FN3KRP | RFC5 | ATAD2 |
| | cycle(G1/S) | | | | | | | | | | | |
| 11 | Cell | 28 | RPA2 | SERPINB3 | MAGEA1 | TK1 | C19orf48 | RFC2 | GBP5 | SERPINB4 | UNG | RAD51 |
| | cycle(G1/S) | | | | | | | | | | | |
| 12 | Cell | 5 | TYMS | CCNB2 | PTTG1 | KIAA0101 | GINS2 | ASF1B | CDC45 | C19orf48 | OIP5 | CDCA5 |
| | cycle(G1/S) | | | | | | | | | | | |
| 13 | Cell | 17 | MCM4 | ASF1B | MTHFD1 | RNASEH2A | RPA2 | RAD51 | UNG | KIAA0101 | LIG1 | C19orf48 |
| | cycle(G1/S) | | | | | | | | | | | |
| 14 | Cell | 20 | MCM6 | CDCA7 | RFC5 | SLC29A1 | KIAA0101 | SNRNP25 | TMEM106C | UNG | MAD2L1 | MLF1IP |
| | cycle(G1/S) | | | | | | | | | | | |
| 15 | Cell | 26 | UNG | FEN1 | HMGB2 | UBE2C | GMNN | MAD2L1 | CTPS1 | RRM2 | MLF1IP | CDK2 |
| | cycle(G1/S) | | | | | | | | | | | |
| 16 | Cell | 6 | CDC20 | SPAG5 | STMN1 | CCNB2 | TUBA1B | CDKN3 | TPX2 | APOBEC3B | PLK1 | CMSS1 |
| | cycle(G1/S + G2/ | | | | | | | | | | | |
| | M) | | | | | | | | | | | |
| 17 | Cell | 18 | TYMS | KIF22 | PTTG1 | NUDT1 | CENPM | PCNA | STRA13 | CKS1B | BIRC5 | RFC2 |
| | cycle(G1/S + G2/ | | | | | | | | | | | |
| | M) | | | | | | | | | | | |
| 18 | Cell | 16 | RUVBL1 | TRIP13 | KPNA2 | KIAA0101 | UBE2T | NUSAP1 | ZCCHC17 | NUDCD2 | DDX39A | CKS2 |
| | cycle(G1/S + G2/ | | | | | | | | | | | |
| | M) | | | | | | | | | | | |
| 19 | EMT-like | 25 | ITGA5 | ODC1 | SERPINE2 | AREG | BNIP3 | MMP3 | P4HA1 | SLC2A1 | FHL2 | NDRG1 |
| 20 | EMT-like | 5 | KLK5 | THBS2 | CAV1 | CXCR7 | LGALS1 | ADM | ANXA5 | PTHLH | FSTL3 | F3 |
| 21 | EMT-like | 16 | CTGF | KDELR3 | ITGA5 | CDH11 | SLC31A2 | BPGM | COL5A2 | CXCL13 | HTRA1 | AMTN |
| 22 | EMT-like | 17 | TLR2 | C1S | TNFSF10 | PLAU | MMP2 | INHBA | GSN | LAMA3 | CXCR7 | SLC7A8 |
| 23 | EMT-like | 22 | TMEM154 | CYB5R1 | LOC100862671 | IL1RN | FEZ1 | TRIM16 | KYNU | GJB5 | DHCR7 | MBOAT2 |
| 24 | EMT-like | 18 | DNPH1 | NDRG1 | MMP28 | TCIRG1 | CTSH | MMP13 | IGFL1 | CCDC115 | SERPINE1 | KRT8 |
| 25 | EMT-like | 6 | CTHRC1 | SAT1 | SPP1 | CCL2 | SOD2 | S100A16 | MT2A | SERPINE1 | TIMP3 | TPPP3 |
| 26 | EMT-like | 18 | HSPA5 | GALNT3 | SERPINB5 | DDX47 | ITGB1 | NANS | TVP23B | ADAM9 | TM9SF2 | PAFAH1B2 |
| 27 | MHC-II | 17 | TPPP3 | SLC04A1 | AVPI1 | CRIP1 | RARRES3 | ADM | LAMB1 | APOL1 | BNIP3 | MX1 |
| 28 | Hypoxia | 18 | LPIN3 | MIR205HG | ADM | CLCA2 | SNAI2 | ERO1L | ITGA5 | HIST1H1C | DDIT4 | INHBA |
| 29 | Hypoxia | 20 | BIK | SLCO1B3 | MT1X | PFKFB3 | WDR45B | BNIP3L | IGFBP2 | BHLHE40 | P4HA2 | ELF3 |
| 30 | Epi-dif | 25 | PRSS3 | IGFL2 | KLK9 | CLDN4 | KRT16 | S100A9 | KLK5 | CSTA | CTSL2 | NCCRP1 |
| 31 | Epi-dif | 17 | SBSN | KRT6C | GLTP | PVRL4 | TMEM79 | NDUFA4L2 | CDA | TGM1 | RHCG | KRT6B |
| 32 | Epi-dif | 25 | KLK11 | GPRC5A | NPEPPS | EHF | FXYD5 | TGFA | DSC2 | PLAUR | KLK10 | NCCRP1 |
| 33 | Epi-dif | 26 | GRHL1 | FXYD5 | LGALS3 | TMPRSS11E | PPL | NDRG2 | KRT6A | SCNN1A | ECM1 | GRB7 |
| 34 | Epi-dif | 18 | IL1RN | NDRG2 | CFB | PDZKIIP1 | MX1 | ELF3 | SLPI | TMPRSS11D | ERO1L | IFI6 |
| 35 | Epi-dif | 22 | SDCBP2 | TACSTD2 | SPRR3 | SAA1 | IL8 | ZFP36 | TMPRSS11D | SPRR1A | PSCA | TMPRSS11A |
| 36 | Epi-dif | 5 | TMEM45A | IGFL2 | FABP4 | HIST2H2AA3 | HIST2H2AA4 | BNIPL | SDR16C5 | SERPING1 | KRT16 | ERV3-1 |
| 37 | Epi-dif | 6 | LGALS7B | TACSTD2 | ALDH3B2 | DSC2 | CLDN4 | CDKN1A | GLTP | FAM57A | S100A16 | IL20RB |
| 38 | Epi-dif | 18 | GRHL3 | KRT16 | CEACAM6 | KRT23 | HIST2H2AA3 | HIST2H2AA4 | PI3 | GRHL1 | OVOL1 | S100A8 |
| 39 | Epi-dif | 25 | ALDH2 | RAB38 | NSG1 | DSC2 | GBP2 | IDH2 | FAM213A | EPCAM | CALML5 | KRT6A |
| 40 | Epi-dif | 22 | TMEM109 | SEPHS2 | COQ9 | TRAP1 | S100A8 | LSM10 | CLTB | WBSCR22 | IMPDH2 | DBNDD2 |
| 41 | Epi-dif | 5 | SLC25A1 | IFI30 | TOMM40 | TYMP | MRPS24 | PDHB | FOS | CCDC109B | CA9 | IFITM1 |
| 42 | Epi-dif + MHC-II | 26 | PHGDH | FOS | TNFSF10 | STAT1 | C1S | FKBP5 | KRT5 | GPNMB | CXCL14 | SGK1 |
| 43 | Epi-dif | 6 | FIS1 | CYP26A1 | ISG15 | GBP6 | DSG3 | SFN | FDXR | PCIF1 | JOSD2 | DAXX |
| 44 | Epi-dif | 20 | LGALS7 | LGALS7B | LEMD1 | HMOX2 | FGFBP1 | PLAUR | EMP3 | TUBB6 | ISG15 | CAP1 |
| 45 | stress | 25 | OVOL1 | ADAMTS1 | PPP1R15A | ZFP36 | TRIB1 | TGIF1 | ICAM1 | IRF1 | DNAJA1 | HAS2 |
| 46 | stress | 17 | HCAR3 | DUSP2 | CTGF | FOSB | JUNB | ZC3H12A | NEDD9 | NCOA7 | SERTAD1 | SGK1 |
| 47 | stress | 20 | ID2 | JUN | RASD1 | PMAIP1 | TNC | TUBA1A | BTG2 | CDKN1A | SLC7A8 | ZFP36 |
| 48 | stress | 28 | SAT1 | IER2 | SGK1 | HBEGF | NXF1 | FOSB | FOS | CDKN1A | FOSL1 | SLPI |
| 49 | stress | 26 | UBD | MOXD1 | VRK2 | TACSTD2 | RND3 | IL1R2 | SERTAD3 | PPP1R15A | TSC22D1 | IFNGR1 |
| 50 | stress | 18 | IFITM1 | S100A8 | CLDN1 | NUPR1 | NR4A1 | LCN2 | TRAPPC6A | NCOA7 | KLF10 | DUSP23 |
| 51 | stress | 26 | IFIT3 | GADD45B | HLA- | ZC3H12A | MT1X | HLA- | IDO1 | IFI35 | IFITM1 | HLA- |
| | | | | | DPA1 | | | DRB1 | | | | DMB |
| 52 | stress | 16 | CXCL10 | RUSC1 | MOV10 | IMP3 | NR1H2 | C10orf54 | MST1R | CTNNBL1 | EGLN2 | PLK2 |
| 53 | | 5 | DGCR6L | EFNA4 | CRIP2 | MMP7 | MDP1 | TP53TG1 | ALKBH3 | ABHD11 | ISOC2 | POP7 |
| 54 | | 5 | DMKN | LYPD3 | NDRG2 | LGALS7 | FZD6 | TM4SF1 | LGALS7B | PYGB | MYLK | GSN |
| 55 | detoxification | 20 | ALDH3A1 | SCIN | CBR1 | ALDOC | CHP2 | NDRG4 | ENTPD3 | RAB25 | EPAS1 | ZNF750 |
| 56 | | 6 | PHB | HRASLS2 | PPT1 | CLDN7 | TMEM230 | GCSH | ENTPD3 | VSNL1 | NELFCD | PIN1 |
| 57 | | 20 | SCPEP1 | MEST | ARPC1B | ODC1 | SND1 | DDC | EBNA1BP2 | FGFBP2 | PPT1 | MYADM |
| 58 | cell cycle + EMT | 16 | PHF5A | HIST1H4C | MARCKSL1 | SMS | VKORC1 | TCTA | AGPAT2 | TPM1 | PTTG1 | COA6 |
| 59 | | 28 | TNC | SERPINB2 | KRT16 | KLK9 | FST | TUBB2A | TUBA1B | PDLIM4 | EMP3 | AXL |
| 60 | cell cycle + EMT | 28 | PLAU | TNC | CXCL11 | TAGLN | PPID | CCNB2 | ATP5SL | ERLIN1 | TAF9 | RCN2 |
| | | | | Genes 31-40 | | | | | | | | |
| 1 | Cell | 17 | TACC3 | NCAPD2 | BUB1 | DTYMK | TRIP13 | ARL6IP1 | TROAP | CENPF | UBE2C | RNF26 |
| | cycle(G2/m) | | | | | | | | | | | |
| 2 | Cell | 20 | CCNA2 | BUB1 | NEK2 | KIF20A | TROAP | TACC3 | CKAP2 | DDX39A | UBE2T | PBK |
| | cycle(G2/m) | | | | | | | | | | | |
| 3 | Cell | 26 | CENPF | NMU | KPNA2 | DTYMK | CKAP2 | KIF23 | BUB1 | PRC1 | CENPW | HMGB3 |
| | cycle(G2/m) | | | | | | | | | | | |
| 4 | Cell | 25 | CDK1 | RNASEH2A | NMU | ANLN | CKS2 | TROAP | NCAPD2 | TUBB6 | HIST1H4C | UBE2T |
| | cycle(G2/m) | | | | | | | | | | | |
| 5 | Cell | 28 | NTAN1 | DLGAP5 | KIF2C | TYMS | CENPF | PKMYT1 | C16orf91 | KIAA0101 | SNHG3 | RNF26 |
| | cycle(G2/m) | | | | | | | | | | | |
| 6 | Cell | 18 | CRELD2 | AGR2 | KIF20A | TUBB6 | KPNA2 | LGALS1 | CTSC | TPX2 | EBP | NUP37 |
| | cycle(G2/m) | | | | | | | | | | | |
| 7 | Cell | 22 | DTYMK | THBS1 | CENPM | CKS1B | ZWINT | TNFRSF12A | TRIP13 | APOBEC3B | VRK1 | RNF26 |
| | cycle(G2/m) | | | | | | | | | | | |
| 8 | Cell | 22 | GGH | KIF22 | ODF2 | HAX1 | RPA2 | RNF26 | MBTPS1 | NUSAP1 | SPAG5 | YTHDF3 |
| | cycle(G2/m) | | | | | | | | | | | |
| 9 | Cell | 25 | TK1 | HLA- | RNASEH2A | SLBP | GMPS | CDCA4 | TALDO1 | LDHB | PHGDH | TRAP1 |
| | cycle(G1/S) | | | DRA | | | | | | | | |
| 10 | Cell | 22 | ZWINT | CCND3 | RRM1 | TUBG1 | MGME1 | FANCI | RFC3 | GGCT | POLA2 | USP18 |
| | cycle(G1/S) | | | | | | | | | | | |
| 11 | Cell | 28 | POLD1 | LIG1 | CD14 | CRELD2 | KIF22 | WDR34 | GYG1 | UHRF1 | FDPS | SDF2L1 |
| | cycle(G1/S) | | | | | | | | | | | |
| 12 | Cell | 5 | H2AFZ | STMN1 | HIST1H4C | AURKB | UBE2T | CENPW | DSN1 | DTL | CKS1B | CDCA8 |
| | cycle(G1/S) | | | | | | | | | | | |
| 13 | Cell | 17 | STMN1 | CDCA7 | RFC5 | DTL | PSMC3IP | GGCT | CDK4 | CDC6 | SLBP | NUSAP1 |
| | cycle(G1/S) | | | | | | | | | | | |
| 14 | Cell | 20 | RRM2 | RFC2 | MSH6 | MSH2 | CKS1B | VRK1 | KNTC1 | UHRF1 | HMGB2 | ORC6 |
| | cycle(G1/S) | | | | | | | | | | | |
| 15 | Cell | 26 | PKMYT1 | DNAJC9 | RFC2 | POLD1 | MCM2 | RECQL | CKS1B | DTL | MCM6 | DUT |
| | cycle(G1/S) | | | | | | | | | | | |
| 16 | Cell | 6 | RFC4 | PRC1 | RFC5 | CKS2 | UBE2T | VRK1 | KIF23 | SIVA1 | POLR2H | HAT1 |
| | cycle(G1/S + G2/ | | | | | | | | | | | |
| | M) | | | | | | | | | | | |
| 17 | Cell | 18 | CCNB1 | MCM2 | CDK1 | LDHB | TRAPPC2L | MCM4 | RFC4 | CKS2 | H2AFZ | NUSAP1 |
| | cycle(G1/S + G2/ | | | | | | | | | | | |
| | M) | | | | | | | | | | | |
| 18 | Cell | 16 | KNSTRN | DTYMK | PLK1 | DNAJC9 | CENPM | VRK1 | TUBB4B | TPX2 | PCNA | CDCA3 |
| | cycle(G1/S + G2/ | | | | | | | | | | | |
| | M) | | | | | | | | | | | |
| 19 | EMT-like | 25 | SDC1 | PRSS23 | NPNT | RAMP1 | CDH13 | DST | MMP2 | ITGB4 | PTK7 | GLB1 |
| 20 | EMT-like | 5 | VIM | PLEK2 | TNFRSF12A | DFNA5 | MT2A | MMP2 | IGFBP6 | SLC3A2 | PFN2 | LEPREL1 |
| 21 | EMT-like | 16 | TCF25 | IL32 | LINC00152 | GLIPR1 | MMP10 | TMEM40 | HIST1H2BG | EDN1 | SPATA20 | HERPUD1 |
| 22 | EMT-like | 17 | WDR91 | ALDH2 | EXT2 | SPHK1 | PRSS8 | NINJ1 | P4HA2 | TNFRSF6B | SLC39A14 | FTSJ1 |
| 23 | EMT-like | 22 | ITGA5 | LAMB3 | ITGB1 | LEPREL1 | MEG3 | EMP3 | DSC2 | SDC4 | UAP1 | RBP1 |
| 24 | EMT-like | 18 | MT2A | GAMT | TGM2 | HTRA1 | PLEKHA1 | ECM1 | SGK1 | GJA1 | COL17A1 | NMRK1 |
| 25 | EMT-like | 6 | MYADM | PLIN2 | SPARCL1 | LSP1 | TSPAN4 | IF130 | SMIM3 | P4HA2 | SERPINA3 | CYR61 |
| 26 | EMT-like | 18 | MAPRE1 | EFNA5 | KLF7 | DKK3 | TP63 | SF3A3 | HERPUD1 | CCL20 | CMTM6 | WDR18 |
| 27 | MHC-II | 17 | HLA- | PLA2R1 | GSTM3 | CAPNS2 | C1R | TXNIP | BCKDHA | C3 | MFAP5 | GBP1 |
| | | | DPB1 | | | | | | | | | |
| 28 | Hypoxia | 18 | PVRL4 | ENO2 | HK2 | HEXA | ANGPTL4 | C1orf43 | PFKP | SLC39A13 | HIST1H2BD | PLOD2 |
| 29 | Hypoxia | 20 | NTS | ALDOA | SEMA4B | DDIT4 | CXADR | IFNGR1 | F3 | PVRL4 | ACVR2A | KDM3A |
| 30 | Epi-dif | 25 | RHCG | KRT75 | S100A8 | ACSL1 | SLPI | APOBEC3A_B | IL1RN | S100A1 4 | ACOT7 | GRHL1 |
| 31 | Epi-dif | 17 | CSTB | KLK11 | PRSS8 | PLAUR | TMEM40 | CRYAB | CA2 | FAM83A | SULT2B1 | LYPD3 |
| 32 | Epi-dif | 25 | ADIRF | DSG3 | CLDN7 | SERPINB2 | ECM1 | CLDN4 | KRT7 | NDRG4 | CA2 | KIFC3 |
| 33 | Epi-dif | 26 | KRT16 | HSPB8 | CSTA | CSTB | RAB11FIP1 | CLDN7 | SPINT1 | CLTB | TSPAN1 | TUBA4A |
| 34 | Epi-dif | 18 | NR4A1 | HIST1H1C | CLDN7 | SLC31A2 | CNFN | TXNRD1 | SAT1 | NEU1 | FTH1 | GBA |
| 35 | Epi-dif | 22 | BHLHE40 | NR4A1 | CEACAM5 | S100P | TNFAIP3 | BIK | GLUL | LYPD3 | KYNU | HIST1H2AC |
| 36 | Epi-dif | 5 | BCL6 | LYPD3 | CDKN1A | ID1 | NEAT1 | GRHL1 | MMP13 | AQP3 | LGALS7B | KRT6C |
| 37 | Epi-dif | 6 | ID1 | RHCG | CXCL14 | SMIM14 | OAS1 | IL1RN | TMEM79 | CDC42EP4 | GRHL3 | NDRG1 |
| 38 | Epi-dif | 18 | RHCG | PVRL4 | CLTB | ZNF750 | DSC2 | DUOXA1 | CLDN4 | PLA2G4B | RHOD | SPSB3 |
| 39 | Epi-dif | 25 | GALK1 | CALML3 | GRHL3 | OVOL1 | MPZL2 | DIMT1 | CDKN1A | PKP1 | GSTA4 | MT1X |
| 40 | Epi-dif | 22 | PGD | AQP3 | IMP4 | AFG3L2 | SPNS1 | MT1X | CECR5 | TMBIM1 | RWDD2B | TLCD1 |
| 41 | Epi-dif | 5 | TMEM54 | FBXO6 | IMPDH2 | SEPHS2 | DRG1 | WDR4 | C12orf75 | DDX49 | TK1 | VBP1 |
| 42 | Epi-dif + MHC-II | 26 | ANAPC15 | CCT7 | PYCARD | ZFP36 | C22orf28 | INPP1 | NDUFV2 | AAMP | GSTA4 | FBXW5 |
| 43 | Epi-dif | 6 | GJB5 | JUP | FAM57A | TMEM179B | GPNMB | FRMD8 | GPR89A | YDJC | DMKN | HIST2H2AA3 |
| 44 | Epi-dif | 20 | CD82 | KLK10 | SERPINB5 | GPR87 | DMKN | GJB2 | C19orf33 | PRKCDBP | MYL12A | CAPN2 |
| 45 | stress | 25 | BHLHE40 | DUSP2 | EFNA1 | NR4A1 | BDKRB1 | RIPK2 | SGK1 | ZFAND2A | HBEGF | SERTAD1 |
| 46 | stress | 17 | SERPINB2 | EDN2 | PLK2 | DNAJA1 | IRF1 | BTG2 | MAFF | ADRB2 | SLC20A1 | TNF |
| 47 | stress | 20 | NR4A1 | ID3 | GPC3 | TSC22D1 | TGIF1 | ARL4D | TOB1 | SNA12 | ELF3 | SGK1 |
| 48 | stress | 28 | IGFBP3 | RND3 | JUNB | DNAJB1 | LOC284454 | HSPB3 | OVOL1 | SPRR2D | ADRB2 | EMP1 |
| 49 | stress | 26 | DST | NCOA7 | SERTAD1 | LGALS1 | BIK | GJB2 | SLC38A2 | KEAP1 | IRF1 | PRNP |
| 50 | stress | 18 | GLUL | TNFRSF18 | CFB | GADD45B | CCL20 | ZC3H12A | HLA- | CYR61 | PARP9 | ID1 |
| | | | | | | | | | DQB1 | | | |
| 51 | stress | 26 | BST2 | RARRES3 | CLK1 | IFI44 | IFITM3 | DDIT3 | ID1 | PILRB | TAP1 | HSPA6 |
| 52 | stress | 16 | BCL6 | DHCR24 | FASTK | GANAB | RARG | CTSD | FLOT1 | GALNT18 | DDX41 | C16orf62 |
| 53 | | 5 | TRIM21 | ECHDC1 | PCIF1 | ELAC2 | NAGK | MKS1 | DPP7 | TMPRSS4 | CIDEB | RARRES3 |
| 54 | | 5 | TFCP2L1 | ALDH1A1 | CKMT1A | SPON2 | CD14 | TMSB4X | FXYD3 | GPRC5B | KRTDAP | TMPRSS4 |
| 55 | detoxification | 20 | ABCB6 | TDP2 | TP53I3 | PVRL4 | SLC16A5 | MUC4 | CLDN8 | NUDT7 | ACSL1 | THBD |
| 56 | | 6 | CHCHD3 | NDUFAB1 | HBEGF | RARRES3 | MED10 | SORD | TFRC | TOMM34 | MCM5 | SDF2L1 |
| 57 | | 20 | SRPRB | POR | ITGB4 | PYGB | KDELR2 | IFRD2 | PDIA4 | YARS | COPG1 | MSN |
| 58 | cell cycle + EMT | 16 | C6orf226 | TMEM205 | TMX4 | MANF | CTSH | KRT18 | PLOD1 | NSMCE4A | CDC20 | CKLF |
| 59 | | 28 | PTHLH | DKK3 | TSPAN4 | CCL5 | KPNA2 | GLIPR1 | RANGRF | RHOD | TNFRSF12A | LINC00152 |
| 60 | cell cycle + EMT | 28 | YES1 | DENR | PRSS23 | KLK9 | PTTG1 | KRT8 | ACTR10 | C1GALT1C1 | C3orf37 | FSTL1 |
| | | | | Genes 41-50 | | | | | | | | |
| 1 | Cell | 17 | NDC80 | H2AFZ | KIF23 | TUBB4B | TUBA1B | AURKB | ANP32E | NUF2 | RRM2 | DEPDC1B |
| | cycle(G2/m) | | | | | | | | | | | |
| 2 | Cell | 20 | CDCA3 | TUBB4B | KIF22 | FAM64A | AURKA | CENPN | KIF23 | H2AFZ | CKAP5 | HSPA8 |
| | cycle(G2/m) | | | | | | | | | | | |
| 3 | Cell | 26 | TACC3 | KNSTRN | ARL6IP1 | CKAP5 | OIP5 | AURKA | SPAG5 | TUBB4B | CNIH4 | UBE2C |
| | cycle(G2/m) | | | | | | | | | | | |
| 4 | Cell | 25 | NDC80 | KIF23 | DLGAP5 | KIF20A | DNAJC9 | TUBB | MLF1IP | KNSTRN | CDCA8 | TUBG1 |
| | cycle(G2/m) | | | | | | | | | | | |
| 5 | Cell | 28 | NDC80 | BRD8 | UBD | WDR54 | CENPM | TROAP | CXCL10 | SPAG5 | REEP4 | SIVA1 |
| | cycle(G2/m) | | | | | | | | | | | |
| 6 | Cell | 18 | RNASEH1 | KARS | CDCA3 | MRPS12 | CD276 | AREG | RAC2 | SLC35A2 | PSENEN | C22orf28 |
| | cycle(G2/m) | | | | | | | | | | | |
| 7 | Cell | 22 | PRC1 | LEPREL1 | DLGAP5 | INHBA | PLAU | ARL6IP1 | AURKA | NOB1 | ANXA1 | AREG |
| | cycle(G2/m) | | | | | | | | | | | |
| 8 | Cell | 22 | RPL13AP5 | ALDH4A1 | MMP1 | COPS8 | RFC3 | TUBB4B | ANP32E | CDCA3 | YEATS4 | NUF2 |
| | cycle(G2/m) | | | | | | | | | | | |
| 9 | Cell cycle(G1/S) | 25 | SHMT1 | WLS | XRN2 | POLD2 | IDH2 | METTL1 | PYCR1 | CXCL14 | IARS | NUBP2 |
| 10 | Cell cycle(G1/S) | 22 | CYB5A | KIF22 | CDCA7 | STAT1 | RAD51C | FANCA | HLA-DRB5 | TEX30 | ALDH9A1 | EARS2 |
| 11 | Cell cycle(G1/S) | 28 | ZWINT | SGSM3 | NASP | DNMT1 | MSRB1 | NXT1 | S100A8 | STMN1 | THEM6 | GBP2 |
| 12 | Cell cycle(G1/S) | 5 | BIRC5 | ORC6 | HAT1 | SPAG5 | WDR34 | TMEM106C | ATAD2 | NDC80 | MCM5 | RRM2 |
| 13 | Cell cycle(G1/S) | 17 | TMEM106C | STRA13 | MAD2L1 | H2AFZ | WDR34 | DUT | CENPM | POLE3 | SIVA1 | SAE1 |
| 14 | Cell cycle(G1/S) | 20 | FANCI | C19orf48 | TUBG1 | TRIP13 | ATAD2 | RNASEH2A | UBE2C | FANCA | RAD51 | LIG1 |
| 15 | Cell cycle(G1/S) | 26 | CENPM | SLBP | RAD51 | PSMG1 | ACOT7 | VRK1 | AP2S1 | CCND1 | CDCA7 | TRIP13 |
| 16 | Cell | 6 | GMNN | CENPW | TMEM106C | PARP2 | NUDT1 | TUBG1 | AC0T7 | PCNA | MCM7 | CENPM |
| | cycle(G1/S + G2/ | | | | | | | | | | | |
| | M) | | | | | | | | | | | |
| 17 | Cell | 18 | GINS2 | DNAJC9 | RANBP1 | RPA2 | UBE2T | TPX2 | GMNN | POLE3 | VRK1 | TUBB |
| | cycle(G1/S + G2/ | | | | | | | | | | | |
| | M) | | | | | | | | | | | |
| 18 | Cell | 16 | TUBB6 | PPIH | RRM1 | VDAC3 | NUDT1 | CCNA2 | LSM4 | APTX | TUBG1 | TYMS |
| | cycle(G1/S + G2/ | | | | | | | | | | | |
| | M) | | | | | | | | | | | |
| 19 | EMT-like | 25 | AKR1C1 | DLK2 | PXN | LEPREL1 | PSMD2 | PFN2 | CSRP2 | SLC16A1 | PFKP | PLS3 |
| 20 | EMT-like | 5 | COL5A2 | ITGB1 | SERINC2 | MMP1 | DST | INHBA | PDLIM1 | CD24 | IVNS1ABP | FTH1 |
| 21 | EMT-like | 16 | IGFL2 | GALNT2 | RALA | FSTL1 | NIPSNAP1 | SEMA3C | CDKN1A | DSG2 | CTSA | MFSD1 |
| 22 | EMT-like | 17 | ATP1B1 | NNMT | COL7A1 | BMP1 | SELM | TNFRSF12A | RTKN | SERINC2 | KYNU | PTK2 |
| 23 | EMT-like | 22 | DHRS7 | OPTN | COL4A2 | ANXA3 | AHNAK2 | ANXA8 | HERPUD1 | CD40 | SERINC1 | TOR1A |
| 24 | EMT-like | 18 | CRABP2 | ANXA4 | TPST1 | SLC2A1 | GLTSCR2 | KRT14 | SDC2 | TMEM14C | GALNT2 | TIMP3 |
| 25 | EMT-like | 6 | ADAMTS1 | SELM | MLKL | NDUFAF3 | HERPUD1 | LUM | NMRK1 | SLC39A1 | TIMP1 | TMEM179B |
| 26 | EMT-like | 18 | USP10 | CLIC4 | ERGIC2 | GMPPA | RPL21P28 | DNAJC3 | C14orf1 | LGALS1 | RAP1B | RAB8A |
| 27 | MHC-II | 17 | IFI35 | BST2 | KLK5 | EDN2 | MOV10 | ARRDC1 | ETS2 | EHD2 | CFB | DDIT4 |
| 28 | Hypoxia | 18 | SPSB3 | TSC22D2 | HDAC3 | TGFBI | AP1G2 | SEMA3C | LDHA | MMP9 | FAM213A | DAAM1 |
| 29 | Hypoxia | 20 | WSB1 | IVNS1ABP | PELI1 | DARS | KCNK1 | GAPDH | TPD52 | CLEC2B | ALDOC | DHRS3 |
| 30 | Epi-dif | 25 | NDRG2 | LCN2 | KLK8 | SDCBP2 | LYPD3 | GRHL3 | HSPB8 | CLDN1 | CD24 | ATL2 |
| 31 | Epi-dif | 17 | ELF3 | ANGPTL4 | RAET1G | GRHL3 | DSC2 | CD24 | ALDH1A3 | THBD | KLK13 | RAB25 |
| 32 | Epi-dif | 25 | KLK5 | GABRP | RAP2B | PRSS8 | CNFN | LBH | ELK3 | CD68 | A2ML1 | S100A8 |
| 33 | Epi-dif | 26 | PDZK1IP1 | ALDH3B2 | SMAGP | LYPD3 | A2ML1 | GJB3 | PLK3 | ZFAND6 | RAB9A | MAFF |
| 34 | Epi-dif | 18 | APOBEC3A | ERV3-1 | ECM1 | SDCBP2 | PTGES | TNFAIP3 | SMAGP | SPRR2E | CTSA | OAS1 |
| 35 | Epi-dif | 22 | EMP1 | SPRR2D | CD46 | DUSP10 | SAA2 | PRSS8 | DHRS3 | PFKFB3 | SERPINB1 | EHF |
| 36 | Epi-dif | 5 | THBD | PIK3IP1 | LY6D | MALAT1 | IRF6 | CYB5A | TMEM91 | GBP2 | PIGC | GIPC1 |
| 37 | Epi-dif | 6 | DSC3 | DSP | PERP | NOL3 | TUBA4A | SULT2B1 | GAA | SLC39A6 | MPZL2 | EHF |
| 38 | Epi-dif | 18 | TBCC | AQP3 | RABGEF1 | LAD1 | DSG3 | H1F0 | NFKBIL1 | TRAPPC5 | GPS2 | HSPH1 |
| 39 | Epi-dif | 25 | NRP1 | DBI | CTSC | PHB2 | S100A14 | GTPBP4 | XPNPEP1 | RAB3D | DSP | MRPL16 |
| 40 | Epi-dif | 22 | EIF4EBP1 | PSMC5 | NMU | SNRNP40 | DSC2 | KLF5 | IAH1 | C12orf10 | UCKL1 | MRPL54 |
| 41 | Epi-dif | 5 | GJB3 | TOMM6 | TNFRSF18 | DCXR | MPG | MAF1 | MT2A | VSNL1 | AAMP | PLTP |
| 42 | Epi-dif + MHC-II | 26 | RARRES3 | IFNGR1 | TOB1 | EIF3I | RAMP1 | COPS7A | SRD5A1 | GPN1 | IFI30 | DUSP2 |
| 43 | Epi-dif | 6 | HIST2H2AA4 | CLDN7 | SCO2 | MMADHC | PPME1 | C1GALT1C1 | CHMP2A | TUBA4A | CTNNBL1 | CHCHD1 |
| 44 | Epi-dif | 20 | DSG3 | B4GALT4 | FOSL1 | HAS3 | CDKN1A | C1orf116 | A2ML1 | PHLDA2 | KANK1 | KRT6A |
| 45 | stress | 25 | PLK3 | FOSL1 | DNAJB1 | BRD2 | PLAUR | TNFAIP8 | CTGF | PLK2 | EGR1 | JUN |
| 46 | stress | 17 | CCNL1 | EPHA2 | KBTBD7 | JUN | LOC284454 | PMAIP1 | EDN1 | DUSP6 | GADD45B | FOSL1 |
| 47 | stress | 20 | PER2 | DUSP6 | ADM | MYADM | CCNA1 | DNAJB14 | DLX5 | KLF10 | TUBB2A | GLUL |
| 48 | stress | 28 | EGR1 | S100A9 | PPP1R15A | NUPR1 | SPRR1B | LY6D | S100A8 | GADD45B | DNAJA1 | PLK2 |
| 49 | stress | 26 | SGK1 | GSTA1 | CCND1 | DUSP1 | SNW1 | C8orf4 | LOC100190986 | CLEC7A | TAPBPL | ZFP36 |
| 50 | stress | 18 | SCPEP1 | IFI30 | S100A9 | CCNL1 | MAFF | CD14 | MFSD2A | ITFG1 | EPHA2 | SERPINB4 |
| 51 | stress | 26 | MT1E | ADM | TRIM22 | HLA- | MT1G | GBP1 | HLA-DQB1 | EGR1 | NLRC5 | IER3 |
| | | | | | | DQA1 | | | | | | |
| 52 | stress | 16 | JUNB | SLC1A5 | RIC8A | CXCL11 | MID1 | TAP1 | ATXN7L3B | ZNF207 | NRBP1 | SSH3 |
| 53 | | 5 | FAM32A | PRPF18 | IFI44L | SMAGP | R3HCC1 | UCKL1 | AIG1 | ETFDH | TMEM205 | RIC8A |
| 54 | | 5 | GLTP | MAGEA4 | C19orf33 | SDC4 | HOPX | SOX4 | DSTN | GABARAP | CMTM6 | PVRL4 |
| 55 | detoxification | 20 | MAGED1 | JUP | TRIM29 | TRAPPC6A | SULT1A1 | LGALS3 | PTPN6 | SDC1 | PEPD | ABHD4 |
| 56 | | 6 | SLC29A1 | RPN1 | PRDX4 | IQCB1 | TXNRD1 | GSN | CDK4 | DHCR24 | GTPBP4 | ASH2L |
| 57 | | 20 | ALDH2 | MRPS24 | GALNT11 | PLTP | PNO1 | GAST | PLA2G4A | UQCRH | AP2B1 | PLOD3 |
| 58 | cell cycle + EMT | 16 | ORC5 | MYADM | YIF1A | ZNF7 | MSRB1 | EMP3 | ISOC2 | GCHFR | LGALS1 | GANC |
| 59 | | 28 | SPHK1 | CDKN3 | KRT75 | LAMA3 | FGFBP1 | P4HA2 | TGFBI | ACLY | GOT2 | LAMB3 |
| 60 | cell cycle + EMT | 28 | NRAS | ELP2 | PPP2R5C | CCNA2 | PJA2 | PTHLH | UBE2V2 | AXL | STMN1 | OAT |

TABLE S7
Six meta-signatures, each derived from multiple related NNMF
programs, Related to FIG. 3. Genes in each program are ordered from most to least
significant.

| Cell Cycle | | p-EMT | | Epi dif. 1 | |
|---|---|---|---|---|---|
| Genes | Genes | Genes | Genes | Genes | Genes |
| 1-50 | 51-100 | 1-50 | 51-100 | 1-50 | 51-100 |
| TK1 | MCM5 | SERPINE1 | ARPC1B | IL1RN | MALAT1 |
| HMGB2 | PLK1 | TGFBI | APP | SLPI | TRIP10 |
| ZWINT | GGH | MMP10 | MFAP2 | CLDN4 | CAST |
| MAD2L1 | MCM4 | LAMC2 | MPZL1 | S100A9 | TMPRSS4 |
| TUBA1B | CENPN | P4HA2 | DFNA5 | SPRR16 | TOM1 |
| STMN1 | TMPO | PDPN | MT2A | PVRL4 | A2ML1 |
| KIF22 | CDCA3 | ITGA5 | MAGED2 | RHCG | MBOAT2 |
| CKS1B | DEK | LAMA3 | ITGA6 | SDCBP2 | LGAL53 |
| H2AFZ | RPA2 | CDH13 | FSTL1 | S100A8 | ERO1L |
| CENPW | KIF2C | TNC | TNFRSF12A | APOBEC3A | EHF |
| CDC20 | CDK1 | MMP2 | IL32 | GRHL1 | LCN2 |
| DTYMK | CDCA5 | EMP3 | COPB2 | SULT2B1 | YPEL5 |
| UBE2C | LSM4 | INHBA | PTK7 | ELF3 | ALDH3B2 |
| UBE2T | KNSTRN | LAMB3 | OCIAD2 | KRT18 | DMKN |
| NUSAP1 | TUBG1 | VM | TAX1BP3 | PRSS8 | PIK3P1 |
| RRM2 | SMC4 | SEMA3C | SEC13 | MXD1 | CEACAM6 |
| BIRC5 | CSE1L | PRKCDBP | SERPINH1 | S100A7 | OVCL1 |
| RNASEH2A | UHRF1 | ANXA5 | TPM4 | KRT6B | TMPRSS11E |
| PCNA | RANBP1 | DHRS7 | MYH9 | LYPD3 | CD55 |
| TUB8 | CDCA8 | ITGB1 | ANXASL1 | TACSTD2 | KLK8 |
| KPNA2 | MCM2 | ACTN1 | PLOD2 | CDKN1A | SPRR2D |
| ASF1B | RFC2 | CXCR7 | GALNT2 | KLK11 | NDRG2 |
| TRIP13 | HMGN2 | ITG86 | LEPREL1 | GPRC5A | CD24 |
| CCNB1 | ATAD2 | IGFBP7 | MAGED1 | KLK10 | HIST1H1C |
| TPX2 | HAT1 | THBS1 | SLC38A5 | TMBIM1 | LY6D |
| CCNB2 | PKMYT1 | PTHLH | FSTL3 | PLAUR | CLIP1 |
| TYMS | SIVA1 | TNFRSF6B | CD99 | CLDN7 | HIST1H2AC |
| PTTG1 | FANC1 | PDLIM7 | F3 | DUOXA1 | BNIPL |
| KIAA0101 | ECT2 | CAV1 | PSAP | PDZK1IP1 | QSOX1 |
| GMNN | POLE3 | DKK3 | NMRK1 | NCCRP1 | ECM1 |
| DNAJC9 | WDR34 | COLI7A1 | FKBP3 | IDS | DHRS3 |
| CCNA2 | MCM3 | LTBP1 | DSG2 | PPL | PPP1R15A |
| CKS2 | NCAPG2 | COL5A2 | ECN1 | ZNF750 | TRIM16 |
| MLF1P | TUBB6 | COL1A1 | HTRA1 | EMP1 | AQP3 |
| VRK1 | NCAPD2 | FHL2 | SERINC1 | CLDN1 | IRF6 |
| CENPM | GINS2 | TIMP3 | CALU | CRB3 | CSTA |
| PRC1 | TIMELESS | PLAU | TPST1 | CYB5R1 | RAB25 |
| SFAG5 | RAD51 | LGALS1 | PLOD3 | DSC2 | HOPX |
| TOP2A | CMC2 | PSMD2 | IGFBP3 | S100P | GIPC1 |
| AURKB | OIP5 | CD63 | FRMD6 | GRHL3 | RAB11FIP1 |
| FEN1 | TUB84B | HERPUD1 | CXCL14 | SPINT1 | CSTB |
| TMEM106C | APOBEC3B | TPM1 | SERPINE2 | SDR18C5 | KRT6C |
| RRM1 | ORC6 | SLC38A14 | RABAC1 | SPRR1A | PKP1 |
| RFC4 | C19orf48 | C1S | TMED9 | WBP2 | JUP |
| MCM7 | SNRNP25 | MMP1 | NAGK | GRB7 | MAFF |
| CDKN3 | RFC3 | EXT2 | BMP1 | KLK7 | DSG3 |
| NUDT1 | TROAP | COL4A2 | ESYT1 | TMEM79 | AKTIP |
| PBK | EBP | PRSS23 | STON2 | SBSN | KLF3 |
| MELK | DKC1 | SLC7A6 | TAGLN | PIN1 | HSPB8 |
| ANLN | H2AFV | SLC31A2 | GJA1 | CLIC3 | H1FD |
| Epi dif. 2 | | Stress | | Hypoxia | |
| Genes | Genes | Genes | Genes | Genes | Genes |
| 1-50 | 51-100 | 1-50 | 51-100 | 1-50 | 51-100 |
| LY6D | UBE2L5 | FOS | C1R | NDRG1 | ZFP36L1 |
| KRT16 | WDR74 | ATF3 | PHLDA2 | IGFBP3 | HLA-E |
| KRT6B | PPIF | NR4A1 | DNAJB14 | PTHLH | PIK3IF1 |
| LYPD3 | PRMT6 | DUSP1 | MCL1 | EGLN3 | CLK3 |
| KRT6C | VSNL1 | ZFP36 | HERPUD1 | BNIP3 | POLR1D |
| TYMP | VPS25 | PPP1R15A | ADRB2 | NDUFA4L2 | BTG1 |
| FABP5 | SNRNP40 | SGK1 | EIF4A3 | ERO1L | NPC2 |
| SCO2 | ADRM1 | EGR1 | TAC5TD2 | P4HA1 | LAMP2 |
| FGFBP1 | NDUFS6 | ZC3H12A | ID1 | SLC2A1 | DSG2 |
| JUP | TUBA1C | JUNB | ETS2 | ENOQ | SAT1 |
| IMP4 | TMEM79 | FOSB | CD74 | HK2 | AK4 |
| DSC2 | UOCRF51 | IER2 | TRIB1 | PGF | SMS |
| TMBIM1 | EIF3K | NFKBIA | SLC20A1 | LDHA | FRMD6 |
| KRT14 | NME2 | NFKBIZ | LOC284454 | PGK1 | CLDND1 |
| C1QBP | PKP3 | HBEGF | EIF1 | PDX1 | ACP6 |
| SFN | SERPINB1 | BTG2 | CXCL2 | DHRS3 | AP1G2 |
| S100A14 | RPL26L1 | SOD2 | BRD2 | DDIT4 | TPI1 |
| RAB38 | EIF6 | CDKN1A | RASD1 | PVRL4 | PLAUR |
| GJB5 | DSP | NCOA7 | LDLR | GPNMB | BCL10 |
| MRPL14 | PHLDA2 | JUN | EGR2 | BIK | TMEM59 |
| TRIM29 | S100A16 | NYC | TFRC | GJB6 | HA53 |
| ANXA8L2 | LGAL57 | SERTAD1 | ADM | C4ORF3 | SERINC1 |
| KRT6A | MT1X | CCNL1 | TGIF1 | IGFBP2 | C1orf43 |
| PDHB | UQCRC2 | RND3 | HLA-DRB1 | FAM162A | END1 |
| AKR1B10 | EIF3I | PLK2 | OSR2 | GPI | CSDA |
| LAD1 | MRPL24 | SOCS3 | SAA1 | LPIM3 | PFKP |
| DSG3 | CCT7 | DNAJB1 | ELF3 | PLAL1 | KLHL24 |
| MRPL21 | RHOV | DUSP2 | CLK1 | ADM | HIST1H1C |
| NDUFS7 | ECE2 | TSC22D1 | PER2 | ANGPTL4 | RBP4 |
| PSMD6 | SSBP1 | KLF10 | KLF1 | DARS | BHLHE40 |
| AHCY | PCLDIP2 | GADD45B | GPNMB | NUPR1 | GAPDH |
| GBF2 | FIS1 | PMAIP1 | MXD1 | SERPINE1 | UPK3BL |
| TXN2 | CKMT1A | MAFF | UBC | FGAN1 | LTBP1 |
| PSMD13 | GJB3 | ERRFI1 | HLA-DRA | ALDOA | P4HA2 |
| NOP16 | NME1 | SLC38A2 | SLC3A2 | DAAM1 | HBP1 |
| EIF4EBP1 | MRFS12 | IRF1 | OVOL1 | CXADR | GRHL1 |
| WRPL12 | GPS1 | TOB1 | HIST1H2BK | SEMA4B | DDIT3 |
| HSD17B10 | ALG3 | ID2 | DDX3X | CA9 | ANXA1 |
| LGALS7B | MRPL20 | KLF6 | LAMB3 | CIB1 | ITGA5 |
| THBD | EMC6 | DNAJA1 | ZNF622 | SPRR1B | LOC100862671 |
| EXOSC4 | SRD5A1 | TNFAIP3 | TUBB2A | PLIN2 | PLS3 |
| APRT | PA2G4 | BHLHE40 | ZFAND5 | WSB1 | TSC22D2 |
| ANXABL1 | ECSIT | NXF1 | IRF6 | HILPDA | GLTP |
| ATP5G1 | MRPL23 | FOSL1 | TNF | NOL3 | PLCD2 |
| S10DA2 | NAA20 | IER3 | BTG1 | PFKFB3 | PERF |
| TBRG4 | HMOX2 | DUSP6 | LMNA | IFNGR1 | MALL |
| MAL2 | COA4 | HCAR2 | MAP1LC3B | H1F0 | CTNND1 |
| MHP2L1 | DCXR | IL6 | TSC22D3 | KDM3A | KDM5B |
| DDX3BA | FSMD6 | CYR61 | PLK3 | BCL6 | AHNAK2 |
| ZNF750 | WBSCR22 | EFNA1 | KLHL21 | BNIP3L | PNRC1 |

TABLE S8
Frequencies of fibroblast subpopulations in distinct patients (top) and
matched primary and LN samples (bottom).

| | Myofib. | Resting | CAFs | CAF1 | CAF2 |
|---|---|---|---|---|---|
| MEEI28 | 212 | 47 | 145 | 34 | 111 |
| MEEI25 | 172 | 25 | 65 | 26 | 39 |
| MEEI16 | 99 | 7 | 40 | 35 | 5 |
| MEEI26 | 86 | 12 | 31 | 19 | 12 |
| MEEI6 | 7 | 13 | 68 | 16 | 52 |
| MEEI5 | 27 | 2 | 39 | 34 | 5 |
| MEEI17 | 5 | 7 | 31 | 31 | 0 |
| MEEI18 | 14 | 9 | 25 | 8 | 17 |
| MEEI22 | 46 | 3 | 5 | 4 | 1 |
| MEEI10 | 5 | 2 | 23 | 23 | 0 |
| MEEI8 | 2 | 0 | 24 | 17 | 7 |
| MEEI24 | 24 | 4 | 8 | 8 | 0 |
| MEEI20 | 10 | 0 | 4 | 4 | 0 |
| MEEI13 | 5 | 0 | 5 | 4 | 1 |
| MEEI12 | 1 | 1 | 1 | 1 | 0 |
| MEEI7 | 0 | 0 | 1 | 1 | 0 |
| MEEI9 | 0 | 0 | 0 | 0 | 0 |
| MEEI23 | 0 | 0 | 0 | 0 | 0 |

| | | Myofib. | Resting | CAFs | CAF1 | CAF2 |
|---|---|---|---|---|---|---|
| MEEI28 | Pri | 139 | 47 | 111 | 4 | 107 |
| | LN | 73 | 0 | 34 | 30 | 4 |
| MEEI25 | Pri | 82 | 22 | 54 | 17 | 37 |
| | LN | 90 | 3 | 11 | 9 | 2 |
| MEEI26 | Pri | 42 | 4 | 18 | 10 | 8 |
| | LN | 44 | 8 | 13 | 9 | 4 |
| MEEI5 | Pri | 4 | 2 | 35 | 30 | 5 |
| | LN | 23 | 0 | 4 | 4 | 0 |
| MEEI10 | Pri | 1 | 2 | 23 | 23 | 0 |
| | LN | 4 | 0 | 0 | 0 | 0 |
| MEEI20 | Pri | 10 | 0 | 3 | 3 | 0 |
| | LN | 0 | 0 | 1 | 1 | 0 |
| Total | Pri | 278 | 77 | 244 | 87 | 157 |
| | LN | 234 | 11 | 63 | 53 | 10 |

Myofib = myofibroblasts;
CAF = cancer-associated fibroblast

Key Resources Table

| | | |
|---|---|---|
| REAGENT or RESOURCE | SOURCE | IDENTIFIER |

Antibodiess

| | | |
|---|---|---|
| Monoclonal mouse CD45-vioblue, clone 5B1 | Miltenyi Blotec | Cat#130-092-880, |
| | | RRID:AB_1103220 |
| Monoclonal mouse CD90-PE, clone 5E10, lot | BD Biosciences | Cat#555596, |
| #4343763 | | RRID:AB_395970 |
| Monoclonal mouse CD31-PE-cy7, clone WM59, lot | BD Biosciences | Cat#563651 |
| #4357750 | | |
| Monoclonal mouse CD3-PE-cy7, clone UCHT1, lot | ThermoFisher | Cat#25-0038-42 |
| #E09903-1631 | | |
| Calcein AM | ThermoFisher | Cat#C3100MP |
| TO-PRO-3 iodide | ThermoFisher | Cat#T3605 |
| Monoclonal mouse p63, clone 4A4, lot #031915, | Biocare Medical | Cat#CM 163 A/B, |
| 040416 | | RRID:AB_10582730 |
| Monoclonal mouse LAMC2, clone CL2980, lot | Novus Biologicals | Cat#NBP2-42388 |
| #CL2980 | | |
| Polyclonal rabbit Beta Ig-h3/TGFBI, lot | Novus Biologicals | Cat#NBP1-60049, |
| #QC14319-41943 | | RRID:AB_11005227 |
| Polyclonal rabbit CLDN4, lot #AA43131 | Novus Biologicals | Cat#NB100-91712, |
| | | RRID:AB_1216500 |
| Monoclonal mouse MMP-10, clone 110304, lot | R&D Systems | Cat#MAB910, |
| #DRA0215031 | | RRID:AB_2144566 |
| Polyclonal goat p63, lot #KFX0115111 | R&D Systems | Cat#AF1916, |
| | | RRID:AB_2207174 |
| Polyclonal sheep PDPN, lot #XXO0115071 | R&D Systems | Cat#AF3670, |
| | | RRID:AB_2162070 |
| Polyclonal rabbit LAMB3, lot #A74251 | Sigma-Aldrich | Cat#HPA008069, |
| | | RRID:AB_1079228 |
| Polyclonal rabbit ITGA5, lot #B74062 | Sigma-Aldrich | Cat#HPA002642, |
| | | RRID:AB_1078469 |
| Polyclonal rabbit SPRR1B, lot #SA100223AI | Sigma-Aldrich | Cat#SAB1301567 |
| Polyclonal rabbit FAP, lot #R84355 | Sigma-Aldrich | Cat#HPA059739 |
| Monoclonal mouse CXADR-PE clone RmcB, lot | EMD Millipore | Cat#FCMAB418PE, |
| #2766468 | | RRID:AB_10807695 |
| Polyclonal rabbit TGFBI, lot #75709 | LifeSpan Biosciences | Cat#LS-C325695 |
| Monoclonal mouse p16, clone E6H2 | Roche Tissue Diagnostics | Cat#725-4713 |
| RNAscope Probe HPV-HR18 | Advanced Cell Diagnostics | Cat#312591 |
| R-PE Rabbit IgG Labeling Kit | ThermoFisher | Cat#Z25355 |

Bacterial and Virus Strains
Biological Samples
See Table S1 for a list of patients included in the study.
Chemicals, Peptides, and Recombinant Proteins

| | | |
|---|---|---|
| A-83-01 | Tocris Bioscience | Cat#2939 |
| DMH-1 | Tocris Bioscience | Cat#4126 |
| CHIR99021 | Tocris Bioscience | Cat#4423 |
| Y-27632 | Selleck Chemicals | Cat#S1049 |
| Recombinant TGFβ1 | R&D Systems | Cat#240-B-010 |
| Recombinant TGFβ3 | R&D Systems | Cat#243-B3-010 |

Critical Commercial Assays

| | | |
|---|---|---|
| Human Tumor Dissociation Kit | Miltenyi Biotec | Cat#130-095-929 |
| CellTiter-Glo | Promega | Cat#G7572 |
| BioCoat Matrigel Invasion Chambers | Corning | Cat#354480 |
| RNeasy Micro Kit | Qiagen | Cat#74004 |
| QIAamp DNA Blood Mini Kit | Qiagen | Cat#51106 |
| pENTR/D-TOPO Cloning Kt | ThermoFisher | Cat#K240020 |
| Gateway LR clonase Enzyme Mix | ThermoFsher | Cat#11791019 |
| FuGENE HD Transfection Reagent | Promega | Cat#E2312 |
| PCR Supermix | ThemoFisher | Cat#10572014 |

Deposited Data

| | |
|---|---|
| Raw and analyzed data | This paper |

Experimental Models: Cell Lines

| | | |
|---|---|---|
| Cal27 | Ohio State University, | RRID:CVCL_1107 |
| | James Rocco Lab | |
| SCC9 | Ohio State University, | RRID:CVCL_1685 |
| | James Rocco Lab | |
| SCC4 | Ohio State University, | RRID:CVCL_1684 |
| | James Rocco Lab | |
| SCC25 | Ohio State University, | RRID:CVCL_1682 |
| | James Rocco Lab | |
| JHU-006 | Ohio State University, | RRID:CVCL_5985 |
| | James Rocco Lab | |
| HEK293T | MGH, Bradley Bernstein | RRID:CVCL_0063 |
| | Lab | |

Experimental Models: Organisms/Strains
Oligonucleotides

| | | |
|---|---|---|
| TGFBI forward: 5′-CAC CAT GGC GCT CTT CGT GCG | IDT | Ref#150615285 |
| G-3′ | | |
| TGFBI reverse: 5′-CTA ATG CTT CAT CCT CTC-3′ | IDT | Ref#150615286 |
| TGFBI sgRNA1 forward: 5′-CAC CGA GCT GGT AGG | IDT | Ref#150619894 |
| GCG ACT TGG C-3′ | | |
| TGFBI sgRNA1 reverse: 5′-AAA CGC CAA GTC GCC | IDT | Ref#150619895 |
| CTA CCA GCT C-3′ | | |
| TGFBI sgRNA2 forward: 5′-CAC CGC GAC TTG GCG | IDT | Ref#150619896 |
| GGA CCC GCC A-3′ | | |
| TGFB1 sgRNA2 reverse: 5′-AAA CTG GCG GGT CCC | IDT | Ref#150619897 |
| GCC AAG TCG C-3′ | | |
| TGIBI sgRNA3 forward: 5′-CAC CGC ATG CTC ACT | IDT | Ref#150619898 |
| ATC AAC GGG A-3′ | | |
| TGFB1 sgRNA3 reverse: 5′-AAA CTC CCG TTG ATA | IDT | Ref#150619899 |
| GTG AGC ATG C-3′ | | |
| TGFBI NG5 forward (sgRNA 1 and 2): 5-TCC ATG | IDT | Ref#160658478 |
| GCG CTC TTC GTG-3′ | | |
| TGFBI NGS reverse (sgRNA 1 and 2): 5′-GAC TAC | IDT | Ref#160658479 |
| CTG ACC TTC CGC AG-3′ | | |
| TGFBI NGS forward (sgRNA3): 5′-GTG GAC CCT GAC | IDT | Ref#160658480 |
| TTG ACC TG-3′ | | |
| TGFBI NGS reverse (sgRNA3): 5′-GTA GTG GAT CAC | IDT | Ref#160658481 |
| CCC GTT GG-3′ | | |

Recombinant DNA

| | | |
|---|---|---|
| pDNR-Dual-TGFBI | Harvard Plasmid | Cat#HsCD00003120 |
| | Consortium | |
| pMAL | MGH, Bradley Bernstein | van Galen et al. |
| | Lab | (2014) |
| pMAL-Luc | MGH, Bradley Bernstein | van Galen et al. |
| | Lab | (2014) |
| pMAX-GFP | MGH, BradLey Bernstein | van Galen et al. |
| | Lab | (2014) |
| lentiCRISPRv2 | Addgene | 52961 |
| Non-targeting control plasmid | Broad Institute | BRDN0001478216 |

Software and Algorithms

| | |
|---|---|
| Flowjo version 10.2 | TreeStar |
| NIS-Elements Advanced Research version 3.10 | Nikon |
| GraphPad Prism version 4.0 | GraphPad Software |
| MatLab version 2014b | MathWorks |

Other
TGFBI oligonucleotides above are identified as SEQ. I.D. Nos. 8-19.

### REFERENCES

Agrawal, N., Frederick, M. J., Pickering, C. R., Bettegowda, C., Chang, K., Li, R. J., Fakhry, C., Xie, T. X., Zhang, J., Wang, J., et al. (2011). Exome sequencing of head and neck squamous cell carcinoma reveals inactivating mutations in NOTCH1. Science 333, 1154-1157.
Bacher, R., Chu, L. F., Leng, N., Gasch, A. P., Thomson, J. A., Stewart, R. M., Newton, M., and Kendziorski, C. (2017). SCnorm: robust normalization of single-cell RNA-seq data. Nat Methods 14, 584-586.
Cancer Genome Atlas, N. (2015). Comprehensive genomic characterization of head and neck squamous cell carcinomas. Nature 517, 576-582.
Cancer Genome Atlas Research, N. (2011). Integrated genomic analyses of ovarian carcinoma. Nature 474, 609-615.
Cibulskis, K., Lawrence, M. S., Carter, S. L., Sivachenko, A., Jaffe, D., Sougnez, C., Gabriel, S., Meyerson, M., Lander, E. S., and Getz, G. (2013). Sensitive detection of somatic point mutations in impure and heterogeneous cancer samples. Nat Biotechnol 31, 213-219.
Clark, A. G., and Vignjevic, D. M. (2015). Modes of cancer cell invasion and the role of the microenvironment. Curr Opin Cell Biol 36, 13-22.
Colella, S., Richards, K. L., Bachinski, L. L., Baggerly, K. A., Tsavachidis, S., Lang, J. C., Schuller, D. E., and Krahe, R. (2008). Molecular signatures of metastasis in head and neck cancer. Head Neck 30, 1273-1283.
DePristo, M. A., Banks, E., Poplin, R., Garimella, K. V., Maguire, J. R., Hartl, C., Philippakis, A. A., del Angel, G., Rivas, M. A., Hanna, M., et al. (2011). A framework for variation discovery and genotyping using next-generation DNA sequencing data. Nat Genet 43, 491-498.
Fisher, S., Barry, A., Abreu, J., Minie, B., Nolan, J., Delorey, T. M., Young, G., Fennell, T. J., Allen, A., Ambrogio, L., et al. (2011). A scalable, fully automated process for construction of sequence-ready human exome targeted capture libraries. Genome Biol 12, R1.
Giustacchini, A., Thongjuea, S., Barkas, N., Woll, P. S., Povinelli, B. J., Booth, C. A. G., Sopp, P., Norfo, R., Rodriguez-Meira, A., Ashley, N., et al. (2017). Single-cell transcriptomics uncovers distinct molecular signatures of stem cells in chronic myeloid leukemia. Nat Med 23, 692-702.
Gupta, G. P., and Massague, J. (2006). Cancer metastasis: building a framework. Cell 127, 679-695.
Hong, T., Watanabe, K., Ta, C. H., Villarreal-Ponce, A., Nie, Q., and Dai, X. (2015). An Ovol2-Zeb1 Mutual Inhibitory Circuit Governs Bidirectional and Multi-step Transition between Epithelial and Mesenchymal States. PLoS Comput Biol 11, e1004569.
Kim, K. T., Lee, H. W., Lee, H. O., Song, H. J., Jeong da, E., Shin, S., Kim, H., Shin, Y., Nam, D. H., Jeong, B. C., et al. (2016). Application of single-cell RNA sequencing in optimizing a combinatorial therapeutic strategy in metastatic renal cell carcinoma. Genome Biol 17, 80.
Lambert, A. W., Pattabiraman, D. R., and Weinberg, R. A. (2017). Emerging Biological Principles of Metastasis. Cell 168, 670-691.
Li, B., and Dewey, C. N. (2011). RSEM: accurate transcript quantification from RNA-Seq data with or without a reference genome. In BMC Bioinformatics (England), p. 323.
Li, H., Courtois, E. T., Sengupta, D., Tan, Y., Chen, K. H., Goh, J. J., Kong, S. L., Chua, C., Hon, L. K., Tan, W. S., et al. (2017). Reference component analysis of single-cell transcriptomes elucidates cellular heterogeneity in human colorectal tumors. Nat Genet.
Lundgren, K., Nordenskj old, B., and Landberg, G. (2009). Hypoxia, Snail and incomplete epithelial-mesenchymal transition in breast cancer. Br J Cancer 101, 1769-1781.
Madar, S., Goldstein, I., and Rotter, V. (2013). ‘Cancer associated fibroblasts’—more than meets the eye. Trends Mol Med 19, 447-453.
McKenna, A., Hanna, M., Banks, E., Sivachenko, A., Cibulskis, K., Kernytsky, A., Garimella, K., Altshuler, D., Gabriel, S., Daly, M., et al. (2010). The Genome Analysis Toolkit: a MapReduce framework for analyzing next-generation DNA sequencing data. Genome Res 20, 1297-1303.
Meacham, C. E., and Morrison, S. J. (2013). Tumour heterogeneity and cancer cell plasticity. Nature 501, 328-337.
Mellman, I., Coukos, G., and Dranoff, G. (2011). Cancer immunotherapy comes of age. Nature 480, 480-489.
Monroe, M. M., and Gross, N. D. (2012). Evidence-based practice: management of the clinical node-negative neck in early-stage oral cavity squamous cell carcinoma. Otolaryngol Clin North Am 45, 1181-1193.
Moustakas, A., and Heldin, C. H. (2016). Mechanisms of TGFbeta-Induced Epithelial-Mesenchymal Transition. J Clin Med 5.
Muller, S., Liu, S. J., Di Lullo, E., Malatesta, M., Pollen, A. A., Nowakowski, T. J., Kohanbash, G., Aghi, M., Kriegstein, A. R., Lim, D. A., et al. (2016). Single-cell sequencing maps gene expression to mutational phylogenies in PDGF- and EGF-driven gliomas. Mol Syst Biol 12, 889.
Navin, N. E. (2015). The first five years of single-cell cancer genomics and beyond. Genome Res 25, 1499-1507.
Nieto, M. A., Huang, R. Y., Jackson, R. A., and Thiery, J. P. (2016). Emt: 2016. Cell 166, 21-45.
Patel, A. P., Tirosh, I., Trombetta, J. J., Shalek, A. K., Gillespie, S. M., Wakimoto, H., Cahill, D. P., Nahed, B. V., Curry, W. T., Martuza, R. L., et al. (2014). Single-cell RNA-seq highlights intratumoral heterogeneity in primary glioblastoma. Science 344, 1396-1401.
Picelli, S., Faridani, O. R., Bjorklund, A. K., Winberg, G., Sagasser, S., and Sandberg, R. (2014). Full-length RNA-seq from single cells using Smart-seq2. Nat Protoc 9, 171-181.
Pinello, L., Canver, M. C., Hoban, M. D., Orkin, S. H., Kohn, D. B., Bauer, D. E., and Yuan, G. C. (2016). Analyzing CRISPR genome-editing experiments with CRISPResso. Nat Biotechnol 34, 695-697.
Puram, S. V., and Rocco, J. W. (2015). Molecular Aspects of Head and Neck Cancer Therapy. Hematol Oncol Clin North Am 29, 971-992.
Puram, S. V., Yeung, C. M., Jahani-Asl, A., Lin, C., de la Iglesia, N., Konopka, G., Jackson-Grusby, L., and Bonni, A. (2012). STAT3-iNOS Signaling Mediates EGFRvIII-Induced Glial Proliferation and Transformation. J Neurosci 32, 7806-7818.
Ramilowski, J. A., Goldberg, T., Harshbarger, J., Kloppmann, E., Lizio, M., Satagopam, V. P., Itoh, M., Kawaji, H., Carninci, P., Rost, B., et al. (2015). A draft network of ligand-receptor-mediated multicellular signalling in human. Nat Commun 6, 7866.
Ranieri, D., Rosato, B., Nanni, M., Magenta, A., Belleudi, F., and Torrisi, M. R. (2016). Expression of the FGFR2 mesenchymal splicing variant in epithelial cells drives epithelial-mesenchymal transition. Oncotarget 7, 5440-5460.
Rockey, D. C., Weymouth, N., and Shi, Z. (2013). Smooth muscle alpha actin (Acta2) and myofibroblast function during hepatic wound healing. PLoS One 8, e77166.
Roepman, P., de Jager, A., Groot Koerkamp, M. J., Kummer, J. A., Slootweg, P. J., and Holstege, F. C. (2006). Maintenance of head and neck tumor gene expression profiles upon lymph node metastasis. Cancer Res 66, 11110-11114.
Savagner, P., Kusewitt, D. F., Carver, E. A., Magnino, F., Choi, C., Gridley, T., and Hudson, L. G. (2005). Developmental transcription factor slug is required for effective re-epithelialization by adult keratinocytes. J Cell Physiol 202, 858-866.
Stransky, N., Egloff, A. M., Tward, A. D., Kostic, A. D., Cibulskis, K., Sivachenko, A., Kryukov, G. V., Lawrence, M. S., Sougnez, C., McKenna, A., et al. (2011). The mutational landscape of head and neck squamous cell carcinoma. Science 333, 1157-1160.
Stratton, M. R., Campbell, P. J., and Futreal, P. A. (2009). The cancer genome. Nature 458, 719-724.
Tan, T. Z., Miow, Q. H., Miki, Y., Noda, T., Mori, S., Huang, R. Y., and Thiery, J. P. (2014). Epithelial-mesenchymal transition spectrum quantification and its efficacy in deciphering survival and drug responses of cancer patients. EMBO Mol Med 6, 1279-1293.
Tanay, A., and Regev, A. (2017). Scaling single-cell genomics from phenomenology to mechanism. Nature 541, 331-338.
Thiery, J. P., Acloque, H., Huang, R. Y., and Nieto, M. A. (2009). Epithelial-mesenchymal transitions in development and disease. Cell 139, 871-890.
Ting, D. T., Wittner, B. S., Ligorio, M., Vincent Jordan, N., Shah, A. M., Miyamoto, D. T., Aceto, N., Bersani, F., Brannigan, B. W., Xega, K., et al. (2014). Single-cell RNA sequencing identifies extracellular matrix gene expression by pancreatic circulating tumor cells. Cell Rep 8, 1905-1918.
Tirosh, I., Izar, B., Prakadan, S. M., Wadsworth, M. H., 2nd, Treacy, D., Trombetta, J. J., Rotem, A., Rodman, C., Lian, C., Murphy, G., et al. (2016a). Dissecting the multicellular ecosystem of metastatic melanoma by single-cell RNA-seq. Science 352, 189-196.
Tirosh, I., Venteicher, A. S., Hebert, C., Escalante, L. E., Patel, A. P., Yizhak, K., Fisher, J. M., Rodman, C., Mount, C., Filbin, M. G., et al. (2016b). Single-cell RNA-seq supports a developmental hierarchy in human oligodendroglioma. Nature 539, 309-313.
Van der Auwera, G. A., Carneiro, M. O., Hartl, C., Poplin, R., Del Angel, G., Levy-Moonshine, A., Jordan, T., Shakir, K., Roazen, D., Thibault, J., et al. (2013). From FastQ data to high confidence variant calls: the Genome Analysis Toolkit best practices pipeline. Curr Protoc Bioinformatics 43, 11 10 11-33.
van Dijk, D., Nainys, J., Sharma, R., Kathail, P., Carr, A. J., Moon, K. R., Mazutis, L., Wolf, G., Krishnaswamy, S., and Pe'er, D. (Pre-print, 2017). MAGIC: A diffusion-based imputation method reveals gene-gene interactions in single-cell RNA-sequencing data. BioRxiv.
van Galen, P., Kreso, A., Mbong, N., Kent, D. G., Fitzmaurice, T., Chambers, J. E., Xie, S., Laurenti, E., Hermans, K., Eppert, K., et al. (2014). The unfolded protein response governs integrity of the haematopoietic stem-cell pool during stress. Nature 510, 268-272.
Venteicher, A. S., Tirosh, I., Hebert, C., Yizhak, K., C., N., Filbin, M. G., Hoverstadt, V., Escalante, L. E., Saw, M. L., Rodman, C., et al. (2017). Decoupling genetics, lineages and tumor micro-environment in IDH-mutant gliomas by single-cell RNA-seq. Science 355.
Verhaak, R. G., Hoadley, K. A., Purdom, E., Wang, V., Qi, Y., Wilkerson, M. D., Miller, C. R., Ding, L., Golub, T., Mesirov, J. P., et al. (2010). Integrated genomic analysis identifies clinically relevant subtypes of glioblastoma characterized by abnormalities in PDGFRA, IDH1, EGFR, and NF1. Cancer Cell 17, 98-110.
Wagner, A., Regev, A., and Yosef, N. (2016). Revealing the vectors of cellular identity with single-cell genomics. Nat Biotechnol 34, 1145-1160.
Weinberg, R. A. (2014). Coming full circle-from endless complexity to simplicity and back again. Cell 157, 267-271.
Wilm, A., Aw, P. P., Bertrand, D., Yeo, G. H., Ong, S. H., Wong, C. H., Khor, C. C., Petric, R., Hibberd, M. L., and Nagarajan, N. (2012). LoFreq: a sequence-quality aware, ultra-sensitive variant caller for uncovering cell-population heterogeneity from high-throughput sequencing datasets. Nucleic Acids Res 40, 11189-11201.
Yao, C., Li, P., Song, H., Song, F., Qu, Y., Ma, X., Shi, R., and Wu, J. (2016). CXCL12/CXCR4 Axis Upregulates Twist to Induce EMT in Human Glioblastoma. Mol Neurobiol 53, 3948-3953.
Ye, X., and Weinberg, R. A. (2015). Epithelial-Mesenchymal Plasticity: A Central Regulator of Cancer Progression. Trends Cell Biol 25, 675-686.
Yu, M., Bardia, A., Wittner, B. S., Stott, S. L., Smas, M. E., Ting, D. T., Isakoff, S. J., Ciciliano, J. C., Wells, M. N., Shah, A. M., et al. (2013). Circulating breast tumor cells exhibit dynamic changes in epithelial and mesenchymal composition. Science 339, 580-584.
Zheng, Z., Liebers, M., Zhelyazkova, B., Cao, Y., Panditi, D., Lynch, K. D., Chen, J., Robinson, H. E., Shim, H. S., Chmielecki, J., et al. (2014). Anchored multiplex PCR for targeted next-generation sequencing. Nat Med 20, 1479-1484.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of detecting an EMT-like (p-EMT) gene signature in epithelial tumors comprising, detecting in tumor cells obtained from a subject suffering from an epithelial tumor, the expression or activity of a EMT-like (p-EMT) gene signature, said signature comprising one or more genes or polypeptides selected from the group consisting of SERPINE1, TGFBI, MMP10, LAMC2, P4HA2, PDPN, ITGA5, LAMA3, CDH13, TNC, MMP2, EMP3, INHBA, LAMB3, SNAIL2 and VIM, preferably, wherein said signature does not comprise ZEB1/2, TWIST1/2, or SNAIL1.

2. (canceled)

3. The method according to claim 1, wherein detecting a p-EMT gene signature indicates that the subject is less likely to respond to therapy, and/or wherein detecting a p-EMT gene signature indicates that the subject requires more aggressive treatment.

4. (canceled)

5. The method according to claim 1, further comprising treating the subject with one or more of lymph node dissection, adjuvant chemotherapy, adjuvant radiation, neoadjuvant therapy, chemoradiation and an agent that inhibits TGF beta signaling upon detecting the p-EMT gene signature.

6. The method according to claim 1, wherein the epithelial tumor is head and neck squamous cell carcinoma (HNSCC).

7. The method of claim 1, further comprising treating the method of treatment for a subject in need thereof suffering from an epithelial tumor, said method comprising:
a) detecting expression or activity of a p-EMT gene signature for a tumor sample obtained from the subject, wherein the p-EMT signature comprises one or more genes or polypeptides selected from the group consisting of SERPINE1, TGFBI, MMP10, LAMC2, P4HA2, PDPN, ITGA5, LAMA3, CDH13, TNC, MMP2, EMP3, INHBA, LAMB3, SNAIL2 and VIM; and
b) treating the subject, wherein if a p-EMT signature is detected above a p-EMT high reference level the treatment comprises:
i) lymph node dissection of the subject;
ii) adjuvant chemotherapy;
iii) adjuvant radiation or postoperative radiation treatment (PORT);
iv) neoadjuvant therapy;
v) chemoradiation; or
vi) administering an agent that inhibits TGF beta signaling,
wherein if a p-EMT signature is not detected the treatment comprises delaying lymph node dissection.

8. The method according to claim 7, further comprising:
c) detecting expression or activity of an epithelial gene signature for a tumor sample obtained from the subject, wherein the epithelial signature comprises: one or more genes or polypeptides selected from the group consisting of IL1RN, SLPI, CLDN4, CLDN7, S100A9, SPRR1B, PVRL4, RHCG, SDCBP2, S100A8, APOBEC3A, LY6D, KRT16, KRT6B, KRT6A, LYPD3, KRT6C, KLK10, KLK11, TYMP, FABP5, SCO2, FGFBP1 and JUP, or one or more genes or polypeptides selected from the group consisting of SPRR1B, KRT16, KRT6B, KRT6C, KRT6A, KLK10, KLK11 and CLDN7, and
d) treating the subject as in (b) if a p-EMT signature is detected above a p-EMT high reference level and the epithelial signature is detected below an epithelial low reference.

9. The method according to claim 7, wherein chemoradiation comprises cisplatin.

10. The method according to claim 7, wherein treatment comprises administering an agent that inhibits TGF beta signaling.

11. The method according to claim 7, wherein the epithelial tumor is head and neck squamous cell carcinoma (HNSCC).

12. A method of treating an epithelial tumor, comprising administering to a subject in need thereof a therapeutically effective amount of an agent:
a) capable of reducing the expression or inhibiting the activity of one or more p-EMT signature genes or polypeptides; or
b) capable of targeting or binding to one or more cell surface exposed p-EMT signature genes or polypeptides,
wherein the p-EMT signature comprises one or more genes or polypeptides selected from the group consisting of SERPINE1, TGFBI, MMP10, LAMC2, P4HA2, PDPN, ITGA5, LAMA3, CDH13, TNC, MMP2, EMP3, INHBA, LAMB3, SNAIL2 and VIM.

13. The method according to claim 12, wherein the epithelial tumor comprises HNSCC.

14. The method according to claim 12, wherein said agent capable of reducing the expression or inhibiting the activity of one or more p-EMT signature genes or polypeptides comprises a therapeutic antibody, antibody fragment, antibody-like protein scaffold, aptamer, genetic modifying agent or small molecule; or
wherein said agent capable of targeting or binding to one or more cell surface exposed EMT-like signature polypeptides comprises a CAR T cell capable of targeting or binding to one or more cell surface exposed p-EMT signature genes or polypeptides.

15. (canceled)

16. A method of deconvoluting bulk gene expression data obtained from an epithelial tumor, wherein the tumor comprises both malignant and non-malignant cells, said method comprising:
a) defining, by a processor, the relative frequency of a set of cell types in the tumor from the bulk gene expression data,
wherein the frequency of the cell types is determined by cell type specific gene expression, and
wherein the set of cell types comprises one or more cell types selected from the group consisting of T cells, fibroblasts, macrophages, mast cells, B/plasma cells, endothelial cells, myocytes and dendritic cells; and
b) defining, by a processor, a linear relationship between the frequency of the non-malignant cell types and the expression of a set of genes,
wherein the set of genes comprises genes highly expressed by malignant cells and at most two non-malignant cell types,
wherein the set of genes are derived from gene expression analysis of single cells in at least one epithelial tumor, and
wherein the residual of the linear relationship defines the malignant cell-specific (MCS) expression profile.

17. The method according to claim 16, wherein the epithelial tumor is HNSCC.

18. The method according to claim 16, further comprising assigning genes to a specific malignant cell sub-type, preferably, wherein the malignant cell sub-type is a EMT-like subtype; and/or
wherein the method further comprises determining a p-EMT score, wherein said score is based on expression of a p-EMT signature for the malignant cell-specific (MCS) expression profile,
wherein said p-EMT signature comprises one or more genes or polypeptides selected from the group consisting of SERPINE1, TGFBI, MMP10, LAMC2, P4HA2, PDPN, ITGA5, LAMA3, CDH13, TNC, MMP2, EMP3, INHBA, LAMB3, SNAIL2 and VIM, and
wherein a high p-EMT score has higher expression of the p-EMT signature as compared to expression in a reference data set obtained from a subject with a non-invasive epithelial tumor.

19. (canceled)

20. (canceled)

21. The method of claim 18, wherein the method further comprises treating a subject in need thereof suffering from an epithelial tumor, said method comprising:
a) determining a p-EMT score for a tumor sample obtained from the subject; and
b) treating the subject, wherein if a high p-EMT score is determined the treatment comprises:
i) lymph node dissection of the subject;
ii) adjuvant chemotherapy;
iii) adjuvant radiation or postoperative radiation treatment (PORT);
iv) neoadjuvant therapy;
v) chemoradiation; or
vi) administering an agent that inhibits TGF beta signaling,
wherein if the subject does not have a high p-EMT score the treatment comprises delaying lymph node dissection.

22. The method according to claim 21, wherein chemoradiation comprises cisplatin.

23. The method according to claim 21, wherein treatment comprises administering an agent that inhibits TGF beta signaling.

24. (canceled)

25. (canceled)

26. (canceled)

27. (canceled)

28. (canceled)

29. (canceled)

30. A method of detecting an epithelial gene signature in epithelial tumors comprising detecting in tumor cells obtained from a subject suffering from an epithelial tumor, the expression or activity of an epithelial gene signature, said signature comprising:
a) one or more genes or polypeptides selected from the group consisting of IL1RN, SLPI, CLDN4, CLDN7, S100A9, SPRR1B, PVRL4, RHCG, SDCBP2, S100A8, APOBEC3A, LY6D, KRT16, KRT6B, KRT6A, LYPD3, KRT6C, KLK10, KLK11, TYMP, FABP5, SCO2, FGFBP1 and JUP; or
b) one or more genes or polypeptides selected from the group consisting of SPRR1B, KRT16, KRT6B, KRT6C, KRT6A, KLK10, KLK11 and CLDN7, preferably,
wherein detecting an epithelial gene signature indicates that the subject is more likely to respond to therapy; and/or
wherein detecting an epithelial gene signature indicates that the subject does not require more aggressive treatment.

31. (canceled)

32. (canceled)

33. The method according to claim 30, wherein the epithelial tumor is head and neck squamous cell carcinoma (HNSCC).

34. A method for characterizing epithelial tumor composition comprising: detecting the presence of one or more expression programs in a sample, wherein each expression program comprises a set of biomarkers as defined in Table S7.

35. A kit comprising reagents to detect at least one gene or gene expression program as defined in claim 34, preferably,
wherein the gene expression program is a p-EMT program, wherein the p-EMT program comprises one or more genes or polypeptides selected from the group consisting of SERPINE1, TGFBI, MMP10, LAMC2, P4HA2, PDPN, ITGA5, LAMA3, CDH13, TNC, MMP2, EMP3, INHBA, LAMB3, SNAIL2 and VIM; and/or
wherein the kit comprises antibodies and reagents for immunohistochemistry, preferably, an HNSCC specific antibody; and/or
wherein the kit comprises primers and/or probes for quantitative RT-PCR, PCR, and/or sequencing; and/or
wherein the kit comprises fluorescently bar-coded oligonucleotide probes for hybridization to RNA.
